SEQUENCE LISTING (602 sequences)

SEQ ID NO: 1 - protein, 10 aa, Homo sapiens
FLPWHRLFLL

SEQ ID NO: 2 - protein, 529 aa, Homo sapiens [sequence omitted]

SEQ ID NO: 3 - protein, 188 aa, Homo sapiens
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGPKRGEHAWTHRLRERKQLVIYEEISDPEEDDE

SEQ ID NO: 4 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 5 - DNA, 1964 bp, Homo sapiens
atcactgtagtagtagctggaaagagaaatctgtgactccaattagccagttcctgcagaccttgtgaggactagaggaagaatgctcctggctgttttgtactgcctgctgtggagtttccagacctccgctggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtcatggctccttggggcggcgatggtaggggccgtcctcactgccctgctggcagggcttgtgagcttgctgtgtcgtcacaagagaaagcagcttcctgaagaaaagcagccactcctcatggagaaagaggattaccacagcttgtatcagagccatttataaaaggcttaggcaatagagtagggccaaaaagcctgacctcactctaactcaaagtaatgtccaggttcccagagaatatctgctggtatttttctgtaaagaccatttgcaaaattgtaacctaatacaaagtgtagccttcttccaactcaggtagaacacacctgtctttgtcttgctgttttcactcagcccttttaacattttcccctaagcccatatgtctaaggaaaggatgctatttggtaatgaggaactgttatttgtatgtgaattaaagtgctcttatttt

SEQ ID NO: 6 - DNA, 766 bp, Homo sapiens
ctctctttcgattcttccatactcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggacccaaaaggggggaacatgcctggacccacagactgcgtgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtg

SEQ ID NO: 7 - DNA, 2653 bp, Homo sapiens
ctcaaaaggggccggatttccttctcctggaggcagatgttgcctctctctctcgctcggattggttcagtgcactctagaaacactgctgtggtggagaaactggaccccaggtctggagcgaattccagcctgcagggctgataagcgaggcattagtgagattgagagagactttaccccgccgtggtggttggagggcgcgcagtagagcagcagcacaggcgcgggtcccgggaggccggctctgctcgcgccgagatgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaagaggattctttagagaatccgtattgaatttgtgtggtatgtcactcagaaagaatcgtaatgggtatattgataaattttaaaattggtatatttgaaataaagttgaatattatatataaaaaaaaaaaaaaaaaaa

SEQ ID NO: 8 - protein, 9 aa, Homo sapiens
FLPWHRLFL

SEQ ID NO: 9 - protein, 9 aa, Homo sapiens
LPWHRLFLL

SEQ ID NO: 10 - protein, 38 aa, Homo sapiens
YFSKEEWEKMKASEKIFYVYMKRKYEAMTKLGFKATLP

SEQ ID NO: 11 - protein, 9 aa, Homo sapiens
FSKEEWEKM

SEQ ID NO: 12 - protein, 9 aa, Homo sapiens
KMKASEKIF

SEQ ID NO: 13 - protein, 9 aa, Homo sapiens
MKASEKIFY

SEQ ID NO: 14 - protein, 10 aa, Homo sapiens
KMKASEKIFY

SEQ ID NO: 15 - protein, 9 aa, Homo sapiens
KASEKIFYV

SEQ ID NO: 16 - protein, 10 aa, Homo sapiens
MKASEKIFYV

SEQ ID NO: 17 - protein, 10 aa, Homo sapiens
KASEKIFYVY

SEQ ID NO: 18 - protein, 9 aa, Homo sapiens
ASEKIFYVY

SEQ ID NO: 19 - protein, 9 aa, Homo sapiens
RKYEAMTKL

SEQ ID NO: 20 - protein, 10 aa, Homo sapiens
KRKYEAMTKL

SEQ ID NO: 21 - protein, 10 aa, Homo sapiens
KYEAMTKLGF

SEQ ID NO: 22 - protein, 9 aa, Homo sapiens
YEAMTKLGF

SEQ ID NO: 23 - protein, 8 aa, Homo sapiens
EAMTKLGF

SEQ ID NO: 24 - protein, 10 aa, Homo sapiens
FLPSDYFPSV

SEQ ID NO: 25 - protein, 9 aa, Homo sapiens
AEMGKYSFY

SEQ ID NO: 26 - protein, 9 aa, Homo sapiens
KYSEKISYV

SEQ ID NO: 27 - protein, 9 aa, Homo sapiens
KVSEKIVYV

SEQ ID NO: 28 - protein, 9 aa, Homo sapiens
KSSEKIVYV

SEQ ID NO: 29 - protein, 9 aa, Homo sapiens
KASEKIIYV

SEQ ID NO: 30 - protein, 30 aa, Homo sapiens
AFSPQGMPEGDLVYVNYARTEDFFKLERDM

SEQ ID NO: 31 - protein, 23 aa, Homo sapiens
GMPEGDLVYVNYARTEDFFKLER

SEQ ID NO: 32 - protein, 9 aa, Homo sapiens
MPEGDLVYV

SEQ ID NO: 33 - protein, 10 aa, Homo sapiens
GMPEGDLVYV

SEQ ID NO: 34 - protein, 9 aa, Homo sapiens
GMPEGDLVY

SEQ ID NO: 35 - protein, 10 aa, Homo sapiens
QGMPEGDLVY

SEQ ID NO: 36 - protein, 8 aa, Homo sapiens
MPEGDLVY

SEQ ID NO: 37 - protein, 9 aa, Homo sapiens
EGDLVYVNY

SEQ ID NO: 38 - protein, 10 aa, Homo sapiens
PEGDLVYVNY

SEQ ID NO: 39 - protein, 10 aa, Homo sapiens
LVYVNYARTE

SEQ ID NO: 40 - protein, 9 aa, Homo sapiens
VNYARTEDF

SEQ ID NO: 41 - protein, 10 aa, Homo sapiens
YVNYARTEDF

SEQ ID NO: 42 - protein, 9 aa, Homo sapiens
NYARTEDFF

SEQ ID NO: 43 - protein, 8 aa, Homo sapiens
YARTEDFF

SEQ ID NO: 44 - protein, 9 aa, Homo sapiens
RTEDFFKLE

SEQ ID NO: 45 - protein, 30 aa, Homo sapiens
RGIAEAVGLPSIPVHPIGYYDAQKLLEKMG

SEQ ID NO: 46 - protein, 25 aa, Homo sapiens
IAEAVGLPSIPVHPIGYYDAQKLLE

SEQ ID NO: 47 - protein, 9 aa, Homo sapiens
LPSIPVHPI

SEQ ID NO: 48 - protein, 10 aa, Homo sapiens
GLPSIPVHPI

SEQ ID NO: 49 - protein, 9 aa, Homo sapiens
IGYYDAQKL

SEQ ID NO: 50 - protein, 10 aa, Homo sapiens
PIGYYDAQKL

SEQ ID NO: 51 - protein, 9 aa, Homo sapiens
SIPVHPIGY

SEQ ID NO: 52 - protein, 10 aa, Homo sapiens
PSIPVHPIGY

SEQ ID NO: 53 - protein, 8 aa, Homo sapiens
IPVHPIGY

SEQ ID NO: 54 - protein, 9 aa, Homo sapiens
YYDAQKLLE

SEQ ID NO: 55 - protein, 27 aa, Homo sapiens
SSIEGNYTLRVDCTPLMYSLVHLTKEL

SEQ ID NO: 56 - protein, 9 aa, Homo sapiens
IEGNYTLRV

SEQ ID NO: 57 - protein, 10 aa, Homo sapiens
SIEGNYTLRV

SEQ ID NO: 58 - protein, 8 aa, Homo sapiens
EGNYTLRV

SEQ ID NO: 59 - protein, 9 aa, Homo sapiens
TLRVDCTPL

SEQ ID NO: 60 - protein, 10 aa, Homo sapiens
YTLRVDCTPL

SEQ ID NO: 61 - protein, 9 aa, Homo sapiens
LRVDCTPLM

SEQ ID NO: 62 - protein, 9 aa, Homo sapiens
RVDCTPLMY

SEQ ID NO: 63 - protein, 10 aa, Homo sapiens
LRVDCTPLMY

SEQ ID NO: 64 - protein, 35 aa, Homo sapiens
FDKSNPIVLRMMNDQLMFLERAFIDPLGLPDRPFY

SEQ ID NO: 65 - protein, 22 aa, Homo sapiens
VLRMMNDQLMFLERAFIDPLGL

SEQ ID NO: 66 - protein, 9 aa, Homo sapiens
MMNDQLMFL

SEQ ID NO: 67 - protein, 10 aa, Homo sapiens
RMMNDQLMFL

SEQ ID NO: 68 - protein, 9 aa, Homo sapiens
RMMNDQLMF

SEQ ID NO: 69 - protein, 17 aa, Homo sapiens
MLLAVLYCLLWSFQTSA

SEQ ID NO: 70 - protein, 661 aa, Homo sapiens [sequence omitted]

SEQ ID NO: 71 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 72 - protein, 314 aa, Homo sapiens
MPLEQRSQHCKPEEGLEARGEALGLVGAQAPATEEQQTASSSSTLVEVTLGEVPAADSPSPPHSPQGASSFSTTINYTLWRQSDEGSSNQEEEGPRMFPDLESEFQAAISRKMVELVHFLLLKYRAREPVTKAEMLESVLRNCQDFFPVIFSKASEYLQLVFGIEVVEVVPISHLYILVTCLGLSYDGLLGDNQVMPKTGLLIIVLAIIAIEGDCAPEEKIWEELSMLEVFEGREDSVFAHPRKLLMQDLVQENYLEYRQVPGSDPACYEFLWGPRALIETSYVKVLHHTLKIGGEPHISYPPLHERALREGEE

SEQ ID NO: 73 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 74 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 75 - protein, 180 aa, Homo sapiens
MQAEGRGTGGSTGDADGPGGPGIPDGPGGNAGGPGEAGATGGRGPRGAGAARASGPRGGAPRGPHGGAASAQDGRCPCGARRPDSRLLELHITMPFSSPMEAELVRRILSRDAAPLPRPGAVLKDFTVSGNLLFIRLTAADHRQLQLSISSCLQQLSLLMWITQCFLPVFLAQAPSGQRR

SEQ ID NO: 76 - protein, 210 aa, Homo sapiens [sequence omitted]

SEQ ID NO: 77 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 78 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 79 - protein, 123 aa, Homo sapiens
MKAVLLALLMAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 80 - DNA, 2817 bp, Homo sapiens
gtgctaaaaagatgccttcttcatttggctgtgataggtgctttgtggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggtgagggactcccttctcagcctatcatccacacttgtgtttacttctttctacctgatcacctttcttttggccgccccttccaccttaacttctgtgattttctctaatcttcattttcctcttagatcttttctctttcttagcacctagcccccttcaagctctatcataattctttctggcaactcttggcctcaattgtagtcctaccccatggaatgcctcattaggaccccttccctgtccccccatatcacagccttccaaacaccctcagaagtaatcatacttcctgacctcccatctccagtgccgtttcgaagcctgtccctcagtcccctttgaccagtaatctcttcttccttgcttttcattccaaaaatgcttcaggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactggtaagggttcaggaagggcaaggccagttgtagggcaaagagaaggcagggaggcttggatggactgcaaaggagaaaggtgaaatgctgtgcaaacttaaagtagaagggccaggaagacctaggcagagaaatgtgaggcttagtgccagtgaagggccagccagtcagcttggagttggagggtgtggctgtgaaaggagaagctgtggctcaggcctggttctcaccttttctggctccaatcccagaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacctgtggtcactcatacttacctggagcctggcccagtcactgcccaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtaggtccttggacagagactaagtgaggagggaagtggatagaggggacagctggcaagcagcagacatgagtgaagcagtgcctgggattcttctcacaggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtctgagtactctcatatgatgctgtgattttcctggagttgacagaaacacctatatttcccccagtcttccctgggagactactattaactgaaataaa

SEQ ID NO: 81 - DNA, 2420 bp, Homo sapiens
ggatccaggccctgccaggaaaaatataagggccctgcgtgagaacagagggggtcatccactgcatgagagtggggatgtcacagagtccagcccaccctcctggtagcactgagaagccagggctgtgcttgcggtctgcaccctgagggcccgtggattcctcttcctggagctccaggaaccaggcagtgaggccttggtctgagacagtatcctcaggtcacagagcagaggatgcacagggtgtgccagcagtgaatgtttgccctgaatgcacaccaagggccccacctgccacaggacacataggactccacagagtctggcctcacctccctactgtcagtcctgtagaatcgacctctgctggccggctgtaccctgagtaccctctcacttcctccttcaggttttcaggggacaggccaacccagaggacaggattccctggaggccacagaggagcaccaaggagaagatctgtaagtaggcctttgttagagtctccaaggttcagttctcagctgaggcctctcacacactccctctctccccaggcctgtgggtcttcattgcccagctcctgcccacactcctgcctgctgccctgacgagagtcatcatgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctgagcatgagttgcagccaaggccagtgggagggggactgggccagtgcaccttccagggccgcgtccagcagcttcccctgcctcgtgtgacatgaggcccattcttcactctgaagagagcggtcagtgttctcagtagtaggtttctgttctattgggtgacttggagatttatctttgttctcttttggaattgttcaaatgtttttttttaagggatggttgaatgaacttcagcatccaagtttatgaatgacagcagtcacacagttctgtgtatatagtttaagggtaagagtcttgtgttttattcagattgggaaatccattctattttgtgaattgggataataacagcagtggaataagtacttagaaatgtgaaaaatgagcagtaaaatagatgagataaagaactaaagaaattaagagatagtcaattcttgccttatacctcagtctattctgtaaaatttttaaagatatatgcatacctggatttccttggcttctttgagaatgtaagagaaattaaatctgaataaagaattcttcctgttcactggctcttttcttctccatgcactgagcatctgctttttggaaggccctgggttagtagtggagatgctaaggtaagccagactcatacccacccatagggtcgtagagtctaggagctgcagtcacgtaatcgaggtggcaagatgtcctctaaagatgtagggaaaagtgagagaggggtgagggtgtggggctccgggtgagagtggtggagtgtcaatgccctgagctggggcattttgggctttgggaaactgcagttccttctgggggagctgattgtaatgatcttgggtggatcc

SEQ ID NO: 82 - DNA, 4559 bp, Homo sapiens
attccttcatcaaacagccaggagtgaggaagaggaccctcctgagtgaggactgaggatccaccctcaccacatagtgggaccacagaatccagctcagcccctcttgtcagccctggtacacactggcaatgatctcaccccgagcacacccctccccccaatgccacttcgggccgactcagagtcagagacttggtctgaggggagcagacacaatcggcagaggatggcggtccaggctcagtctggcatccaagtcaggaccttgagggatgaccaaaggcccctcccacccccaactcccccgaccccaccaggatctacagcctcaggatccccgtcccaatccctacccctacaccaacaccatcttcatgcttacccccacccccccatccagatccccatccgggcagaatccggttccacccttgccgtgaacccagggaagtcacgggcccggatgtgacgccactgacttgcacattggaggtcagaggacagcgagattctcgccctgagcaacggcctgacgtcggcggagggaagcaggcgcaggctccgtgaggaggcaaggtaagacgccgagggaggactgaggcgggcctcaccccagacagagggcccccaataatccagcgctgcctctgctgccgggcctggaccaccctgcaggggaagacttctcaggctcagtcgccaccacctcaccccgccaccccccgccgctttaaccgcagggaactctggcgtaagagctttgtgtgaccagggcagggctggttagaagtgctcagggcccagactcagccaggaatcaaggtcaggaccccaagaggggactgagggcaacccaccccctaccctcactaccaatcccatcccccaacaccaaccccacccccatccctcaaacaccaaccccacccccaaaccccattcccatctcctcccccaccaccatcctggcagaatccggctttgcccctgcaatcaacccacggaagctccgggaatggcggccaagcacgcggatcctgacgttcacatgtacggctaagggagggaaggggttgggtctcgtgagtatggcctttgggatgcagaggaagggcccaggcctcctggaagacagtggagtccttaggggacccagcatgccaggacagggggcccactgtacccctgtctcaaactgagccaccttttcattcagccgagggaatcctagggatgcagacccacttcagcagggggttggggcccagcctgcgaggagtcaaggggaggaagaagagggaggactgaggggaccttggagtccagatcagtggcaaccttgggctgggggatcctgggcacagtggccgaatgtgccccgtgctcattgcaccttcagggtgacagagagttgagggctgtggtctgagggctgggacttcaggtcagcagagggaggaatcccaggatctgccggacccaaggtgtgcccccttcatgaggactggggatacccccggcccagaaagaagggatgccacagagtctggaagtcccttgttcttagctctgggggaacctgatcagggatggccctaagtgacaatctcatttgtaccacaggcaggaggttggggaaccctcagggagataaggtgttggtgtaaagaggagctgtctgctcatttcagggggttgggggttgagaaagggcagtccctggcaggagtaaagatgagtaacccacaggaggccatcataacgttcaccctagaaccaaaggggtcagccctggacaacgcacgtgggggtaacaggatgtggcccctcctcacttgtctttccagatctcagggagttgatgaccttgttttcagaaggtgactcaggtcaacacaggggccccatctggtcgacagatgcagtggttctaggatctgccaagcatccaggtggagagcctgaggtaggattgagggtacccctgggccagaatgcagcaagggggccccatagaaatctgccctgcccctgcggttacttcagagaccctgggcagggctgtcagctgaagtccctccattatcctgggatctttgatgtcagggaaggggaggccttggtctgaaggggctggagtcaggtcagtagagggagggtctcaggccctgccaggagtggacgtgaggaccaagcggactcgtcacccaggacacctggactccaatgaatttggacatctctcgttgtccttcgcgggaggacctggtcacgtatggccagatgtgggtcccctcatatccttctgtaccatatcagggatgtgagttcttgacatgagagattctcaagccagcaaaagggtgggattaggccctacaaggagaaaggtgagggccctgagtgagcacagaggggaccctccacccaagtagagtggggacctcacggagtctggccaaccctgctgagacttctgggaatccgtggctgtgcttgcagtctgcacactgaaggcccgtgcattcctctcccaggaatcaggagctccaggaaccaggcagtgaggccttggtctgagtcagtgtcctcaggtcacagagcagaggggacgcagacagtgccaacactgaaggtttgcctggaatgcacaccaagggccccacccgcccagaacaaatgggactccagagggcctggcctcaccctccctattctcagtcctgcagcctgagcatgtgctggccggctgtaccctgaggtgccctcccacttcctccttcaggttctgagggggacaggctgacaagtaggacccgaggcactggaggagcattgaaggagaagatctgtaagtaagcctttgtcagagcctccaaggttcagttcagttctcacctaaggcctcacacacgctccttctctccccaggcctgtgggtcttcattgcccagctcctgcccgcactcctgcctgctgccctgaccagagtcatcatgcctcttgagcagaggagtcagcactgcaagcctgaagaaggccttgaggcccgaggagaggccctgggcctggtgggtgcgcaggctcctgctactgaggagcagcagaccgcttcttcctcttctactctagtggaagttaccctgggggaggtgcctgctgccgactcaccgagtcctccccacagtcctcagggagcctccagcttctcgactaccatcaactacactctttggagacaatccgatgagggctccagcaaccaagaagaggaggggccaagaatgtttcccgacctggagtccgagttccaagcagcaatcagtaggaagatggttgagttggttcattttctgctcctcaagtatcgagccagggagccggtcacaaaggcagaaatgctggagagtgtcctcagaaattgccaggacttctttcccgtgatcttcagcaaagcctccgagtacttgcagctggtctttggcatcgaggtggtggaagtggtccccatcagccacttgtacatccttgtcacctgcctgggcctctcctacgatggcctgctgggcgacaatcaggtcatgcccaagacaggcctcctgataatcgtcctggccataatcgcaatagagggcgactgtgcccctgaggagaaaatctgggaggagctgagtatgttggaggtgtttgaggggagggaggacagtgtcttcgcacatcccaggaagctgctcatgcaagatctggtgcaggaaaactacctggagtaccggcaggtgcccggcagtgatcctgcatgctacgagttcctgtggggtccaagggccctcattgaaaccagctatgtgaaagtcctgcaccatacactaaagatcggtggagaacctcacatttcctacccacccctgcatgaacgggctttgagagagggagaagagtgagtctcagcacatgttgcagccagggccagtgggagggggtctgggccagtgcaccttccagggccccatccattagcttccactgcctcgtgtgatatgaggcccattcctgcctctttgaagagagcagtcagcattcttagcagtgagtttctgttctgttggatgactttgagatttatctttctttcctgttggaattgttcaaatgttccttttaacaaatggttggatgaacttcagcatccaagtttatgaatgacagtagtcacacatagtgctgtttatatagtttaggggtaagagtcctgttttttattcagattgggaaatccattccattttgtgagttgtcacataataacagcagtggaatatgtatttgcctatattgtgaacgaattagcagtaaaatacatgatacaaggaactcaaaagatagttaattcttgccttatacctcagtctattatgtaaaattaaaaatatgtgtatgtttttgcttctttgagaatgcaaaagaaattaaatctgaataaattcttcctgttcactggctcatttctttaccattcactcagcatctgctctgtggaaggccctggtagtagtggg

SEQ ID NO: 83 - DNA, 4204 bp, Homo sapiens
acgcaggcagtgatgtcacccagaccacaccccttcccccaatgccacttcagggggtactcagagtcagagacttggtctgaggggagcagaagcaatctgcagaggatggcggtccaggctcagccaggcatcaacttcaggaccctgagggatgaccgaaggccccgcccacccacccccaactcccccgaccccaccaggatctacagcctcaggacccccgtcccaatccttaccccttgccccatcaccatcttcatgcttacctccacccccatccgatccccatccaggcagaatccagttccacccctgcccggaacccagggtagtaccgttgccaggatgtgacgccactgacttgcgcattggaggtcagaagaccgcgagattctcgccctgagcaacgagcgacggcctgacgtcggcggagggaagccggcccaggctcggtgaggaggcaaggtaagacgctgagggaggactgaggcgggcctcacctcagacagagggcctcaaataatccagtgctgcctctgctgccgggcctgggccaccccgcaggggaagacttccaggctgggtcgccactacctcaccccgccgacccccgccgctttagccacggggaactctggggacagagcttaatgtggccagggcagggctggttagaagaggtcagggcccacgctgtggcaggaatcaaggtcaggaccccgagagggaactgagggcagcctaaccaccaccctcaccaccattcccgtcccccaacacccaaccccacccccatcccccattcccatccccacccccacccctatcctggcagaatccgggctttgcccctggtatcaagtcacggaagctccgggaatggcggccaggcacgtgagtcctgaggttcacatctacggctaagggagggaaggggttcggtatcgcgagtatggccgttgggaggcagcgaaagggcccaggcctcctggaagacagtggagtcctgaggggacccagcatgccaggacagggggcccactgtacccctgtctcaaaccgaggcaccttttcattcggctacgggaatcctagggatgcagacccacttcagcagggggttggggcccagccctgcgaggagtcatggggaggaagaagagggaggactgaggggaccttggagtccagatcagtggcaaccttgggctgggggatgctgggcacagtggccaaatgtgctctgtgctcattgcgccttcagggtgaccagagagttgagggctgtggtctgaagagtgggacttcaggtcagcagagggaggaatcccaggatctgcagggcccaaggtgtacccccaaggggcccctatgtggtggacagatgcagtggtcctaggatctgccaagcatccaggtgaagagactgagggaggattgagggtacccctgggacagaatgcggactgggggccccataaaaatctgccctgctcctgctgttacctcagagagcctgggcagggctgtcagctgaggtccctccattatcctaggatcactgatgtcagggaaggggaagccttggtctgagggggctgcactcagggcagtagagggaggctctcagaccctactaggagtggaggtgaggaccaagcagtctcctcacccagggtacatggacttcaataaatttggacatctctcgttgtcctttccgggaggacctgggaatgtatggccagatgtgggtcccctcatgtttttctgtaccatatcaggtatgtgagttcttgacatgagagattctcaggccagcagaagggagggattaggccctataaggagaaaggtgagggccctgagtgagcacagaggggatcctccaccccagtagagtggggacctcacagagtctggccaaccctcctgacagttctgggaatccgtggctgcgtttgctgtctgcacattgggggcccgtggattcctctcccaggaatcaggagctccaggaacaaggcagtgaggacttggtctgaggcagtgtcctcaggtcacagagtagagggggctcagatagtgccaacggtgaaggtttgccttggattcaaaccaagggccccacctgccccagaacacatggactccagagcgcctggcctcaccctcaatactttcagtcctgcagcctcagcatgcgctggccggatgtaccctgaggtgccctctcacttcctccttcaggttctgaggggacaggctgacctggaggaccagaggcccccggaggagcactgaaggagaagatctgtaagtaagcctttgttagagcctccaaggttccattcagtactcagctgaggtctctcacatgctccctctctccccaggccagtgggtctccattgcccagctcctgcccacactcccgcctgttgccctgaccagagtcatcatgcctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagtgagtctgagcacgagttgcagccagggccagtgggagggggtctgggccagtgcaccttccggggccgcatcccttagtttccactgcctcctgtgacgtgaggcccattcttcactctttgaagcgagcagtcagcattcttagtagtgggtttctgttctgttggatgactttgagattattctttgtttcctgttggagttgttcaaatgttccttttaacggatggttgaatgagcgtcagcatccaggtttatgaatgacagtagtcacacatagtgctgtttatatagtttaggagtaagagtcttgttttttactcaaattgggaaatccattccattttgtgaattgtgacataataatagcagtggtaaaagtatttgcttaaaattgtgagcgaattagcaataacatacatgagataactcaagaaatcaaaagatagttgattcttgccttgtacctcaatctattctgtaaaattaaacaaatatgcaaaccaggatttccttgacttctttgagaatgcaagcgaaattaaatctgaataaataattcttcctcttcactggctcgtttcttttccgttcactcagcatctgctctgtgggaggccctgggttagtagtggggatgctaaggtaagccagactcacgcctacccatagggctgtagagcctaggacctgcagtcatataattaaggtggtgagaagtcctgtaagatgtagaggaaatgtaagagaggggtgagggtgtggcgctccgggtgagagtagtggagtgtcagtgc

SEQ ID NO: 84 - DNA, 752 bp, Homo sapiens
atcctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaaactgagctacgaaaaa

SEQ ID NO: 85 - DNA, 2148 bp, Homo sapiens
  misc_feature (1)...(2): n = A,T,C or G
gcttcagggtacagctcccccgcagccagaagccgggcctgcagcccctcagcaccgctccgggacaccccacccgcttcccaggcgtgacctgtcaacagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaactagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttggggataggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcaaaaaaaaaaaaaaaaaa

SEQ ID NO: 86 - DNA, 1466 bp, Homo sapiens
agccccaagcttaccacctgcacccggagagctgtgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 87 - DNA, 990 bp, Homo sapiens
  misc_feature (1)...(990): n = A,T,C or G
agggagaggcagtgaccatgaaggctgtgctgcttgccctgttgatggcaggcttggccctgcagccaggcactgccctgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagcggggcccatgccctgcagccggctgccgccatccttgcgctgctccctgcactcggcctgctgctctggggacccggccagctataggctctggggggccccgctgcagcccacactgggtgtggtgccccaggcctttgtgccactcctcacagaacctggcccagtgggagcctgtcctggttcctgaggcacatcctaacgcaagtttgaccatgtatgtttgcaccccttttccccnaaccctgaccttcccatgggccttttccaggattccnaccnggcagatcagttttagtganacanatccgcntgcagatggcccctccaaccntttntgttgntgtttccatggcccagcattttccacccttaaccctgtgttcaggcacttnttcccccaggaagccttccctgcccaccccatttatgaattgagccaggtttggtccgtggtgtcccccgcacccagcaggggacaggcaatcaggagggcccagtaaaggctgagatgaagtggactgagtagaactggaggacaagagttgacgtgagttcctgggagtttccagagatggggcctggaggcctggaggaaggggccaggcctcacatttgtggggntcccgaatggcagcctgagcacagcgtaggcccttaataaacacctgttggataagccaaaaaa

SEQ ID NO: 88 - protein, 9 aa, Homo sapiens
LPHSSSHWL

SEQ ID NO: 89 - protein, 10 aa, Homo sapiens
QLPHSSSHWL

SEQ ID NO: 90 - protein, 9 aa, Homo sapiens
LIYRRRLMK

SEQ ID NO: 91 - protein, 10 aa, Homo sapiens
SLIYRRRLMK

SEQ ID NO: 92 - protein, 8 aa, Homo sapiens
IYRRRLMK

SEQ ID NO: 93 - protein, 9 aa, Homo sapiens
LPHSSSHWL

SEQ ID NO: 94 - protein, 10 aa, Homo sapiens
QLPHSSSHWL

SEQ ID NO: 95 - protein, 8 aa, Homo sapiens
ESLFRAVI

SEQ ID NO: 96 - protein, 10 aa, Homo sapiens
ILESLFRAVI

SEQ ID NO: 97 - protein, 9 aa, Homo sapiens
ILESLFRAV

SEQ ID NO: 98 - protein, 10 aa, Homo sapiens
CILESLFRAV

SEQ ID NO: 99 - protein, 9 aa, Homo sapiens
CILESLFRA

SEQ ID NO: 100 - protein, 9 aa, Homo sapiens
EFLWGPRAL

SEQ ID NO: 101 - protein, 8 aa, Homo sapiens
FLWGPRAL

SEQ ID NO: 102 - protein, 10 aa, Homo sapiens
FLWGPRALAE

SEQ ID NO: 103 - protein, 10 aa, Homo sapiens
LWGPRALAET

SEQ ID NO: 104 - protein, 9 aa, Homo sapiens
PRALAETSY

SEQ ID NO: 105 - protein, 10 aa, Homo sapiens
GPRALAETSY

SEQ ID NO: 106 - protein, 9 aa, Homo sapiens
RALAETSYV

SEQ ID NO: 107 - protein, 9 aa, Homo sapiens
LAETSYVKV

SEQ ID NO: 108 - protein, 10 aa, Homo sapiens
ALAETSYVKV

SEQ ID NO: 109 - protein, 9 aa, Homo sapiens
AETSYVKVL

SEQ ID NO: 110 - protein, 10 aa, Homo sapiens
LAETSYVKVL

SEQ ID NO: 111 - protein, 9 aa, Homo sapiens
TSYVKVLEY

SEQ ID NO: 112 - protein, 10 aa, Homo sapiens
ETSYVKVLEY

SEQ ID NO: 113 - protein, 9 aa, Homo sapiens
KVLEYVIKV

SEQ ID NO: 114 - protein, 10 aa, Homo sapiens
SYVLVTCLGL

SEQ ID NO: 115 - protein, 9 aa, Homo sapiens
YVLVTCLGL

SEQ ID NO: 116 - protein, 8 aa, Homo sapiens
VLVTCLGL

SEQ ID NO: 117 - protein, 9 aa, Homo sapiens
TQDLVQEKY

SEQ ID NO: 118 - protein, 10 aa, Homo sapiens
LTQDLVQEKY

SEQ ID NO: 119 - protein, 9 aa, Homo sapiens
YGEPRKLLT

SEQ ID NO: 120 - protein, 9 aa, Homo sapiens
LVQEKYLEY

SEQ ID NO: 121 - protein, 10 aa, Homo sapiens
DLVQEKYLEY

SEQ ID NO: 122 - protein, 9 aa, Homo sapiens
SAYGEPRKL

SEQ ID NO: 123 - protein, 9 aa, Homo sapiens
KVLEYVIKV

SEQ ID NO: 124 - protein, 10 aa, Homo sapiens
VKVLEYVIKV

SEQ ID NO: 125 - protein, 9 aa, Homo sapiens
YVKVLEYVI

SEQ ID NO: 126 - protein, 9 aa, Homo sapiens
TSYVKVLEY

SEQ ID NO: 127 - protein, 10 aa, Homo sapiens
ETSYVKVLEY

SEQ ID NO: 128 - protein, 9 aa, Homo sapiens
VIKVSARVR

SEQ ID NO: 129 - protein, 10 aa, Homo sapiens
YVIKVSARVR

SEQ ID NO: 130 - protein, 8 aa, Homo sapiens
ELVHFLLL

SEQ ID NO: 131 - protein, 10 aa, Homo sapiens
MVELVHFLLL

SEQ ID NO: 132 - protein, 8 aa, Homo sapiens
ISRKMVEL

SEQ ID NO: 133 - protein, 9 aa, Homo sapiens
AISRKMVEL

SEQ ID NO: 134 - protein, 10 aa, Homo sapiens
AAISRKMVEL

SEQ ID NO: 135 - protein, 9 aa, Homo sapiens
KMVELVHFL

SEQ ID NO: 136 - protein, 9 aa, Homo sapiens
ISRKMVELV

SEQ ID NO: 137 - protein, 10 aa, Homo sapiens
AISRKMVELV

SEQ ID NO: 138 - protein, 9 aa, Homo sapiens
LVHFLLLKY

SEQ ID NO: 139 - protein, 10 aa, Homo sapiens
ELVHFLLLKY

SEQ ID NO: 140 - protein, 9 aa, Homo sapiens
RKMVELVHF

SEQ ID NO: 141 - protein, 9 aa, Homo sapiens
LQLVFGIEV

SEQ ID NO: 142 - protein, 10 aa, Homo sapiens
YLQLVFGIEV

SEQ ID NO: 143 - protein, 9 aa, Homo sapiens
QLVFGIEVV

SEQ ID NO: 144 - protein, 10 aa, Homo sapiens
LQLVFGIEVV

SEQ ID NO: 145 - protein, 9 aa, Homo sapiens
IEVVEVVPI

SEQ ID NO: 146 - protein, 10 aa, Homo sapiens
GIEVVEVVPI

SEQ ID NO: 147 - protein, 9 aa, Homo sapiens
FGIEVVEVV

SEQ ID NO: 148 - protein, 9 aa, Homo sapiens
ASEYLQLVF

SEQ ID NO: 149 - protein, 10 aa, Homo sapiens
KASEYLQLVF

SEQ ID NO: 150 - protein, 8 aa, Homo sapiens
EEKIWEEL

SEQ ID NO: 151 - protein, 10 aa, Homo sapiens
APEEKIWEEL

SEQ ID NO: 152 - protein, 8 aa, Homo sapiens
APEEKIWE

SEQ ID NO: 153 - protein, 9 aa, Homo sapiens
KIWEELSML

SEQ ID NO: 154 - protein, 10 aa, Homo sapiens
EKIWEELSML

SEQ ID NO: 155 - protein, 8 aa, Homo sapiens
FLWGPRAL

SEQ ID NO: 156 - protein, 9 aa, Homo sapiens
FLWGPRALI

SEQ ID NO: 157 - protein, 9 aa, Homo sapiens
LIETSYVKV

SEQ ID NO: 158 - protein, 10 aa, Homo sapiens
ALIETSYVKV

SEQ ID NO: 159 - protein, 9 aa, Homo sapiens
RALIETSYV

SEQ ID NO: 160 - protein, 9 aa, Homo sapiens
IETSYVKVL

SEQ ID NO: 161 - protein, 10 aa, Homo sapiens
LIETSYVKVL

SEQ ID NO: 162 - protein, 8 aa, Homo sapiens
FLWGPRAL

SEQ ID NO: 163 - protein, 9 aa, Homo sapiens
EFLWGPRAL

SEQ ID NO: 164 - protein, 9 aa, Homo sapiens
FLWGPRALV

SEQ ID NO: 165 - protein, 9 aa, Homo sapiens
RALVETSYV

SEQ ID NO: 166 - protein, 9 aa, Homo sapiens
LWGPRALVE

SEQ ID NO: 167 - protein, 10 aa, Homo sapiens
FLWGPRALVE

SEQ ID NO: 168 - protein, 10 aa, Homo sapiens
LWGPRALVET

SEQ ID NO: 169 - protein, 9 aa, Homo sapiens
GPESRLLEF

SEQ ID NO: 170 - protein, 9 aa, Homo sapiens
PESRLLEFY

SEQ ID NO: 171 - protein, 10 aa, Homo sapiens
GPESRLLEFY

SEQ ID NO: 172 - protein, 9 aa, Homo sapiens
ESRLLEFYL

SEQ ID NO: 173 - protein, 9 aa, Homo sapiens
RLLEFYLAM

SEQ ID NO: 174 - protein, 9 aa, Homo sapiens
LEFYLAMPF

SEQ ID NO: 175 - protein, 10 aa, Homo sapiens
LLEFYLAMPF

SEQ ID NO: 176 - protein, 10 aa, Homo sapiens
AMPFATPMEA

SEQ ID NO: 177 - protein, 9 aa, Homo sapiens
MPFATPMEA

SEQ ID NO: 178 - protein, 9 aa, Homo sapiens
PLPVPGVLL

SEQ ID NO: 179 - protein, 10 aa, Homo sapiens
PPLPVPGVLL

SEQ ID NO: 180 - protein, 8 aa, Homo sapiens
LPVPGVLL

SEQ ID NO: 181 - protein, 10 aa, Homo sapiens
ELARRSLAQD

SEQ ID NO: 182 - protein, 9 aa, Homo sapiens
VPGVLLKEF

SEQ ID NO: 183 - protein, 10 aa, Homo sapiens
PVPGVLLKEF

SEQ ID NO: 184 - protein, 8 aa, Homo sapiens
LPVPGVLL

SEQ ID NO: 185 - protein, 9 aa, Homo sapiens
TVSGNILTI

SEQ ID NO: 186 - protein, 10 aa, Homo sapiens
FTVSGNILTI

SEQ ID NO: 187 - protein, 9 aa, Homo sapiens
GVLLKEFTV

SEQ ID NO: 188 - protein, 10 aa, Homo sapiens
VLLKEFTVSG

SEQ ID NO: 189 - protein, 9 aa, Homo sapiens
LLKEFTVSG

SEQ ID NO: 190 - protein, 9 aa, Homo sapiens
VPGVLLKEF

SEQ ID NO: 191 - protein, 10 aa, Homo sapiens
PVPGVLLKEF

SEQ ID NO: 192 - protein, 9 aa, Homo sapiens
AADHRQLQL

SEQ ID NO: 193 - protein, 9 aa, Homo sapiens
SISSCLQQL

SEQ ID NO: 194 - protein, 10 aa, Homo sapiens
LSISSCLQQL

SEQ ID NO: 195 - protein, 10 aa, Homo sapiens
TAADHRQLQL

SEQ ID NO: 196 - protein, 9 aa, Homo sapiens
WITQCFLPV

SEQ ID NO: 197 - protein, 9 aa, Homo sapiens
SLLMWITQC

SEQ ID NO: 198 - protein, 9 aa, Homo sapiens
SSCLQQLSL

SEQ ID NO: 199 - protein, 9 aa, Homo sapiens
QQLSLLMWI

SEQ ID NO: 200 - protein, 9 aa, Homo sapiens
SCLQQLSLL

SEQ ID NO: 201 - protein, 10 aa, Homo sapiens
SSCLQQLSLL

SEQ ID NO: 202 - protein, 9 aa, Homo sapiens
TQCFLPVFL

SEQ ID NO: 203 - protein, 10 aa, Homo sapiens
ITQCFLPVFL

SEQ ID NO: 204 - protein, 9 aa, Homo sapiens
PMQDIKMIL

SEQ ID NO: 205 - protein, 10 aa, Homo sapiens
MPMQDIKMIL

SEQ ID NO: 206 - protein, 9 aa, Homo sapiens
QHLIGLSNL

SEQ ID NO: 207 - protein, 10 aa, Homo sapiens
LQHLIGLSNL

SEQ ID NO: 208 - protein, 8 aa, Homo sapiens
HLIGLSNL

SEQ ID NO: 209 - protein, 9 aa, Homo sapiens
IGLSNLTHV

SEQ ID NO: 210 - protein, 10 aa, Homo sapiens
LIGLSNLTHV

SEQ ID NO: 211 - protein, 9 aa, Homo sapiens
VLVHPQWVL

SEQ ID NO: 212 - protein, 10 aa, Homo sapiens
GVLVHPQWVL

SEQ ID NO: 213 - protein, 9 aa, Homo sapiens
GVLVHPQWV

SEQ ID NO: 214 - protein, 9 aa, Homo sapiens
WVLTAAHCI

SEQ ID NO: 215 - protein, 10 aa, Homo sapiens
LVHPQWVLTA

SEQ ID NO: 216 - protein, 10 aa, Homo sapiens
VLVHPQWVLT

SEQ ID NO: 217 - protein, 9 aa, Homo sapiens
LVHPQWVLT

SEQ ID NO: 218 - protein, 8 aa, Homo sapiens
CIRNKSVI

SEQ ID NO: 219 - protein, 9 aa, Homo sapiens
HCIRNKSVI

SEQ ID NO: 220 - protein, 9 aa, Homo sapiens
HPQWVLTAA

SEQ ID NO: 221 - protein, 10 aa, Homo sapiens
AAHCIRNKSV

SEQ ID NO: 222 - protein, 8 aa, Homo sapiens
LLWGPGQL

SEQ ID NO: 223 - protein, 9 aa, Homo sapiens
LLLWGPGQL

SEQ ID NO: 224 - protein, 10 aa, Homo sapiens
GLLLWGPGQL

SEQ ID NO: 225 - protein, 9 aa, Homo sapiens
ALQPAAAIL

SEQ ID NO: 226 - protein, 10 aa, Homo sapiens
HALQPAAAIL

SEQ ID NO: 227 - protein, 10 aa, Homo sapiens
APEKDKFFAY

SEQ ID NO: 228 - protein, 9 aa, Homo sapiens
PEKDKFFAY

SEQ ID NO: 229 - protein, 9 aa, Homo sapiens
EKDKFFAYL

SEQ ID NO: 230 - protein, 8 aa, Homo sapiens
KDKFFAYL

SEQ ID NO: 231 - protein, 9 aa, Homo sapiens
PAFLPWHRL

SEQ ID NO: 232 - protein, 10 aa, Homo sapiens
APAFLPWHRL

SEQ ID NO: 233 - protein, 10 aa, Homo sapiens
FLLRWEQEIQ

SEQ ID NO: 234 - protein, 9 aa, Homo sapiens
RLFLLRWEQ

SEQ ID NO: 235 - protein, 10 aa, Homo sapiens
GSEIWRDIDF

SEQ ID NO: 236 - protein, 9 aa, Homo sapiens
SEIWRDIDF

SEQ ID NO: 237 - protein, 9 aa, Homo sapiens
RIWSWLLGA

SEQ ID NO: 238 - protein, 9 aa, Homo sapiens
SWLLGAAMV

SEQ ID NO: 239 - protein, 10 aa, Homo sapiens
WLLGAAMVGA

SEQ ID NO: 240 - protein, 9 aa, Homo sapiens
LLGAAMVGA

SEQ ID NO: 241 - protein, 9 aa, Homo sapiens
LLHETDSAV

SEQ ID NO: 242 - protein, 9 aa, Homo sapiens
ATARRPRWL

SEQ ID NO: 243 - protein, 9 aa, Homo sapiens
TPKHNMKAF

SEQ ID NO: 244 - protein, 10 aa, Homo sapiens
ELKAENIKKF

SEQ ID NO: 245 - protein, 9 aa, Homo sapiens
  VARIANT (7)...(7): Xaa= His or Tyr
NIKKFLXNF

SEQ ID NO: 246 - protein, 10 aa, Homo sapiens
  VARIANT (8)...(8): Xaa = His or Tyr
ENIKKFLXNF

SEQ ID NO: 247 - protein, 9 aa, Homo sapiens
AGAKGVILY

SEQ ID NO: 248 - protein, 10 aa, Homo sapiens
PLMYSLVHNL

SEQ ID NO: 249 - protein, 9 aa, Homo sapiens
LMYSLVHNL

SEQ ID NO: 250 - protein, 9 aa, Homo sapiens
RVDCTPLMY

SEQ ID NO: 251 - protein, 9 aa, Homo sapiens
DCTPLMYSL

SEQ ID NO: 252 - protein, 9 aa, Homo sapiens
SGMPRISKL

SEQ ID NO: 253 - protein, 10 aa, Homo sapiens
FSGMPRISKL

SEQ ID NO: 254 - protein, 28 aa, Homo sapiens
RLTAADHRQLQLSISSCLQQLSLLMWIT

SEQ ID NO: 255 - protein, 28 aa, Homo sapiens
SSCLQQLSLLMWITQCFLPVFLAQPPSG

SEQ ID NO: 256 - protein, 8 aa, Homo sapiens
KAEMLESV

SEQ ID NO: 257 - protein, 9 aa, Homo sapiens
TKAEMLESV

SEQ ID NO: 258 - protein, 10 aa, Homo sapiens
VTKAEMLESV

SEQ ID NO: 259 - protein, 9 aa, Homo sapiens
MLESVIKNY

SEQ ID NO: 260 - protein, 10 aa, Homo sapiens
EMLESVIKNY

SEQ ID NO: 261 - protein, 9 aa, Homo sapiens
KAEMLESVI

SEQ ID NO: 262 - protein, 8 aa, Homo sapiens
KASESLQL

SEQ ID NO: 263 - protein, 9 aa, Homo sapiens
GKASESLQL

SEQ ID NO: 264 - protein, 9 aa, Homo sapiens
ASESLQLVF

SEQ ID NO: 265 - protein, 9 aa, Homo sapiens
LVFGIDVKE

SEQ ID NO: 266 - protein, 8 aa, Homo sapiens
LLKYRARE

SEQ ID NO: 267 - protein, 8 aa, Homo sapiens
VADLVGFL

SEQ ID NO: 268 - protein, 9 aa, Homo sapiens
KVADLVGFL

SEQ ID NO: 269 - protein, 9 aa, Homo sapiens
ADLVGFLLL

SEQ ID NO: 270 - protein, 10 aa, Homo sapiens
VADLVGFLLL

SEQ ID NO: 271 - protein, 10 aa, Homo sapiens
LLKYRAREPV

SEQ ID NO: 272 - protein, 9 aa, Homo sapiens
LVETSYVKV

SEQ ID NO: 273 - protein, 10 aa, Homo sapiens
ALVETSYVKV

SEQ ID NO: 274 - protein, 9 aa, Homo sapiens
KVLHHMVKI

SEQ ID NO: 275 - protein, 9 aa, Homo sapiens
YVKVLHHMV

SEQ ID NO: 276 - protein, 9 aa, Homo sapiens
PRALVETSY

SEQ ID NO: 277 - protein, 10 aa, Homo sapiens
GPRALVETSY

SEQ ID NO: 278 - protein, 10 aa, Homo sapiens
LVETSYVKVL

SEQ ID NO: 279 - protein, 9 aa, Homo sapiens
TIIPEVPQL

SEQ ID NO: 280 - protein, 10 aa, Homo sapiens
DTIIPEVPQL

SEQ ID NO: 281 - protein, 10 aa, Homo sapiens
EVPQLTDLSF

SEQ ID NO: 282 - protein, 8 aa, Homo sapiens
TPLNSSTI

SEQ ID NO: 283 - protein, 8 aa, Homo sapiens
IGLRWTPL

SEQ ID NO: 284 - protein, 9 aa, Homo sapiens
SIGLRWTPL

SEQ ID NO: 285 - protein, 9 aa, Homo sapiens
LNSSTIIGY

SEQ ID NO: 286 - protein, 10 aa, Homo sapiens
PLNSSTIIGY

SEQ ID NO: 287 - protein, 9 aa, Homo sapiens
TPLNSSTII

SEQ ID NO: 288 - protein, 8 aa, Homo sapiens
IGYRITVV

SEQ ID NO: 289 - protein, 9 aa, Homo sapiens
IIGYRITVV

SEQ ID NO: 290 - protein, 10 aa, Homo sapiens
TIIGYRITVV

SEQ ID NO: 291 - protein, 9 aa, Homo sapiens
IGYRITVVA

SEQ ID NO: 292 - protein, 10 aa, Homo sapiens
IIGYRITVVA

SEQ ID NO: 293 - protein, 8 aa, Homo sapiens
SLPVSPRL

SEQ ID NO: 294 - protein, 9 aa, Homo sapiens
QSLPVSPRL

SEQ ID NO: 295 - protein, 8 aa, Homo sapiens
PVSPRLQL

SEQ ID NO: 296 - protein, 9 aa, Homo sapiens
LPVSPRLQL

SEQ ID NO: 297 - protein, 10 aa, Homo sapiens
SLPVSPRLQL

SEQ ID NO: 298 - protein, 8 aa, Homo sapiens
LPVSPRLQ

SEQ ID NO: 299 - protein, 9 aa, Homo sapiens
QLSNGNRTL

SEQ ID NO: 300 - protein, 10 aa, Homo sapiens
LQLSNGNRTL

SEQ ID NO: 301 - protein, 9 aa, Homo sapiens
WVNNQSLPV

SEQ ID NO: 302 - protein, 9 aa, Homo sapiens
PVSPRLQLS

SEQ ID NO: 303 - protein, 8 aa, Homo sapiens
SLPVSPRL

SEQ ID NO: 304 - protein, 9 aa, Homo sapiens
QSLPVSPRL

SEQ ID NO: 305 - protein, 8 aa, Homo sapiens
PVSPRLQL

SEQ ID NO: 306 - protein, 9 aa, Homo sapiens
LPVSPRLQL

SEQ ID NO: 307 - protein, 10 aa, Homo sapiens
SLPVSPRLQL

SEQ ID NO: 308 - protein, 8 aa, Homo sapiens
LPVSPRLQ

SEQ ID NO: 309 - protein, 9 aa, Homo sapiens
QLSNDNRTL

SEQ ID NO: 310 - protein, 10 aa, Homo sapiens
LQLSNDNRTL

SEQ ID NO: 311 - protein, 9 aa, Homo sapiens
WVNNQSLPV

SEQ ID NO: 312 - protein, 9 aa, Homo sapiens
NQSLPVSPR

SEQ ID NO: 313 - protein, 8 aa, Homo sapiens
SLPVSPRL

SEQ ID NO: 314 - protein, 9 aa, Homo sapiens
QSLPVSPRL

SEQ ID NO: 315 - protein, 8 aa, Homo sapiens
PVSPRLQL

SEQ ID NO: 316 - protein, 9 aa, Homo sapiens
LPVSPRLQL

SEQ ID NO: 317 - protein, 10 aa, Homo sapiens
SLPVSPRLQL

SEQ ID NO: 318 - protein, 8 aa, Homo sapiens
LPVSPRLQ

SEQ ID NO: 319 - protein, 9 aa, Homo sapiens
QLSNGNRTL

SEQ ID NO: 320 - protein, 10 aa, Homo sapiens
LQLSNGNRTL

SEQ ID NO: 321 - protein, 9 aa, Homo sapiens
WVNGQSLPV

SEQ ID NO: 322 - protein, 9 aa, Homo sapiens
LWWVNGQSL

SEQ ID NO: 323 - protein, 10 aa, Homo sapiens
YLWWVNGQSL

SEQ ID NO: 324 - protein, 9 aa, Homo sapiens
GQSLPVSPR

SEQ ID NO: 325 - protein, 8 aa, Homo sapiens
DMKLRLPA

SEQ ID NO: 326 - protein, 10 aa, Homo sapiens
GTDMKLRLPA

SEQ ID NO: 327 - protein, 8 aa, Homo sapiens
HLDMLRHL

SEQ ID NO: 328 - protein, 9 aa, Homo sapiens
THLDMLRHL

SEQ ID NO: 329 - protein, 10 aa, Homo sapiens
ETHLDMLRHL

SEQ ID NO: 330 - protein, 8 aa, Homo sapiens
PASPETHL

SEQ ID NO: 331 - protein, 9 aa, Homo sapiens
LPASPETHL

SEQ ID NO: 332 - protein, 10 aa, Homo sapiens
RLPASPETHL

SEQ ID NO: 333 - protein, 9 aa, Homo sapiens
SPETHLDML

SEQ ID NO: 334 - protein, 10 aa, Homo sapiens
ASPETHLDML

SEQ ID NO: 335 - protein, 9 aa, Homo sapiens
HLDMLRHLY

SEQ ID NO: 336 - protein, 10 aa, Homo sapiens
THLDMLRHLY

SEQ ID NO: 337 - protein, 8 aa, Homo sapiens
ELRKVKVL

SEQ ID NO: 338 - protein, 9 aa, Homo sapiens
TELRKVKVL

SEQ ID NO: 339 - protein, 10 aa, Homo sapiens
ETELRKVKVL

SEQ ID NO: 340 - protein, 9 aa, Homo sapiens
LKETELRKV

SEQ ID NO: 341 - protein, 10 aa, Homo sapiens
ILKETELRKV

SEQ ID NO: 342 - protein, 9 aa, Homo sapiens
MRILKETEL

SEQ ID NO: 343 - protein, 10 aa, Homo sapiens
QMRILKETEL

SEQ ID NO: 344 - protein, 9 aa, Homo sapiens
ETELRKVKV

SEQ ID NO: 345 - protein, 10 aa, Homo sapiens
KETELRKVKV

SEQ ID NO: 346 - protein, 9 aa, Homo sapiens
MPNQAQMRI

SEQ ID NO: 347 - protein, 10 aa, Homo sapiens
AMPNQAQMRI

SEQ ID NO: 348 - protein, 10 aa, Homo sapiens
MPNQAQMRIL

SEQ ID NO: 349 - protein, 8 aa, Homo sapiens
RPRFRELV

SEQ ID NO: 350 - protein, 9 aa, Homo sapiens
CRPRFRELV

SEQ ID NO: 351 - protein, 9 aa, Homo sapiens
RFRELVSEF

SEQ ID NO: 352 - protein, 10 aa, Homo sapiens
PRFRELVSEF

SEQ ID NO: 353 - protein, 9 aa, Homo sapiens
ECRPRFREL

SEQ ID NO: 354 - protein, 9 aa, Homo sapiens
GAASGLNGC

SEQ ID NO: 355 - protein, 9 aa, Homo sapiens
RASGPGGGA

SEQ ID NO: 356 - protein, 9 aa, Homo sapiens
PHGGAASGL

SEQ ID NO: 357 - protein, 10 aa, Homo sapiens
GPHGGAASGL

SEQ ID NO: 358 - protein, 10 aa, Homo sapiens
APRGPHGGAA

SEQ ID NO: 359 - protein, 8 aa, Homo sapiens
VRPRRWKL

SEQ ID NO: 360 - protein, 9 aa, Homo sapiens
EVRPRRWKL

SEQ ID NO: 361 - protein, 9 aa, Homo sapiens
RPRRWKLQV

SEQ ID NO: 362 - protein, 9 aa, Homo sapiens
PRRWKLQVL

SEQ ID NO: 363 - protein, 10 aa, Homo sapiens
RPRRWKLQVL

SEQ ID NO: 364 - protein, 9 aa, Homo sapiens
RWKLQVLDL

SEQ ID NO: 365 - protein, 10 aa, Homo sapiens
RRWKLQVLDL

SEQ ID NO: 366 - protein, 9 aa, Homo sapiens
PVEVLVDLF

SEQ ID NO: 367 - protein, 8 aa, Homo sapiens
VKRKKNVL

SEQ ID NO: 368 - protein, 9 aa, Homo sapiens
KVKRKKNVL

SEQ ID NO: 369 - protein, 10 aa, Homo sapiens
EKVKRKKNVL

SEQ ID NO: 370 - protein, 8 aa, Homo sapiens
KVKRKKNV

SEQ ID NO: 371 - protein, 8 aa, Homo sapiens
RKKNVLRL

SEQ ID NO: 372 - protein, 9 aa, Homo sapiens
KRKKNVLRL

SEQ ID NO: 373 - protein, 10 aa, Homo sapiens
VKRKKNVLRL

SEQ ID NO: 374 - protein, 8 aa, Homo sapiens
DELFSYLI

SEQ ID NO: 375 - protein, 9 aa, Homo sapiens
VLRLCCKKL

SEQ ID NO: 376 - protein, 10 aa, Homo sapiens
NVLRLCCKKL

SEQ ID NO: 377 - protein, 9 aa, Homo sapiens
YLIEKVKRK

SEQ ID NO: 378 - protein, 8 aa, Homo sapiens
QAWPFTCL

SEQ ID NO: 379 - protein, 9 aa, Homo sapiens
VQAWPFTCL

SEQ ID NO: 380 - protein, 10 aa, Homo sapiens
MVQAWPFTCL

SEQ ID NO: 381 - protein, 8 aa, Homo sapiens
LPLGVLMK

SEQ ID NO: 382 - protein, 9 aa, Homo sapiens
CLPLGVLMK

SEQ ID NO: 383 - protein, 10 aa, Homo sapiens
TCLPLGVLMK

SEQ ID NO: 384 - protein, 9 aa, Homo sapiens
GVLMKGQHL

SEQ ID NO: 385 - protein, 9 aa, Homo sapiens
LPLGVLMKG

SEQ ID NO: 386 - protein, 10 aa, Homo sapiens
CLPLGVLMKG

SEQ ID NO: 387 - protein, 10 aa, Homo sapiens
WPFTCLPLGV

SEQ ID NO: 388 - protein, 9 aa, Homo sapiens
ELFPPLFMA

SEQ ID NO: 389 - protein, 9 aa, Homo sapiens
PRELFPPLF

SEQ ID NO: 390 - protein, 10 aa, Homo sapiens
LPRELFPPLF

SEQ ID NO: 391 - protein, 9 aa, Homo sapiens
RELFPPLFM

SEQ ID NO: 392 - protein, 10 aa, Homo sapiens
PRELFPPLFM

SEQ ID NO: 393 - protein, 8 aa, Homo sapiens
RPSLYTKV

SEQ ID NO: 394 - protein, 9 aa, Homo sapiens
ERPSLYTKV

SEQ ID NO: 395 - protein, 8 aa, Homo sapiens
LPERPSLY

SEQ ID NO: 396 - protein, 9 aa, Homo sapiens
ALPERPSLY

SEQ ID NO: 397 - protein, 9 aa, Homo sapiens
SLYTKVVHY

SEQ ID NO: 398 - protein, 10 aa, Homo sapiens
PSLYTKVVHY

SEQ ID NO: 399 - protein, 9 aa, Homo sapiens
RPSLYTKVV

SEQ ID NO: 400 - protein, 8 aa, Homo sapiens
GNKVKNAQ

SEQ ID NO: 401 - protein, 8 aa, Homo sapiens
IARYGKVF

SEQ ID NO: 402 - protein, 9 aa, Homo sapiens
AQLAGAKGV

SEQ ID NO: 403 - protein, 9 aa, Homo sapiens
KVFRGNKVK

SEQ ID NO: 404 - protein, 9 aa, Homo sapiens
GNKVKNAQL

SEQ ID NO: 405 - protein, 9 aa, Homo sapiens
TPGYPANEY

SEQ ID NO: 406 - protein, 10 aa, Homo sapiens
LTPGYPANEY

SEQ ID NO: 407 - protein, 9 aa, Homo sapiens
GYPANEYAY

SEQ ID NO: 408 - protein, 10 aa, Homo sapiens
PGYPANEYAY

SEQ ID NO: 409 - protein, 9 aa, Homo sapiens
DPLTPGYPA

SEQ ID NO: 410 - protein, 9 aa, Homo sapiens
SLYESWTKK

SEQ ID NO: 411 - protein, 10 aa, Homo sapiens
KSLYESWTKK

SEQ ID NO: 412 - protein, 9 aa, Homo sapiens
EGFEGKSLY

SEQ ID NO: 413 - protein, 10 aa, Homo sapiens
DEGFEGKSLY

SEQ ID NO: 414 - protein, 9 aa, Homo sapiens
TKKSPSPEF

SEQ ID NO: 415 - protein, 10 aa, Homo sapiens
WTKKSPSPEF

SEQ ID NO: 416 - protein, 10 aa, Homo sapiens
SLYESWTKKS

SEQ ID NO: 417 - protein, 8 aa, Homo sapiens
WGEVKRQI

SEQ ID NO: 418 - protein, 9 aa, Homo sapiens
AWGEVKRQI

SEQ ID NO: 419 - protein, 10 aa, Homo sapiens
KAWGEVKRQI

SEQ ID NO: 420 - protein, 8 aa, Homo sapiens
KAWGEVKR

SEQ ID NO: 421 - protein, 9 aa, Homo sapiens
SKAWGEVKR

SEQ ID NO: 422 - protein, 9 aa, Homo sapiens
QIYVAAFTV

SEQ ID NO: 423 - protein, 9 aa, Homo sapiens
YVAAFTVQA

SEQ ID NO: 424 - protein, 9 aa, Homo sapiens
WGEVKRQIY

SEQ ID NO: 425 - protein, 9 aa, Homo sapiens
EVKRQIYVA

SEQ ID NO: 426 - protein, 9 aa, Homo sapiens
TVQAAAETL

SEQ ID NO: 427 - protein, 10 aa, Homo sapiens
FTVQAAAETL

SEQ ID NO: 428 - protein, 9 aa, Homo sapiens
KRQIYVAAF

SEQ ID NO: 429 - protein, 9 aa, Homo sapiens
PSKAWGEVK

SEQ ID NO: 430 - protein, 9 aa, Homo sapiens
KAWGEVKRQ

SEQ ID NO: 431 - protein, 9 aa, Homo sapiens
WKEFGLDSV

SEQ ID NO: 432 - protein, 10 aa, Homo sapiens
QWKEFGLDSV

SEQ ID NO: 433 - protein, 10 aa, Homo sapiens
EFGLDSVELA

SEQ ID NO: 434 - protein, 9 aa, Homo sapiens
ELRQKESKL

SEQ ID NO: 435 - protein, 10 aa, Homo sapiens
AELRQKESKL

SEQ ID NO: 436 - protein, 9 aa, Homo sapiens
KLQENRKII

SEQ ID NO: 437 - protein, 8 aa, Homo sapiens
QLEEKTKL

SEQ ID NO: 438 - protein, 9 aa, Homo sapiens
NQLEEKTKL

SEQ ID NO: 439 - protein, 9 aa, Homo sapiens
LLEESRDKV

SEQ ID NO: 440 - protein, 10 aa, Homo sapiens
FLLEESRDKV

SEQ ID NO: 441 - protein, 9 aa, Homo sapiens
ESRDKVNQL

SEQ ID NO: 442 - protein, 10 aa, Homo sapiens
EESRDKVNQL

SEQ ID NO: 443 - protein, 9 aa, Homo sapiens
EKEVHDLEY

SEQ ID NO: 444 - protein, 10 aa, Homo sapiens
REKEVHDLEY

SEQ ID NO: 445 - protein, 9 aa, Homo sapiens
DLEYSYCHY

SEQ ID NO: 446 - protein, 9 aa, Homo sapiens
EVHDLEYSY

SEQ ID NO: 447 - protein, 10 aa, Homo sapiens
EVHDLEYSYC

SEQ ID NO: 448 - protein, 8 aa, Homo sapiens
KLSSKREL

SEQ ID NO: 449 - protein, 8 aa, Homo sapiens
ELKNTEYF

SEQ ID NO: 450 - protein, 9 aa, Homo sapiens
RELKNTEYF

SEQ ID NO: 451 - protein, 8 aa, Homo sapiens
KRGQRPKL

SEQ ID NO: 452 - protein, 10 aa, Homo sapiens
LPKRGQRPKL

SEQ ID NO: 453 - protein, 9 aa, Homo sapiens
LKNTEYFTL

SEQ ID NO: 454 - protein, 10 aa, Homo sapiens
ELKNTEYFTL

SEQ ID NO: 455 - protein, 9 aa, Homo sapiens
KRELKNTEY

SEQ ID NO: 456 - protein, 9 aa, Homo sapiens
KLSSKRELK

SEQ ID NO: 457 - protein, 9 aa, Homo sapiens
GQRPKLSSK

SEQ ID NO: 458 - protein, 10 aa, Homo sapiens
RGQRPKLSSK

SEQ ID NO: 459 - protein, 9 aa, Homo sapiens
RPKLSSKRE

SEQ ID NO: 460 - protein, 8 aa, Homo sapiens
LEYVREEL

SEQ ID NO: 461 - protein, 9 aa, Homo sapiens
ELEYVREEL

SEQ ID NO: 462 - protein, 10 aa, Homo sapiens
NELEYVREEL

SEQ ID NO: 463 - protein, 10 aa, Homo sapiens
ELKQKREDEV

SEQ ID NO: 464 - protein, 9 aa, Homo sapiens
YVREELKQK

SEQ ID NO: 465 - protein, 9 aa, Homo sapiens
QLNVYEIKV

SEQ ID NO: 466 - protein, 9 aa, Homo sapiens
SKQLNVYEI

SEQ ID NO: 467 - protein, 9 aa, Homo sapiens
AESKQLNVY

SEQ ID NO: 468 - protein, 10 aa, Homo sapiens
TAESKQLNVY

SEQ ID NO: 469 - protein, 8 aa, Homo sapiens
IKVNKLEL

SEQ ID NO: 470 - protein, 9 aa, Homo sapiens
EIKVNKLEL

SEQ ID NO: 471 - protein, 10 aa, Homo sapiens
YEIKVNKLEL

SEQ ID NO: 472 - protein, 9 aa, Homo sapiens
KLELELESA

SEQ ID NO: 473 - protein, 9 aa, Homo sapiens
VYEIKVNKL

SEQ ID NO: 474 - protein, 10 aa, Homo sapiens
NVYEIKVNKL

SEQ ID NO: 475 - protein, 9 aa, Homo sapiens
ELESAKQKF

SEQ ID NO: 476 - protein, 9 aa, Homo sapiens
KLELELESA

SEQ ID NO: 477 - protein, 9 aa, Homo sapiens
ELESAKQKF

SEQ ID NO: 478 - protein, 8 aa, Homo sapiens
KEKLKREA

SEQ ID NO: 479 - protein, 9 aa, Homo sapiens
EAKENTATL

SEQ ID NO: 480 - protein, 10 aa, Homo sapiens
REAKENTATL

SEQ ID NO: 481 - protein, 10 aa, Homo sapiens
KLKREAKENT

SEQ ID NO: 482 - protein, 8 aa, Homo sapiens
EAEKIKKW

SEQ ID NO: 483 - protein, 9 aa, Homo sapiens
GLSRVYSKL

SEQ ID NO: 484 - protein, 10 aa, Homo sapiens
EGLSRVYSKL

SEQ ID NO: 485 - protein, 9 aa, Homo sapiens
KLYKEAEKI

SEQ ID NO: 486 - protein, 9 aa, Homo sapiens
NSEGLSRVY

SEQ ID NO: 487 - protein, 10 aa, Homo sapiens
ENSEGLSRVY

SEQ ID NO: 488 - protein, 9 aa, Homo sapiens
LSRVYSKLY

SEQ ID NO: 489 - protein, 10 aa, Homo sapiens
GLSRVYSKLY

SEQ ID NO: 490 - protein, 10 aa, Homo sapiens
LENSEGLSRV

SEQ ID NO: 491 - protein, 10 aa, Homo sapiens
KLYKEAEKIK

SEQ ID NO: 492 - protein, 8 aa, Homo sapiens
REDRWAVI

SEQ ID NO: 493 - protein, 9 aa, Homo sapiens
MREDRWAVI

SEQ ID NO: 494 - protein, 10 aa, Homo sapiens
KMREDRWAVI

SEQ ID NO: 495 - protein, 9 aa, Homo sapiens
KMREDRWAV

SEQ ID NO: 496 - protein, 9 aa, Homo sapiens
TTPGSTLKF

SEQ ID NO: 497 - protein, 10 aa, Homo sapiens
LTTPGSTLKF

SEQ ID NO: 498 - protein, 8 aa, Homo sapiens
GSTLKGAI

SEQ ID NO: 499 - protein, 9 aa, Homo sapiens
IRKMREDRW

SEQ ID NO: 500 - protein, 8 aa, Homo sapiens
RLEMHFKL

SEQ ID NO: 501 - protein, 9 aa, Homo sapiens
SRLEMHFKL

SEQ ID NO: 502 - protein, 9 aa, Homo sapiens
KLKEDYEKI

SEQ ID NO: 503 - protein, 9 aa, Homo sapiens
KIQHLEQEY

SEQ ID NO: 504 - protein, 10 aa, Homo sapiens
EKIQHLEQEY

SEQ ID NO: 505 - protein, 9 aa, Homo sapiens
ENSRLEMHF

SEQ ID NO: 506 - protein, 10 aa, Homo sapiens
RLEMHFKLKE

SEQ ID NO: 507 - protein, 8 aa, Homo sapiens
LEDIKVSL

SEQ ID NO: 508 - protein, 9 aa, Homo sapiens
ELEDIKVSL

SEQ ID NO: 509 - protein, 10 aa, Homo sapiens
KELEDIKVSL

SEQ ID NO: 510 - protein, 8 aa, Homo sapiens
LTKELEDI

SEQ ID NO: 511 - protein, 9 aa, Homo sapiens
HLTKELEDI

SEQ ID NO: 512 - protein, 9 aa, Homo sapiens
SLQRSVSTQ

SEQ ID NO: 513 - protein, 9 aa, Homo sapiens
TKELEDIKV

SEQ ID NO: 514 - protein, 10 aa, Homo sapiens
LTKELEDIKV

SEQ ID NO: 515 - protein, 10 aa, Homo sapiens
DIKVSLQRSV

SEQ ID NO: 516 - protein, 8 aa, Homo sapiens
KMKDLTFL

SEQ ID NO: 517 - protein, 9 aa, Homo sapiens
NKMKDLTFL

SEQ ID NO: 518 - protein, 10 aa, Homo sapiens
ENKMKDLTFL

SEQ ID NO: 519 - protein, 9 aa, Homo sapiens
LLEESRDKV

SEQ ID NO: 520 - protein, 10 aa, Homo sapiens
FLLEESRDKV

SEQ ID NO: 521 - protein, 9 aa, Homo sapiens
ESRDKVNQL

SEQ ID NO: 522 - protein, 10 aa, Homo sapiens
EESRDKVNQL

SEQ ID NO: 523 - protein, 9 aa, Homo sapiens
EKENKMKDL

SEQ ID NO: 524 - protein, 10 aa, Homo sapiens
TEKENKMKDL

SEQ ID NO: 525 - protein, 9 aa, Homo sapiens
ENKMKDLTF

SEQ ID NO: 526 - protein, 8 aa, Homo sapiens
IEKMITAF

SEQ ID NO: 527 - protein, 9 aa, Homo sapiens
NIEKMITAF

SEQ ID NO: 528 - protein, 10 aa, Homo sapiens
SNIEKMITAF

SEQ ID NO: 529 - protein, 8 aa, Homo sapiens
TAFEELRV

SEQ ID NO: 530 - protein, 9 aa, Homo sapiens
ITAFEELRV

SEQ ID NO: 531 - protein, 10 aa, Homo sapiens
MITAFEELRV

SEQ ID NO: 532 - protein, 9 aa, Homo sapiens
KMITAFEEL

SEQ ID NO: 533 - protein, 10 aa, Homo sapiens
EKMITAFEEL

SEQ ID NO: 534 - protein, 9 aa, Homo sapiens
ELRVQAENS

SEQ ID NO: 535 - protein, 10 aa, Homo sapiens
DLNSNIEKMI

SEQ ID NO: 536 - protein, 8 aa, Homo sapiens
WTSAKNTL

SEQ ID NO: 537 - protein, 9 aa, Homo sapiens
TPLPKAYTV

SEQ ID NO: 538 - protein, 10 aa, Homo sapiens
STPLPKAYTV

SEQ ID NO: 539 - protein, 9 aa, Homo sapiens
LSTPLPKAY

SEQ ID NO: 540 - protein, 10 aa, Homo sapiens
TLSTPLPKAY

SEQ ID NO: 541 - protein, 9 aa, Homo sapiens
NTLSTPLPK

SEQ ID NO: 542 - protein, 10 aa, Homo sapiens
KNTLSTPLPK

SEQ ID NO: 543 - protein, 8 aa, Homo sapiens
ISKDKRDY

SEQ ID NO: 544 - protein, 10 aa, Homo sapiens
HGISKDKRDY

SEQ ID NO: 545 - protein, 9 aa, Homo sapiens
KRDYLWTSA

SEQ ID NO: 546 - protein, 10 aa, Homo sapiens
SKDKRDYLWT

SEQ ID NO: 547 - protein, 8 aa, Homo sapiens
ENKMKDLT

SEQ ID NO: 548 - protein, 9 aa, Homo sapiens
EINDKEKQV

SEQ ID NO: 549 - protein, 9 aa, Homo sapiens
QITEKENKM

SEQ ID NO: 550 - protein, 9 aa, Homo sapiens
SLLLIQITE

SEQ ID NO: 551 - protein, 8 aa, Homo sapiens
FEKIAEEL

SEQ ID NO: 552 - protein, 9 aa, Homo sapiens
QFEKIAEEL

SEQ ID NO: 553 - protein, 10 aa, Homo sapiens
KQFEKIAEEL

SEQ ID NO: 554 - protein, 8 aa, Homo sapiens
DNKQFEKI

SEQ ID NO: 555 - protein, 9 aa, Homo sapiens
YDNKQFEKI

SEQ ID NO: 556 - protein, 10 aa, Homo sapiens
LYDNKQFEKI

SEQ ID NO: 557 - protein, 8 aa, Homo sapiens
LGEKETLL

SEQ ID NO: 558 - protein, 9 aa, Homo sapiens
VLGEKETLL

SEQ ID NO: 559 - protein, 10 aa, Homo sapiens
KVLGEKETLL

SEQ ID NO: 560 - protein, 9 aa, Homo sapiens
LLRTEQQRL

SEQ ID NO: 561 - protein, 10 aa, Homo sapiens
ELLRTEQQRL

SEQ ID NO: 562 - protein, 9 aa, Homo sapiens
TEQQRLENY

SEQ ID NO: 563 - protein, 10 aa, Homo sapiens
RTEQQRLENY

SEQ ID NO: 564 - protein, 9 aa, Homo sapiens
EDQLIILTM

SEQ ID NO: 565 - protein, 10 aa, Homo sapiens
RLENYEDQLI

SEQ ID NO: 566 - protein, 8 aa, Homo sapiens
KARAAHSF

SEQ ID NO: 567 - protein, 9 aa, Homo sapiens
VVTEFETTV

SEQ ID NO: 568 - protein, 10 aa, Homo sapiens
FVVTEFETTV

SEQ ID NO: 569 - protein, 9 aa, Homo sapiens
VTEFETTVC

SEQ ID NO: 570 - protein, 10 aa, Homo sapiens
VVTEFETTVC

SEQ ID NO: 571 - protein, 9 aa, Homo sapiens
DLQIATNTI

SEQ ID NO: 572 - protein, 9 aa, Homo sapiens
IATNTICQL

SEQ ID NO: 573 - protein, 10 aa, Homo sapiens
QIATNTICQL

SEQ ID NO: 574 - protein, 9 aa, Homo sapiens
VMTKLGFKY

SEQ ID NO: 575 - protein, 9 aa, Homo sapiens
LNYEVMTKL

SEQ ID NO: 576 - protein, 10 aa, Homo sapiens
KLNYEVMTKL

SEQ ID NO: 577 - protein, 9 aa, Homo sapiens
TLPPFMRSK

SEQ ID NO: 578 - protein, 9 aa, Homo sapiens
KIMPKKPAE

SEQ ID NO: 579 - protein, 10 aa, Homo sapiens
SLQRIFPKIM

SEQ ID NO: 580 - protein, 9 aa, Homo sapiens
YIKSYLEQA

SEQ ID NO: 581 - protein, 9 aa, Homo sapiens
SFQDYIKSY

SEQ ID NO: 582 - protein, 10 aa, Homo sapiens
DSFQDYIKSY

SEQ ID NO: 583 - protein, 8 aa, Homo sapiens
LPEEKQPL

SEQ ID NO: 584 - protein, 9 aa, Homo sapiens
QLPEEKQPL

SEQ ID NO: 585 - protein, 10 aa, Homo sapiens
KQLPEEKQPL

SEQ ID NO: 586 - protein, 9 aa, Homo sapiens
LPEEKQPLL

SEQ ID NO: 587 - protein, 10 aa, Homo sapiens
QLPEEKQPLL

SEQ ID NO: 588 - protein, 9 aa, Homo sapiens
SLLCRHKRK

SEQ ID NO: 589 - protein, 91 aa, Homo sapiens
EVPQLTDLSFVDITDSSIGLRWTPLNSSTIIGYRITVVAAGEGIPIFEDFVDSSVGYYTVTGLEPGIDYDISVITLINGGESAPTTLTQQT

SEQ ID NO: 590 - protein, 147 aa, Homo sapiens
CTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTW

SEQ ID NO: 591 - DNA, 2823 bp, Homo sapiens
ctgcacttttgataacctgagtcccggcctggagtacaatgtcagtgtttacactgtcaaggatgacaaggaaagtgtccctatctctgataccatcatcccaggtaatagaaaataagctgctatcctgagagtgacattccaataagagtggggattagcatcttaatccccagatgcttaagggtgtcaactatatttgggatttaattccgatctcccagctgcactttccaaaaccaagaagtcaaagcagcgatttggacaaaatgcttgctgttaacactgctttactgtctgtgcttcactgggatgctgtgtgttgcagcgagtatgtaatggagtggcagccatggctttaactctgtattgtctgctcacatggaagtatgactaaaacactgtcacgtgtctgtactcagtactgataggctcaaagtaatatggtaaatgcatcccatcagtacatttctgcccgattttacaatccatatcaatttccaacagctgcctatttcatcttgcagtttcaaatccttctttttgaaaattggattttaaaaaaaagttaagtaaaagtcacaccttcagggttgttctttcttgtggccttgaaagacaacattgcaaaggcctgtcctaaggataggcttgtttgtccattgggttataacataatgaaagcattggacagatcgtgtccccctttggactcttcagtagaatgcttttactaacgctaattacatgttttgattatgaatgaacctaaaatagtggcaatggccttaacctaggcctgtctttcctcagcctgaatgtgcttttgaatggcacatttcacaccatacattcataatgcattagcgttatggccatgatgttgtcatgagttttgtatgggagaaaaaaaatcaatttatcacccatttattattttttccggttgttcatgcaagcttattttctactaaaacagttttggaattattaaaagcattgctgatacttacttcagatattatgtctaggctctaagaatggtttcgacatcctaaacagccatatgatttttaggaatctgaacagttcaaattgtaccctttaaggatgttttcaaaatgtaaaaaatatatatatatatatatattccctaaaagaatattcctgtttattcttctagggaagcaaactgttcatgatgcttaggaagtcttttcagagaatttaaaacagattgcatattaccatcattgctttaacattccaccaattttactactagtaacctgatatacactgctttattttttcctctttttttccctctattttccttttgcctccccctccctttgctttgtaactcaatagaggtgccccaactcactgacctaagctttgttgatataaccgattcaagcatcggcctgaggtggaccccgctaaactcttccaccattattgggtaccgcatcacagtagttgcggcaggagaaggtatccctatttttgaagattttgtggactcctcagtaggatactacacagtcacagggctggagccgggcattgactatgatatcagcgttatcactctcattaatggcggcgagagtgcccctactacactgacacaacaaacgggtgaattttgaaaacttctgcgtttgagacatagatggtgttgcatgctgccaccagttactccggttaaatatggatgtttcatgggggaagtcagcaattggccaaagattcagataggtggaattggggggataaggaatcaaatgcatctgctaaactgattggagaaaaacacatgcaatatcttcagtacactctcatttaaaccacaagtagatataaagcctagagaaatacagatgtctgctctgttaaatataaaatagcaaatgttcattcaatttgaagacctagaatttttcttcttaaataccaaacacgaataccaaattgcgtaagtaccaattgataagaatatatcaccaaaatgtaccatcatgctcttccttctaccctttgataaactctaccatgctccttctttgtagctaaaaacccatcaaaatttagggtagagtggatgggcattgttttgaggtaggagaaaagtaaacttgggaccattctaggttttgttgctgtcactaggtaaagaaacacctctttaaccacagtctggggacaagcatgcaacattttaaaggttctctgctgtgcatgggaaaagaaacatgctgagaaccaatttgcatgaacatgttcacttgtaagtagaattcactgaatggaactgtagctctagatatctcacatggggggaagtttaggaccctcttgtctttttgtctgtgtgcatgtatttctttgtaaagtactgctatgtttctctttgctgtgtggcaacttaagcctcttcggcctgggataaaataatctgcagtggtattaataatgtacataaagtcaacatatttgaaagtagattaaaatcttttttaaatatatcaatgatggcaaaaaggttaaagggggcctaacagtactgtgtgtagtgttttatttttaacagtagtacactataacttaaaatagacttagattagactgtttgcatgattatgattctgtttcctttatgcatgaaatattgattttacctttccagctacttcgttagctttaattttaaaatacattaactgagtcttccttcttgttcgaaaccagctgttcctcctcccactgacctgcgattcaccaacattggtccagacaccatgcgtgtcacctggg

SEQ ID NO: 592 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 593 - DNA, 2974 bp, Homo sapiens
ctcagggcagagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggattgctgattctttaaatgtcttgtttcccagatttcaggaaactttttttcttttaagctatccactcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtataacagaaaaa

SEQ ID NO: 594 - protein, 1255 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIVSAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 595 - DNA, 4530 bp, Homo sapiens
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

SEQ ID NO: 596 - protein, 976 aa, Homo sapiens [sequence omitted]

SEQ ID NO: 597 - DNA, 3393 bp, Homo sapiens
gccctcatagaccgtttgttgtagttcgcgtgggaacagcaacccacggtttcccgatagttcttcaaagatatttacaaccgtaacagagaaaatggaaaagcaaaagccctttgcattgttcgtaccaccgagatcaagcagcagtcaggtgtctgcggtgaaacctcagaccctgggaggcgattccactttcttcaagagtttcaacaaatgtactgaagatgatttggagtttccatttgcaaagactaatctctccaaaaatggggaaaacattgattcagatcctgctttacaaaaagttaatttcttgcccgtgcttgagcaggttggtaattctgactgtcactatcaggaaggactaaaagactctgatttggagaattcagagggattgagcagagtgttttcaaaactgtataaggaggctgaaaagataaaaaaatggaaagtaagtacagaagctgaactgagacagaaagaaagtaagttgcaagaaaacagaaagataattgaagcacagcgaaaagccattcaggaactgcaatttggaaatgaaaaagtaagtttgaaattagaagaaggaatacaagaaaataaagatttaataaaagagaataatgccacaaggcatttatgtaatctactcaaagaaacctgtgctagatctgcagaaaagacaaagaaatatgaatatgaacgggaagaaaccaggcaagtttatatggatctaaataataacattgagaaaatgataacagctcatggggaacttcgtgtgcaagctgagaattccagactggaaatgcattttaagttaaaggaagattatgaaaaaatccaacaccttgaacaagaatacaagaaggaaataaatgacaaggaaaagcaggtatcactactattgatccaaatcactgagaaagaaaataaaatgaaagatttaacatttctgctagaggaatccagagataaagttaatcaattagaggaaaagacaaaattacagagtgaaaacttaaaacaatcaattgagaaacagcatcatttgactaaagaactagaagatattaaagtgtcattacaaagaagtgtgagtactcaaaaggctttagaggaagatttacagatagcaacaaaaacaatttgtcagctaactgaagaaaaagaaactcaaatggaagaatctaataaagctagagctgctcattcgtttgtggttactgaatttgaaactactgtctgcagcttggaagaattattgagaacagaacagcaaagattggaaaaaaatgaagatcaattgaaaatacttaccatggagcttcaaaagaaatcaagtgagctggaagagatgactaagcttacaaataacaaagaagtagaacttgaagaattgaaaaaagtcttgggagaaaaggaaacacttttatatgaaaataaacaatttgagaagattgctgaagaattaaaaggaacagaacaagaactaattggtcttctccaagccagagagaaagaagtacatgatttggaaatacagttaactgccattaccacaagtgaacagtattattcaaaagaggttaaagatctaaaaactgagcttgaaaacgagaagcttaagaatactgaattaacttcacactgcaacaagctttcactagaaaacaaagagctcacacaggaaacaagtgatatgaccctagaactcaagaatcagcaagaagatattaataataacaaaaagcaagaagaaaggatgttgaaacaaatagaaaatcttcaagaaacagaaacccaattaagaaatgaactagaatatgtgagagaagagctaaaacagaaaagagatgaagttaaatgtaaattggacaagagtgaagaaaattgtaacaatttaaggaaacaagttgaaaataaaaacaagtatattgaagaacttcagcaggagaataaggccttgaaaaaaaaaggtacagcagaaagcaagcaactgaatgtttatgagataaaggtcaataaattagagttagaactagaaagtgccaaacagaaatttggagaaatcacagacacctatcagaaagaaattgaggacaaaaagatatcagaagaaaatcttttggaagaggttgagaaagcaaaagtaatagctgatgaagcagtaaaattacagaaagaaattgataagcgatgtcaacataaaatagctgaaatggtagcacttatggaaaaacataagcaccaatatgataagatcattgaagaaagagactcagaattaggactttataagagcaaagaacaagaacagtcatcactgagagcatctttggagattgaactatccaatctcaaagctgaacttttgtctgttaagaagcaacttgaaatagaaagagaagagaaggaaaaactcaaaagagaggcaaaagaaaacacagctactcttaaagaaaaaaaagacaagaaaacacaaacatttttattggaaacacctgaaatttattggaaattggattctaaagcagttccttcacaaactgtatctcgaaatttcacatcagttgatcatggcatatccaaagataaaagagactatctgtggacatctgccaaaaatactttatctacaccattgccaaaggcatatacagtgaagacaccaacaaaaccaaaactacagcaaagagaaaacttgaatatacccattgaagaaagtaaaaaaaagagaaaaatggcctttgaatttgatattaattcagatagttcagaaactactgatcttttgagcatggtttcagaagaagagacattgaaaacactgtataggaacaataatccaccagcttctcatctttgtgtcaaaacaccaaaaaaggccccttcatctctaacaacccctggacctacactgaagtttggagctataagaaaaatgcgggaggaccgttgggctgtaattgctaaaatggatagaaaaaaaaaactaaaagaagctgaaaagttatttgtttaatttcagagaatcagtgtagttaaggagcctaataacgtgaaacttatagttaatattttgttcttatttgccagagccacattttatctggaagttgagacttaaaaaatacttgcatgaatgatttgtgtttctttatatttttagcctaaatgttaactacatattgtctggaaacctgtcattgtattcagataattagatgattatatattgttgttactttttcttgtattcatgaaaactgtttttactaagttttcaaatttgtaaagttagcctttgaatgctaggaatgcattattgagggtcattctttattctttactattaaaatattttggatgcaaaaaaaaaaaaaaaaaaaa

SEQ ID NO: 598 - protein, 188 aa, Homo sapiens
MNGDDAFARRPRDDAQISEKLRKAFDDIAKYFSKKEWEKMKSSEKIVYVYMKLNYEVMTKLGFKVTLPPFMRSKRAADFHGNDFGNDRNHRNQVERPQMTFGSLQRIFPKIMPKKPAEEENGLKEVPEASGPQNDGKQLCPPGNPSTLEKINKTSGPKRGKHAWTHRLRERKQLVVYEEISDPEEDDE

SEQ ID NO: 599 - DNA, 576 bp, Homo sapiens
atgaacggagacgacgcctttgcaaggagacccagggatgatgctcaaatatcagagaagttacgaaaggccttcgatgatattgccaaatacttctctaagaaagagtgggaaaagatgaaatcctcggagaaaatcgtctatgtgtatatgaagctaaactatgaggtcatgactaaactaggtttcaaggtcaccctcccacctttcatgcgtagtaaacgggctgcagacttccacgggaatgattttggtaacgatcgaaaccacaggaatcaggttgaacgtcctcagatgactttcggcagcctccagagaatcttcccgaagatcatgcccaagaagccagcagaggaagaaaatggtttgaaggaagtgccagaggcatctggcccacaaaatgatgggaaacagctgtgccccccgggaaatccaagtaccttggagaagattaacaagacatctggacccaaaagggggaaacatgcctggacccacagactgcgtgagagaaagcagctggtggtttatgaagagatcagcgaccctgaggaagatgacgagtaactcccctcg

SEQ ID NO: 600 - protein, 262 aa, Homo sapiens [sequence omitted]

SEQ ID NO: 601 - protein, 269 aa, Homo sapiens
MIRTLLLSTLVAGALSCGDPTYPPYVTRVVGGEEARPNSWPWQVSLQYSSNGKWYHTCGGSLIANSWVLTAAHCISSSRTYRVGLGRHNLYVAESGSLAVSVSKIVVHKDWNSNQISKGNDIALLKLANPVSLTDKIQLACLPPAGTILPNNYPCYVTGWGRLQTNGAVPDVLQQGRLLVVDYATCSSSAWWGSSVKTSMICAGGDGVISSCNGDSGGPLNCQASDGRWQVHGIVSFGSRLGCNYYHKPSVFTRVSNYIDWINSVIANN

SEQ ID NO: 602 - protein, 269 aa, Homo sapiens
MIRTLLLSTLVAGALSCGVSTYAPDMSRMLGGEEARPNSWPWQVSLQYSSNGQWYHTCGGSLIANSWVLTAAHCISSSRIYRVMLGQHNLYVAESGSLAVSVSKIVVHKDWNSNQVSKGNDIALLKLANPVSLTDKIQLACLPPAGTILPNNYPCYVTGWGRLQTNGALPDDLKQGRLLVVDYATCSSSGWWGSTVKTNMICAGGDGVICTCNGDSGGPLNCQASDGRWEVHGIGSLTSVLGCNYYYKPSIFTRVSNYNDWINSVIANN

(19) United States
(12) Patent Application Publication
(10) Pub. No.: US 2003/0220239 A1
(43) Pub. Date: Nov. 27, 2003

(54) Epitope sequences

(76) Inventors: John J. L. Simard, Northridge, CA (US); David C. Diamond, West Hills, CA (US); Liping Liu, Woodland Hills, CA (US); Zhidong Xie, Woodland Hills, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/117,937

(22) Filed: Apr. 4, 2002

Related U.S. Application Data
(60) Provisional application No. 60/282,211, filed on Apr. 6, 2001.
(60) Provisional application No. 60/337,017, filed on Nov. 7, 2001.
(60) Provisional application No. 60/363,210, filed on Mar. 7, 2002.

(51) Int. Cl. C12Q 1/68; G01N 33/574; A61K 48/00; A61K 38/17

(52) U.S. Cl. 514/12; 514/44; 435/226; 435/69.1; 435/320.1; 435/325; 435/6; 435/7.23; 702/19

(57) ABSTRACT

Disclosed herein are polypeptides, including epitopes, clusters, and antigens. Also disclosed are compositions that include said polypeptides and methods for their use.

### CROSS REFERENCE

This application claims priority under 35 U.S.C. §119(e) to U.S. Provisional Patent Application Serial No. 60/282,211, filed on Apr. 6, 2001; U.S. Provisional Patent Application Serial No. 60/337,017, filed on Nov. 7, 2001; and U.S. Provisional Patent Application Serial No. 60/363,210, filed on Mar. 7, 2002; all entitled “EPITOPE SEQUENCES,” and all of which are hereby incorporated by reference in their entirety.

### BACKGROUND OF THE INVENTION

1. Field of the Invention

The present invention generally relates to peptides, and nucleic acids encoding peptides, that are useful epitopes of target-associated antigens. More specifically, the invention relates to epitopes that have a high affinity for MHC class I and that are produced by target-specific proteasomes.

2. Description of the Related Art

Neoplasia and the Immune System

The neoplastic disease state commonly known as cancer is thought to result generally from a single cell growing out of control. The uncontrolled growth state typically results from a multi-step process in which a series of cellular systems fail, resulting in the genesis of a neoplastic cell. The resulting neoplastic cell rapidly reproduces itself, forms one or more tumors, and eventually may cause the death of the host.

Because the progenitor of the neoplastic cell shares the host's genetic material, neoplastic cells are largely unassailed by the host's immune system. During immune surveillance, the process in which the host's immune system surveys and localizes foreign materials, a neoplastic cell will appear to the host's immune surveillance machinery as a “self” cell.

Viruses and the Immune System

In contrast to cancer cells, virus infection involves the expression of clearly non-self antigens. As a result, many virus infections are successfully dealt with by the immune system with minimal clinical sequela. Moreover, it has been possible to develop effective vaccines for many of those infections that do cause serious disease. A variety of vaccine approaches have been used successfully to combat various diseases. These approaches include subunit vaccines consisting of individual proteins produced through recombinant DNA technology. Notwithstanding these advances, the selection and effective administration of minimal epitopes for use as viral vaccines has remained problematic.

In addition to the difficulties involved in epitope selection stands the problem of viruses that have evolved the capability of evading a host's immune system. Many viruses, especially viruses that establish persistent infections, such as members of the herpes and pox virus families, produce immunomodulatory molecules that permit the virus to evade the host's immune system. The effects of these immunomodulatory molecules on antigen presentation may be overcome by the targeting of select epitopes for administration as immunogenic compositions. To better understand the interaction of neoplastic cells and virally infected cells with the host's immune system, a discussion of the system's components follows below.

The immune system functions to discriminate molecules endogenous to an organism (“self” molecules) from material exogenous or foreign to the organism (“non-self” molecules). The immune system has two types of adaptive responses to foreign bodies based on the components that mediate the response: a humoral response and a cell-mediated response. The humoral response is mediated by antibodies, while the cell-mediated response involves cells classified as lymphocytes. Recent anticancer and antiviral strategies have focused on mobilizing the host immune system as a means of anticancer or antiviral treatment or therapy.

The immune system functions in three phases to protect the host from foreign bodies: the cognitive phase, the activation phase, and the effector phase. In the cognitive phase, the immune system recognizes and signals the presence of a foreign antigen or invader in the body. The foreign antigen can be, for example, a cell surface marker from a neoplastic cell or a viral protein. Once the system is aware of an invading body, antigen specific cells of the immune system proliferate and differentiate in response to the invader-triggered signals. The last stage is the effector stage in which the effector cells of the immune system respond to and neutralize the detected invader.

An array of effector cells implements an immune response to an invader. One type of effector cell, the B cell, generates antibodies targeted against foreign antigens encountered by the host. In combination with the complement system, antibodies direct the destruction of cells or organisms bearing the targeted antigen. Another type of effector cell is the natural killer cell (NK cell), a type of lymphocyte having the capacity to spontaneously recognize and destroy a variety of virus infected cells as well as malignant cell types. The method used by NK cells to recognize target cells is poorly understood.

Another type of effector cell, the T cell, has members classified into three subcategories, each playing a different role in the immune response. Helper T cells secrete cytokines which stimulate the proliferation of other cells necessary for mounting an effective immune response, while suppressor T cells down-regulate the immune response. A third category of T cell, the cytotoxic T cell (CTL), is capable of directly lysing a targeted cell presenting a foreign antigen on its surface.

The Major Histocompatibility Complex and T Cell Target Recognition

T cells are antigen-specific immune cells that function in response to specific antigen signals. B lymphocytes and the antibodies they produce are also antigen-specific entities. However, unlike B lymphocytes, T cells do not respond to antigens in a free or soluble form. For a T cell to respond to an antigen, it requires the antigen to be processed to peptides which are then bound to a presenting structure encoded in the major histocompatibility complex (MHC). This requirement is called “MHC restriction” and it is the mechanism by which T cells differentiate “self” from “non-self” cells. If an antigen is not displayed by a recognizable MHC molecule, the T cell will not recognize and act on the antigen signal. T cells specific for a peptide bound to a recognizable MHC molecule bind to these MHC-peptide complexes and proceed to the next stages of the immune response.

There are two types of MHC, class I MHC and class II MHC. T Helper cells (CD4⁺) predominately interact with class II MHC proteins while cytolytic T cells (CD8⁺) predominately interact with class I MHC proteins. Both classes of MHC protein are transmembrane proteins with a majority of their structure on the external surface of the cell. Additionally, both classes of MHC proteins have a peptide binding cleft on their external portions. It is in this cleft that small fragments of proteins, endogenous or foreign, are bound and presented to the extracellular environment.

Cells called “professional antigen presenting cells” (pAPCs) display antigens to T cells using the MHC proteins but additionally express various co-stimulatory molecules depending on the particular state of differentiation/activation of the pAPC. When T cells, specific for the peptide bound to a recognizable MHC protein, bind to these MHC-peptide complexes on pAPCs, the specific co-stimulatory molecules that act upon the T cell direct the path of differentiation/activation taken by the T cell. That is, the co-stimulation molecules affect how the T cell will act on antigenic signals in future encounters as it proceeds to the next stages of the immune response.

As discussed above, neoplastic cells are largely ignored by the immune system. A great deal of effort is now being expended in an attempt to harness a host's immune system to aid in combating the presence of neoplastic cells in a host. One such area of research involves the formulation of anticancer vaccines.

Anticancer Vaccines

Among the various weapons available to an oncologist in the battle against cancer is the immune system of the patient. Work has been done in various attempts to cause the immune system to combat cancer or neoplastic diseases. Unfortunately, the results to date have been largely disappointing. One area of particular interest involves the generation and use of anticancer vaccines.

To generate a vaccine or other immunogenic composition, it is necessary to introduce to a subject an antigen or epitope against which an immune response may be mounted. Although neoplastic cells are derived from and therefore are substantially identical to normal cells on a genetic level, many neoplastic cells are known to present tumor-associated antigens (TuAAs). In theory, these antigens could be used by a subject's immune system to recognize these antigens and attack the neoplastic cells. In reality, however, neoplastic cells generally appear to be ignored by the host's immune system.

A number of different strategies have been developed in an attempt to generate vaccines with activity against neoplastic cells. These strategies include the use of tumor-associated antigens as immunogens. For example, U.S. Pat. No. 5,993,828, describes a method for producing an immune response against a particular subunit of the Urinary Tumor Associated Antigen by administering to a subject an effective dose of a composition comprising inactivated tumor cells having the Urinary Tumor Associated Antigen on the cell surface and at least one tumor associated antigen selected from the group consisting of GM-2, GD-2, Fetal Antigen and Melanoma Associated Antigen. Accordingly, this patent describes using whole, inactivated tumor cells as the immunogen in an anticancer vaccine.

Another strategy used with anticancer vaccines involves administering a composition containing isolated tumor antigens. In one approach, MAGE-A1 antigenic peptides were used as an immunogen. (See Chaux, P., et al., “Identification of Five MAGE-A1 Epitopes Recognized by Cytolytic T Lymphocytes Obtained by In Vitro Stimulation with Dendritic Cells Transduced with MAGE-A1,” J. Immunol., 163(5):2928-2936 (1999)). There have been several therapeutic trials using MAGE-A1 peptides for vaccination, although the effectiveness of the vaccination regimes was limited. The results of some of these trials are discussed in Vose, J. M., “Tumor Antigens Recognized by T Lymphocytes,” 10^{th} European Cancer Conference, Day 2, Sept. 14, 1999.

In another example of tumor associated antigens used as vaccines, Scheinberg, et al. treated 12 chronic myelogenous leukemia (CML) patients already receiving interferon (IFN) or hydroxyurea with 5 injections of class I-associated bcr-abl peptides with a helper peptide plus the adjuvant QS-21. Scheinberg, D. A., et al, “BCR-ABL Breakpoint Derived Oncogene Fusion Peptide Vaccines Generate Specific Immune Responses in Patients with Chronic Myelogenous Leukemia (CML) [Abstract 1665], American Society of Clinical Oncology 35^{h} Annual Meeting, Atlanta (1999). Proliferative and delayed type hypersensitivity (DTH) T cell responses indicative of T-helper activity were elicited, but no cytolytic killer T cell activity was observed within the fresh blood samples.

Additional examples of attempts to identify TuAAs for use as vaccines are seen in the recent work of Cebon, et al. and Scheibenbogen, et al. Cebon, et al. immunized patients with metastatic melanoma using intradermallly administered MART-1₂₆₋₃₅ peptide with IL-12 in increasing doses given either subcutaneously or intravenously. Of the first 15 patients, 1 complete remission, 1 partial remission, and 1 mixed response were noted. Immune assays for T cell generation included DTH, which was seen in patients with or without IL-12. Positive CTL assays were seen in patients with evidence of clinical benefit, but not in patients without tumor regression. Cebon, et al., “Phase I Studies of Immunization with Melan-A and IL-12 in HLA A2+ Positive Patients with Stage III and IV Malignant Melanoma,” [Abstract 1671], American Society of Clinical Oncology 35^{th} Annual Meeting, Atlanta (1999).

Scheibenbogen, et al. immunized 18 patients with 4 HLA class I restricted tyrosinase peptides, 16 with metastatic melanoma and 2 adjuvant patients. Scheibenbogen, et al., “Vaccination with Tyrosinase peptides and GM-CSF in Metastatic Melanoma: a Phase II Trial,” [Abstract 1680], American Society of Clinical Oncology 35^{h} Annual Meeting, Atlanta (1999). Increased CTL activity was observed in 4/15 patients, 2 adjuvant patients, and 2 patients with evidence of tumor regression. As in the trial by Cebon, et al., patients with progressive disease did not show boosted immunity. In spite of the various efforts expended to date to generate efficacious anticancer vaccines, no such composition has yet been developed.

Antiviral Vaccines

Vaccine strategies to protect against viral diseases have had many successes. Perhaps the most notable of these is the progress that has been made against the disease small pox, which has been driven to extinction. The success of the polio vaccine is of a similar magnitude.

Viral vaccines can be grouped into three classifications: live attenuated virus vaccines, such as vaccinia for small pox, the Sabin poliovirus vaccine, and measles mumps and rubella; whole killed or inactivated virus vaccines, such as the Salk poliovirus vaccine, hepatitis A virus vaccine and the typical influenza virus vaccines; and subunit vaccines, such as hepatitis B. Due to their lack of a complete viral genome, subunit vaccines offer a greater degree of safety than those based on whole viruses.

The paradigm of a successful subunit vaccine is the recombinant hepatitis B vaccine based on the viruses envelope protein. Despite much academic interest in pushing the reductionist subunit concept beyond single proteins to individual epitopes, the efforts have yet to bear much fruit. Viral vaccine research has also concentrated on the induction of an antibody response although cellular responses also occur. However, many of the subunit formulations are particularly poor at generating a CTL response.

### SUMMARY OF THE INVENTION

Previous methods of priming professional antigen presenting cells (pAPCs) to display target cell epitopes have relied simply on causing the pAPCs to express target-associated antigens (TAAs), or epitopes of those antigens which are thought to have a high affinity for MHC I molecules. However, the proteasomal processing of such antigens results in presentation of epitopes on the pAPC that do not correspond to the epitopes present on the target cells.

Using the knowledge that an effective cellular immune response requires that pAPCs present the same epitope that is presented by the target cells, the present invention provides epitopes that have a high affinity for MHC I, and that correspond to the processing specificity of the housekeeping proteasome, which is active in peripheral cells. These epitopes thus correspond to those presented on target cells. The use of such epitopes in vaccines can activate the cellular immune response to recognize the correctly processed TAA and can result in removal of target cells that present such epitopes. In some embodiments, the housekeeping epitopes provided herein can be used in combination with immune epitopes, generating a cellular immune response that is competent to attack target cells both before and after interferon induction. In other embodiments the epitopes are useful in the diagnosis and monitoring of the target-associated disease and in the generation of immunological reagents for such purposes.

Embodiments of the invention relate to isolated epitopes, and antigens or polypeptides that comprise the epitopes. Preferred embodiments include an epitope or antigen having the sequence as disclosed in Table 1. Other embodiments can include an epitope cluster comprising a polypeptide from Table 1. Further, embodiments include a polypeptide having substantial similarity to the already mentioned epitopes, polypeptides, antigens, or clusters. Other preferred embodiments include a polypeptide having functional similarity to any of the above. Still further embodiments relate to a nucleic acid encoding the polypeptide of any of the epitopes, clusters, antigens, and polypeptides from Table 1 and mentioned herein. For purposes of the following summary, discussions of other embodiments of the invention, when making reference to “the epitope,” or “the epitopes” may refer without limitation to all of the foregoing forms of the epitope.

The epitope can be immunologically active. The polypeptide comprising the epitope can be less than about 30 amino acids in length, more preferably, the polypeptide is 8 to 10 amino acids in length, for example. Substantial or functional similarity can include addition of at least one amino acid, for example, and the at least one additional amino acid can be at an N-terminus of the polypeptide. The substantial or functional similarity can include a substitution of at least one amino acid.

The epitope, cluster, or polypeptide comprising the same can have affinity to an HLA-A2 molecule. The affinity can be determined by an assay of binding, by an assay of restriction of epitope recognition, by a prediction algorithm, and the like. The epitope, cluster, or polypeptide comprising the same can have affinity to an HLA-B7, HLA-B51 molecule, and the like.

In preferred embodiments the polypeptide can be a housekeeping epitope. The epitope or polypeptide can correspond to an epitope displayed on a tumor cell, to an epitope displayed on a neovasculature cell, and the like. The epitope or polypeptide can be an immune epitope. The epitope, cluster and/or polypeptide can be a nucleic acid.

Other embodiments relate to pharmaceutical compositions comprising the polypeptides, including an epitope from Table 1, a cluster, or a polypeptide comprising the same, and a pharmaceutically acceptable adjuvant, carrier, diluent, excipient, and the like. The adjuvant can be a polynucleotide. The polynucleotide can include a dinucleotide, which can be CpG, for example. The adjuvant can be encoded by a polynucleotide. The adjuvant can be a cytokine and the cytokine can be, for example, GM-CSF.

The pharmaceutical compositions can further include a professional antigen-presenting cell (pAPC). The pAPC can be a dendritic cell, for example. The pharmaceutical composition can further include a second epitope. The second epitope can be a polypeptide, a nucleic acid, a housekeeping epitope, an immune epitope, and the like.

Still further embodiments relate to pharmaceutical compositions that include any of the nucleic acids discussed herein, including those that encode polypeptides that comprise epitopes or antigens from Table 1. Such compositions can include a pharmaceutically acceptable adjuvant, carrier, diluent, excipient, and the like.

Other embodiments relate to recombinant constructs that include such a nucleic acid as described herein, including those that encode polypeptides that comprise epitopes or antigens from Table 1. The constructs can further include a plasmid, a viral vector, an artificial chromosome, and the like. The construct can further include a sequence encoding at least one feature, such as for example, a second epitope, an IRES, an ISS, an NIS, a ubiquitin, and the like.

Further embodiments relate to purified antibodies that specifically bind to at least one of the epitopes in Table 1. Other embodiments relate to purified antibodies that specifically bind to a peptide-MHC protein complex comprising an epitope disclosed in Table 1 or any other suitable epitope. The antibody from any embodiment can be a monoclonal antibody or a polyclonal antibody.

Still other embodiments relate to multimeric MHC-peptide complexes that include an epitope, such as, for example, an epitope disclosed in Table 1. Also, contemplated are antibodies specific for the complexes.

Embodiments relate to isolated T cells expressing a T cell receptor specific for an MHC-peptide complex. The complex can include an epitope, such as, for example, an epitope disclosed in Table 1. The T cell can be produced by an in vitro immunization and can be isolated from an immunized animal. Embodiments relate to T cell clones, including cloned T cells, such as those discussed above. Embodiments also relate to polyclonal population of T cells. Such populations can include a T cell, as described above, for example.

Still further embodiments relate to pharmaceutical compositions that include a T cell, such as those described above, for example, and a pharmaceutically acceptable adjuvant, carrier, diluent, excipient, and the like.

Embodiments of the invention relate to isolated protein molecules comprising the binding domain of a T cell receptor specific for an MHC-peptide complex. The complex can include an epitope as disclosed in Table 1. The protein can be multivalent. Other embodiments relate to isolated nucleic acids encoding such proteins. Still further embodiments relate to recombinant constructs that include such nucleic acids.

Other embodiments of the invention relate to host cells expressing a recombinant construct as described herein, including constructs encoding an epitope, cluster or polypeptide comprising the same, disclosed in Table 1, for example. The host cell can be a dendritic cell, macrophage, tumor cell, tumor-derived cell, a bacterium, fungus, protozoan, and the like. Embodiments also relate to pharmaceutical compositions that include a host cell, such as those discussed herein, and a pharmaceutically acceptable adjuvant, carrier, diluent, excipient, and the like.

Still other embodiments relate to vaccines or immunotherapeutic compositions that include at least one component, such as, for example, an epitope disclosed in Table 1 or otherwise described herein; a cluster that includes such an epitope, an antigen or polypeptide that includes such an epitope; a composition as described above and herein; a construct as described above and herein, a T cell, or a host cell as described above and herein.

Further embodiments relate to methods of treating an animal. The methods can include administering to an animal a pharmaceutical composition, such as, a vaccine or immunotherapeutic composition, including those disclosed above and herein. The administering step can include a mode of delivery, such as, for example, transdermal, intranodal, perinodal, oral, intravenous, intradermal, intramuscular, intraperitoneal, mucosal, aerosol inhalation, instillation, and the like. The method can further include a step of assaying to determine a characteristic indicative of a state of a target cell or target cells. The method can include a first assaying step and a second assaying step, wherein the first assaying step precedes the administering step, and wherein the second assaying step follows the administering step. The method can further include a step of comparing the characteristic determined in the first assaying step with the characteristic determined in the second assaying step to obtain a result. The result can be for example, evidence of an immune response, a diminution in number of target cells, a loss of mass or size of a tumor comprising target cells, a decrease in number or concentration of an intracellular parasite infecting target cells, and the like.

Embodiments relate to methods of evaluating immunogenicity of a vaccine or immunotherapeutic composition. The methods can include administering to an animal a vaccine or immunotherapeutic, such as those described above and elsewhere herein, and evaluating immunogenicity based on a characteristic of the animal. The animal can be HLA-transgenic.

Other embodiments relate to methods of evaluating immunogenicity that include in vitro stimulation of a T cell with the vaccine or immunotherapeutic composition, such as those described above and elsewhere herein, and evaluating immunogenicity based on a characteristic of the T cell. The stimulation can be a primary stimulation.

Still further embodiments relate to methods of making a passive/adoptive immunotherapeutic. The methods can include combining a T cell or a host cell, such as those described above and elsewhere herein, with a pharmaceutically acceptable adjuvant, carrier, diluent, excipient, and the like.

Other embodiments relate to methods of determining specific T cell frequency, and can include the step of contacting T cells with a MHC-peptide complex comprising an epitope disclosed in Table 1, or a complex comprising a cluster or antigen comprising such an epitope. The contacting step can include at least one feature, such as, for example, immunization, restimulation, detection, enumeration, and the like. The method can further include ELISPOT analysis, limiting dilution analysis, flow cytometry, in situ hybridization, the polymerase chain reaction, any combination thereof, and the like.

Embodiments relate to methods of evaluating immunologic response. The methods can include the above-described methods of determining specific T cell frequency carried out prior to and subsequent to an immunization step.

Other embodiments relate to methods of evaluating immunologic response. The methods can include determining frequency, cytokine production, or cytolytic activity of T cells, prior to and subsequent to a step of stimulation with MHC-peptide complexes comprising an epitope, such as, for example an epitope from Table 1, a cluster or a polypeptide comprising such an epitope.

Further embodiments relate to methods of diagnosing a disease. The methods can include contacting a subject tissue with at least one component, including, for example, a T cell, a host cell, an antibody, a protein, including those described above and elsewhere herein; and diagnosing the disease based on a characteristic of the tissue or of the component. The contacting step can take place in vivo or in vitro, for example.

Still other embodiments relate to methods of making a vaccine. The methods can include combining at least one component, an epitope, a composition, a construct, a T cell, a host cell; including any of those described above and elsewhere herein, with a pharmaceutically acceptable adjuvant, carrier, diluent, excipient, and the like.

Embodiments relate to computer readable media having recorded thereon the sequence of any one of SEQ ID NOS: 1-602, in a machine having a hardware or software that calculates the physical, biochemical, immunologic, molecular genetic properties of a molecule embodying said sequence, and the like.

Still other embodiments relate to methods of treating an animal. The methods can include combining the method of treating an animal that includes administering to the animal a vaccine or immunotherapeutic composition, such as described above and elsewhere herein, combined with at least one mode of treatment, including, for example, radiation therapy, chemotherapy, biochemotherapy, surgery, and the like.

Further embodiments relate to isolated polypeptides that include an epitope cluster. In preferred embodiments the cluster can be from a target-associated antigen having the sequence as disclosed in any one of Tables 25-44, wherein the amino acid sequence includes not more than about 80% of the amino acid sequence of the antigen. Other embodiments relate to vaccines or immunotherapeutic products that include an isolated peptide as described above and elsewhere herein. Still other embodiments relate to isolated polynucleotides encoding a polypeptide as described above and elsewhere herein. Other embodiments relate vaccines or immunotherapeutic products that include these polynucleotides. The polynucleotide can be DNA, RNA, and the like.

Still further embodiments relate to kits comprising a delivery device and any of the embodiments mentioned above and elsewhere herein. The delivery device can be a catheter, a syringe, an internal or external pump, a reservoir, an inhaler, microinjector, a patch, and any other like device suitable for any route of delivery. As mentioned, the kit, in addition to the delivery device also includes any of the embodiments disclosed herein. For example, without limitations, the kit can include an isolated epitope, a polypeptide, a cluster, a nucleic acid, an antigen, a pharmaceutical composition that includes any of the foregoing, an antibody, a T cell, a T cell receptor, an epitope-MHC complex, a vaccine, an immunotherapeutic, and the like. The kit can also include items such as detailed instructions for use and any other like item.

### BRIEF DESCRIPTION OF THE DRAWINGS

FIG. 1 is a sequence alignment of NY-ESO-1 and several similar protein sequences.

FIG. 2 graphically represents a plasmid vaccine backbone useful for delivering nucleic acid-encoded epitopes.

FIGS. 3A and 3B are FACS profiles showing results of HLA-A2 binding assays for tyrosinase₂₀₇₋₂₁₅ and tyrosinase₂₀₈₋₂₁₆.

FIG. 3C shows cytolytic activity against a tyrosinase epitope by human CTL induced by in vitro immunization.

FIG. 4 is a T=120 min. time point mass spectrum of the fragments produced by proteasomal cleavage of SSX-2₃₁₋₆₈.

FIG. 5 shows a binding curve for HLA-A2:SSX-2₄₁₋₄₉ with controls.

FIG. 6 shows specific lysis of SSX-2₄₁₋₄₉-pulsed targets by CTL from SSX-2₄₁₋₄₉-immunized HLA-A2 transgenic mice.

FIG. 7A, B, and C show results of N-terminal pool sequencing of a T=60 min. time point aliquot of the PSMA₁₆₃₋₁₉₂ proteasomal digest.

FIG. 8 shows binding curves for HLA-A2:PSMA₁₆₈₋₁₇₇ and HLA-A2:PSMA₂₈₈₋₂₉₇ with controls.

FIG. 9 shows results of N-terminal pool sequencing of a T=60 min. time point aliquot of the PSMA₂₈₁₋₃₁₀ proteasomal digest.

FIG. 10 shows binding curves for HLA-A2:PSMA₄₆₁₋₄₆₉, HLA-A2:PSMA₄₆₀₋₄₆₉, and HLA-A2:PSMA₆₆₃₋₆₇₁, with controls.

FIG. 11 shows the results of a γ-IFN-based ELISPOT assay detecting PSMA₄₆₃₋₄₇₁-reactive HLA-A1⁺ CD8⁺ T cells.

FIG. 12 shows blocking of reactivity of the T cells used in FIG. 10 by anti-HLA-A1 mAb, demonstrating HLA-A1-restricted recognition.

FIG. 13 shows a binding curve for HLA-A2:PSMA₆₆₃₋₆₇₁, with controls.

FIG. 14 shows a binding curve for HLA-A2:PSMA₆₆₂₋₆₇₁, with controls.

FIG. 15. Comparison of anti-peptide CTL responses following immunization with various doses of DNA by different routes of injection.

FIG. 16. Growth of transplanted gp33 expressing tumor in mice immunized by i.ln. injection of gp33 epitope-expressing, or control, plasmid.

FIG. 17. Amount of plasmid DNA detected by real-time PCR in injected or draining lymph nodes at various times after i.ln. of i.m. injection, respectively.

### DETAILED DESCRIPTION OF THE PREFERRED EMBODIMENT

Definitions

Unless otherwise clear from the context of the use of a term herein, the following listed terms shall generally have the indicated meanings for purposes of this description.

PROFESSIONAL ANTIGEN-PRESENTING CELL (pAPC)—a cell that possesses T cell costimulatory molecules and is able to induce a T cell response. Well characterized pAPCs include dendritic cells, B cells, and macrophages.

PERIPHERAL CELL—a cell that is not a pAPC.

HOUSEKEEPING PROTEASOME—a proteasome normally active in peripheral cells, and generally not present or not strongly active in pAPCs.

IMMUNE PROTEASOME—a proteasome normally active in pAPCs; the immune proteasome is also active in some peripheral cells in infected tissues.

EPITOPE—a molecule or substance capable of stimulating an immune response. In preferred embodiments, epitopes according to this definition include but are not necessarily limited to a polypeptide and a nucleic acid encoding a polypeptide, wherein the polypeptide is capable of stimulating an immune response. In other preferred embodiments, epitopes according to this definition include but are not necessarily limited to peptides presented on the surface of cells, the peptides being non-covalently bound to the binding cleft of class I MHC, such that they can interact with T cell receptors.

MHC EPITOPE—a polypeptide having a known or predicted binding affinity for a mammalian class I or class II major histocompatibility complex (MHC) molecule.

HOUSEKEEPING EPITOPE—In a preferred embodiment, a housekeeping epitope is defined as a polypeptide fragment that is an MHC epitope, and that is displayed on a cell in which housekeeping proteasomes are predominantly active. In another preferred embodiment, a housekeeping epitope is defined as a polypeptide containing a housekeeping epitope according to the foregoing definition, that is flanked by one to several additional amino acids. In another preferred embodiment, a housekeeping epitope is defined as a nucleic acid that encodes a housekeeping epitope according to the foregoing definitions.

IMMUNE EPITOPE—In a preferred embodiment, an immune epitope is defined as a polypeptide fragment that is an MHC epitope, and that is displayed on a cell in which immune proteasomes are predominantly active. In another preferred embodiment, an immune epitope is defined as a polypeptide containing an immune epitope according to the foregoing definition, that is flanked by one to several additional amino acids. In another preferred embodiment, an immune epitope is defined as a polypeptide including an epitope cluster sequence, having at least two polypeptide sequences having a known or predicted affinity for a class I MHC. In yet another preferred embodiment, an immune epitope is defined as a nucleic acid that encodes an immune epitope according to any of the foregoing definitions.

TARGET CELL—a cell to be targeted by the vaccines and methods of the invention. Examples of target cells according to this definition include but are not necessarily limited to: a neoplastic cell and a cell harboring an intracellular parasite, such as, for example, a virus, a bacterium, or a protozoan.

TARGET-ASSOCIATED ANTIGEN (TAA)—a protein or polypeptide present in a target cell.

TUMOR-ASSOCIATED ANTIGENS (TuAA)—a TAA, wherein the target cell is a neoplastic cell.

HLA EPITOPE—a polypeptide having a known or predicted binding affinity for a human class I or class II HLA complex molecule.

ANTIBODY—a natural immunoglobulin (Ig), poly- or monoclonal, or any molecule composed in whole or in part of an Ig binding domain, whether derived biochemically or by use of recombinant DNA. Examples include inter alia, F(ab), single chain Fv, and Ig variable region-phage coat protein fusions.

ENCODE—an open-ended term such that a nucleic acid encoding a particular amino acid sequence can consist of codons specifying that (poly)peptide, but can also comprise additional sequences either translatable, or for the control of transcription, translation, or replication, or to facilitate manipulation of some host nucleic acid construct.

SUBSTANTIAL SIMILARITY—this term is used to refer to sequences that differ from a reference sequence in an inconsequential way as judged by examination of the sequence. Nucleic acid sequences encoding the same amino acid sequence are substantially similar despite differences in degenerate positions or modest differences in length or composition of any non-coding regions. Amino acid sequences differing only by conservative substitution or minor length variations are substantially similar. Additionally, amino acid sequences comprising housekeeping epitopes that differ in the number of N-terminal flanking residues, or immune epitopes and epitope clusters that differ in the number of flanking residues at either terminus, are substantially similar. Nucleic acids that encode substantially similar amino acid sequences are themselves also substantially similar.

FUNCTIONAL SIMILARITY—this term is used to refer to sequences that differ from a reference sequence in an inconsequential way as judged by examination of a biological or biochemical property, although the sequences may not be substantially similar. For example, two nucleic acids can be useful as hybridization probes for the same sequence but encode differing amino acid sequences. Two peptides that induce cross-reactive CTL responses are functionally similar even if they differ by non-conservative amino acid substitutions (and thus do not meet the substantial similarity definition). Pairs of antibodies, or TCRs, that recognize the same epitope can be functionally similar to each other despite whatever structural differences exist. In testing for functional similarity of immunogenicity one would generally immunize with the “altered” antigen and test the ability of the elicited response (Ab, CTL, cytokine production, etc.) to recognize the target antigen. Accordingly, two sequences may be designed to differ in certain respects while retaining the same function. Such designed sequence variants are among the embodiments of the present invention.

TABLE 1A
SEQ ID NOS.* including epitopes in Examples 1-7,
13.

| SEQ | | |
|---|---|---|
| ID | | |
| NO | ENTITY | SEQUENCE |
| 1 | Tyr 207-216 | FLPWHRLFLL |
| 2 | Tyrosinase | Accession number**: P14679 |
| | protein | |
| 3 | SSX-2 protein | Accession number: NP_003138 |
| 4 | PSMA protein | Accession number: NP_004467 |
| 5 | Tyrosinase cDNA | Accession number: NM_000372 |
| 6 | SSX-2 cDNA | Accession number: NM_003147 |
| 7 | PSMA cDNA | Accession number: NM_004476 |
| 8 | Tyr 207-215 | LPWHRLFL |
| 9 | Tyr 208-216 | LPWHRLFLL |
| 10 | SSX-2 31-68 | YFSKEEWEKMKASEKIFYVYMKRKYEAMT |
| | | KLGFKATLP |
| 11 | SSX-2 32-40 | FSKEEWEKM |
| 12 | SSX-2 39-47 | KMKASEKIF |
| 13 | SSX-2 40-48 | MKASEKIFY |
| 14 | SSX-2 39-48 | KMKASEKIFY |
| 15 | SSX-2 41-49 | KASEKIFYV |
| 16 | SSX-2 40-49 | MKASEKLFYV |
| 17 | SSX-2 41-50 | KASEKIFYVY |
| 18 | SSX-2 42-49 | ASEKIFYVY |
| 19 | SSX-2 53-61 | RYEAMTKL |
| 20 | SSX-2 52-61 | KRYEAMTKL |
| 21 | SSX-2 54-63 | KYEAMTKLGF |
| 22 | SSX-2 55-63 | YEAMTKLGF |
| 23 | SSX-2 56-63 | EAMTKLGF |
| 24 | HBV18-27 | FLPSDYFPSV |
| 25 | HLA-B44 binder | AEMGKYSFY |
| 26 | SSX-141-49 | KYSEKJSYV |
| 27 | SSX-341-49 | KVSEKJVYV |
| 28 | SSX-441-49 | KSSEKIVYV |
| 29 | SSX-541-49 | KASEKIIYV |
| 30 | PSMA163-192 | AFSPQGMPEGDLVYVNYARTEDFFKLERD |

M

| | | |
|---|---|---|
| 31 | PSMA 168-190 | GMPEGDLVYVNYARTEDFFKLER |
| 32 | PSMA 169-177 | MPEGDLVYV |
| 33 | PSMA 168-177 | GMPEGDLVYV |
| 34 | PSMA 168-176 | GMPEGDLVY |
| 35 | PSMA 167-176 | QGMPEGDLVY |
| 36 | PSMA 169-176 | MPEGDLVY |
| 37 | PSMA 171-179 | EGDLVYVNY |
| 38 | PSMA 170-179 | PEGDLVYVNY |
| 39 | PSMA 174-183 | LVYVNYARTE |
| 40 | PSMA 177-185 | VNYARTEDF |
| 41 | PSMA 176-185 | YVNYARTEDF |
| 42 | PSMA 178-186 | NYARTEDFF |
| 43 | PSMA 179-186 | YARTEDFF |
| 44 | PSMA 181-189 | RTEDFFKLE |
| 45 | PSMA 281-310 | RGIAEAVGLPSIPVHPIGYYDAQKLLEKM |

G

| | | |
|---|---|---|
| 46 | PSMA 283-307 | IAEAVGLPSIPVHPIGYYDAQKLLE |
| 47 | PSMA 289-297 | LPSIPVHPI |
| 48 | PSMA 288-297 | GLPSIPVHPI |
| 49 | PSMA 297-305 | IGYYDAQKL |
| 50 | PSMA 296-305 | PIGYYDAQKL |
| 51 | PSMA 291-299 | SIPVHPIGY |
| 52 | PSMA 290-299 | PSIPVHPIGY |
| 53 | PSMA 292-299 | IPVHPIGY |
| 54 | PSMA 299-307 | YYDAQKLLE |
| 55 | PSMA 454-481 | SSIEGNYTLRVDCTPLMYSLVHLTKEL |
| 56 | PSMA 456-464 | IEGNYTLRV |
| 57 | PSMA 455-464 | SIEGNYTLRV |
| 58 | PSMA 457-464 | EGNYTLRV |
| 59 | PSMA 461-469 | TLRVDCTPL |
| 60 | PSMA 460-469 | YTLRVDCTPL |
| 61 | PSMA 462-470 | LRVDCTPLM |
| 62 | PSMA 463-471 | RVDCTPLMY |
| 63 | PSMA 462-471 | LRVDCTPLMY |
| 64 | PSMA 653-687 | FDKSNPIVLRMMNDQLMFLERAFIDPLGL |

PDRPFY

| | | |
|---|---|---|
| 65 | PSMA 660-681 | VLRMMNDQLMFLERAFIIDPLGL |
| 66 | PSMA 663-671 | MMNDQLMFL |
| 67 | PSMA 662-671 | RMMNDQLMFL |
| 68 | PSMA 662-670 | RMMNDQLMF |
| 69 | Tyr 1-17 | MLLAVLYCLLWSFQTSA |

TABLE 1B
SEQ ID NOS.* including epitopes in Examples 14 and
15.

| SEQ | | |
|---|---|---|
| ID | | |
| NO | ENTITY | SEQUENCE |
| 70 | GP100 protein² | **Accession number: P40967 |
| 71 | MAGE-1 protein | Accession number: P43355 |
| 72 | MAGE-2 protein | Accession number: P43356 |
| 73 | MAGE-3 protein | Accession number: P43357 |
| 74 | NY-ESO-1 protein | Accession number: P78358 |
| 75 | LAGE-1a protein | Accession number: CAA11116 |
| 76 | LAGE-1b protein | Accession number: CAA11117 |
| 77 | PRAME protein | Accession number: NP 006106 |
| 78 | PSA protein | Accession number: P07288 |
| 79 | PSCA protein | Accession number: O43653 |
| 80 | GP100 cds | Accession number: U20093 |
| 81 | MAGE-1 cds | Accession number: M77481 |
| 82 | MAGE-2 cds | Accession number: L18920 |
| 83 | MAGE-3 cds | Accession number: U03735 |
| 84 | NY-ESO-1 cDNA | Accession number: U87459 |
| 85 | PRAME cDNA | Accession number: NM_006115 |
| 86 | PSA cDNA | Accession number: NM_001648 |
| 87 | PSCA cDNA | Accession number: AE043498 |
| 88 | GP100 630-638 | LPHSSSHWL |
| 89 | GP100 629-638 | QLPHSSSHWL |
| 90 | GP100 614-622 | LIYRRRLMK |
| 91 | GP100 613-622 | SLIYRRRLMK |
| 92 | GP100 615-622 | TYRRRLMK |
| 93 | GP100 630-638 | LPHSSSHWL |
| 94 | GP100 629-638 | QLPHSSSHWL |
| 95 | MAGE-1 95-102 ESLFRAVI | |
| 96 | MAGE-1 93-102 ILESLFRAVI | |
| 97 | MAGE-1 93-101 | ILESLFRAV |
| 98 | MAGE-1 92-101 | CILESLFRAV |
| 99 | MAGE-1 92-100 | CILESLFRA |
| 100 | MAGE-1 263-271 | EFLWGPRAL |
| 101 | MAGE-1 264-271 | FLWGPRAL |
| 102 | MAGE-1 264-273 | FLWGPRALAE |
| 103 | MAGE-1 265-274 | LWGPRALAET |
| 104 | MAGE-1 268-276 | PRALAETSY |
| 105 | MAGE-1 267-276 | GPRALAETSY |
| 106 | MAGE-1 269-277 | RALAETSYV |
| 107 | MAGE-1 271-279 | LAETSYVKV |
| 108 | MAGE-1 270-279 | ALAETSYVKV |
| 109 | MAGE-1 272-280 | AETSYVKVL |
| 110 | MAGE-1 271-280 | LAETSYVKVL |
| 111 | MAGE-1 274-282 | TSYVKVLEY |
| 112 | MAGE-1 273-282 | ETSYVKVLEY |
| 113 | MAGE-1 278-286 | KVLEYVIKV |
| 114 | MAGE-1 168-177 | SYVLVTCLGL |
| 115 | MAGE-1 169-177 | YLVTCLGL |
| 116 | MAGE-1 170-177 | VLVTCLGL |
| 117 | MAGE-1 240-248 | TQDLVQEKY |
| 118 | MAGE-1 239-248 | LTQDLVQEKY |
| 119 | MAGE-1 232-240 | YGEPRKLLT |
| 120 | MAGE-1 243-251 | LVQEKYLEY |
| 121 | MAGE-1 242-251 | DLVQEKYLEY |
| 122 | MAGE-1 230-238 | SAYGEPRKL |
| 123 | MAGE-1 278-286 | KVLEYVIKV |
| 124 | MAGE-1 277-286 | VKVLEYVIKV |
| 125 | MAGE-1 276-284 | YVKVLEYVI |
| 126 | MAGE-1 274-282 | TSYVKVLEY |
| 127 | MAGE-1 273-282 | ETSYVKVLEY |
| 128 | MAGE-1 283-291 | VIKVSARVR |
| 129 | MAGE-1 282-291 | YVIKVSARVR |
| 130 | MAGE-2 115-122 | ELVHFLLL |
| 131 | MAGE-2 113-122 | MVELVHFLLL |
| 132 | MAGE-2 109-116 | ISRKMVEL |
| 133 | MAGE-2 108-116 | AISRKMVEL |
| 134 | MAGE-2 107-116 | AAISRKMVEL |
| 135 | MAGE-2 112-120 | KMVELVHIFL |
| 136 | MAGE-2 109-117 | ISRKMVELV |
| 137 | MAGE-2 108-117 | AISRKMVELV |
| 138 | MAGE-2 116-124 | LVHFLLLKY |
| 139 | MAGE-2 115-124 | ELVHFLLLKY |
| 140 | MAGE-2 111-119 | RKMVELVHF |
| 141 | MAGE-2 158-166 | LQLVFGIEV |
| 142 | MAGE-2 157-166 | YLQLVFGIEV |
| 143 | MAGE-2 159-167 | QLVFGIEVV |
| 144 | MAGE-2 158-167 | LQLVFGIEVV |
| 145 | MAGE-2 164-172 | IEVVEVVPI |
| 146 | MAGE-2 163-172 | GIEVVEVVPI |
| 147 | MAGE-2 162-170 | FGIEVVEVV |
| 148 | MAGE-2 154-162 | ASEYLQLVF |
| 149 | MAGE-2 153-162 | KASEYLQLVF |
| 150 | MAGE-2 218-225 | EEKIWEEL |
| 151 | MAGE-2 216-225 | APEEKIWEEL |
| 152 | MAGE-2 216-223 | APEEKIWE |
| 153 | MAGE-2 220-228 | KILWEELSML |
| 154 | MAGE-2 219-228 | EKIWEELSML |
| 155 | MAGE-2 271-278 | FLWGPRAL |
| 156 | MAGE-2 271-279 | FLWGPRALI |
| 157 | MAGE-2 278-286 | LIETSYVKV |
| 158 | MAGE-2 277-286 | ALIETSYVKV |
| 159 | MAGE-2 276-284 | RALIETSYV |
| 160 | MAGE-2 279-287 | IETSYVKVL |
| 161 | MAGE-2 278-287 | LIETSYVKVL |
| 162 | MAGE-3 271-278 | FLWGPRAL |
| 163 | MAGE-3 270-278 | EFLWGPRAL |
| 164 | MAGE-3 271-279 | FLWGPRALV |
| 165 | MAGE-3 276-284 | RALVETSYV |
| 166 | MAGE-3 272-280 | LWGPRALVE |
| 167 | MAGE-3 271-280 | FLWGPRALVE |
| 168 | MAGE-3 272-281 | LWGPRALVET |
| 169 | NY-ESO-1 82-90 | GPESRLLEF |
| 170 | NY-ESO-1 83-91 | PESRLLEFY |
| 171 | NY-ESO-1 82-91 | GPESRLLEFY |
| 172 | NY-ESO-1 84-92 | ESRLLEFYL |
| 173 | NY-ESO-1 86-94 | RLLEFYLAM |
| 174 | NY-ESO-1 88-96 | LEFYLAMPF |
| 175 | NY-ESO-1 87-96 | LLEFYLAMPF |
| 176 | NY-ESO-1 93-102 | AMPFATPMEA |
| 177 | NY-ESO-1 94-102 | MPFATPMEA |
| 178 | NY-ESO-1 115-123 | PLPVPGVLL |
| 179 | NY-ESO-1 114-123 | PPLPVPGVLL |
| 180 | NY-ESO-1 116-123 | LPVPGVLL |
| 181 | NY-ESO-1 103-112 | ELARRSLAQD |
| 182 | NY-ESO-1 118-126 | VPGVLLKEF |
| 183 | NY-ESO-1 117-126 | PVPGVLLKEF |
| 184 | NY-ESO-1 116-123 | LPVPGVLL |
| 185 | NY-ESO-1 127-135 | TVSGNILTI |
| 186 | NY-ESO-1 126-135 | FTVSGNILTI |
| 187 | NY-ESO-1 120-128 | GVLLKEFTV |
| 188 | NY-ESO-1 121-130 | VLLKEFTVSG |
| 189 | NY-ESO-1 122-130 | LLKEFTVSG |
| 190 | NY-ESO-1 118-126 | VPGVLLKEF |
| 191 | NY-ESO-1 117-126 | PVPGVLLKEF |
| 192 | NY-ESO-1 139-147 | AADHRQLQL |
| 193 | NY-ESO-1 148-156 | SISSCLQQL |
| 194 | NY-ESO-1 147-156 | LSISSCLQQL |
| 195 | NY-ESO-1 138-147 | TAADHRQLQL |
| 196 | NY-ESO-1 161-169 | WITQCFLPV |
| 197 | NY-ESO-1 157-165 | SLLMW1TQC |
| 198 | NY-ESO-1 150-158 | SSCLQQLSL |
| 199 | NY-ESO-1 154-162 | QQLSLLMWI |
| 200 | NY-ESO-1 151-159 | SCLQQLSLL |
| 201 | NY-ESO-1 150-159 | SSGLQQLSLL |
| 202 | NY-ESO-1 163-171 | TQCFLPVFL |
| 203 | NY-ESO-1 162-171 | ITQCFLPVFL |
| 204 | PRAME 219-227 | PMQDII(MIL |
| 205 | PRAME 218-227 | MPMQDIKMIL |
| 206 | PRAME 428-436 | QHLIGLSNL |
| 207 | PRAME 427-436 | LQHLIGLSNL |
| 208 | PRAME 429-436 | HLIGLSNL |
| 209 | PRAME 431-439 | IGLSNLTHV |
| 210 | PRAME 43O-439 | LIGLSNLTHV |
| 211 | PSA 53-61 | VLVHPQWVL |
| 212 | PSA 52-61 | GVLVHPQWVL |
| 213 | PSA 52-60 | GVLVHPQWV |
| 214 | PSA 59-67 | WVLTAAHCI |
| 215 | PSA 54-63 | LVHPQWVLTA |
| 216 | PSA 53-62 | VLVHPQWVLT |
| 217 | PSA 54-62 | LVHPQWVLT |
| 218 | PSA 66-73 | CIRNKSVI |
| 219 | PSA 65-73 | HCIRNKSVI |
| 220 | PSA 56-64 | HPQWVLTAA |
| 221 | PSA 63-72 | AAHCIRNKSV |
| 222 | PSCA 116-123 | LLWGPGQL |
| 223 | PSCA 115-123 | LLLWGPGQL |
| 224 | PSCA 114-123 | GLLLWGPGQL |
| 225 | PSCA 99-107 | ALQPAAATL |
| 226 | PSCA 98-107 | ALQPAAAIL |
| 227 | Tyr 128-137 | APEKDKFFAY |
| 228 | Tyr 129-137 | PEKDKFFAY |
| 229 | Tyr 130-138 | EKDKFFAYL |
| 230 | Tyr 131-138 | KDKFFAYL |
| 231 | Tyr 205-213 | PAFLPWHRL |
| 232 | Tyr 204-213 | APAFLPWHRL |
| 233 | Tyr 214-223 | FLLRWEQEIQ |
| 234 | Tyr 212-220 | RLFLLRWEQ |
| 235 | Tyr 191-200 | GSEIWRDIDF |
| 236 | Tyr 192-200 | SEIWRDIDF |
| 237 | Tyr 473-481 | RIWSWLLGA |
| 238 | Tyr 476-484 | SWLLGAAMV |
| 239 | Tyr 477-486 | WLLGAAMVGA |
| 240 | Tyr 478-486 | LLGAAMVGA |
| 241 | PSMA 4-12 | LLHETDSAV |
| 242 | PSMA 13-21 | ATARRPRWL |
| 243 | PSMA 53-61 | TPKHNMKAF |
| 244 | PSMA 64-73 | ELKAENIKKF |
| 245 | PSMA 69-77 | NIKKFLH¹NF |
| 246 | PSMA 68-77 | ENIKKFLH¹NF |
| 247 | PSMA 220-228 | AGAKGVILY |
| 248 | PSMA 468-477 | PLMYSLVHNL |
| 249 | PSMA 469-477 | LMYSLVHNL |
| 250 | PSMA 463-471 | RVDCTPLMY |
| 251 | PSMA 465-473 | DCTPLMYSL |
| 252 | PSMA 507-515 | SGMPRISKL |
| 253 | PSMA 506-515 | FSGMPRISKL |
| 254 | NY-ESO-1 136-163 | RLTAADHRQLQLSJSSCLQQLSLLMWIT |
| 255 | NY-ESO-1 150-177 | SSCLQQLSLLMWITQCFLPVFLAQPPSG |

¹This H was reported as Y in the SWISSPROT database.
²The amino acid at position 274 may be Pro or Leu depending upon the database. The particular analysis presented herein used the Pro.

TABLE 1C
SEQ ID NOS.* including epitopes in Example 14.

| SEQ | | |
|---|---|---|
| ID | | |
| NO. | IDENTITY | SEQUENCE |
| 256 | Mage-1 125-132 | KAEMLESV |
| 257 | Mage-1 124-132 | TKAEMLESV |
| 258 | Mage-1 123-132 | VTKAEMLESV |
| 259 | Mage-1 128-136 | MLESVIKNY |
| 260 | Mage-1 127-136 | EMLESVIKNY |
| 261 | Mage-1 125-133 | KAEMLESVI |
| 262 | Mage-1 146-153 | KASESLQL |
| 263 | Mage-1 145-153 | GKASESLQL |
| 264 | Mage-1 147-155 | ASESLQLVF |
| 265 | Mage-1 153-161 | LVFGTDVKE |
| 266 | Mage-1 114-121 | LLKYRARE |
| 267 | Mage-1 106-113 | VADLVGFL |
| 268 | Mage-1 105-113 | KVADLVGFL |
| 269 | Mage-1 107-115 | ADLVGFLLL |
| 270 | Mage-1 106-115 | VADLVGFLLL |
| 271 | Mage-1 114-123 | LLKYRAREPV |
| 272 | Mage-3 278-286 | LVETSYVKV |
| 273 | Mage-3 277-286 | ALVETSYVKV |
| 274 | Mage-3 285-293 | KVLHHMVKI |
| 275 | Mage-3 283-291 | YVKVLIIIIMV |
| 276 | Mage-3 275-283 | PRALVETSY |
| 277 | Mage-3 274-283 | GPRALVETSY |
| 278 | Mage-3 278-287 | LVETSYVKVL |
| 279 | ED-B 4′-5 | TIIPEVPQL |
| 280 | ED-B 5′-5 | DTIIPEVPQL |
| 281 | ED-B 1-10 | EVPQLTDLSF |
| 282 | ED-B 23-30 | TPLNSSTI |
| 283 | ED-B 18-25 | IGLRWTPL |
| 284 | ED-B 17-25 | SIGLRWTPL |
| 285 | ED-B 25-33 | LNSSTIIGY |
| 286 | ED-B 24-33 | PLNSSTIIGY |
| 287 | ED-B 23-31 | TPLNSSTII |
| 288 | ED-B 31-38 | IGYRITVV |
| 289 | ED-B 30-38 | IIGYRITVV |
| 290 | ED-B 29-38 | TIIGYRJTVV |
| 291 | ED-B 31-39 | IGXTRITVVA |
| 292 | ED-B 30-39 | IIGYRITVVA |
| 293 | CEA 184-191 | SLPVSPRL |
| 294 | CEA 183-191 | QSLPVSPRL |
| 295 | CEA 186-193 | PVSPRLQL |
| 296 | CEA 185-193 | LPVSPRLQL |
| 297 | CEA 184-193 | SLPVSPRLQL |
| 298 | CEA 185-192 | LPVSPRLQ |
| 299 | GEA 192-200 | QLSNGNRTL |
| 300 | CEA 191-200 | LQLSNGNRTL |
| 301 | CEA 179-187 | WVNNQSLPV |
| 302 | GEA 186-194 | PVSPRLQLS |
| 303 | CEA 362-369 | SLPVSPRL |
| 304 | CEA 361-369 | QSLPVSPRL |
| 305 | CEA 364-371 | PVSPRLQL |
| 306 | CEA 363-371 | LPVSPRLQL |
| 307 | CEA 362-371 | SLPVSPRLQL |
| 308 | CEA 363-370 | LPVSPRLQ |
| 309 | CEA 370-378 | QLSNDNRTL |
| 310 | CEA 369-378 | LQLSNDNRTL |
| 311 | CEA 357-365 | WVNNQSLPV |
| 312 | CEA 360-368 | NQSLPVSPR |
| 313 | CEA 540-547 | SLPVSPRL |
| 314 | CEA 539-547 | QSLPVSPRL |
| 315 | CEA 542-549 | PVSPRLQL |
| 316 | CEA 541-549 | LPVSPRLQL |
| 317 | CEA 540-549 | SLPVSPRLQL |
| 318 | CEA 541-548 | LPVSPRLQ |
| 319 | CEA 548-556 | QLSNGNRTL |
| 320 | CEA 547-556 | LQLSNGNRTL |
| 321 | CEA 535-543 | WVNGQSLPV |
| 322 | CEA 533-541 | LWWVNGQSL |
| 323 | CEA 532-541 | YLWWVNGQSL |
| 324 | CEA 538-546 | GQSLPVSPR |
| 325 | Her-2 30-37 | DMKLRLPA |
| 326 | Her-2 28-37 | GTDMKLRLPA |
| 327 | Her-2 42-49 | HLDMLRITL |
| 328 | Her-2 41-49 | THLDMLRJIL |
| 329 | Her-2 40-49 | ETHLDMLRHL |
| 330 | Her-2 36-43 | PASPETHL |
| 331 | Her-2 35-43 | LPASPETHL |
| 332 | Her-2 34-43 | RLPASPETHL |
| 333 | Her-2 38-46 | SPETHLDML |
| 334 | Her-2 37-46 | ASPETHLDML |
| 335 | Her-2 42-50 | HLDMLRIILY |
| 336 | Her-2 41-50 | THLDMLRIILY |
| 337 | Her-2 719-726 | ELRKVKVL |
| 338 | Her-2 718-726 | TELRKVKVL |
| 339 | Her-2 717-726 | ETELRKVKVL |
| 340 | Her-2 715-723 | LKETELRKV |
| 341 | Her-2 714-723 | ILKETELRKiV |
| 342 | Her-2 712-720 | MR1LKETEL |
| 343 | Her-2 711-720 | QMRILKETEL |
| 344 | Her-2 717-725 | ETELRKVKV |
| 345 | Her-2 716-725 | KETELRKVKV |
| 346 | Her-2 706-714 | MPNQAQMRI |
| 347 | Her-2 705-714 | AMPNQAQMRI |
| 348 | Her-2 706-715 | MPNQAQMRIL |
| 349 | HER-2 966-973 | RPRFRELV |
| 350 | HER-2 965-973 | CRPRFRELV |
| 351 | HER-2 968-976 | RFRELVSEF |
| 352 | HER-2 967-976 | PRFRELVSEF |
| 353 | HER-2 964-972 | ECRPRFREL |
| 354 | NY-ESO-1 67-75 | GAASGLNGC |
| 355 | NY-ESO-1 52-60 | RASGPGGGA |
| 356 | NY-ESO-1 64-72 | PHGGAASGL |
| 357 | NY-ESO-1 63-72 | GPHGGAASGL |
| 358 | NY-ESO-1 60-69 | APRGPHGGAA |
| 359 | PRAME 112-119 | VRPRRWKL |
| 360 | PRAME 111-119 | EVRPRRWKL |
| 361 | PRAME 113-121 | RPRRWKLQV |
| 362 | PRAME 114-122 | PRRWKLQVL |
| 363 | PRAME 113-122 | RPRRWKLQVL |
| 364 | PRAME 116-124 | RWKLQVLDL |
| 365 | PRAME 115-124 | RRWKLQVLDL |
| 366 | PRAME 174-182 | PVEVLVDLF |
| 367 | PRAME 199-206 | VKRKKNVL |
| 368 | PRAME 198-206 | KVKRKKNVL |
| 369 | PRAME 197-206 | EKVKRiKKNVL |
| 370 | PRAME 198-205 | KVKRKKNV |
| 371 | PRAME 201-208 | RKKNVLRL |
| 372 | PRAME 200-208 | KRKKNVLRL |
| 373 | PRAME 199-208 | VKRKKNVLRL |
| 374 | PRAME 189-196 | DELESYLI |
| 375 | PRAME 205-213 | VLRLCCKKL |
| 376 | PRAME 204-213 | NVLRLGCKKL |
| 377 | PRAME 194-202 | YLLEKVKRK |
| 378 | PRAME 74-81 | QAWPFTCL |
| 379 | PRAME 73-81 | VQAWPFTCL |
| 380 | PRAME 72-81 | MVQAWPFTCL |
| 381 | PRAME 81-88 | LPLGVLMK |
| 382 | PRAME 80-88 | CLPLGVLMK |
| 383 | PRAME 79-88 | TCLPLGVLMK |
| 384 | PRAME 84-92 | GVLMKGQHL |
| 385 | PRAME 81-89 | LPLGVLMKG |
| 386 | PRAME 80-89 | CLPLGVLMKG |
| 387 | PRAME 76-85 | WPFTCLPLGV |
| 388 | PRAME 51-59 | ELFPPLFMA |
| 389 | PRAME 49-57 | PRELFPPLF |
| 390 | PRAME 48-57 | LPRELFPPLF |
| 391 | PRAME 50-58 | RELFPPLFM |
| 392 | PRAME 49-58 | PRELFPPLFM |
| 393 | PSA 239-246 | RPSLYTKV |
| 394 | PSA 238-246 | ERPSLYTKV |
| 395 | PSA 236-243 | LPERPSLY |
| 396 | PSA 235-243 | ALPERPSLY |
| 397 | PSA 241-249 | SLYTKVVHY |
| 398 | PSA 240-249 | PSLYTKVVHY |
| 399 | PSA 239-247 | RPSLYTKVV |
| 400 | PSMA 211-218 | GNKVKNAQ |
| 401 | PSMA 202-209 | IARYGKVF |
| 402 | PSMA 217-225 | AQLAGAKGV |
| 403 | PSMA 207-215 | KVFRGNKVK |
| 404 | PSMA 211-219 | GNKVKNAQL |
| 405 | PSMA 269-277 | TPGYPANEY |
| 406 | PSMA 268-277 | LTPGYPANEY |
| 407 | PSMA 271-279 | GYPANEYAY |
| 408 | PSMA 270-279 | PGYPANEYAY |
| 409 | PSMA 266-274 | DPLTPGYPA |
| 410 | PSMA 492-500 | SLYESWTKK |
| 411 | PSMA 491-500 | KSLYESWTKK |
| 412 | PSMA 486-494 | EGFEGKSLY |
| 413 | PSMA 485-494 | DEGFEGKSLY |
| 414 | PSMA 498-506 | TKKSPSPEF |
| 415 | PSMA 497-506 | WTKKSPSPEF |
| 416 | PSMA 492-501 | SLYESWTKKS |
| 417 | PSMA 725-732 | WGEVKRQI |
| 418 | PSMA 724-732 | AWGEVKRQI |
| 419 | PSMA 723-732 | KAWGEVKRQI |
| 420 | PSMA 723-730 | KAWGEVKR |
| 421 | PSMA 722-730 | SKAWGEVKR |
| 422 | PSMA 731-739 | QIYVAAIFTV |
| 423 | PSMA 733-741 | YVAAFTVQA |
| 424 | PSMA 725-733 | WGEVKRQIY |
| 425 | PSMA 727-735 | EVKRQ1YVA |
| 426 | PSMA 738-746 | TVQAAAETL |
| 427 | PSMA 737-746 | FTVQAAAETL |
| 428 | PSMA 729-737 | KRQIYVAAF |
| 429 | PSMA 721-729 | PSKAWGEVK |
| 430 | PSMA 723-731 | KAWGEVKRQ |
| 431 | PSMA 100-108 | WKEFGLDSV |
| 432 | PSMA 99-108 | QWKEFGLDSV |
| 433 | PSMA 102-111 | EFGLDSVELA |
| 434 | SCP-1 126-134 | ELRQKESKL |
| 435 | SCP-1 125-134 | AELRQKESKL |
| 436 | SCP-1 133-141 | KLQENRKII |
| 437 | SCP-1 298-305 | QLEEKTKL |
| 438 | SCP-1 297-305 | NQLEEKTKL |
| 439 | SCP-1 288-296 | LLEESRDKV |
| 440 | SCP-1 287-296 | FLLEESRDKV |
| 441 | SCP-1 291-299 | ESRDKVNQL |
| 442 | SCP-1 290-299 | EESRDKVNQL |
| 443 | SCP-1 475-483 | EKBVHIDLEY |
| 444 | SCP-1 474-483 | RFKEVHDLEY |
| 445 | SCP-1 480-488 | DLEYSYCIIY |
| 446 | SCP-1 477-485 | EVHDLEYSY |
| 447 | SCP-1 477-486 | EVUDLEYSYC |
| 448 | SCP-1 502-509 | KLSSKREL |
| 449 | SCP-1 508-515 | ELKNTEYF |
| 450 | SCP-1 507-515 | RELKNTEYF |
| 451 | SCP-1 496-503 | KRGQRPKL |
| 452 | SCP-1 494-503 | LPKRGQRPKL |
| 453 | SCP-1 509-517 | LKNTEYFTL |
| 454 | SCP-1 508-517 | ELKNTEYFTL |
| 455 | SCP-1 506-514 | KRELKNTEY |
| 456 | SCP-1 502-510 | KLSSKRELK |
| 457 | SCP-1 498-506 | GQRPKLSSK |
| 458 | SCP-1 497-506 | RGQRPKLSSK |
| 459 | SCP-1 500-508 | RPKLSSKRE |
| 460 | SCP-1 573-580 | LEYVREEL |
| 461 | SCP-1 572-580 | ELEYVREEL |
| 462 | SCP-1 571-580 | NELEYVREEL |
| 463 | SCP-1 579-587 | ELKQKREDEV |
| 464 | SCP-1 575-583 | YVREELKQK |
| 465 | SCP-1 632-640 | QLNVYEIIKV |
| 466 | SCP-1 630-638 | SKQLNVYEI |
| 467 | SCP-1 628-636 | AESKQLNVY |
| 468 | SCP-1 627-636 | TAESKQLNYY |
| 469 | SCP-1 638-645 | JKVNKLEL |
| 470 | SCP-1 637-645 | EIIKVNKLEL |
| 471 | SCP-1 636-645 | YEIKVNKLEL |
| 472 | SCP-1 642-650 | KLELELESA |
| 473 | SCP-1 635-643 | VYETKVNIKL |
| 474 | SCP-1 634-643 | NVYETKVNKL |
| 475 | SCP-1 646-654 | ELESAKQKF |
| 476 | SCP-1 642-650 | KLELELESA |
| 477 | SCP-1 646-654 | ELESAKiQKF |
| 478 | SCP-1 771-778 | KEKLKREA |
| 479 | SCP-1 777-785 | EAKENTATL |
| 480 | SCP-1 776-785 | REAKENTATL |
| 481 | SCP-1 773-782 | KLKREAKENT |
| 482 | SCP-1 112-119 | EAEKTKKW |
| 483 | SCP-1 101-109 | GLSRVYSKL |
| 484 | SCP-1 100-109 | EGLSRVYSKL |
| 485 | SCP-1 108-116 | KLYKEAEKI |
| 486 | SCP-1 98-106 | NSEGLSRVY |
| 487 | SGP-1 97-106 | ENSEGLSRVY |
| 488 | SCP-1 102-110 | LSRVYSKLY |
| 489 | SCP-1 101-110 | GLSRVYSKLY |
| 490 | SCP-1 96-105 | LENSEGLSRV |
| 491 | SCP-1 108-117 | KLYKEAEKIIK |
| 492 | SCP-1 949-956 | REDRWAVI |
| 493 | SCP-1 948-956 | MREDRWAVI |
| 494 | SCP-1 947-956 | KMREDRWAVI |
| 495 | SCP-1 947-955 | KMREDRWAV |
| 496 | SCP-1 934-942 | TTPGSTLKF |
| 497 | SGP-1 933-942 | LTTPGSTLKF |
| 498 | SCP-1 937-945 | GSTLKGAI |
| 499 | SCP-1 945-953 | IRKMREDRW |
| 500 | SCP-1 236-243 | RLEMIIFKL |
| 501 | SCP-1 235-243 | SRLEMHFKL |
| 502 | SCP-1 242-250 | KLKEDYEKI |
| 503 | SCP-1 249-257 | KIQHLEQEY |
| 504 | SCP-1 248-257 | EKIQHLEQEY |
| 505 | SCP-1 233-242 | ENSRLEMHF |
| 506 | SCP-1 236-245 | RLEMHIFKLKE |
| 507 | SCP-1 324-331 | LEDIKVSL |
| 508 | SCP-1 323-331 | ELEDIKVSL |
| 509 | SCP-1 322-331 | KELEDIKVSL |
| 510 | SCP-1 320-327 | LTKELEDI |
| 511 | SCP-1 319-327 | HLTKELEDI |
| 512 | SCP-1 330-338 | SLQRSVSTQ |
| 513 | SCP-1 321-329 | TKELEDIKV |
| 514 | SCP-1 320-329 | LTKELEDIKV |
| 515 | SCP-1 326-335 | DIKVSLQRSV |
| 516 | SCP-1 281-288 | KMKDLTFL |
| 517 | SCP-1 280-288 | NKMKDLTFL |
| 518 | SCP-1 279-288 | ENKMKDLTFL |
| 519 | SCP-1 288-296 | LLEESRDKV |
| 520 | SCP-1 287-296 | FLLEESRDKV |
| 521 | SCP-1 291-299 | ESRDKVNQL |
| 522 | SCP-1 290-299 | EESRDKVNQL |
| 523 | SCP-1 277-285 | EKENKMKDL |
| 524 | SCP-1 276-285 | TEKENKMKDL |
| 525 | SCP-1 279-287 | ENKMKDLTF |
| 526 | SCP-1 218-225 | IEKMITAF |
| 527 | SCP-1 217-225 | NIEKIVHTAF |
| 528 | SCP-1 216-225 | SNIEKMITAF |
| 529 | SCP-1 223-230 | TAFEELRV |
| 530 | SCP-1 222-230 | ITAFEELRV |
| 531 | SCP-1 221-230 | MITAFEELRV |
| 532 | SCP-1 220-228 | KMITAFEEL |
| 533 | SCP-1 219-228 | EKMITAFEEL |
| 534 | SCP-1 227-235 | ELRVQAENS |
| 535 | SCP-1 213-222 | DLNSNIEKMI |
| 536 | SCP-1 837-844 | WTSAKNTL |
| 537 | SCP-1 846-854 | TPLPKAYTV |
| 538 | SCP-1 845-854 | STPLPKAYTV |
| 539 | SCP-1 844-852 | LSTPLPKAY |
| 540 | SCP-1 843-852 | TLSTPLPKAY |
| 541 | SCP-1 842-850 | NTLSTPLPK |
| 542 | SCP-1 841-850 | KNTLSTPLPK |
| 543 | SCP-1 828-835 | ISKDKRDY |
| 544 | SCP-1 826-835 | HGISKDKRDY |
| 545 | SCP-1 832-840 | KRDYLWTSA |
| 546 | SCP-1 829-838 | SKDKRDYLWT |
| 547 | SCP-1 279-286 | ENKMKDLT |
| 548 | SCP-1 260-268 | EINDKEKQV |
| 549 | SCP-1 274-282 | QITEKENKM |
| 550 | SCP-1 269-277 | SLLLIQITE |
| 551 | SCP-1 453-460 | FEKIAEEL |
| 552 | SCP-1 452-460 | QFEKIAEEL |
| 553 | SCP-1 451-460 | KQFEKIAEEL |
| 554 | SCP-1 449-456 | DNKQFEKI |
| 555 | SCP-1 448-456 | YDNKQFEKI |
| 556 | SCP-1 447-456 | LYDNKQFEKI |
| 557 | SCP-1 440-447 | LGEKETLL |
| 558 | SCP-1 439-447 | VLGEKETLL |
| 559 | SCP-1 438-447 | KVLGEKETLL |
| 560 | SCP-1 390-398 | LLRTEQQRL |
| 561 | SCP-1 389-398 | ELLRTEQQRL |
| 562 | SCP-1 393-401 | TEQQRLENY |
| 563 | SCP-1 392-401 | RTEQQRLENY |
| 564 | SCP-1 402-410 | EDQLIILTM |
| 565 | SCP-1 397-406 | RLENYEDQLI |
| 566 | SCP-1 368-375 | KARAAHSF |
| 567 | SCP-1 376-384 | VVTEFETTV |
| 568 | SCP-1 375-384 | FVVTEFETTV |
| 569 | SCP-1 377-385 | VTEFETTVC |
| 570 | SCP-1 376-385 | VVTEFETTVC |
| 571 | SCP-1 344-352 | DLQIATNTI |
| 572 | SCP-1 347-355 | IATNTICQL |
| 573 | SCP-1 346-355 | QIATNTICQL |
| 574 | SSX4 57-65 | VMTKLGFKY |
| 575 | SSX4 53-61 | LNYEVMTKL |
| 576 | SSX4 52-61 | KLNYEVMTKL |
| 577 | SSX4 66-74 | TLPPFMRSK |
| 578 | SSX4 110-118 | KTMPKKPAE |
| 579 | SSX4 103-112 | SLQRTFPKJM |
| 580 | Tyr 463-471 | YIKSYLEQA |
| 581 | Tyr 459-467 | SFQDYIKSY |
| 582 | Tyr 458-467 | DSFQDYIKSY |
| 583 | Tyr 507-514 | LPEEKQPL |
| 584 | Tyr 506-514 | QLPEEKQPL |
| 585 | Tyr 505-514 | KQLPEEKQPL |
| 586 | Tyr 507-515 | LPEEKQPLL |
| 587 | Tyr 506-515 | QLPEEKQPLL |
| 588 | Tyr 497-505 | SLLCRHKRK |
| 589 | ED-B domain of | EVIPQLTDLSFVDITDSSIGLRWTPLN |
| | Fibronectin | SSTIIGYRITVVAAGEGIPIFEDFVDS |
| | | SVGYYTVTGLEPGIDYDISVITLINGG |
| | | ESAPTTLTQQT |
| 590 | ED-B domain of | CTFDNLSPGLEYNVSVYTVKDDKESVP |
| | Fibronectin with | ISDTIIPEVPQLTDLSFVDITDSSIGL |
| | flanking sequence | RWTPLNSSTIIGYRITVVAAGEGIIPI |
| | from Fribronectin | FEDFVDSSVGYYTVTGLEPGIDYDISV |
| | | ITLINGGESAPTTLTQQTAVPPPTDLR |
| | | FTNIGPDTMRVTW |
| 591 | ED-B domain of | Accession number: X07717 |
| | Fibronectin cds | |
| 592 | CEA protein | Accession number: P06731 |
| 593 | CEA cDNA | Accession number: NM_004363 |
| 594 | Her2/Neu protein | Accession number: P04626 |
| 595 | Her2/Neu cDNA | Accession number: M11730 |
| 596 | SCP-1 protein | Accession number: Q15431 |
| 597 | SCP-1 cDNA | Accession number: X95654 |
| 598 | SSX-4 protein | Accession number: 060224 |
| 599 | SSX-4 cDNA | Accession number: NM_005636 |

*Any of SEQ ID NOS. 1, 8, 9, 11-23, 26-29, 32-44, 47-54, 56-63, 66-68 88-253, and 256-588 can be useful as epitopes in any of the various embodiments of the invention. Any of SEQ ID NOS. 10, 30, 31, 45, 46, 55, 64, 65, 69, 254, and 255 can be useful as sequences containing epitopes or epitope clusters, as described in various embodiments of the invention.
**All accession numbers used here and throughout can be accessed through the NCBI databases, for example, through the Entrez seek and retrieval system on the world wide web.
Note that the following discussion sets forth the inventors' understanding of the operation of the invention. However, it is not intended that this discussion limit the patent to any particular theory of operation not set forth in the claims.

In pursuing the development of epitope vaccines others have generated lists of predicted epitopes based on MHC binding motifs. Such peptides can be immunogenic, but may not correspond to any naturally produced antigenic fragment. Therefore, whole antigen will not elicit a similar response or sensitize a target cell to cytolysis by CTL. Therefore such lists do not differentiate between those sequences that can be useful as vaccines and those that cannot. Efforts to determine which of these predicted epitopes are in fact naturally produced have often relied on screening their reactivity with tumor infiltrating lymphocytes (TIL). However, TIL are strongly biased to recognize immune epitopes whereas tumors (and chronically infected cells) will generally present housekeeping epitopes. Thus, unless the epitope is produced by both the housekeeping and immunoproteasomes, the target cell will generally not be recognized by CTL induced with TIL-identified epitopes. The epitopes of the present invention, in contrast, are generated by the action of a specified proteasome, indicating that they can be naturally produced, and enabling their appropriate use. The importance of the distinction between housekeeping and immune epitopes to vaccine design is more fully set forth in PCT publication WO 01/82963 A, which is hereby incorporated by reference in its entirety.

The epitopes of the invention include or encode polypeptide fragments of TAAs that are precursors or products of proteasomal cleavage by a housekeeping or immune proteasome, and that contain or consist of a sequence having a known or predicted affinity for at least one allele of MHC I. In some embodiments, the epitopes include or encode a polypeptide of about 6 to 25 amino acids in length, preferably about 7 to 20 amino acids in length, more preferably about 8 to 15 amino acids in length, and still more preferably 9 or 10 amino acids in length. However, it is understood that the polypeptides can be larger as long as N-terminal trimming can produce the MHC epitope or that they do not contain sequences that cause the polypeptides to be directed away from the proteasome or to be destroyed by the proteasome. For immune epitopes, if the larger peptides do not contain such sequences, they can be processed in the pAPC by the immune proteasome. Housekeeping epitopes may also be embedded in longer sequences provided that the sequence is adapted to facilitate liberation of the epitope's C-terminus by action of the immunoproteasome. The foregoing discussion has assumed that processing of longer epitopes proceeds through action of the immunoproteasome of the pAPC. However, processing can also be accomplished through the contrivance of some other mechanism, such as providing an exogenous protease activity and a sequence adapted so that action of the protease liberates the MHC epitope. The sequences of these epitopes can be subjected to computer analysis in order to calculate physical, biochemical, immunologic, or molecular genetic properties such as mass, isoelectric point, predicted mobility in electrophoresis, predicted binding to other MHC molecules, melting temperature of nucleic acid probes, reverse translations, similarity or homology to other sequences, and the like.

In constructing the polynucleotides encoding the polypeptide epitopes of the invention, the gene sequence of the associated TAA can be used, or the polynucleotide can be assembled from any of the corresponding codons. For a 10 amino acid epitope this can constitute on the order of 10⁶ different sequences, depending on the particular amino acid composition. While large, this is a distinct and readily definable set representing a miniscule fraction of the >10¹⁸ possible polynucleotides of this length, and thus in some embodiments, equivalents of a particular sequence disclosed herein encompass such distinct and readily definable variations on the listed sequence. In choosing a particular one of these sequences to use in a vaccine, considerations such as codon usage, self-complementarity, restriction sites, chemical stability, etc. can be used as will be apparent to one skilled in the art.

The invention contemplates producing peptide epitopes. Specifically these epitopes are derived from the sequence of a TAA, and have known or predicted affinity for at least one allele of MHC I. Such epitopes are typically identical to those produced on target cells or pAPCs.

Compositions Containing Active Epitopes

Embodiments of the present invention provide polypeptide compositions, including vaccines, therapeutics, diagnostics, pharmacological and pharmaceutical compositions. The various compositions include newly identified epitopes of TAAs, as well as variants of these epitopes. Other embodiments of the invention provide polynucleotides encoding the polypeptide epitopes of the invention. The invention further provides vectors for expression of the polypeptide epitopes for purification. In addition, the invention provides vectors for the expression of the polypeptide epitopes in an APC for use as an anti-tumor vaccine. Any of the epitopes or antigens, or nucleic acids encoding the same, from Table 1 can be used. Other embodiments relate to methods of making and using the various compositions.

A general architecture for a class I MHC-binding epitope can be described, and has been reviewed more extensively in Madden, D. R. Annu. Rev. Immunol. 13:587-622, 1995, which is hereby incorporated by reference in its entirety. Much of the binding energy arises from main chain contacts between conserved residues in the MHC molecule and the N- and C-termini of the peptide. Additional main chain contacts are made but vary among MHC alleles. Sequence specificity is conferred by side chain contacts of so-called anchor residues with pockets that, again, vary among MHC alleles. Anchor residues can be divided into primary and secondary. Primary anchor positions exhibit strong preferences for relatively well-defined sets of amino acid residues. Secondary positions show weaker and/or less well-defined preferences that can often be better described in terms of less favored, rather than more favored, residues. Additionally, residues in some secondary anchor positions are not always positioned to contact the pocket on the MHC molecule at all. Thus, a subset of peptides exists that bind to a particular MHC molecule and have a side chain-pocket contact at the position in question and another subset exists that show binding to the same MHC molecule that does not depend on the conformation the peptide assumes in the peptide-binding groove of the MHC molecule. The C-terminal residue (P; omega) is preferably a primary anchor residue. For many of the better studied HLA molecules (e.g. A2, A68, B27, B7, B35, and B53) the second position (P2) is also an anchor residue. However, central anchor residues have also been observed including P3 and P5 in HLA-B8, as well as P5 and P (omega)-3 in the murine MHC molecules H-2D^{b} and H-2 K^{b}, respectively. Since more stable binding will generally improve immunogenicity, anchor residues are preferably conserved or optimized in the design of variants, regardless of their position.

Because the anchor residues are generally located near the ends of the epitope, the peptide can buckle upward out of the peptide-binding groove allowing some variation in length. Epitopes ranging from 8-11 amino acids have been found for HLA-A68, and up to 13 amino acids for HLA-A2. In addition to length variation between the anchor positions, single residue truncations and extensions have been reported and the N- and C-termini, respectively. Of the non-anchor residues, some point up out of the groove, making no contact with the MHC molecule but being available to contact the TCR, very often P1, P4, and P (omega)-1 for HLA-A2. Others of the non-anchor residues can become interposed between the upper edges of the peptide-binding groove and the TCR, contacting both. The exact positioning of these side chain residues, and thus their effects on binding, MHC fine conformation, and ultimately immunogenicity, are highly sequence dependent. For an epitope to be highly immunogenic it must not only promote stable enough TCR binding for activation to occur, but the TCR must also have a high enough off-rate that multiple TCR molecules can interact sequentially with the same peptide-MHC complex (Kalergis, A. M. et al., Nature Immunol. 2:229-234, 2001, which is hereby incorporated by reference in its entirety). Thus, without further information about the ternary complex, both conservative and non-conservative substitutions at these positions merit consideration when designing variants.

The polypeptide epitope variants can be made, for example, using any of the techniques and guidelines for conservative and non-conservative mutations. Variants can be derived from substitution, deletion or insertion of one or more amino acids as compared with the native sequence. Amino acid substitutions can be the result of replacing one amino acid with another amino acid having similar structural and/or chemical properties, such as the replacement of a threonine with a serine, for example. Such replacements are referred to as conservative amino acid replacements, and all appropriate conservative amino acid replacements are considered to be embodiments of one invention. Insertions or deletions can optionally be in the range of about 1 to 4, preferably 1 to 2, amino acids. It is generally preferable to maintain the “anchor positions” of the peptide which are responsible for binding to the MHC molecule in question. Indeed, immunogenicity of peptides can be improved in many cases by substituting more preferred residues at the anchor positions (Franco, et al., Nature Immunology, 1(2):145-150, 2000, which is hereby incorporated by reference in its entirety). Immunogenicity of a peptide can also often be improved by substituting bulkier amino acids for small amino acids found in non-anchor positions while maintaining sufficient cross-reactivity with the original epitope to constitute a useful vaccine. The variation allowed can be determined by routine insertions, deletions or substitutions of amino acids in the sequence and testing the resulting variants for activity exhibited by the polypeptide epitope. Because the polypeptide epitope is often 9 amino acids, the substitutions preferably are made to the shortest active epitope, for example, an epitope of 9 amino acids.

Variants can also be made by adding any sequence onto the N-terminus of the polypeptide epitope variant. Such N-terminal additions can be from 1 amino acid up to at least 25 amino acids. Because peptide epitopes are often trimmed by N-terminal exopeptidases active in the pAPC, it is understood that variations in the added sequence can have no effect on the activity of the epitope. In preferred embodiments, the amino acid residues between the last upstream proteasomal cleavage site and the N-terminus of the MHC epitope do not include a proline residue. Serwold, T. at al., Nature Immunol. 2:644-651, 2001, which is hereby incorporated by reference in its entirety. Accordingly, effective epitopes can be generated from precursors larger than the preferred 9-mer class I motif.

Generally, peptides are useful to the extent that they correspond to epitopes actually displayed by MHC I on the surface of a target cell or a pACP. A single peptide can have varying affinities for different MHC molecules, binding some well, others adequately, and still others not appreciably (Table 2). MHC alleles have traditionally been grouped according to serologic reactivity which does not reflect the structure of the peptide-binding groove, which can differ among different alleles of the same type. Similarly, binding properties can be shared across types; groups based on shared binding properties have been termed supertypes. There are numerous alleles of MHC I in the human population; epitopes specific to certain alleles can be selected based on the genotype of the patient.

TABLE 2
Predicted Binding of Tyrosinase₂₀₇₋₂₁₆
(SEQ ID NO. 1) to Various MHC types

| | *Half time of |
|---|---|
| MHC I type | dissociation (min) |
| A1 | 0.05 |
| A*0201 | 1311. |
| A*0205 | 50.4 |
| A3 | 2.7 |
| A*1101 | 0.012 |
| (part of the A3 supertype) | |
| A24 | 6.0 |
| B7 | 4.0 |
| B8 | 8.0 |
| B14 | 60.0 |
| (part of the B27 supertype) | |
| B*2702 | 0.9 |
| B*2705 | 30.0 |
| B*3501 | 2.0 |
| (part of the B7 supertype) | |
| B*4403 | 0.1 |
| B*5101 | 26.0 |
| (part of the B7 supertype) | |
| B*5102 | 55.0 |
| B*5801 | 0.20 |
| B60 | 0.40 |
| B62 | 2.0 |

*HLA Peptide Binding Predictions (world wide web hypertext transfer protocol “access at bimas.dcrt.nih.gov/molbio/hla_bin”).

In further embodiments of the invention, the epitope, as peptide or encoding polynucleotide, can be administered as a pharmaceutical composition, such as, for example, a vaccine or an immunogenic composition, alone or in combination with various adjuvants, carriers, or excipients. It should be noted that although the term vaccine may be used throughout the discussion herein, the concepts can be applied and used with any other pharmaceutical composition, including those mentioned herein. Particularly advantageous adjuvants include various cytokines and oligonucleotides containing immunostimulatory sequences (as set forth in greater detail in the co-pending applications referenced herein). Additionally the polynucleotide encoded epitope can be contained in a virus (e.g. vaccinia or adenovirus) or in a microbial host cell (e.g. Salmonella or Listeria monocytogenes) which is then used as a vector for the polynucleotide (Dietrich, G. et al. Nat. Biotech. 16:181-185, 1998, which is hereby incorporated by reference in its entirety). Alternatively a pAPC can be transformed, ex vivo, to express the epitope, or pulsed with peptide epitope, to be itself administered as a vaccine. To increase efficiency of these processes, the encoded epitope can be carried by a viral or bacterial vector, or complexed with a ligand of a receptor found on pAPC. Similarly the peptide epitope can be complexed with or conjugated to a pAPC ligand. A vaccine can be composed of more than a single epitope.

Particularly advantageous strategies for incorporating epitopes and/or epitope clusters, into a vaccine or pharmaceutical composition are disclosed in U.S. patent application Ser. No. 09/560,465 entitled “EPITOPE SYNCHRONIZATION IN ANTIGEN PRESENTING CELLS,” filed on Apr. 28, 2000, which is hereby incorporated by reference in its entirety. Epitope clusters for use in connection with this invention are disclosed in U.S. patent application Ser. No. 09/561,571 entitled “EPITOPE CLUSTERS,” filed on Apr. 28, 2000, which is hereby incorporated by reference in its entirety.

Preferred embodiments of the present invention are directed to vaccines and methods for causing a pAPC or population of pAPCs to present housekeeping epitopes that correspond to the epitopes displayed on a particular target cell. Any of the epitopes or antigens in Table 1, can be used for example. In one embodiment, the housekeeping epitope is a TuAA epitope processed by the housekeeping proteasome of a particular tumor type. In another embodiment, the housekeeping epitope is a virus-associated epitope processed by the housekeeping proteasome of a cell infected with a virus. This facilitates a specific T cell response to the target cells. Concurrent expression by the pAPCs of multiple epitopes, corresponding to different induction states (pre- and post-attack), can drive a CTL response effective against target cells as they display either housekeeping epitopes or immune epitopes.

By having both housekeeping and immune epitopes present on the pAPC, this embodiment can optimize the cytotoxic T cell response to a target cell. With dual epitope expression, the pAPCs can continue to sustain a CTL response to the immune-type epitope when the tumor cell switches from the housekeeping proteasome to the immune proteasome with induction by IFN, which, for example, may be produced by tumor-infiltrating CTLs.

In a preferred embodiment, immunization of a patient is with a vaccine that includes a housekeeping epitope. Many preferred TAAs are associated exclusively with a target cell, particularly in the case of infected cells. In another embodiment, many preferred TAAs are the result of deregulated gene expression in transformed cells, but are found also in tissues of the testis, ovaries and fetus. In another embodiment, useful TAAs are expressed at higher levels in the target cell than in other cells. In still other embodiments, TAAs are not differentially expressed in the target cell compare to other cells, but are still useful since they are involved in a particular function of the cell and differentiate the target cell from most other peripheral cells; in such embodiments, healthy cells also displaying the TAA may be collaterally attacked by the induced T cell response, but such collateral damage is considered to be far preferable to the condition caused by the target cell.

The vaccine contains a housekeeping epitope in a concentration effective to cause a pAPC or populations of pAPCs to display housekeeping epitopes. Advantageously, the vaccine can include a plurality of housekeeping epitopes or one or more housekeeping epitopes optionally in combination with one or more immune epitopes. Formulations of the vaccine contain peptides and/or nucleic acids in a concentration sufficient to cause pAPCs to present the epitopes. The formulations preferably contain epitopes in a total concentration of about 1 μg-1 mg/100 μl of vaccine preparation. Conventional dosages and dosing for peptide vaccines and/or nucleic acid vaccines can be used with the present invention, and such dosing regimens are well understood in the art. In one embodiment, a single dosage for an adult human may advantageously be from about 1 to about 5000 μl of such a composition, administered one time or multiple times, e.g., in 2, 3, 4 or more dosages separated by 1 week, 2 weeks, 1 month, or more. insulin pump delivers 1 ul per hour (lowest frequency) ref intranodal method patent.

The compositions and methods of the invention disclosed herein further contemplate incorporating adjuvants into the formulations in order to enhance the performance of the vaccines. Specifically, the addition of adjuvants to the formulations is designed to enhance the delivery or uptake of the epitopes by the pAPCs. The adjuvants contemplated by the present invention are known by those of skill in the art and include, for example, GMCSF, GCSF, IL-2, IL-12, BCG, tetanus toxoid, osteopontin, and ETA-1.

In some embodiments of the invention, the vaccines can include a recombinant organism, such as a virus, bacterium or parasite, genetically engineered to express an epitope in a host. For example, Listeria monocytogenes, a gram-positive, facultative intracellular bacterium, is a potent vector for targeting TuAAs to the immune system. In a preferred embodiment, this vector can be engineered to express a housekeeping epitope to induce therapeutic responses. The normal route of infection of this organism is through the gut and can be delivered orally. In another embodiment, an adenovirus (Ad) vector encoding a housekeeping epitope for a TuAA can be used to induce anti-virus or anti-tumor responses. Bone marrow-derived dendritic cells can be transduced with the virus construct and then injected, or the virus can be delivered directly via subcutaneous injection into an animal to induce potent T-cell responses. Another embodiment employs a recombinant vaccinia virus engineered to encode amino acid sequences corresponding to a housekeeping epitope for a TAA. Vaccinia viruses carrying constructs with the appropriate nucleotide substitutions in the form of a minigene construct can direct the expression of a housekeeping epitope, leading to a therapeutic T cell response against the epitope.

The immunization with DNA requires that APCs take up the DNA and express the encoded proteins or peptides. It is possible to encode a discrete class I peptide on the DNA. By immunizing with this construct, APCs can be caused to express a housekeeping epitope, which is then displayed on class I MHC on the surface of the cell for stimulating an appropriate CTL response. Constructs generally relying on termination of translation or non-proteasomal proteases for generation of proper termini of housekeeping epitopes have been described in U.S. patent application Ser. No. 09/561,572 entitled EXPRESSION VECTORS ENCODING EPITOPES OF TARGET-ASSOCIATED ANTIGENS, filed on Apr. 28, 2000.

As mentioned, it can be desirable to express housekeeping peptides in the context of a larger protein. Processing can be detected even when a small number of amino acids are present beyond the terminus of an epitope. Small peptide hormones are usually proteolytically processed from longer translation products, often in the size range of approximately 60-120 amino acids. This fact has led some to assume that this is the minimum size that can be efficiently translated. In some embodiments, the housekeeping peptide can be embedded in a translation product of at least about 60 amino acids. In other embodiments the housekeeping peptide can be embedded in a translation product of at least about 50, 30, or 15 amino acids.

Due to differential proteasomal processing, the immune proteasome of the pAPC produces peptides that are different from those produced by the housekeeping proteasome in peripheral body cells. Thus, in expressing a housekeeping peptide in the context of a larger protein, it is preferably expressed in the APC in a context other than its full length native sequence, because, as a housekeeping epitope, it is generally only efficiently processed from the native protein by the housekeeping proteasome, which is not active in the APC. In order to encode the housekeeping epitope in a DNA sequence encoding a larger protein, it is useful to find flanking areas on either side of the sequence encoding the epitope that permit appropriate cleavage by the immune proteasome in order to liberate that housekeeping epitope. Altering flanking amino acid residues at the N-terminus and C-terminus of the desired housekeeping epitope can facilitate appropriate cleavage and generation of the housekeeping epitope in the APC. Sequences embedding housekeeping epitopes can be designed de novo and screened to determine which can be successfully processed by immune proteasomes to liberate housekeeping epitopes.

Alternatively, another strategy is very effective for identifying sequences allowing production of housekeeping epitopes in APC. A contiguous sequence of amino acids can be generated from head to tail arrangement of one or more housekeeping epitopes. A construct expressing this sequence is used to immunize an animal, and the resulting T cell response is evaluated to determine its specificity to one or more of the epitopes in the array. By definition, these immune responses indicate housekeeping epitopes that are processed in the pAPC effectively. The necessary flanking areas around this epitope are thereby defined. The use of flanking regions of about 4-6 amino acids on either side of the desired peptide can provide the necessary information to facilitate proteasome processing of the housekeeping epitope by the immune proteasome. Therefore, a sequence ensuring epitope synchronization of approximately 16-22 amino acids can be inserted into, or fused to, any protein sequence effectively to result in that housekeeping epitope being produced in an APC. In alternate embodiments the whole head-to-tail array of epitopes, or just the epitopes immediately adjacent to the correctly processed housekeeping epitope can be similarly transferred from a test construct to a vaccine vector.

In a preferred embodiment, the housekeeping epitopes can be embedded between known immune epitopes, or segments of such, thereby providing an appropriate context for processing. The abutment of housekeeping and immune epitopes can generate the necessary context to enable the immune proteasome to liberate the housekeeping epitope, or a larger fragment, preferably including a correct C-terminus. It can be useful to screen constructs to verify that the desired epitope is produced. The abutment of housekeeping epitopes can generate a site cleavable by the immune proteasome. Some embodiments of the invention employ known epitopes to flank housekeeping epitopes in test substrates; in others, screening as described below are used whether the flanking regions are arbitrary sequences or mutants of the natural flanking sequence, and whether or not knowledge of proteasomal cleavage preferences are used in designing the substrates.

Cleavage at the mature N-terminus of the epitope, while advantageous, is not required, since a variety of N-terminal trimming activities exist in the cell that can generate the mature N-terminus of the epitope subsequent to proteasomal processing. It is preferred that such N-terminal extension be less than about 25 amino acids in length and it is further preferred that the extension have few or no proline residues. Preferably, in screening, consideration is given not only to cleavage at the ends of the epitope (or at least at its C-terminus), but consideration also can be given to ensure limited cleavage within the epitope.

Shotgun approaches can be used in designing test substrates and can increase the efficiency of screening. In one embodiment multiple epitopes can be assembled one after the other, with individual epitopes possibly appearing more than once. The substrate can be screened to determine which epitopes can be produced. In the case where a particular epitope is of concern a substrate can be designed in which it appears in multiple different contexts. When a single epitope appearing in more than one context is liberated from the substrate additional secondary test substrates, in which individual instances of the epitope are removed, disabled, or are unique, can be used to determine which are being liberated and truly constitute sequences ensuring epitope synchronization.

Several readily practicable screens exist. A preferred in vitro screen utilizes proteasomal digestion analysis, using purified immune proteasomes, to determine if the desired housekeeping epitope can be liberated from a synthetic peptide embodying the sequence in question. The position of the cleavages obtained can be determined by techniques such as mass spectrometry, HPLC, and N-terminal pool sequencing; as described in greater detail in U.S. Patent Applications entitled METHOD OF EPITOPE DISCOVERY, EPITOPE SYNCHRONIZATION IN ANTIGEN PRESENTING CELLS, two Provisional U.S. Patent Applications entitled EPITOPE SEQUENCES, which are all cited and incorporated by reference above.

Alternatively, in vivo screens such as immunization or target sensitization can be employed. For immunization a nucleic acid construct capable of expressing the sequence in question is used. Harvested CTL can be tested for their ability to recognize target cells presenting the housekeeping epitope in question. Such targets cells are most readily obtained by pulsing cells expressing the appropriate MHC molecule with synthetic peptide embodying the mature housekeeping epitope. Alternatively, cells known to express housekeeping proteasome and the antigen from which the housekeeping epitope is derived, either endogenously or through genetic engineering, can be used. To use target sensitization as a screen, CTL, or preferably a CTL clone, that recognizes the housekeeping epitope can be used. In this case it is the target cell that expresses the embedded housekeeping epitope (instead of the pAPC during immunization) and it must express immune proteasome. Generally, the target cell can be transformed with an appropriate nucleic acid construct to confer expression of the embedded housekeeping epitope. Loading with a synthetic peptide embodying the embedded epitope using peptide loaded liposomes or a protein transfer reagent such as BIOPORTER™ (Gene Therapy Systems, San Diego, Calif.) represents an alternative.

Additional guidance on nucleic acid constructs useful as vaccines in accordance with the present invention are disclosed in U.S. patent application Ser. No. 09/561,572 entitled “EXPRESSION VECTORS ENCODING EPITOPES OF TARGET-ASSOCIATED ANTIGENS,” filed on Apr. 28, 2000. Further, expression vectors and methods for their design, which are useful in accordance with the present invention are disclosed in U.S. Patent Application No. 60/336,968 (attorney docket number CTLIMM.022PR) entitled “EXPRESSION VECTORS ENCODING EPITOPES OF TARGET-ASSOCIATED ANTIGENS AND METHODS FOR THEIR DESIGN,” filed on Nov. 7, 2001, which is incorporated by reference in its entirety.

A preferred embodiment of the present invention includes a method of administering a vaccine including an epitope (or epitopes) to induce a therapeutic immune response. The vaccine is administered to a patient in a manner consistent with the standard vaccine delivery protocols that are known in the art. Methods of administering epitopes of TAAs including, without limitation, transdermal, intranodal, perinodal, oral, intravenous, intradermal, intramuscular, intraperitoneal, and mucosal administration, including delivery by injection, instillation or inhalation. A particularly useful method of vaccine delivery to elicit a CTL response is disclosed in Australian Patent No. 739189 issued Jan. 17, 2002; U.S. patent application Ser. No. 09/380,534, filed on Sep. 1, 1999; and a Continuation-in-Part thereof U.S. patent application Ser. No. 09/776,232 both entitled “A METHOD OF INDUCING A CTL RESPONSE,” filed on Feb. 2, 2001.

Reagents Recognizing Epitopes

In another aspect of the invention, proteins with binding specificity for the epitope and/or the epitope-MHC molecule complex are contemplated, as well as the isolated cells by which they can be expressed. In one set of embodiments these reagents take the form of immunoglobulins: polyclonal sera or monoclonal antibodies (mAb), methods for the generation of which are well know in the art. Generation of mAb with specificity for peptide-MHC molecule complexes is known in the art. See, for example, Aharoni et al. Nature 351:147-150, 1991; Andersen et al. Proc. Natl. Acad. Sci. USA 93:1820-1824, 1996; Dadaglio et al. Immunity 6:727-738, 1997; Duc et al. Int. Immunol. 5:427-431,1993; Eastman et al. Eur. J. Immunol. 26:385-393, 1996; Engberg et al. Immunotechnology 4:273-278, 1999; Porgdor et al. Immunity 6:715-726, 1997; Puri et al. J. Immunol. 158:2471-2476, 1997; and Polakova, K., et al. J. Immunol. 165 342-348, 2000; all of which are hereby incorporated by reference in their entirety.

In other embodiments the compositions can be used to induce and generate, in vivo and in vitro, T-cells specific for the any of the epitopes and/or epitope-MHC complexes. In preferred embodiments the epitope can be any one or more of those listed in TABLE 1, for example. Thus, embodiments also relate to and include isolated T cells, T cell clones, T cell hybridomas, or a protein containing the T cell receptor (TCR) binding domain derived from the cloned gene, as well as a recombinant cell expressing such a protein. Such TCR derived proteins can be simply the extra-cellular domains of the TCR, or a fusion with portions of another protein to confer a desired property or function. One example of such a fusion is the attachment of TCR binding domains to the constant regions of an antibody molecule so as to create a divalent molecule. The construction and activity of molecules following this general pattern have been reported, for example, Plaksin, D. et al. J. Immunol. 158:2218-2227, 1997 and Lebowitz, M. S. et al. Cell Immunol. 192:175-184, 1999, which are hereby incorporated by reference in their entirety. The more general construction and use of such molecules is also treated in U.S. Pat. No. 5,830,755 entitled T CELL RECEPTORS AND THEIR USE IN THERAPEUTIC AND DIAGNOSTIC METHODS, which is hereby incorporated by reference in its entirety.

The generation of such T cells can be readily accomplished by standard immunization of laboratory animals, and reactivity to human target cells can be obtained by immunizing with human target cells or by immunizing HLA-transgenic animals with the antigen/epitope. For some therapeutic approaches T cells derived from the same species are desirable. While such a cell can be created by cloning, for example, a murine TCR into a human T cell as contemplated above, in vitro immunization of human cells offers a potentially faster option. Techniques for in vitro immunization, even using naive donors, are know in the field, for example, Stauss et al., Proc. Natl. Acad. Sci. USA 89:7871-7875, 1992; Salgaller et al. Cancer Res. 55:4972-4979, 1995; Tsai et al., J. Immunol. 158:1796-1802, 1997; and Chung et al., J. Immunother. 22:279-287, 1999; which are hereby incorporated by reference in their entirety.

Any of these molecules can be conjugated to enzymes, radiochemicals, fluorescent tags, and toxins, so as to be used in the diagnosis (imaging or other detection), monitoring, and treatment of the pathogenic condition associated with the epitope. Thus a toxin conjugate can be administered to kill tumor cells, radiolabeling can facilitate imaging of epitope positive tumor, an enzyme conjugate can be used in an ELISA-like assay to diagnose cancer and confirm epitope expression in biopsied tissue. In a further embodiment, such T cells as set forth above, following expansion accomplished through stimulation with the epitope and/or cytokines, can be administered to a patient as an adoptive immunotherapy.

Reagents Comprising Epitopes

A further aspect of the invention provides isolated epitope-MHC complexes. In a particularly advantageous embodiment of this aspect of the invention, the complexes can be soluble, multimeric proteins such as those described in U.S. Pat. No. 5,635,363 (tetramers) or U.S. Pat. No. 6,015,884 (Ig-dimers), both of which are hereby incorporated by reference in their entirety. Such reagents are useful in detecting and monitoring specific T cell responses, and in purifying such T cells.

Isolated MHC molecules complexed with epitopic peptides can also be incorporated into planar lipid bilayers or liposomes. Such compositions can be used to stimulate T cells in vitro or, in the case of liposomes, in vivo. Co-stimulatory molecules (e.g. B7, CD40, LFA-3) can be incorporated into the same compositions or, especially for in vitro work, co-stimulation can be provided by anti-co-receptor antibodies (e.g. anti-CD28, anti-CD154, anti-CD2) or cytokines (e.g. IL-2, IL-12). Such stimulation of T cells can constitute vaccination, drive expansion of T cells in vitro for subsequent infusion in an immuotherapy, or constitute a step in an assay of T cell function.

The epitope, or more directly its complex with an MHC molecule, can be an important constituent of functional assays of antigen-specific T cells at either an activation or readout step or both. Of the many assays of T cell function current in the art (detailed procedures can be found in standard immunological references such as Current Protocols in Immunology 1999 John Wiley & Sons Inc., N.Y., which is hereby incorporated by reference in its entirety) two broad classes can be defined, those that measure the response of a pool of cells and those that measure the response of individual cells. Whereas the former conveys a global measure of the strength of a response, the latter allows determination of the relative frequency of responding cells. Examples of assays measuring global response are cytotoxicity assays, ELISA, and proliferation assays detecting cytokine secretion. Assays measuring the responses of individual cells (or small clones derived from them) include limiting dilution analysis (LDA), ELISPOT, flow cytometric detection of unsecreted cytokine (described in U.S. Pat. No. 5,445,939, entitled “METHOD FOR ASSESSMENT OF THE MONONUCLEAR LEUKOCYTE IMMUNE SYSTEM” and U.S. Patent Nos 5,656,446; and 5,843,689, both entitled “METHOD FOR THE ASSESSMENT OF THE MONONUCLEAR LEUKOCYTE IMMUNE SYSTEM,” reagents for which are sold by Becton, Dickinson & Company under the tradename ‘FASTIMMUNE’, which patents are hereby incorporated by reference in their entirety) and detection of specific TCR with tetramers or Ig-dimers as stated and referenced above. The comparative virtues of these techniques have been reviewed in Yee, C. et al. Current Opinion in Immunology, 13:141-146, 2001, which is hereby incorporated by reference in its entirety. Additionally detection of a specific TCR rearrangement or expression can be accomplished through a variety of established nucleic acid based techniques, particularly in situ and single-cell PCR techniques, as will be apparent to one of skill in the art.

These functional assays are used to assess endogenous levels of immunity, response to an immunologic stimulus (e.g. a vaccine), and to monitor immune status through the course of a disease and treatment. Except when measuring endogenous levels of immunity, any of these assays presume a preliminary step of immunization, whether in vivo or in vitro depending on the nature of the issue being addressed. Such immunization can be carried out with the various embodiments of the invention described above or with other forms of immunogen (e.g., pAPC-tumor cell fusions) that can provoke similar immunity. With the exception of PCR and tetramer/Ig-dimer type analyses which can detect expression of the cognate TCR, these assays generally benefit from a step of in vitro antigenic stimulation which can advantageously use various embodiments of the invention as described above in order to detect the particular functional activity (highly cytolytic responses can sometimes be detected directly). Finally, detection of cytolytic activity requires epitope-displaying target cells, which can be generated using various embodiments of the invention. The particular embodiment chosen for any particular step depends on the question to be addressed, ease of use, cost, and the like, but the advantages of one embodiment over another for any particular set of circumstances will be apparent to one of skill in the art.

The peptide MHC complexes described in this section have traditionally been understood to be non-covalent associations. However it is possible, and can be advantageous, to create a covalent linkages, for example by encoding the epitope and MHC heavy chain or the epitope, β2-microglobulin, and MHC heavy chain as a single protein (Yu, Y. L. Y., et al., J. Immunol. 168:3145-3149, 2002; Mottez, E., et at., J. Exp. Med. 181:493,1995; Dela Cruz, C. S., et al., Int. Immunol. 12:1293, 2000; Mage, M. G., et al., Proc. Natl. Acad. Sci. USA 89:10658,1992; Toshitani, K., et al., Proc. Natl. Acad. Sci. USA 93:236,1996; Lee, L., et al., Eur. J. Immunol. 24:2633,1994; Chung, D. H., et al., J. Immunol. 163:3699,1999; Uger, R. A. and B. H. Barber, J. Immunol. 160:1598, 1998; Uger, R. A., et al., J. Immunol. 162:6024,1999; and White, J., et al., J. Immunol. 162:2671, 1999; which are incorporated herein by reference in their entirety). Such constructs can have superior stability and overcome roadblocks in the processing-presentation pathway. They can be used in the already described vaccines, reagents, and assays in similar fashion.

Tumor Associated Antigens

Epitopes of the present invention are derived from the TuAAs tyrosinase (SEQ ID NO. 2), SSX-2, (SEQ ID NO. 3), PSMA (prostate-specific membrane antigen) (SEQ ID NO. 4), GP100, (SEQ ID NO. 70), MAGE-1, (SEQ ID NO. 71), MAGE-2, (SEQ ID NO. 72), MAGE-3, (SEQ ID NO. 73), NY-ESO-1, (SEQ ID NO. 74), PRAME, (SEQ ID NO. 77), PSA, (SEQ ID NO. 78), PSCA, (SEQ ID NO. 79), the ED-B domain of fibronectin (SEQ ID NOS 589 and 590), CEA (carcinoembryonic antigen) (SEQ ID NO. 592), Her2/Neu (SEQ ID NO. 594), SCP-1 (SEQ ID NO. 596) and SSX-4 (SEQ ID NO. 598). The natural coding sequences for these eleven proteins, or any segments within them, can be determined from their cDNA or complete coding (cds) sequences, SEQ ID NOS. 5-7, 80-87, 591, 593, 595, 597, and 599, respectively.

Tyrosinase is a melanin biosynthetic enzyme that is considered one of the most specific markers of melanocytic differentiation. Tyrosinase is expressed in few cell types, primarily in melanocytes, and high levels are often found in melanomas. The usefulness of tyrosinase as a TuAA is taught in U.S. Pat. No. 5,747,271 entitled “METHOD FOR IDENTIFYING INDIVIDUALS SUFFERING FROM A CELLULAR ABNORMALITY SOME OF WHOSE ABNORMAL CELLS PRESENT COMPLEXES OF HLA-A2/TYROSINASE DERIVED PEPTIDES, AND METHODS FOR TREATING SAID INDIVIDUALS” which is hereby incorporated by reference in its entirety.

GP100, also known as PMe117, also is a melanin biosynthetic protein expressed at high levels in melanomas. GP100 as a TuAA is disclosed in U.S. Pat. No. 5,844,075 entitled “MELANOMA ANTIGENS AND THEIR USE IN DIAGNOSTIC AND THERAPEUTIC METHODS,” which is hereby incorporated by reference in its entirety.

SSX-2, also know as Hom-Mel-40, is a member of a family of highly conserved cancer-testis antigens (Gure, A. O. et al. Int. J. Cancer 72:965-971, 1997, which is hereby incorporated by reference in its entirety). Its identification as a TuAA is taught in U.S. Pat. No. 6,025,191 entitled “ISOLATED NUCLEIC ACID MOLECULES WHICH ENCODE A MELANOMA SPECIFIC ANTIGEN AND USES THEREOF,” which is hereby incorporated by reference in its entirety. Cancer-testis antigens are found in a variety of tumors, but are generally absent from normal adult tissues except testis. Expression of different members of the SSX family have been found variously in tumor cell lines. Due to the high degree of sequence identity among SSX family members, similar epitopes from more than one member of the family will be generated and able to bind to an MHC molecule, so that some vaccines directed against one member of this family can cross-react and be effective against other members of this family (see example 3 below).

MAGE-1, MAGE-2, and MAGE-3 are members of another family of cancer-testis antigens originally discovered in melanoma (MAGE is a contraction of melanoma-associated antigen) but found in a variety of tumors. The identification of MAGE proteins as TuAAs is taught in U.S. Pat. No. 5,342,774 entitled NUCLEOTIDE SEQUENCE ENCODING THE TUMOR REJECTION ANTIGEN PRECURSOR, MAGE-1, which is hereby incorporated by reference in its entirety, and in numerous subsequent patents. Currently there are 17 entries for (human) MAGE in the SWISS Protein database. There is extensive similarity among these proteins so in many cases, an epitope from one can induce a cross-reactive response to other members of the family. A few of these have not been observed in tumors, most notably MAGE-H1 and MAGE-D1, which are expressed in testes and brain, and bone marrow stromal cells, respectively. The possibility of cross-reactivity on normal tissue is ameliorated by the fact that they are among the least similar to the other MAGE proteins.

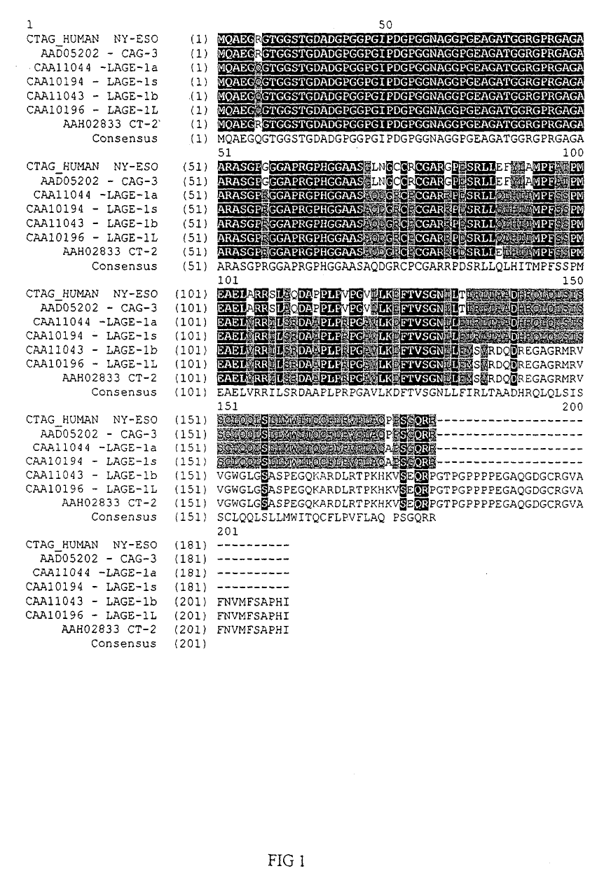

NY-ESO-1, is a cancer-testis antigen found in a wide variety of tumors, also known as CTAG-1 (Cancer-Testis Antigen-1) and CAG-3 (Cancer Antigen-3). NY-ESO-1 as a TuAA is disclosed in U.S. Pat. No. 5,804,381 entitled ISOLATED NUCLEIC ACID MOLECULE ENCODING AN ESOPHAGEAL CANCER ASSOCIATED ANTIGEN, THE ANTIGEN ITSELF, AND USES THEREOF which is hereby incorporated by reference in its entirety. A paralogous locus encoding antigens with extensive sequence identity, LAGE-1a/s (SEQ ID NO. 75) and LAGE-1b/L (SEQ ID NO. 76), have been disclosed in publicly available assemblies of the human genome, and have been concluded to arise through alternate splicing. Additionally, CT-2 (or CTAG-2, Cancer-Testis Antigen-2) appears to be either an allele, a mutant, or a sequencing discrepancy of LAGE-1b/L. Due to the extensive sequence identity, many epitopes from NY-ESO-1 can also induce immunity to tumors expressing these other antigens. See FIG. 1. The proteins are virtually identical through amino acid 70. From 71-134 the longest run of identities between NY-ESO-1 and LAGE is 6 residues, but potentially cross-reactive sequences are present. And from 135-180 NY-ESO and LAGE-1a/s are identical except for a single residue, but LAGE-1b/L is unrelated due to the alternate splice. The CAMEL and LAGE-2 antigens appear to derive from the LAGE-1 mRNA, but from alternate reading frames, thus giving rise to unrelated protein sequences. More recently, GenBank Accession AF277315.5, Homo sapiens chromosome X clone RP5-865E18, RP5-1087L19, complete sequence, reports three independent loci in this region which are labeled as LAGE1 (corresponding to CTAG-2 in the genome assemblies), plus LAGE2-A and LAGE2-B (both corresponding to CTAG-1 in the genome assemblies).

PSMA (prostate-specific membranes antigen), a TuAA described in U.S. Pat. No. 5,538,866 entitled “PROSTATE-SPECIFIC MEMBRANES ANTIGEN” which is hereby incorporated by reference in its entirety, is expressed by normal prostate epithelium and, at a higher level, in prostatic cancer. It has also been found in the neovasculature of non-prostatic tumors. PSMA can thus form the basis for vaccines directed to both prostate cancer and to the neovasculature of other tumors. This later concept is more fully described in a provisional U.S. Patent application No. 60/274,063 entitled ANTI-NEOVASCULAR VACCINES FOR CANCER, filed Mar. 7, 2001, and U.S. application Ser. No. 10/094,699, attorney docket number CTLIMM.015A, filed on Mar. 7, 2002, entitled “ANTI-NEOVASCULAR PREPARATIONS FOR CANCER,” both of which are hereby incorporated by reference in their entirety. Briefly, as tumors grow they recruit ingrowth of new blood vessels. This is understood to be necessary to sustain growth as the centers of unvascularized tumors are generally necrotic and angiogenesis inhibitors have been reported to cause tumor regression. Such new blood vessels, or neovasculature, express antigens not found in established vessels, and thus can be specifically targeted. By inducing CTL against neovascular antigens the vessels can be disrupted, interrupting the flow of nutrients to (and removal of wastes from) tumors, leading to regression.

Alternate splicing of the PSMA mRNA also leads to a protein with an apparent start at Met₅₈, thereby deleting the putative membrane anchor region of PSMA as described in U.S. Pat. No. 5,935,818 entitled “ISOLATED NUCLEIC ACID MOLECULE ENCODING ALTERNATIVELY SPLICED PROSTATE-SPECIFIC MEMBRANES ANTIGEN AND USES THEREOF” which is hereby incorporated by reference in its entirety. A protein termed PSMA-like protein, Genbank accession number AF261715, is nearly identical to amino acids 309-750 of PSMA and has a different expression profile. Thus the most preferred epitopes are those with an N-terminus located from amino acid 58 to 308.

PRAME, also know as MAPE, DAGE, and OIP4, was originally observed as a melanoma antigen. Subsequently, it has been recognized as a CT antigen, but unlike many CT antigens (e.g., MAGE, GAGE, and BAGE) it is expressed in acute myeloid leukemias. PRAME is a member of the MAPE family which consists largely of hypothetical proteins with which it shares limited sequence similarity. The usefulness of PRAME as a TuAA is taught in U.S. Pat. No. 5,830,753 entitled “ISOLATED NUCLEIC ACID MOLECULES CODING FOR TUMOR REJECTION ANTIGEN PRECURSOR DAGE AND USES THEREOF” which is hereby incorporated by reference in its entirety.

PSA, prostate specific antigen, is a peptidase of the kallikrein family and a differentiation antigen of the prostate. Expression in breast tissue has also been reported. Alternate names include gamma-seminoprotein, kallikrein 3, seminogelase, seminin, and P-30 antigen. PSA has a high degree of sequence identity with the various alternate splicing products prostatic/glandular kallikrein-1and -2, as well as kalilrein 4, which is also expressed in prostate and breast tissue. Other kallikreins generally share less sequence identity and have different expression profiles. Nonetheless, cross-reactivity that might be provoked by any particular epitope, along with the likelihood that that epitope would be liberated by processing in non-target tissues (most generally by the housekeeping proteasome), should be considered in designing a vaccine.

PSCA, prostate stem cell antigen, and also known as SCAH-2, is a differentiation antigen preferentially expressed in prostate epithelial cells, and overexpresssed in prostate cancers. Lower level expression is seen in some normal tissues including neuroendocrine cells of the digestive tract and collecting ducts of the kidney. PSCA is described in U.S. Pat. No. 5,856,136 entitled “HUMAN STEM CELL ANTIGENS” which is hereby incorporated by reference in its entirety.

Synaptonemal complex protein 1 (SCP-1), also known as HOM-TES-14, is a meiosis-associated protein and also a cancer-testis antigen (Tureci, O., et al. Proc. Natl. Acad. Sci. USA 95:5211-5216, 1998). As a cancer antigen its expression is not cell-cycle regulated and it is found frequently in gliomas, breast, renal cell, and ovarian carcinomas. It has some similarity to myosins, but with few enough identities that cross-reactive epitopes are not an immediate prospect.

The ED-B domain of fibronectin is also a potential target. Fibronectin is subject to developmentally regulated alternative splicing, with the ED-B domain being encoded by a single exon that is used primarily in oncofetal tissues (Matsuura, H. and S. Hakomori Proc. Natl. Acad. Sci. USA 82:6517-6521, 1985; Carnemolla, B. et al. J. Cell Biol. 108:1139-1148, 1989; Loridon-Rosa, B. et al. Cancer Res.50:1608-1612, 1990; Nicolo, G. et al. Cell Differ. Dev. 32:401-408, 1990; Borsi, L. et al. Exp. Cell Res. 199:98-105, 1992; Oyama, F. et al. Cancer Res. 53:2005-2011, 1993; Mandel, U. et al. APMIS 102:695-702, 1994; Farnoud, M. R. et al. Int. J. Cancer 61:27-34, 1995; Pujuguet, P. et al. Am. J. Pathol. 148:579-592, 1996; Gabler, U. et al. Heart 75:358-362, 1996;Chevalier, X. Br. J. Rheumatol. 35:407415, 1996; Midulla, M. Cancer Res. 60:164-169, 2000).

The ED-B domain is also expressed in fibronectin of the neovasculature (Kaczmarek, J. et al. Int. J. Cancer 59:11-16, 1994; Castellani, P. et al. Int. J. Cancer 59:612-618, 1994; Neri, D. et al. Nat. Biotech. 15:1271-1275, 1997; Karelina, T. V. and A. Z. Eisen Cancer Detect. Prev. 22:438-444, 1998; Tarli, L. et al. Blood 94:192-198, 1999; Castellani, P. et al. Acta Neurochir. (Wien) 142:277-282, 2000). As an oncofetal domain, the ED-B domain is commonly found in the fibronectin expressed by neoplastic cells in addition to being expressed by the neovasculature. Thus, CTL-inducing vaccines targeting the ED-B domain can exhibit two mechanisms of action: direct lysis of tumor cells, and disruption of the tumor's blood supply through destruction of the tumor-associated neovasculature. As CTL activity can decay rapidly after withdrawal of vaccine, interference with normal angiogenesis can be minimal. The design and testing of vaccines targeted to neovasculature is described in Provisional U.S. Patent Application No. 60/274,063 entitled “ANTI-NEOVASCULATURE VACCINES FOR CANCER” and in U.S. patent application Ser. No. 10/094,699, attorney docket number CTLIMM.015A, entitled “ANTI-NEOVASCULATURE PREPARATIONS FOR CANCER, filed on date even with this application (Mar. 7, 2002). A tumor cell line is disclosed in Provisional U.S. Application No. 60/363,131, filed on Mar. 7, 2002, attorney docket number CTLIMM.028PR, entitled “HLA-TRANSGENIC MURINE TUMOR CELL LINE,” which is hereby incorporated by reference in its entirety.

Carcinoembryonic antigen (CEA) is a paradigmatic oncofetal protein first described in 1965 (Gold and Freedman, J. Exp. Med. 121: 439462, 1965. Fuller references can be found in the Online Medelian Inheritance in Man; record * 114890). It has officially been renamed carcinoembryonic antigen-related cell adhesion molecule 5 (CEACAM5). Its expression is most strongly associated with adenocarcinomas of the epithelial lining of the digestive tract and in fetal colon. CEA is a member of the immunoglobulin supergene family and the defining member of the CEA subfamily.

HER2/NEU is an oncogene related to the epidermal growth factor receptor (van de Vijver, et al., New Eng. J. Med. 319:1239-1245, 1988), and apparently identical to the c-ERBB2 oncogene (Di Fiore, et al., Science 237: 178-182, 1987). The over-expression of ERBB2 has been implicated in the neoplastic transformation of prostate cancer. As HER2 it is amplified and over-expressed in 25-30% of breast cancers among other tumors where expression level is correlated with the aggressiveness of the tumor (Slamon, et al., New Eng. J. Med. 344:783-792, 2001). A more detailed description is available in the Online Medelian Inheritance in Man; record *164870.

All references mentioned herein are hereby incorporated by reference in their entirety. Further, incorporated by reference in its entirety is U.S. patent application Ser. No. 10/005,905 (attorney docket number CTLIMM.021CP1) entitled “EPITOPE SYNCHRONIZATION IN ANTIGEN PRESENTING CELLS,” filed on Nov. 7, 2001 and a continuation thereof, U.S. Application No. ______, filed on Dec. 7, 2000, attorney docket number CTLIMM.21CP1C, also entitled “EPITOPE SYNCHRONIZATION IN ANTIGEN PRESENTING CELLS.”

Useful epitopes were identified and tested as described in the following examples. However, these examples are intended for illustration purposes only, and should not be construed as limiting the scope of the invention in any way.

### EXAMPLES

Sequences of Specific Preferred Epitopes

### Example 1

Manufacture of Epitopes.

A. Synthetic Production of Epitopes

Peptides having an amino acid sequence of any of SEQ ID NO: 1, 8, 9, 11-23, 26-29, 32-44, 47-54, 56-63, 66-68 88-253, or 256-588 are synthesized using either FMOC or tBOC solid phase synthesis methodologies. After synthesis, the peptides are cleaved from their supports with either trifluoroacetic acid or hydrogen fluoride, respectively, in the presence of appropriate protective scavengers. After removing the acid by evaporation, the peptides are extracted with ether to remove the scavengers and the crude, precipitated peptide is then lyophilized. Purity of the crude peptides is determined by HPLC, sequence analysis, amino acid analysis, counterion content analysis and other suitable means. If the crude peptides are pure enough (greater than or equal to about 90% pure), they can be used as is. If purification is required to meet drug substance specifications, the peptides are purified using one or a combination of the following: re-precipitation; reverse-phase, ion exchange, size exclusion or hydrophobic interaction chromatography; or counter-current distribution.

Drug Product Formulation

GMP-grade peptides are formulated in a parenterally acceptable aqueous, organic, or aqueous-organic buffer or solvent system in which they remain both physically and chemically stable and biologically potent. Generally, buffers or combinations of buffers or combinations of buffers and organic solvents are appropriate. The pH range is typically between 6 and 9. Organic modifiers or other excipients can be added to help solubilize and stabilize the peptides. These include detergents, lipids, co-solvents, antioxidants, chelators and reducing agents. In the case of a lyophilized product, sucrose or mannitol or other lyophilization aids can be added. Peptide solutions are sterilized by membrane filtration into their final container-closure system and either lyophilized for dissolution in the clinic, or stored until use.

B. Construction of Expression Vectors for Use as Nucleic Acid Vaccines

The construction of three generic epitope expression vectors is presented below. The particular advantages of these designs are set forth in U.S. patent application Ser. No. 09/561,572 entitled “EXPRESSION VECTORS ENCODING EPITOPES OF TARGET-ASSOCIATED ANTIGENS,” which has been incorporated by reference in its entirety above.

A suitable E. coli strain was then transfected with the plasmid and plated out onto a selective medium. Several colonies were grown up in suspension culture and positive clones were identified by restriction mapping. The positive clone was then grown up and aliquotted into storage vials and stored at −70° C.

A mini-prep (QIAprep Spin Mini-prep: Qiagen, Valencia, Calif.) of the plasmid was then made from a sample of these cells and automated fluorescent dideoxy sequence analysis was used to confirm that the construct had the desired sequence.

B.1 Construction of pVAX-EP1-IRES-EP2

Overview:

The starting plasmid for this construct is pVAX1 purchased from Invitrogen (Carlsbad, Calif.). Epitopes EP 1 and EP2 were synthesized by GIBCO BRL (Rockville, Md.). The IRES was excised from pIRES purchased from Clontech (Palo Alto, Calif.).

Procedure:

1 pIRES was digested with EcoRI and NotI. The digested fragments were separated by agarose gel electrophoresis, and the IRES fragment was purified from the excised band.

2 pVAX1 was digested with EcoRI and NotI, and the pVAX1 fragment was gel-purified.

3 The purified pVAX1 and IRES fragments were then ligated together.

4 Competent E. coli of strain DH5α were transformed with the ligation mixture.

5 Minipreps were made from 4 of the resultant colonies.

6 Restriction enzyme digestion analysis was performed on the miniprep DNA. One recombinant colony having the IRES insert was used for further insertion of EP1 and EP2. This intermediate construct was called pVAX-IRES.

7 Oligonucleotides encoding EP 1 and EP2 were synthesized.

8 EP1 was subcloned into pVAX-IRES between AflII and EcoRI sites, to make pVAX-EP1-IRES;

9 EP2 was subcloned into pVAX-EP1-IRES between SalI and NotI sites, to make the final construct pVAX-EP1-IRES-EP2.

10 The sequence of the EP1-IRES-EP2 insert was confirmed by DNA sequencing.

B 2. Construction of pVAX-EP1-IRES-EP2-ISS-NIS

Overview:

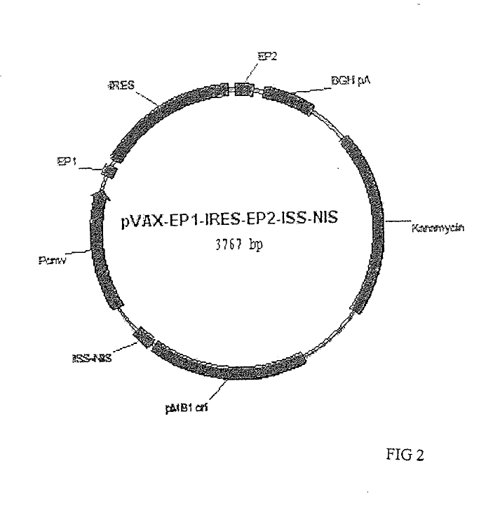

The starting plasmid for this construct was pVAX-EP1-IRES-EP2 (Example 1). The ISS (immunostimulatory sequence) introduced into this construct is AACGTT, and the NIS (standing for nuclear import sequence) used is the SV40 72 bp repeat sequence. ISS-NIS was synthesized by GIBCO BRL. See FIG. 2.

Procedure:

1 pVAX-EP 1-IRES-EP2 was digested with NruI; the linearized plasmid was gel-purified.

2 ISS-NIS oligonucleotide was synthesized.

3 The purified linearized pVAX-EP1-IRES-EP2 and synthesized ISS-NIS were ligated together.

4 Competent E. coli of strain DH5α were transformed with the ligation product.

5 Minipreps were made from resultant colonies.

6 Restriction enzyme digestions of the minipreps were carried out.

7 The plasmid with the insert was sequenced.

B3. Construction of pVAX-EP2-UB-EP1

Overview:

The starting plasmid for this construct was pVAX1 (Invitrogen). EP2 and EP1 were synthesized by GIBCO BRL. Wild type Ubiquitin cDNA encoding the 76 amino acids in the construct was cloned from yeast.

Procedure:

1 RT-PCR was performed using yeast mRNA. Primers were designed to amplify the complete coding sequence of yeast Ubiquitin.

2 The RT-PCR products were analyzed using agarose gel electrophoresis. A band with the predicted size was gel-purified.

3 The purified DNA band was subcloned into pZERO1 at EcoRV site. The resulting clone was named pZERO-UB.

4 Several clones of pZERO-UB were sequenced to confirm the Ubiquitin sequence before further manipulations.

5 EP1 and EP2 were synthesized.

6 EP2, Ubiquitin and EP1 were ligated and the insert cloned into pVAX1 between BamHI and EcoRI, putting it under control of the CMV promoter.

7 The sequence of the insert EP2-UB-EP 1 was confirmed by DNA sequencing.

### Example 2

Identification of Useful Epitope Variants

The 10-mer FLPWHRLFLL (SEQ ID NO. 1) is identified as a useful epitope. Based on this sequence, numerous variants are made. Variants exhibiting activity in HLA binding assays (see Example 3, section 6) are identified as useful, and are subsequently incorporated into vaccines.

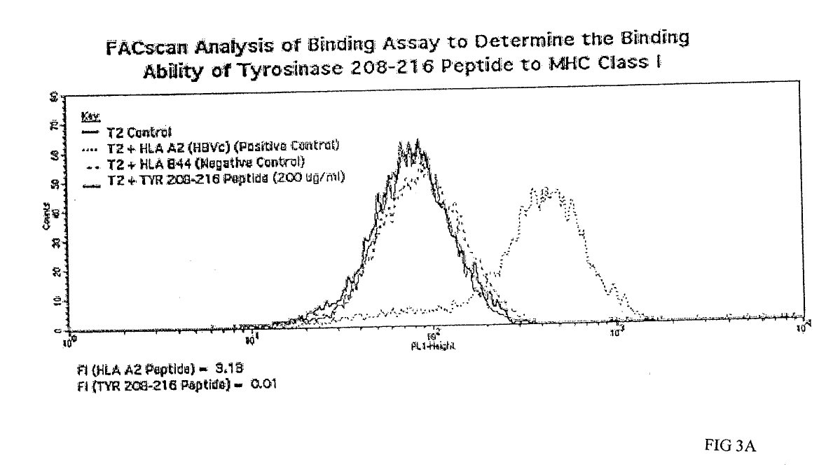

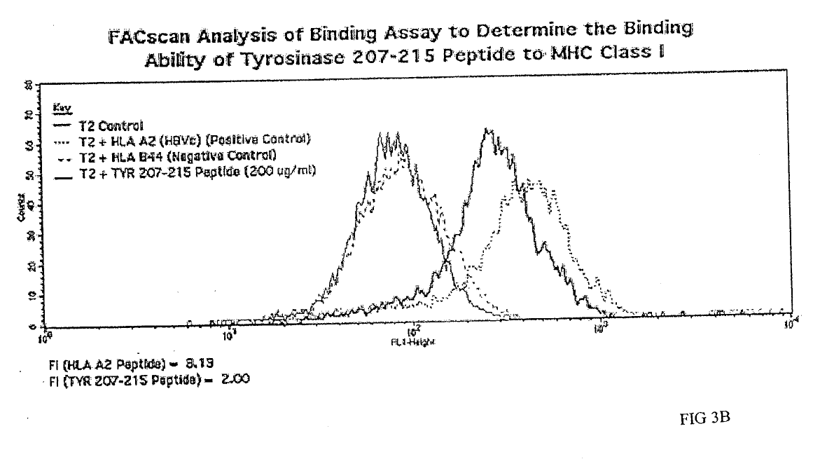

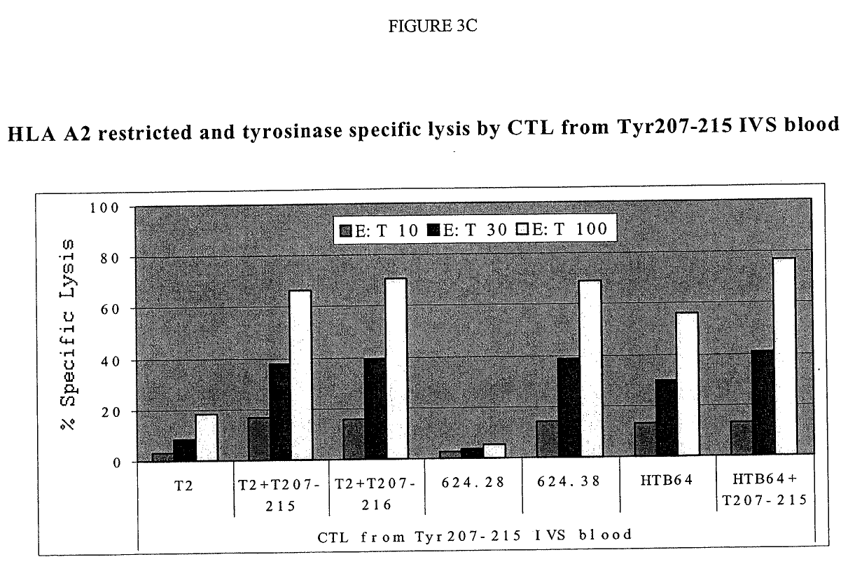

The HLA-A2 binding of length variants of FLPWHRLFLL have been evaluated. Proteasomal digestion analysis indicates that the C-terminus of the 9-mer FLPWHRLFL (SEQ ID NO. 8) is also produced. Additionally the 9-mer LPWHRLFLL (SEQ ID NO. 9) can result from N-terminal trimming of the 10-mer. Both are predicted to bind to the HLA-A*0201 molecule, however of these two 9-mers, FLPWHRLFL displayed more significant binding and is preferred (see FIGS. 3A and B).

In vitro proteasome digestion and N-terminal pool sequencing indicates that tyrosinase₂₀₇₋₂₁₆ (SEQ ID NO. 1) is produced more commonly than tyrosinase₂₀₇₋₂₁₅ (SEQ ID NO. 8), however the latter peptide displays superior immunogenicity, a potential concern in arriving at an optimal vaccine design. FLPWHRLFL, tyrosinase₂₀₇₋₂₁₅ (SEQ ID NO. 8) was used in an in vitro immunization of HLA-A2⁺ blood to generate CTL (see CTL Induction Cultures below). Using peptide pulsed T2 cells as targets in a standard chromium release assay it was found that the CTL induced by tyrosinase₂₀₇₋₂₁₅ (SEQ ID NO. 8) recognize tyrosinase₂₀₇₋₂₁₆ (SEQ ID NO. 1) targets equally well (see FIG. 3C). These CTL also recognize the HLA-A2⁺, tyrosinase⁺ tumor cell lines 624.38 and HTB64, but not 624.28 an HLA-A2⁻ derivative of 624.38 (FIG. 3C). Thus the relative amounts of these two epitopes produced in vivo, does not become a concern in vaccine design.

CTL Induction Cultures

PBMCs from normal donors were purified by centrifugation in Ficoll-Hypaque from buffy coats. All cultures were carried out using the autologous plasma (AP) to avoid exposure to potential xenogeneic pathogens and recognition of FBS peptides. To favor the in vitro generation of peptide-specific CTL, we employed autologous dendritic cells (DC) as APCs. DC were generated and CTL were induced with DC and peptide from PBMCs as described (Keogh et al., 2001). Briefly, monocyte-enriched cell fractions were cultured for 5 days with GM-CSF and IL-4 and were cultured for 2 additional days in culture media with 2 g/ml CD40 ligand to induce maturation. 2×10⁶ CD8+-enriched T lymphocytes/well and 2 ×10⁵ peptide-pulsed DC/well were co-cultured in 24-well plates in 2 ml RPMI supplemented with 10% AP, 10 ng/ml IL-7 and 20 IU/ml IL-2. Cultures were restimulated on days 7 and 14 with autologous irradiated peptide-pulsed DC.

Sequence variants of FLPWHRLFL are constructed as follow. Consistent with the binding coefficient table (see Table 3) from the NIH/BIMAS MHC binding prediction program (see reference in example 3 below), binding can be improved by changing the L at position 9, an anchor position, to V. Binding can also be altered, though generally to a lesser extent, by changes at non-anchor positions. Referring generally to Table 3, binding can be increased by employing residues with relatively larger coefficients. Changes in sequence can also alter immunogenicity independently of their effect on binding to MHC. Thus binding and/or immunogenicity can be improved as follows:

By substituting F, L, M, W, or Y for P at position 3; these are all bulkier residues that can also improve immunogenicity independent of the effect on binding. The amine and hydroxyl-bearing residues, Q and N; and S and T; respectively, can also provoke a stronger, cross-reactive response.

By substituting D or E for W at position 4 to improve binding; this addition of a negative charge can also make the epitope more immunogenic, while in some cases reducing cross-reactivity with the natural epitope. Alternatively the conservative substitutions of F or Y can provoke a cross-reactive response.

By substituting F for H at position 5 to improve binding. H can be viewed as partially charged, thus in some cases the loss of charge can hinder cross-reactivity. Substitution of the fully charged residues R or K at this position can enhance immunogenicity without disrupting charge-dependent cross-reactivity.

By substituting I, L, M, V, F, W, or Y for R at position 6. The same caveats and alternatives apply here as at position 5.

By substituting W or F for L at position 7 to improve binding. Substitution of V, I, S, T, Q, or N at this position are not generally predicted to reduce binding affinity by this model (the NIH algorithm), yet can be advantageous as discussed above.

Y and W, which are equally preferred as the Fs at positions 1 and 8, can provoke a useful cross-reactivity. Finally, while substitutions in the direction of bulkiness are generally favored to improve immunogenicity, the substitution of smaller residues such as A, S, and C, at positions 3-7 can be useful according to the theory that contrast in size, rather than bulkiness per se, is an important factor in immunogenicity. The reactivity of the thiol group in C can introduce other properties as discussed in Chen, J.-L., et al. J. Immunol. 165:948-955, 2000.

TABLE 3
9-mer Coefficient Table for HLA-A*0201*
HLA Coefficient table for file “A_0201_standard”

| Amino Acid Type | 1^{st} | 2^{nd} | 3rd | 4th | 5th | 6th | 7th | 8th | 9th |
|---|---|---|---|---|---|---|---|---|---|
| A | 1.000 | 1.000 | 1.000 | 1.000 | 1.000 | 1.000 | 1.000 | 1.000 | 1.000 |
| C | 1.000 | 0.470 | 1.000 | 1.000 | 1.000 | 1.000 | 1.000 | 1.000 | 1.000 |
| D | 0.075 | 0.100 | 0.400 | 4.100 | 1.000 | 1.000 | 0.490 | 1.000 | 0.003 |
| E | 0.075 | 1.400 | 0.064 | 4.100 | 1.000 | 1.000 | 0.490 | 1.000 | 0.003 |
| F | 4.600 | 0.050 | 3.700 | 1.000 | 3.800 | 1.900 | 5.800 | 5.500 | 0.015 |
| G | 1.000 | 0.470 | 1.000 | 1.000 | 1.000 | 1.000 | 0.130 | 1.000 | 0.015 |
| H | 0.034 | 0.050 | 1.000 | 1.000 | 1.000 | 1.000 | 1.000 | 1.000 | 0.015 |
| I | 1.700 | 9.900 | 1.000 | 1.000 | 1.000 | 2.300 | 1.000 | 0.410 | 2.100 |
| K | 3.500 | 0.100 | 0.035 | 1.000 | 1.000 | 1.000 | 1.000 | 1.000 | 0.003 |
| L | 1.700 | 72.000 | 3.700 | 1.000 | 1.000 | 2.300 | 1.000 | 1.000 | 4.300 |
| M | 1.700 | 52.000 | 3.700 | 1.000 | 1.000 | 2.300 | 1.000 | 1.000 | 1.000 |
| N | 1.000 | 0.470 | 1.000 | 1.000 | 1.000 | 1.000 | 1.000 | 1.000 | 0.015 |
| P | 0.022 | 0.470 | 1.000 | 1.000 | 1.000 | 1.000 | 1.000 | 1.000 | 0.003 |
| Q | 1.000 | 7.300 | 1.000 | 1.000 | 1.000 | 1.000 | 1.000 | 1.000 | 0.003 |
| R | 1.000 | 0.010 | 0.076 | 1.000 | 1.000 | 1.000 | 0.200 | 1.000 | 0.003 |
| S | 1.000 | 0.470 | 1.000 | 1.000 | 1.000 | 1.000 | 1.000 | 1.000 | 0.015 |
| T | 1.000 | 1.000 | 1.000 | 1.000 | 1.000 | 1.000 | 1.000 | 1.000 | 1.500 |
| V | 1.700 | 6.300 | 1.000 | 1.000 | 1.000 | 2.300 | 1.000 | 0.410 | 14.000 |
| W | 4.600 | 0.010 | 8.300 | 1.000 | 1.000 | 1.700 | 7.500 | 5.500 | 0.015 |
| Y | 4.600 | 0.010 | 3.200 | 1.000 | 1.000 | 1.500 | 1.000 | 5.500 | 0.015 |

*This table and other comparable data that are publicly available are useful in designing epitope variants and in determining whether a particular variant is substantially similar, or is functionally similar.

### Example 3

Cluster Analysis (SSX-2₃₁₋₆₈).

1. Epitope Cluster Region Prediction:

The computer algorithms: SYFPEITHI (internet http://access at syfpeithi.bmi-heidelberg.com/Scripts/MHCServer.dll/EpPredict.htm), based on the book “MHC Ligands and Peptide Motifs” by H. G. Rammensee, J. Bachmann and S. Stevanovic; and HLA Peptide Binding Predictions (NIH) (internet http://access at bimas.dcrt.nih.gov/molbio/hla_bin), described in Parker, K. C., et al., J. Immunol. 152:163, 1994; were used to analyze the protein sequence of SSX-2 (GI: 10337583). Epitope clusters (regions with higher than average density of peptide fragments with high predicted MHC affinity) were defined as described fully in U.S. patent application Ser. No. 09/561,571 entitled “EPITOPE CLUSTERS,” filed on Apr. 28, 2000. Using a epitope density ratio cutoff of 2, five and two clusters were defined using the SYFPETHI and NIH algorithms, respectively, and peptides score cutoffs of 16 (SYFPETHI) and 5 (NIH) The highest scoring peptide with the NIH algorithm, SSX-2₄₁₋₄₉, with an estimated halftime of dissociation of >1000 min., does not overlap any other predicted epitope but does cluster with SSX-2₅₇₋₆₅ in the NIH analysis.

2. Peptide Synthesis and Characterization:

SSX-2₃₁₋₆₈, YFSKEEWEKMKASEKIFYVYMKRKYEAMTKLGFKATLP (SEQ ID NO. 10) was synthesized by MPS (Multiple Peptide Systems, San Diego, Calif. 92121) using standard solid phase chemistry. According to the provided ‘Certificate of Analysis’, the purity of this peptide was 95%.

3. Proteasome Digestion:

Proteasome was isolated from human red blood cells using the proteasome isolation protocol described in U.S. patent application Ser. No. 09/561,074 entitled “METHOD OF EPITOPE DISCOVERY,” filed on Apr. 28, 2000. SDS-PAGE, western-blotting, and ELISA were used as quality control assays. The final concentration of proteasome was 4 mg/ml, which was determined by non-interfering protein assay (Geno Technologies Inc.). Proteasomes were stored at −70° C. in 25 μl aliquots.

SSX-2₃₁₋₆₈ was dissolved in Milli-Q water, and a 2 mM stock solution prepared and 20 μL aliquots stored at −20° C.

1 tube of proteasome (25 μL) was removed from storage at −70° C. and thawed on ice. It was then mixed thoroughly with 12.5 μL of 2 mM peptide by repipetting (samples were kept on ice). A 5 μL sample was immediately removed after mixing and transferred to a tube containing 1.25 μL 10% TFA (final concentration of TFA was 2%); the T=0 min sample. The proteasome digestion reaction was then started and carried out at 37° C. in a programmable thermal controller. Additional 5 μL samples were taken out at 15, 30, 60, 120, 180 and 240 min respectively, the reaction was stopped by adding the sample to 1.25 μL 10% TFA as before. Samples were kept on ice or frozen until being analyzed by MALDI-MS. All samples were saved and stored at −20° C. for HPLC analysis and N-terminal sequencing. Peptide alone (without proteasome) was used as a blank control: 2 μL peptide +4 μL Tris buffer (20 mM, pH 7.6)+1.5 μL TFA.

4. MALDI-TOF MS Measurements:

For each time point 0.3 μL of matrix solution (10 mg/ml α-cyano4-hydroxycinnamic acid in AcCN/H₂O (70:30)) was first applied on a sample slide, and then an equal volume of digested sample was mixed gently with matrix solution on the slide. The slide was allowed to dry at ambient air for 3-5 min. before acquiring the mass spectra. MS was performed on a Lasermat 2000 MALDI-TOF mass spectrometer that was calibrated with peptide/protein standards. To improve the accuracy of measurement, the molecular ion weight (MH⁺) of the peptide substrate was used as an internal calibration standard. The mass spectrum of the T=120 min. digested sample is shown in FIG. 4.

5. MS Data Analysis and Epitope Identification:

To assign the measured mass peaks, the computer program MS-Product, a tool from the UCSF Mass Spectrometry Facility (http://accessible at prospector.ucsf.edu/ucsfhtml3.4/msprod.htm), was used to generate all possible fragments (N- and C-terminal ions, and internal fragments) and their corresponding molecular weights. Due to the sensitivity of the mass spectrometer, average molecular weight was used. The mass peaks observed over the course of the digestion were identified as summarized in Table 4.

Fragments co-C-terminal with 8-10 amino acid long sequences predicted to bind HLA by the SYFPEITHI or NIH algorithms were chosen for further study. The digestion and prediction steps of the procedure can be usefully practiced in any order. Although the substrate peptide used in proteasomal digest described here was specifically designed to include predicted HLA-A2.1 binding sequences, the actual products of digestion can be checked after the fact for actual or predicted binding to other MHC molecules. Selected results are shown in Table 5.

TABLE 4
SSX-2₃₁₋₆₈ Mass Peak Identification.

| | | | CALCU- |
|---|---|---|---|
| | | | LATED |
| MS PEAK | | | MASS |
| (measured) | PEPTIDE | SEQUENCE | (MH⁺) |
| 988.23 | 31-37 | YFSKEEW | 989.08 |
| 1377.68 ± | 31-40 | YFSKEEWEKM | 1377.68 |
| 2.38 | | | |
| 1662.45 ± | 31-43 | YFSKEEWEKMKAS | 1663.90 |
| 1.30 | | | |
| 2181.72 ± | 31-47 | YFSKEEWEKMKASEKIF | 2181.52 |
| 0.85 | | | |
| 2346.6 | 31-48 | YFSKEEWEKMKASEICIFY | 2344.71 |
| 1472.16 ± | 38-49 | EKMKASEKIFYV | 1473.77 |
| 1.54 | | | |
| 2445.78 ± | 31-49* | YFSKEEWEKMKASEKIFYV | 2443.84 |
| 1.18 | | | |
| 2607. | 31-50 | YFSKEEWEKMKASEKIFYVY | 2607.02 |
| 1563.3 | 50-61 | YMKRKYBAMTKL | 1562.93 |
| 3989.9 | 31-61 | YFSKEEWEKMKASEKIFYVYMK | 3987.77 |
| RKYEAMTKL | | | |
| 1603.74 ± | 51-63 | MKRKYEAMTKLGF | 1603.98 |
| 1.53 | | | |
| 1766.45 ± | 50-63 | YMKRKYEAMTKLGF | 1767.16 |
| 1.53 | | | |
| 1866.32 ± | 49-63 | VYMKRKYEAMTKLGF | 1866.29 |
| 1.22 | | | |
| 4192.6 | 31-63 | YFSKEEWEKMKASEKIFYVYMK | 4192.00 |
| RKYEAMTKLGF | | | |
| 4392.1 | 31-65** | YFSKEEWEKMKASEKIFYVYMK | 4391.25 |

RKYEAMTKLGFKA

Boldface sequence correspond to peptides predicted to bind to MHC.
*On the basis of mass alone this peak could also have been assigned to the peptide 32-50, however proteasomal removal of just the N-terminal amino acid is unlikely. N-terminal sequencing (below) verifies the assignment to 31-49.
**On the basis of mass this fragment might also represent 33-68. N-terminal sequencing below is consistent with the assignment to 31-65.

TABLE 5
Predicted HLA binding by proteasomally generated
fragments

| SEQ ID NO. | PEPTIDE | HLA | SYFPEITHI | NIH |
|---|---|---|---|---|
| 11 | FSKEEWEKM | B*3501 | NP† | 90 |
| 12 | KMKASEKIF | B*08 | 17 | <5 |
| 13 & (14) | (K)MKASEKIFY | A1 | 19(19) | <5 |
| 15 & (16) | (M)KASEKIFYV | A*0201 | 22(16) | 1017 |
| | | B*08 | 17 | <5 |
| | | B*5101 | 22(13) | 60 |
| | | B*5102 | NP | 133 |
| | | B*5103 | NP | 121 |
| 17 & (18) | (K)ASEKTFYVY | A1 | 34(19) | 14 |
| 19 & (20) | (K)RKYEAMTKL | A*0201 | 15 | <5 |
| | | A26 | 15 | NP |
| | | B14 | NP | 45(60) |
| | | B*2705 | 21 | 15 |
| | | B*2709 | 16 | NP |
| | | B*5101 | 15 | <5 |
| 21 | KYEAMTKLGF | A1 | 16 | <5 |
| | | A24 | NP | 300 |
| 22 | YEAMTKLGF | B*4403 | NP | 80 |
| 23 | EAMTKLGF | B*08 | 22 | <5 |

†No prediction

As seen in Table 5, N-terminal addition of authentic sequence to epitopes can generate epitopes for the same or different MHC restriction elements. Note in particular the pairing of (K)RKYEAMTKL (SEQ ID NOS 19 and (20)) with HLA-B14, where the 10-mer has a longer predicted halftime of dissociation than the co-C-terminal 9-mer. Also note the case of the 10-mer KYEAMTKLGF (SEQ ID NO. 21) which can be used as a vaccine useful with several MHC types by relying on N-terminal trimming to create the epitopes for HLA-B*4403 and -B*08.

6. HLA-A0201 Binding Assay:

Binding of the candidate epitope KASEKIFYV, SSX-2₄₁₋₄₉, (SEQ ID NO. 15) to HLA-A2.1 was assayed using a modification of the method of Stauss et al., (Proc Natl Acad Sci USA 89(17):7871-5 (1992)). Specifically, T2 cells, which express empty or unstable MHC molecules on their surface, were washed twice with Iscove's modified Dulbecco's medium (IMDM) and cultured overnight in serum-free AIM-V medium (Life Technologies, Inc., Rockville, Md.) supplemented with human β2-microglobulin at 3 μg/ml (Sigma, St. Louis, Mo.) and added peptide, at 800, 400, 200, 100, 50, 25, 12.5, and 6.25 μg/ml. in a 96-well flat-bottom plate at 3×10⁵ cells/200 μl/well. Peptide was mixed with the cells by repipeting before distributing to the plate (alternatively peptide can be added to individual wells), and the plate was rocked gently for 2 minutes. Incubation was in a 5% CO₂ incubator at 37° C. The next day the unbound peptide was removed by washing twice with serum free RPMI medium and a saturating amount of anti-class I HLA monoclonal antibody, fluorescein isothiocyanate (FITC)-conjugated anti-HLA A2, A28 (One Lambda, Canoga Park, Calif.) was added. After incubation for 30 minutes at 4° C., cells were washed 3 times with PBS supplemented with 0.5% BSA, 0.05% (w/v) sodium azide, pH 7.4-7.6 (staining buffer). (Alternatively W6/32 (Sigma) can be used as the anti-class I HLA monoclonal antibody the cells washed with staining buffer and then incubated with fluorescein isothiocyanate (FITC)-conjugated goat F(ab′) antimouse-IgG (Sigma) for 30 min at 4° C. and washed 3 times as before.) The cells were resuspended in 0.5 ml staining buffer. The analysis of surface HLA-A2.1 molecules stabilized by peptide binding was performed by flow cytometry using a FACScan (Becton Dickinson, San Jose, Calif.). If flow cytometry is not to be performed immediately the cells can be fixed by adding a quarter volume of 2% paraformaldehyde and storing in the dark at 4° C.

The results of the experiment are shown in FIG. 5. SSX-2₄₁₋₄₉ (SEQ ID NO. 15) was found to bind HLA-A2.1 to a similar extent as the known A2.1 binder FLPSDYFPSV (HBV₁₈₋₂₇; SEQ ID NO: 24) used as a positive control. An HLA-B44 binding peptide, AEMGKYSFY (SEQ ID NO: 25), was used as a negative control. The fluoresence obtained from the negative control was similar to the signal obtained when no peptide was used in the assay. Positive and negative control peptides were chosen from Table 18.3.1 in Current Protocols in Immunology p. 18.3.2, John Wiley and Sons, New York, 1998.

7. Immunogenicity:

A. In vivo Immunization of Mice.

HHD1 transgenic A*0201 mice (Pascolo, S., et al. J. Exp. Med. 185:2043-2051, 1997) were anesthetized and injected subcutaneously at the base of the tail, avoiding lateral tail veins, using 100 μl containing 100 nmol of SSX-2₄₁₋₄₉ (SEQ ID NO. 15) and 20 μg of HTL epitope peptide in PBS emulsified with 50 μl of IFA (incomplete Freund's adjuvant).

B. Preparation of Stimulating Cells (LPS Blasts).

Using spleens from 2 naive mice for each group of immunized mice, un-immunized mice were sacrificed and the carcasses were placed in alcohol. Using sterile instruments, the top dermal layer of skin on the mouse's left side (lower mid-section) was cut through, exposing the peritoneum. The peritoneum was saturated with alcohol, and the spleen was aseptically extracted. The spleen was placed in a petri dish with serum-free media. Splenocytes were isolated by using sterile plungers from 3 ml syringes to mash the spleens. Cells were collected in a 50 ml conical tubes in serum-free media, rinsing dish well. Cells were centrifuged (12000 rpm, 7 min) and washed one time with RPMI. Fresh spleen cells were resuspended to a concentration of 1×10⁶ cells per ml in RPMI-100% FCS (fetal calf serum). 25 g/ml lipopolysaccharide and 7 μg/ml Dextran Sulfate were added. Cell were incubated for 3 days in T-75 flasks at 37° C., with 5% CO₂. Splenic blasts were collected in 50 ml tubes pelleted (12000 rpm, 7 min) and resuspended to 3×10⁷/ml in RPMI. The blasts were pulsed with the priming peptide at 50 μg/ml, RT 4 hr. mitomycin C-treated at 25 μg/ml, 37° C., 20 min and washed three times with DMEM.

C. In vitro Stimulation.

3 days after LPS stimulation of the blast cells and the same day as peptide loading, the primed mice were sacrificed (at 14 days post immunization) to remove spleens as above. 3×10⁶ splenocytes were co-cultured with 1×10⁶ LPS blasts/well in 24-well plates at 37° C., with 5% CO₂ in DMEM media supplemented with 10% FCS, 5×10⁻⁵ M β-mercaptoethanol, 100 μg/ml streptomycin and 100 IU/ml penicillin. Cultures were fed 5% (vol/vol) ConA supernatant on day 3 and assayed for cytolytic activity on day 7 in a ⁵¹Cr-release assay.

D. Chromium-Release Assay Measuring CTL Activity.

To assess peptide specific lysis, 2×10⁶ T2 cells were incubated with 100 μCi sodium chromate together with 50 μg/ml peptide at 37 C for 1 hour. During incubation they were gently shaken every 15 minutes. After labeling and loading, cells were washed three times with 10 ml of DMEM-10% FCS, wiping each tube with a fresh Kimwipe after pouring off the supernatant. Target cells were resuspended in DMEM-10% FBS 1×10⁵/ml. Effector cells were adjusted to 1×10⁷/ml in DMEM-10% FCS and 100 μl serial 3-fold dilutions of effectors were prepared in U-bottom 96-well plates. 100 μl of target cells were added per well. In order to determine spontaneous release and maximum release, six additional wells containing 100 μl of target cells were prepared for each target. Spontaneous release was revealed by incubating the target cells with 100 μl medium; maximum release was revealed by incubating the target cells with 100 μl of 2% SDS. Plates were then centrifuged for 5 min at 600 rpm and incubated for 4 hours at 37° C. in 5% CO₂ and 80% humidity. After the incubation, plates were then centrifuged for 5 min at 1200 rpm. Supernatants were harvested and counted using a gamma counter. Specific lysis was determined as follows: % specific release=[(experimental release−spontaneous release)/(maximum release—spontaneous release)]×100.

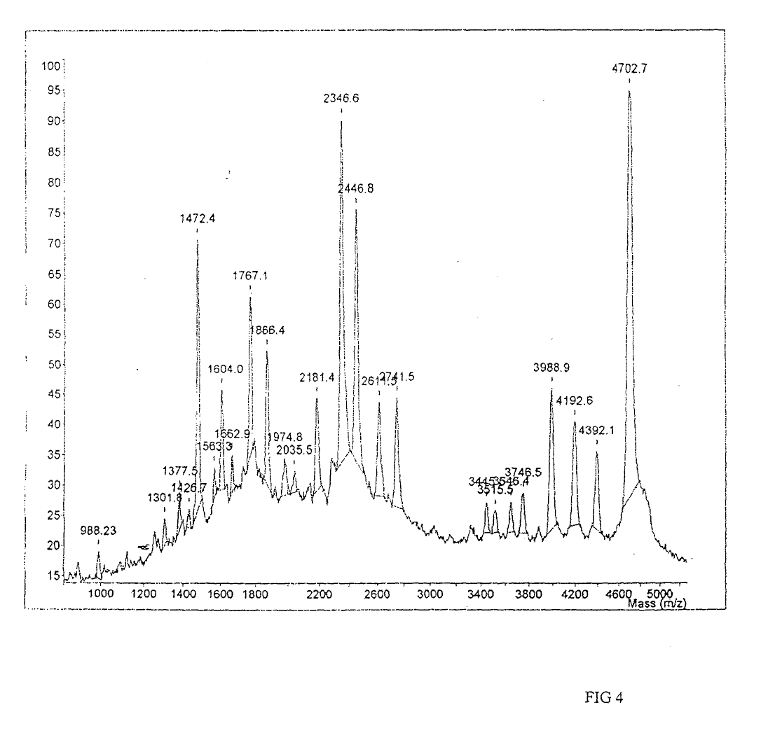

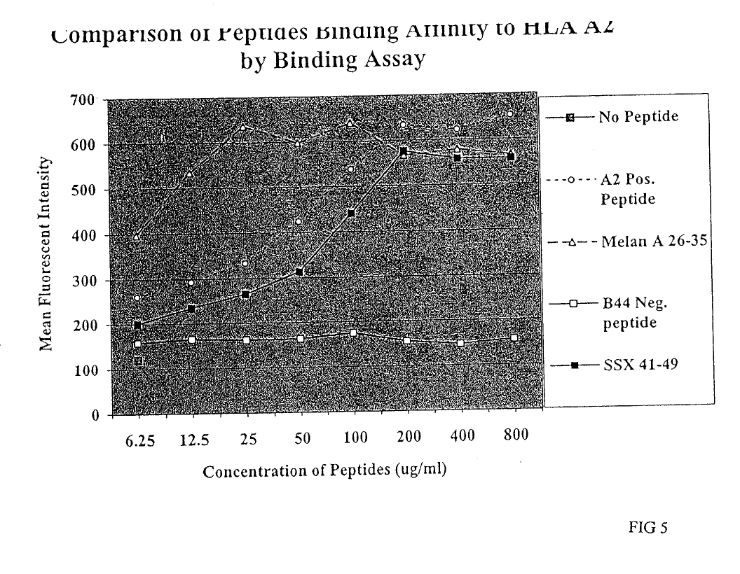

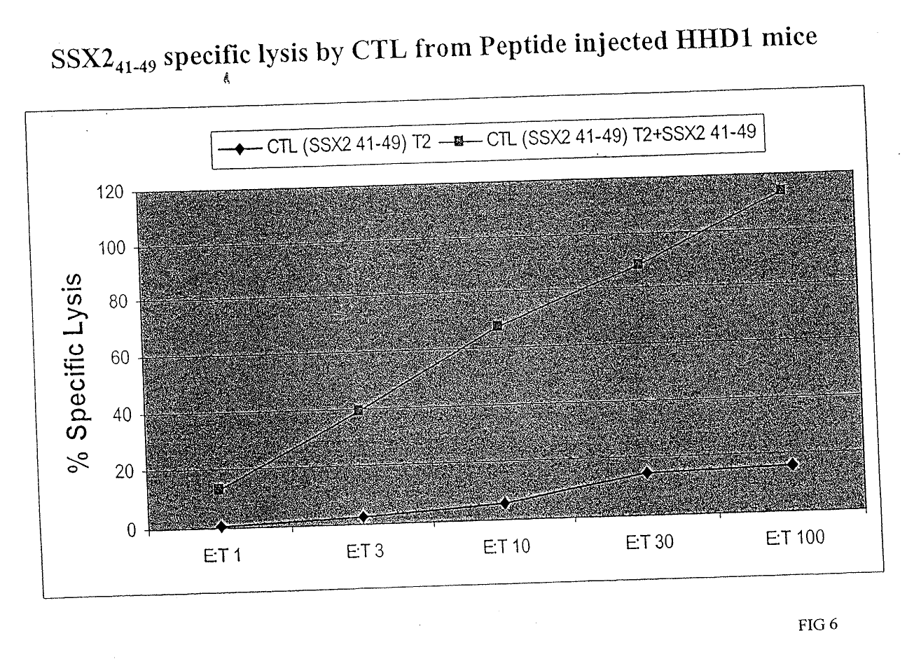

Results of the chromium release assay demonstrating specific lysis of peptide pulsed target cells are shown in FIG. 6.

8. Cross-Reactivity with Other SSX Proteins:

SSX-2₄₁₋₄₉ (SEQ ID NO. 15) shares a high degree of sequence identity with the same region of the other SSX proteins. The surrounding regions have also been generally well conserved. Thus the housekeeping proteasome can cleave following V₄₉ in all five sequences. Moreover, SSX₄₁₋₄₉ is predicted to bind HLA-A*0201 (see Table 6). CTL generated by immunization with SSX-2₄₁₋₄₉ cross-react with tumor cells expressing other SSX proteins.

TABLE 6
SSX₄₁₋₄₉—A*0201 Predicted Binding

| | Family | | SYFPEITHI | NIH |
|---|---|---|---|---|
| SEQ ID NO. | Member | Sequence | Score | Score |
| 15 | SSX-2 | KASEKTFYV | 22 | 1017 |
| 26 | SSX-1 | KYSEKISYV | 18 | 1.7 |
| 27 | SSX-3 | KVSEKIVYV | 24 | 1105 |
| 28 | SSX-4 | KSSEKIVYV | 20 | 82 |
| 29 | SSX-5 | KASEKIIYV | 22 | 175 |

### Example 4

Cluster Analysis (PSMA₁₆₃₋₁₉₂).

A peptide, AFSPQGMPEGDLVYVNYARTEDFFKLERDM, PSMA₁₆₃₋₁₉₂, (SEQ ID NO. 30), containing an A1 epitope cluster from prostate specific membrane antigen, PSMA₁₆₈₋₁₉₀ (SEQ ID NO. 31) was synthesized using standard solid-phase F-moc chemistry on a 433A ABI Peptide synthesizer. After side chain deprotection and cleavage from the resin, peptide first dissolved in formic acid and then diluted into 30% Acetic acid, was run on a reverse-phase preparative HPLC C4 column at following conditions: linear AB gradient (5% B/min) at a flow rate of 4 ml/min, where eluent A is 0.1% aqueous TFA and eluent B is 0.1% TFA in acetonitrile. A fraction at time 16.642 min containing the expected peptide, as judged by mass spectrometry, was pooled and lyophilized. The peptide was then subjected to proteasome digestion and mass spectrum analysis essentially as described above. Prominent peaks from the mass spectra are summarized in Table 7.

TABLE 7
PSMA₁₆₃₋₁₉₂ Mass Peak Identification.

| | | CALCULATED |
|---|---|---|
| PEPTIDE | SEQUENCE | MASS (MU⁺) |
| 163-177 | AFSPQGMPEGDLVYV | 1610.0 |
| 178-189 | NYARTEDFFKLE | 1533.68 |
| 170-189 | PEGDLVYVNYARTEDFFKLE | 2406.66 |
| 178-191 | NYARTEDFFKLERD | 1804.95 |
| 170-191 | PEGDLVYVNYARTEDFFKLERD | 2677.93 |
| 178-192 | NYARTEDFFKLERDM | 1936.17 |
| 163-176 | AFSPQGMPEGDLVY | 1511.70 |
| 177-192 | VNYARTEDFFKLERDM | 2035.30 |
| 163-179 | AFSPQGMPEGDLVYVNY | 1888.12 |
| 180-192 | ARTEDFFKLERDM | 1658.89 |
| 163-183 | AFSPQGMPEGDLVYVNYARTE | 2345.61 |
| 184-192 | DFFKLERDM | 1201.40 |
| 176-192 | YVNYARTEDFFKLERDM | 2198.48 |
| 167-185 | QGMPEGDLVYVNYARTEDF | 2205.41 |
| 178-186 | NYARTEDFF | 1163.22 |

Boldface sequences correspond to peptides predicted to bind to MuG, see Table 8.

N-Terminal Pool Sequence Analysis

One aliquot at one hour of the proteasomal digestion (see Example 3 part 3 above) was subjected to N-terminal amino acid sequence analysis by an ABI 473A Protein Sequencer (Applied Biosystems, Foster City, Calif.). Determination of the sites and efficiencies of cleavage was based on consideration of the sequence cycle, the repetitive yield of the protein sequencer, and the relative yields of amino acids unique in the analyzed sequence. That is if the unique (in the analyzed sequence) residue X appears only in the nth cycle a cleavage site exists n−1 residues before it in the N-terminal direction. In addition to helping resolve any ambiguity in the assignment of mass to sequences, these data also provide a more reliable indication of the relative yield of the various fragments than does mass spectrometry.

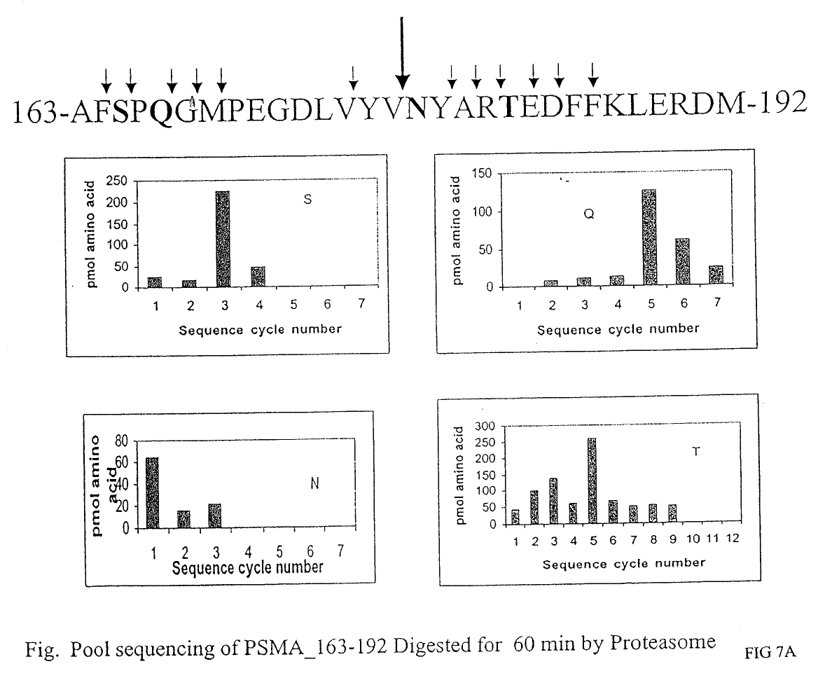

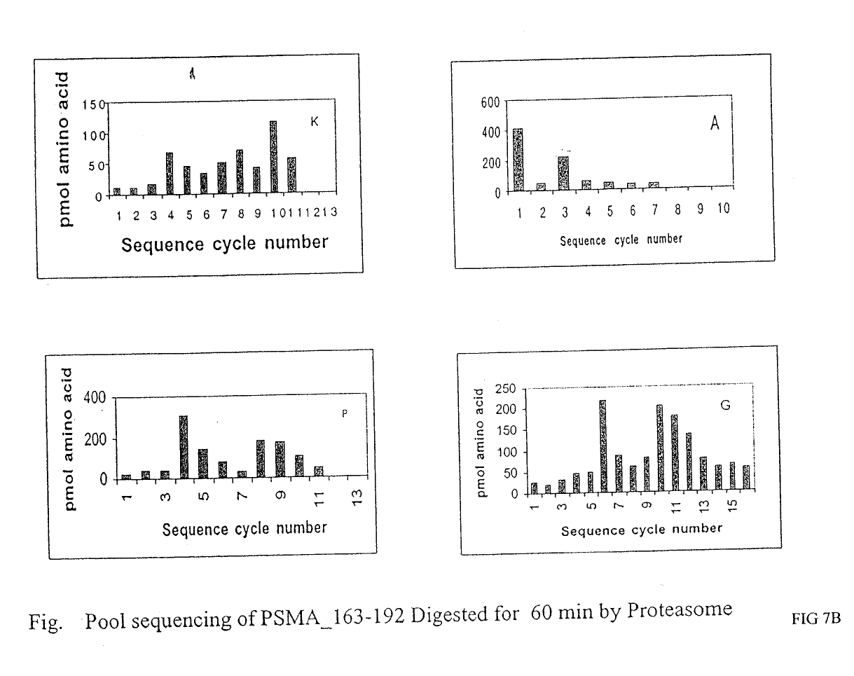

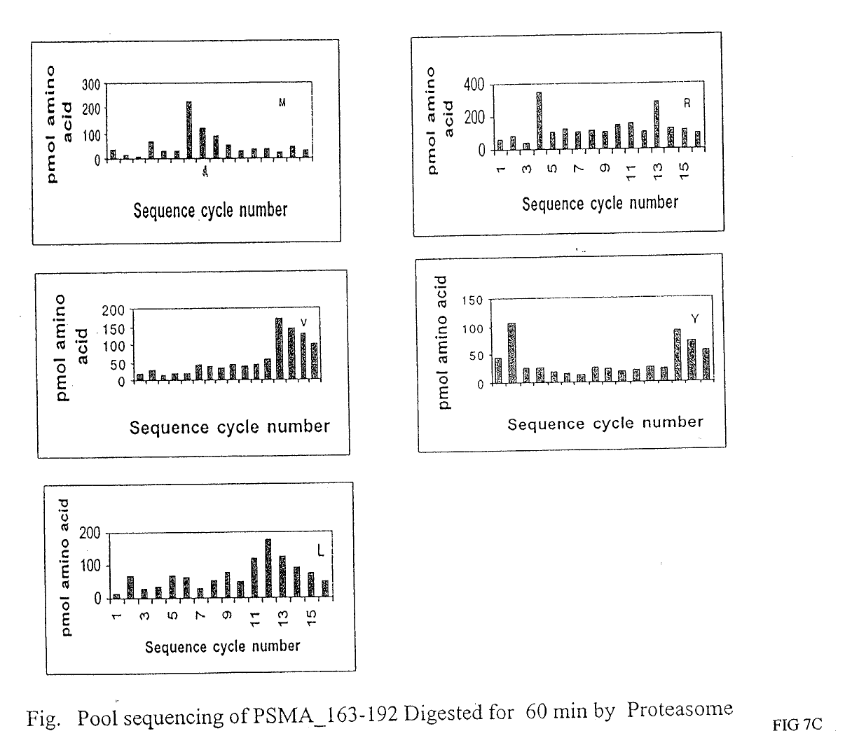

For PSMA₁₆₃₋₁₉₂ (SEQ ID NO. 30) this pool sequencing supports a single major cleavage site after V₁₇₇ and several minor cleavage sites, particularly one after Y₁₇₉. Reviewing the results presented in FIGS. 7A-C reveals the following:

S at the 3^{rd} cycle indicating presence of the N-terminus of the substrate.

Q at the 5^{h} cycle indicating presence of the N-terminus of the substrate.

N at the 1^{st} cycle indicating cleavage after V₁₇₇.

N at the 3^{rd} cycle indicating cleavage after V₁₇₅. Note the fragment 176-192 in Table 7.

T at the 5^{th} cycle indicating cleavage after V₁₇₇.

T at the 1^{st}-3^{rd} cycles, indicating increasingly common cleavages after R₁₈₁, A₁₈₀ and Y₁₇₉. Only the last of these correspond to peaks detected by mass spectrometry; 163-179 and 180-192, see Table 7. The absence of the others can indicate that they are on fragments smaller than were examined in the mass spectrum.

K at the 4^{th}, 8^{th}, and 10^{th} cycles indicating cleavages after E₁₈₃, Y₁₇₉, and V₁₇₇, respectively, all of which correspond to fragments observed by mass spectroscopy. See Table 7.

A at the 1^{st} and 3^{rd} cycles indicating presence of the N-terminus of the substrate and cleavage after V₁₇₇, respectively.

P at the 4^{th} and 8^{h} cycles indicating presence of the N-terminus of the substrate.

G at the 6^{th} and 10^{th} cycles indicating presence of the N-terminus of the substrate.

M at the 7^{th} cycle indicating presence of the N-terminus of the substrate and/or cleavage after F₁₈₅.

M at the 15^{th} cycle indicating cleavage after V₁₇₇.

The 1^{st} cycle can indicate cleavage after D₁₉₁, see Table 7.

R at the 4^{th} and 13^{th} cycle indicating cleavage after V₁₇₇.

R at the 2^{nd} and 11^{th} cycle indicating cleavage after Y₁₇₉.

V at the 2^{nd}, 6^{th}, and 13^{th} cycle indicating cleavage after V₁₇₅, M₁₆₉ and presence of the N-terminus of the substrate, respectively. Note fragments beginning at 176 and 170 in Table 7.

Y at the 1^{st}, 2^{nd}, and 14^{th} cycles indicating cleavage after V₁₇₅, V₁₇₇, and presence of the N-terminus of the substrate, respectively.

L at the 11^{th} and 12^{th} cycles indicating cleavage after V₁₇₇, and presence of the N-terminus of the substrate, respectively, is the interpretation most consistent with the other data. Comparing to the mass spectrometry results we see that L at the 2^{nd}, 5^{th}, and 9^{th} cycles is consistent with cleavage after F₁₈₆, E₁₈₃ or M₁₆₉, and Y₁₇₉, respectively. See Table 7.

Epitope Identification

Fragments co-C-terminal with 8-10 amino acid long sequences predicted to bind HLA by the SYFPEITHI or NIH algorithms were chosen for further analysis. The digestion and prediction steps of the procedure can be usefully practiced in any order. Although the substrate peptide used in proteasomal digest described here was specifically designed to include a predicted HLA-A1 binding sequence, the actual products of digestion can be checked after the fact for actual or predicted binding to other MHC molecules. Selected results are shown in Table 8.

TABLE 8
Predicted HLA binding by proteasomally generated
fragments

| SEQ ID NO | PEPTIDE | HLA | SYFPEITHI | NIH |
|---|---|---|---|---|
| 32 & (33) | (G)MPEGDLVY | A*0201 | 17(27) | (2605) |
| | V | | | |
| | | B*0702 | 20 | <5 |
| | | B*5101 | 22 | 314 |
| 34 & (35) | (Q)GMPEGDLV | A1 | 24(26) | <5 |
| | Y | A3 | 16(18) | 36 |
| | | B*2705 | 17 | 25 |
| 36 | MPEGDLVY | B*5101 | 15 | NP† |
| 37 & (38) | (P)EGDLVYVN | A1 | 27(15) | 12 |
| | Y | A26 | 23(17) | NP |
| 39 | LVYVNYARTE | A3 | 21 | <5 |
| 40 & (41) | (Y)VNYARTED | A26 | (20) | NP |
| | F | B*08 | 15 | <5 |
| | | B*2705 | 12 | 50 |
| 42 | NYARTEDFF | A24 | NP† | 100 |
| | | Cw*0401 | NP | 120 |
| 43 | YARTEDFF | B*08 | 16 | <5 |
| 44 | RTEDFFKLE | A1 | 21 | <5 |
| | | A26 | 15 | NP |

†No prediction

HLA-A*0201 Binding Assay:

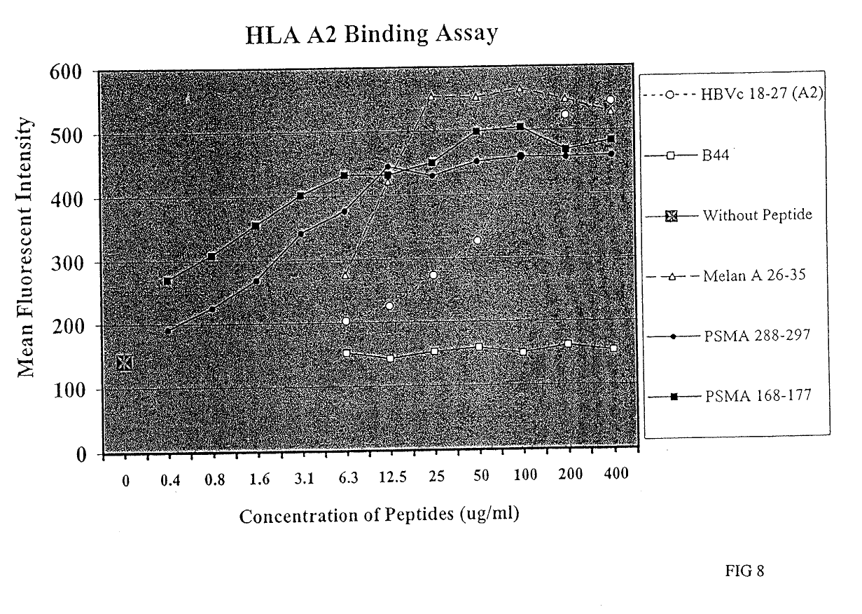

HLA-A*0201 binding studies were preformed with PSMA₁₆₈₋₁₇₇, GMPEGDLVYV, (SEQ ID NO. 33) essentially as described in Example 3 above. As seen in FIG. 8, this epitope exhibits significant binding at even lower concentrations than the positive control peptides. The Melan-A peptide used as a control in this assay (and throughout this disclosure), ELAGIGILTV, is actually a variant of the natural sequence (EAAGIGILTV) and exhibits a high affinity in this assay.

### Example 5

Cluster Analysis (PSMA₂₈₁₋₃₁₀).

Another peptide, RGIAEAVGLPSIPVHPIGYYDAQKLLEKMG, PSMA₂₈₁₋₃₁₀, (SEQ ID NO. 45), containing an A1 epitope cluster from prostate specific membrane antigen, PSMA₂₈₃₋₃₀₇ (SEQ ID NO. 46), was synthesized using standard solid-phase F-moc chemistry on a 433A ABI Peptide synthesizer. After side chain deprotection and cleavage from the resin, peptide in ddH2O was run on a reverse-phase preparative HPLC C18 column at following conditions: linear AB gradient (5% B/min) at a flow rate of 4 ml/min, where eluent A is 0.1% aqueous TFA and eluent B is 0.1% TFA in acetonitrile. A fraction at time 17.061 min containing the expected peptide as judged by mass spectrometry, was pooled and lyophilized. The peptide was then subjected to proteasome digestion and mass spectrum analysis essentially as described above. Prominent peaks from the mass spectra are summarized in Table 9.

TABLE 9
PSMA₂₈₁₋₃₁₀ Mass Peak Identification.

| | | CALCULATED |
|---|---|---|
| PEPTIDE | SEQUENCE | MASS (MH⁺) |
| 281-297 | RGIAEAVGLPSIPVHPI* | 1727.07 |
| 286-297 | AVGLPSIPVHPI** | 1200.46 |
| 287-297 | VGLPSIPVHPI | 1129.38 |
| 288-297 | GLPSIPVHPI† | 1030.25 |
| 298-310 | GYYDAQKLLEKMG‡ | 1516.5 |
| 298-305 | GYYDAQKL§ | 958.05 |
| 281-305 | RGIAEAVGLPSIPVHPIGYYDAQKL | 2666.12 |
| 281-307 | RGIAEAVGLPSIPVHPIGYYDAQKLLE | 2908.39 |
| 286-307 | AVGLPSIPVHPIGYYDAQKLLE¶ | 2381.78 |
| 287-307 | VGLPSTPVHPIGYYDAQKLLE | 2310.70 |
| 288-307 | GLPSIPVHPIGYYDAQKLLE# | 2211.57 |
| 281-299 | RGTAEAVGLPSIPVHPIGY | 1947 |
| 286-299 | AVGLPSIPVHPIGY | 1420.69 |
| 287-299 | VGLPSIPVHPIGY | 1349.61 |
| 288-299 | GLPSIPVHPIGY | 1250.48 |
| 287-310 | VGLPSIPVHPIGYYDAQKLLEKMG | 2627.14 |
| 288-310 | GLPSIPVHPIGYYDAQKLLEKMG | 2528.01 |

Boldface sequences correspond to peptides predicted to bind to MUG, see Table 10.
*By mass alone this peak could also have been 296-310 or 288-303.
**By mass alone this peak could also have been 298-307. Combination of HPLC and mass spectrometry show that at some later time points this peak is a mixture of both species.
†By mass alone this peak could also have been 289-298.
By mass alone this peak could also have been 281-295 or 294-306.
§By mass alone this peak could also have been 297-303.
¶By mass alone this peak could also have been 285-306.
#By mass alone this peak could also have been 288-303.
None of these alternate assignments are supported N-terminal pool sequence analysis.

N-Terminal Pool Sequence Analysis

One aliquot at one hour of the proteasomal digestion (see Example 3 part 3 above) was subjected to N-terminal amino acid sequence analysis by an ABI 473A Protein Sequencer (Applied Biosystems, Foster City, Calif.). Determination of the sites and efficiencies of cleavage was based on consideration of the sequence cycle, the repetitive yield of the protein sequencer, and the relative yields of amino acids unique in the analyzed sequence. That is if the unique (in the analyzed sequence) residue X appears only in the nth cycle a cleavage site exists n−1 residues before it in the N-terminal direction. In addition to helping resolve any ambiguity in the assignment of mass to sequences, these data also provide a more reliable indication of the relative yield of the various fragments than does mass spectrometry.

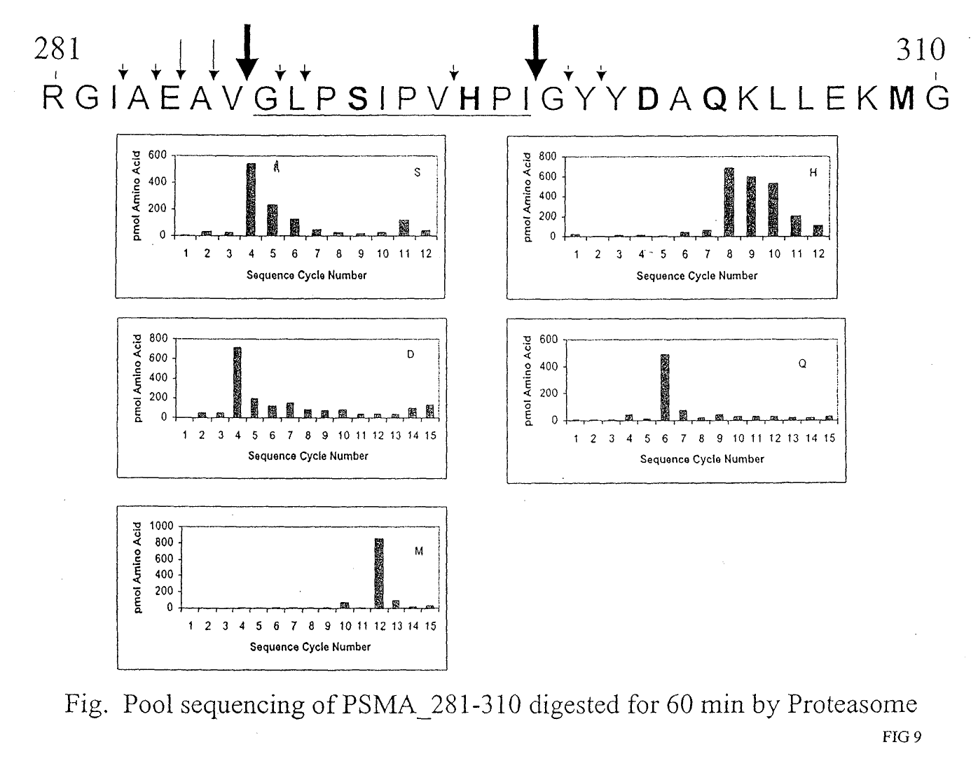

For PSMA₂₈₁₋₃₁₀ (SEQ ID NO. 45) this pool sequencing supports two major cleavage sites after V₂₈₇ and I₂₉₇ among other minor cleavage sites. Reviewing the results presented in FIG. 9 reveals the following:

S at the 4^{th} and 11^{th} cycles indicating cleavage after V₂₈₇ and presence of the N-terminus of the substrate, respectively.

H at the 8^{th} cycle indicating cleavage after V₂₈₇. The lack of decay in peak height at positions 9 and 10 versus the drop in height present going from 10 to 11 can suggest cleavage after A₂₈₆ and E₂₈₅ as well, rather than the peaks representing latency in the sequencing reaction.

D at the 2^{nd}, 4^{th}, and 7^{th} cycles indicating cleavages after Y₂₉₉, I₂₉₇, and V₂₉₄, respectively. This last cleavage is not observed in any of the fragments in Table 10 or in the alternate assignments in the notes below.

Q at the 6^{th} cycle indicating cleavage after I₂₉₇.

M at the 10^{th} and 12^{th} cycle indicating cleavages after Y₂₉₉ and I₂₉₇, respectively.

Epitope Identification

Fragments co-C-terminal with 8-10 amino acid long sequences predicted to bind HLA by the SYFPEITHI or NIH algorithms were chosen for further study. The digestion and prediction steps of the procedure can be usefully practiced in any order. Although the substrate peptide used in proteasomal digest described here was specifically designed to include a predicted HLA-A1 binding sequence, the actual products of digestion can be checked after the fact for actual or predicted binding to other MHC molecules. Selected results are shown in Table 10.

TABLE 10
Predicted HLA binding by proteasomally generated
fragments: PSMA₂₈₁₋₃₁₀

| SEQ ID NO. | PEPTIDE | HLA | SYFPEITHI | NIH |
|---|---|---|---|---|
| 47 & (48) | (G)LPSIPVH | A*0201 | 16(24) | (24) |
| | PI | B*0702/B7 | 23 | 12 |
| | | B*5101 | 24 | 572 |
| | | Cw*0401 | NP† | 20 |
| 49 & (50) | (P)IGYYDAQ | A*0201 | (16) | <5 |
| | KL | A26 | (20) | NP |
| | | B*2705 | 16 | 25 |
| | | B*2709 | 15 | NP |
| | | B*5101 | 21 | 57 |
| | | Cw*0301 | NP | 24 |
| 51 & (52) | (P)SIPVHPI | A1 | 21(27) | <5 |
| | GY | A26 | 22 | NP |
| | | A3 | 16 | <5 |
| 53 | IPVHPIGY | B*5101 | 16 | NP |
| 54 | YYDAQKLLE | A1 | 22 | <5 |

†No prediction

As seen in Table 10, N-terminal addition of authentic sequence to epitopes can often generate still useful, even better epitopes, for the same or different MHC restriction elements. Note for example the pairing of (G)LPSIPVHPI with HLA-A*0201, where the 10-mer can be used as a vaccine useful with several MHC types by relying on N-terminal trimming to create the epitopes for HLA-B7, -B*5101, and Cw*0401.

HLA-A*0201 Binding Assay:

HLA-A*0201 binding studies were preformed with PSMA₂₈₈₋₂₉₇, GLPSIPVHPI, (SEQ ID NO. 48) essentially as described in Examples 3 and 4 above. As seen in FIG. 8, this epitope exhibits significant binding at even lower concentrations than the positive control peptides.

### Example 6

Cluster Analysis (PSMA₄₅₄₋₄₈₁).

Another peptide, SSIEGNYTLRVDCTPLMYSLVHLTKEL, PSMA₄₅₄₋₄₈₁, (SEQ ID NO. 55) containing an epitope cluster from prostate specific membrane antigen, was synthesized by MPS (purity >95%) and subjected to proteasome digestion and mass spectrum analysis as described above. Prominent peaks from the mass spectra are summarized in Table 11.

TABLE 11

PSMA₄₅₄₋₄₈₁ Mass Peak Identification.

| | | | |
|---|---|---|---|
| | PEPTIDE | SEQUENCE | CALCULATED |
| MS PEAK (measured) | | | MASS (MH⁺) |

| | | | |
|---|---|---|---|
| 1238.5 | 454-464 | SSIEGNYTLRV | 1239.78 |
| 1768.38 ± 0.60 | 454-469 | SSIEGNYTLRVDCTPL | 1768.99 |
| 1899.8 | 454-470 | SSIEGNYTLRVDCTPLM | 1900.19 |
| 1097.63 ± 0.91 | 463-471 | RVDCTPLMY | 1098.32 |
| 2062.87 ± 0.68 | 454-471* | SSIEGNYTLRVDCTPLMY | 2063.36 |
| 1153 | 472-481** | SLVHNLTKEL | 1154.36 |
| 1449.93 ± 1.79 | 470-481 | MYSLVHNLTKEL | 1448.73 |

Boldface sequence correspond to peptides predicted to bind to MHC, see Table 12.
*On the basis of mass alone this peak could equally well be assigned to the peptide 455-472 however proteasomal removal of just the N-terminal amino acid is considered unlikely. If the issue were important it could be resolved by N-terminal sequencing.
**On the basis of mass this fragment might also represent 455-464.

Epitope Identification

Fragments co-C-terminal with 8-10 amino acid long sequences predicted to bind HLA by the SYFPEITHI or NIH algorithms were chosen for further study. The digestion and prediction steps of the procedure can be usefully practiced in any order. Although the substrate peptide used in proteasomal digest described here was specifically designed to include predicted HLA-A2.1 binding sequences, the actual products of digestion can be checked after the fact for actual or predicted binding to other MHC molecules. Selected results are shown in Table 12.

TABLE 12

Predicted HLA binding by proteasomally generated
fragments

| SEQ ID NO | PEPTIDE | HLA | SYFPEITHI | NIH |
|---|---|---|---|---|
| 56 & (57) | (S)IEGNYTLRV | A1 | (19) | <5 |
| | | A*0201 | 16(22) | <5 |
| 58 | EGNYTLRV | B*5101 | 15 | NP† |
| 59 & (60) | (Y)TLRVDCTPL | A*0201 | 20(18) | (5) |
| | | A26 | 16(18) | NP |
| | | B7 | 14 | 40 |
| | | B8 | 23 | <5 |
| | | B*2705 | 12 | 30 |
| | | Cw*0301 | NP | (30) |
| 61 | LRVDCTPLM | B*2705 | 20 | 600 |
| | | B*2709 | 20 | NP |
| 62 & (63) | (L)RVTDCTPLMY | A1 | 32(22) | 125 (13.5) |
| | | A3 | 25 | <5 |
| | | A26 | 22 | NP |
| | | B*2702 | NP | (200) |
| | | B*2705 | 13 (NP) | (1000) |

†No prediction

As seen in Table 12, N-terminal addition of authentic sequence to epitopes can often generate still useful, even better epitopes, for the same or different MHC restriction elements. Note for example the pairing of (L)RVDCTPLMY (SEQ ID NOS 62 and (63)) with HLA-B*2702/5, where the 10-mer has substantial predicted halftimes of dissociation and the co-C-terminal 9-mer does not. Also note the case of SIEGNYTLRV (SEQ ID NO 57) a predicted HLA-A*0201 epitope which can be used as a vaccine useful with HLA-B*5101 by relying on N-terminal trimming to create the epitope.

HLA-A*0201 Binding Assay

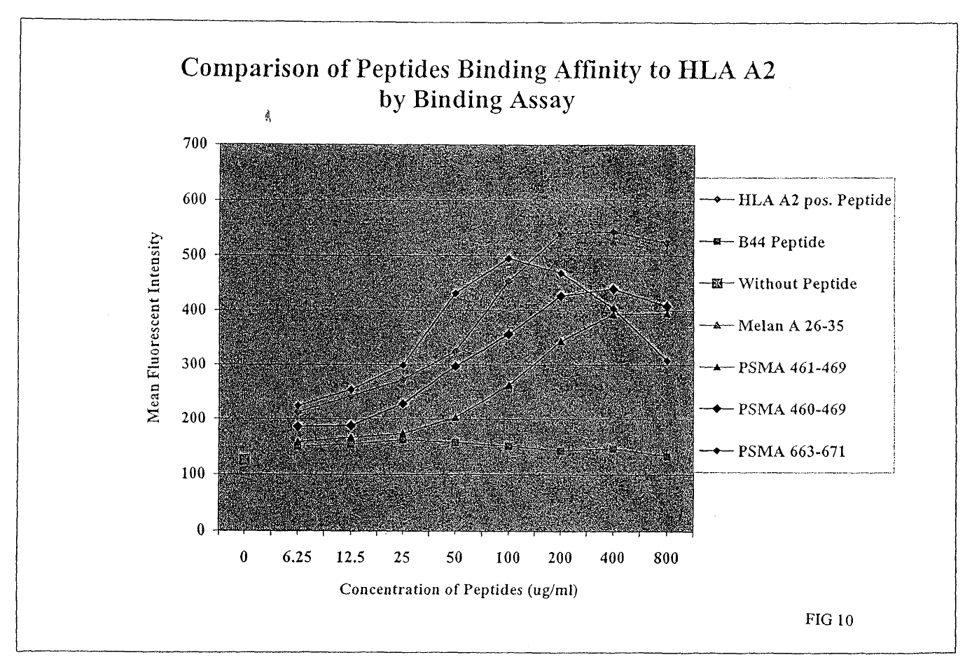

HLA-A*0201 binding studies were preformed, essentially as described in Example 3 above, with PSMA₄₆₀₋₄₆₉, TLRVDCTPL, (SEQ ID NO. 60). As seen in FIG. 10, this epitope was found to bind HLA-A2.1 to a similar extent as the known A2.1 binder FLPSDYFPSV (HBV₁₈₋₂₇; SEQ ID NO: 24) used as a positive control. Additionally, PSMA₄₆₁₋₄₆₉, (SEQ ID NO. 59) binds nearly as well.

ELISPOT Analysis: PSMA₄₆₃₋₄₇₁ (SEQ ID NO. 62)

The wells of a nitrocellulose-backed microtiter plate were coated with capture antibody by incubating overnight at 4° C. using 50 μl/well of 4 μg/ml murine anti-human γ-IFN monoclonal antibody in coating buffer (35 mM sodium bicarbonate, 15 mM sodium carbonate, pH 9.5). Unbound antibody was removed by washing 4 times 5 min. with PBS. Unbound sites on the membrane then were blocked by adding 2001 μl/well of RPMI medium with 10% serum and incubating 1 hr. at room temperature. Antigen stimulated CD8⁺ T cells, in 1:3 serial dilutions, were seeded into the wells of the microtiter plate using 100 μl/well, starting at 2×10⁵ cells/well. (Prior antigen stimulation was essentially as described in Scheibenbogen, C. et al. Int. J. Cancer 71:932-936, 1997. PSMA₄₆₂₋₄₇₁ (SEQ ID NO. 62) was added to a final concentration of 10 μg/ml and IL-2 to 100 U/ml and the cells cultured at 37° C. in a 5% CO₂, water-saturated atmosphere for 40 hrs. Following this incubation the plates were washed with 6 times 200 μl/well of PBS containing 0.05% Tween-20 (PBS-Tween). Detection antibody, 50 μl/well of 2 g/ml biotinylated murine anti-human γ-IFN monoclonal antibody in PBS+10% fetal calf serum, was added and the plate incubated at room temperature for 2 hrs. Unbound detection antibody was removed by washing with 4 times 200 μl of PBS-Tween. 100 μl of avidin-conjugated horseradish peroxidase (Pharmingen, San Diego, Calif.) was added to each well and incubated at room temperature for 1 hr. Unbound enzyme was removed by washing with 6 times 200 μl of PBS-Tween. Substrate was prepared by dissolving a 20 mg tablet of 3-amino 9-ethylcoarbasole in 2.5 ml of N, N-dimethylformamide and adding that solution to 47,5 ml of 0.05 M phosphate-citrate buffer (pH 5.0). 25 μl of 30% H₂O₂ was added to the substrate solution immediately before distributing substrate at 100 μl/well and incubating the plate at room temperature. After color development (generally 15-30 min.), the reaction was stopped by washing the plate with water. The plate was air dried and the spots counted using a stereomicroscope.

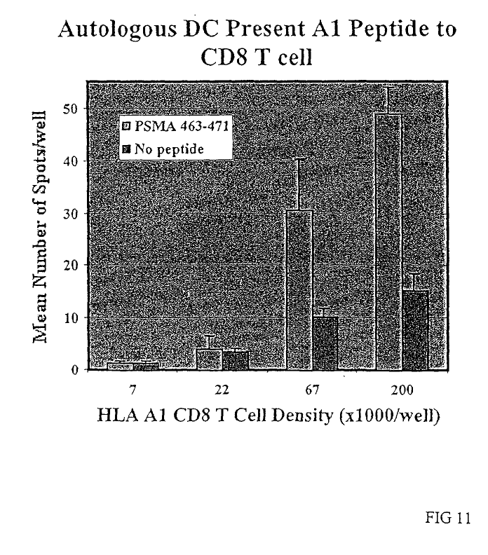

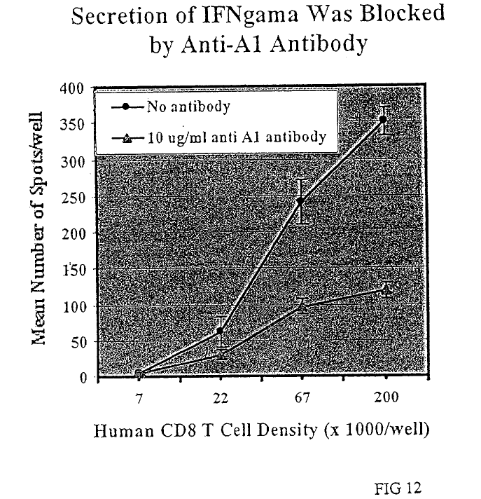

FIG. 11 shows the detection of PSMA₄₆₃₋₄₇₁ (SEQ ID NO. 62)-reactive HLA-A1⁺ CD8⁺ T cells previously generated in cultures of HLA-A1⁺ CD8⁺ T cells with autologous dendritic cells plus the peptide. No reactivity is detected from cultures without peptide (data not shown). In this case it can be seen that the peptide reactive T cells are present in the culture at a frequency between 1 in 2.2×10⁴ and 1 in 6.7×10⁴. That this is truly an HLA-A1-restricted response is demonstrated by the ability of anti-HLA-A1 monoclonal antibody to block γ-IFN production; see FIG. 12.

### Example 7

Cluster Analysis (PSMA₆₅₃₋₆₈₇).

Another peptide, FDKSNPIVLRMMNDQLMFLERAFIDPLGLPDRPFY PSMA₆₅₃₋₆₈₇, (SEQ ID NO. 64) containing an A2 epitope cluster from prostate specific membrane antigen, PSMA₆₆₀₋₆₈₁ (SEQ ID NO 65), was synthesized by MPS (purity >95%) and subjected to proteasome digestion and mass spectrum analysis as described above. Prominent peaks from the mass spectra are summarized in Table 13.

TABLE 13

PSMA₆₅₃₋₆₈₇ Mass Peak Identification.

| | | | |
|---|---|---|---|
| MS PEAK (measured) | PEPTIDE | SEQUENCE | CALCULATED MASS (MH⁺) |

| | | | | |
|---|---|---|---|---|
| 906.17 ± | 0.65 | 681-687** | LPDRPFY | 908.05 |
| 1287.73 ± | 0.76 | 677-687** | DPLGLPDRPFY | 1290.47 |
| 1400.3 ± | 1.79 | 676-687 | IDPLGLPDRPFY | 1403.63 |
| 1548.0 ± | 1.37 | 675-687 | FIDPLGLPDRPFY | 1550.80 |
| 1619.5 ± | 1.51 | 674-687** | AFIDPLGLPDRPFY | 1621.88 |
| 1775.48 ± | 1.32 | 673-687* | RAFIDPLGLPDRPFY | 1778.07 |
| 2440.2 ± | 1.3 | 653-672 | FDKSNPIVLRMMNDQLMFLE | 2442.93 |
| 1904.63 ± | 1.56 | 672-687* | ERAFIDPLGLPDRPFY | 1907.19 |
| 2310.6 ± | 2.5 | 653-671 | FDKSNPIVLRMMNDQLMFL | 2313.82 |
| 2017.4 ± | 1.94 | 671-687 | LERAFIDPLGLPDRPFY | 2020.35 |
| 2197.43 ± | 1.78 | 653-670 | FDKSNPIVLRMMNDQLMF | 2200.66 |

Boldface sequence correspond to peptides predicted to bind to MHC, see Table 13.
*On the basis of mass alone this peak could equally well be assigned to a peptide beginning at 654, however proteasomal removal of just the N-terminal amino acid is considered unlikely. If the issue were important it could be resolved by N-terminal sequencing.
**On the basis of mass alone these peaks could have been assigned to internal fragments, but given the overall pattern of digestion it was considered unlikely.

Epitope Identification

Fragments co-C-terminal with 8-10 amino acid long sequences predicted to bind HLA by the SYFPEITHI or NIH algorithms were chosen for further study. The digestion and prediction steps of the procedure can be usefully practiced in any order. Although the substrate peptide used in proteasomal digest described here was specifically designed to include predicted HLA-A2.1 binding sequences, the actual products of digestion can be checked after the fact for actual or predicted binding to other MHC molecules. Selected results are shown in Table 14.

TABLE 14

Predicted lILA binding by proteasomally generated fragments

| | | | | |
|---|---|---|---|---|
| SEQ ID NO | PEPTIDE | HLA | SYFPEITHI | NIH |

| | | | | |
|---|---|---|---|---|
| 66 & (67) | (R)MMNDQLMF | A*0201 | 24 (23) | 1360 (722) |
| | L | | | |
| | | A*0205 | NP† | 71(42) |
| | | A26 | 15 | NP |
| | | B*2705 | 12 | 50 |
| 68 | RMMNDQLMF | B*2705 | 17 | 75 |

†No prediction

As seen in Table 14, N-terminal addition of authentic sequence to epitopes can generate still useful, even better epitopes, for the same or different MHC restriction elements. Note for example the pairing of (R)MMNDQLMFL (SEQ ID NOS. 66 and (67)) with HLA-A*02, where the 10-mer retains substantial predicted binding potential.

HLA-A*0201 Binding Assay

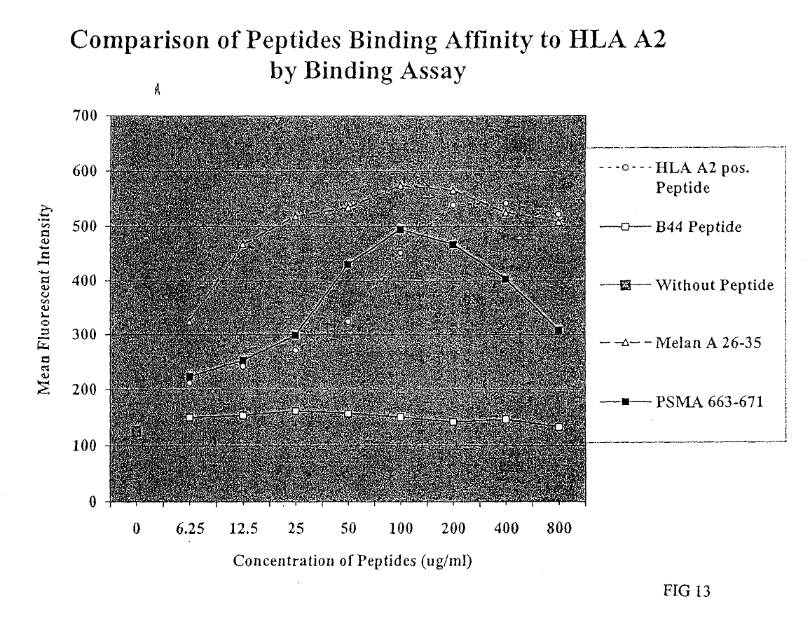

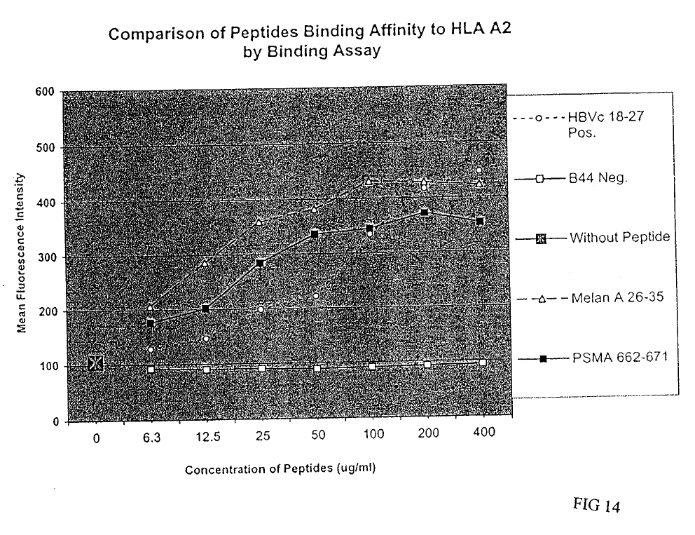

HLA-A*0201 binding studies were preformed, essentially as described in Example 3 above, with PSMA₆₆₃₋₆₇1, (SEQ ID NO. 66) and PSMA₆₆₂₋₆₇₁, RMMNDQLMFL (SEQ NO. 67). As seen in FIGS. 10, 13 and 14, this epitope exhibits significant binding at even lower concentrations than the positive control peptide (FLPSDYFPSV (HBV₁₈₋₂₇); SEQ ID NO: 24). Though not run in parallel, comparison to the controls suggests that PSMA₆₆₂₋₆₇₁ (which approaches the Melan A peptide in affinity) has the superior binding activity of these two PSMA peptides.

### Example 8

Vaccinating with Epitope Vaccines.

1. Vaccination with Peptide Vaccines:

A. Intranodal Delivery

A formulation containing peptide in aqueous buffer with an antimicrobial agent, an antioxidant, and an immunomodulating cytokine, was injected continuously over several days into the inguinal lymph node using a miniature pumping system developed for insulin delivery (MiniMed; Northridge, CA). This infusion cycle was selected in order to mimic the kinetics of antigen presentation during a natural infection.

B. Controlled Release

A peptide formulation is delivered using controlled PLGA microspheres as is known in the art, which alter the pharmacokinetics of the peptide and improve immunogenicity. This formulation is injected or taken orally.

C. Gene Gun Delivery

A peptide formulation is prepared wherein the peptide is adhered to gold microparticles as is known in the art. The particles are delivered in a gene gun, being accelerated at high speed so as to penetrate the skin, carrying the particles into dermal tissues that contain pAPCs.

D. Aerosol Delivery

A peptide formulation is inhaled as an aerosol as is known in the art, for uptake into appropriate vascular or lymphatic tissue in the lungs.

2. Vaccination with Nucleic Acid Vaccines:

A nucleic acid vaccine is injected into a lymph node using a miniature pumping system, such as the MiniMed insulin pump. A nucleic acid construct formulated in an aqueous buffered solution containing an antimicrobial agent, an antioxidant, and an immunomodulating cytokine, is delivered over a several day infusion cycle in order to mimic the kinetics of antigen presentation during a natural infection.

Optionally, the nucleic acid construct is delivered using controlled release substances, such as PLGA microspheres or other biodegradable substances. These substances are injected or taken orally. Nucleic acid vaccines are given using oral delivery, priming the immune response through uptake into GALT tissues. Alternatively, the nucleic acid vaccines are delivered using a gene gun, wherein the nucleic acid vaccine is adhered to minute gold particles. Nucleic acid constructs can also be inhaled as an aerosol, for uptake into appropriate vascular or lymphatic tissue in the lungs.

### Example 9

Assays for the Effectiveness of Epitope Vaccines.

1. Tetramer Analysis:

Class I tetramer analysis is used to determine T cell frequency in an animal before and after administration of a housekeeping epitope. Clonal expansion of T cells in response to an epitope indicates that the epitope is presented to T cells by pAPCs. The specific T cell frequency is measured against the housekeeping epitope before and after administration of the epitope to an animal, to determine if the epitope is present on pAPCs. An increase in frequency of T cells specific to the epitope after administration indicates that the epitope was presented on pAPC.

2. Proliferation Assay:

Approximately 24 hours after vaccination of an animal with housekeeping epitope, pAPCs are harvested from PBMCs, splenocytes, or lymph node cells, using monoclonal antibodies against specific markers present on pAPCs, fixed to magnetic beads for affinity purification. Crude blood or splenoctye preparation is enriched for pAPCs using this technique. The enriched pAPCs are then used in a proliferation assay against a T cell clone that has been generated and is specific for the housekeeping epitope of interest. The pAPCs are coincubated with the T cell clone and the T cells are monitored for proliferation activity by measuring the incorporation of radiolabeled thymidine by T cells. Proliferation indicates that T cells specific for the housekeeping epitope are being stimulated by that epitope on the pAPCs.

3. Chromium Release Assay:

A human patient, or non-human animal genetically engineered to express human class I MHC, is immunized using a housekeeping epitope. T cells from the immunized subject are used in a standard chromium release assay using human tumor targets or targets engineered to express the same class I MHC. T cell killing of the targets indicates that stimulation of T cells in a patient would be effective at killing a tumor expressing a similar TuAA.

### Example 10

Induction of CTL Response with Naked DNA is Efficient by Intra-Lymph Node Immunization.

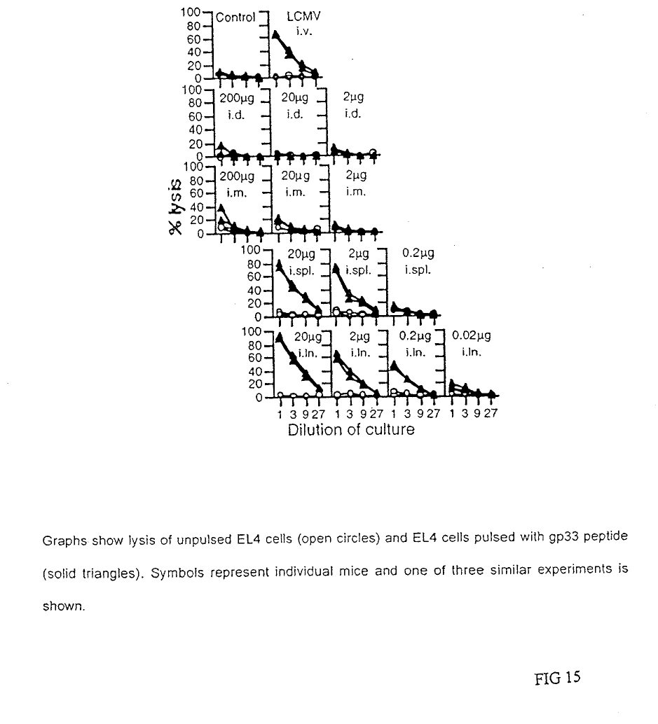

In order to quantitatively compare the CD8⁺ CTL responses induced by different routes of immunization a plasmid DNA vaccine (pEGFPL33A) containing a well-characterized immunodominant CTL epitope from the LCMV-glycoprotein (G) (gp33; amino acids 3341) (Oehen, S., et al. Immunology 99, 163-169 2000) was used, as this system allows a comprehensive assessment of antiviral CTL responses. Groups of 2 C57BL/6 mice were immunized once with titrated doses (200-0.02 μg) of pEGFPL33A DNA or of control plasmid pEGFP-N3, administered i.m. (intramuscular), i.d. (intradermal), i.spl. (intrasplenic), or i.ln. (intra-lymph node). Positive control mice received 500 pfu LCMV i.v. (intravenous). Ten days after immunization spleen cells were isolated and gp33-specific CTL activity was determined after secondary in vitro restimulation. As shown in FIG. 15, i.m. or i.d. immunization induced weakly detectable CTL responses when high doses of pEFGPL33A DNA (200 μg) were administered. In contrast, potent gp33-specific CTL responses were elicited by immunization with only 2 μg pEFGPL33A DNA i.spl. and with as little as 0.2%g pEFGPL33A DNA given i.ln. (FIG. 15; symbols represent individual mice and one of three similar experiments is shown). Immunization with the control pEGFP-N3 DNA did not elicit any detectable gp33-specific CTL responses (data not shown).

### Example 11

Intra-Lymph Node DNA Immunization Elicits Anti-Tumor Immunity.

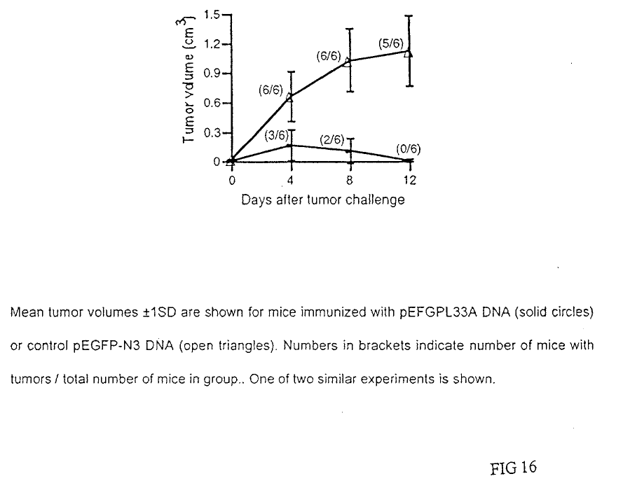

To examine whether the potent CTL responses elicited following i.ln. immunization were able to confer protection against peripheral tumors, groups of 6 C57BL/6 mice were immunized three times at 6-day intervals with 10 μg of pEFGPL33A DNA or control pEGFP-N3 DNA. Five days after the last immunization small pieces of solid tumors expressing the gp33 epitope (EL4-33) were transplanted s.c. into both flanks and tumor growth was measured every 3-4d. Although the EL4-33 tumors grew well in mice that had been repetitively immunized with control pEGFP-N3 DNA (FIG. 16), mice which were immunized with pEFGPL33A DNA i.ln. rapidly eradicated the peripheral EL4-33 tumors (FIG. 16).

### Example 12

Differences in Lymph Node DNA Content Mirrors Differences in CTL Response Following Intra-Lymph Node and Intramuscular Injection.

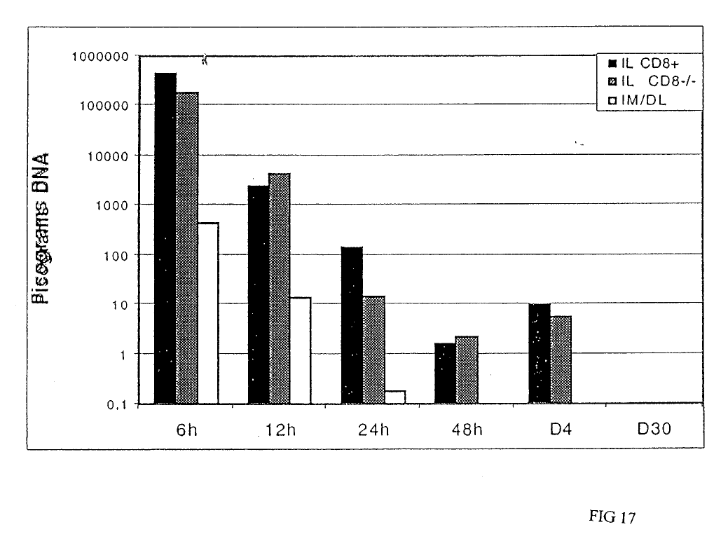

pEFGPL33A DNA was injected i.ln. or i.m. and plasmid content of the injected or draining lymph node was assessed by real time PCR after 6, 12, 24, 48 hours, and 4 and 30 days. At 6, 12, and 24 hours the plasmid DNA content of the injected lymph nodes was approximately three orders of magnitude greater than that of the draining lymph nodes following i.m. injection. No plasmid DNA was detectable in the draining lymph node at subsequent time points (FIG. 17). This is consonant with the three orders of magnitude greater dose needed using i.m. as compared to i.ln. injections to achieve a similar levels of CTL activity. CD8^{-/-} knockout mice, which do not develop a CTL response to this epitope, were also injected i.ln. showing clearance of DNA from the lymph node is not due to CD8⁺ CTL killing of cells in the lymph node. This observation also supports the conclusion that i.ln. administration will not provoke immunopathological damage to the lymph node.

### Example 13

Administration of a DNA Plasmid Formulation of a Therapeutic Vaccine for Melanoma to Humans.

SYNCHROTOPE TA2M, a melanoma vaccine, encoding the HLA-A2-restricted tyrosinase epitope SEQ ID NO. 1 and epitope cluster SEQ ID NO. 69, was formulated in 1% Benzyl alcohol, 1% ethyl alcohol, 0.5 mM EDTA, citrate-phosphate, pH 7.6. Aliquots of 80, 160, and 320 μg DNA/ml were prepared for loading into MINIMED 407C infusion pumps. The catheter of a SILHOUETTE infusion set was placed into an inguinal lymph node visualized by ultrasound imaging. The assembly of pump and infusion set was originally designed for the delivery of insulin to diabetics and the usual 17 mm catheter was substituted with a 31 mm catheter for this application. The infusion set was kept patent for 4 days (approximately 96 hours) with an infusion rate of about 25 μl/hour resulting in a total infused volume of approximately 2.4 ml. Thus the total administered dose per infusion was approximately 200, and 400 μg; and can be 800 μg, respectively, for the three concentrations described above. Following an infusion subjects were given a 10 day rest period before starting a subsequent infusion. Given the continued residency of plasmid DNA in the lymph node after administration (as in example 12) and the usual kinetics of CTL response following disappearance of antigen, this schedule will be sufficient to maintain the immunologic CTL response.

### Example 14

Additional Epitopes.

The methodologies described above, and in particular in examples 3-7, have been applied to additional synthetic peptide substrates, leading to the identification of further epitopes as set for the in tables 15-36 below. The substrates used here were designed to identify products of housekeeping proteasomal processing that give rise to HLA-A*0201 binding epitopes, but additional MHC-binding reactivities can be predicted, as discussed above. Many such reactivities are disclosed, however, these listings are meant to be exemplary, not exhaustive or limiting. As also discussed above, individual components of the analyses can be used in varying combinations and orders. The digests of the NY-ESO-1 substrates 136-163 and 150-177 (SEQ ID NOS. 254 and 255, respectively) yielded fragments that did not fly well in MALDI-TOF mass spectrometry. However, they were quite amenable to N-terminal peptide pool sequencing, thereby allowing identification of cleavage sites. Not all of the substrates necessarily meet the formal definition of an epitope cluster as referenced in example 3. Some clusters are so large, e.g. NY-ESO-1₈₆₋₁₇₁, that it was more convenient to use substrates spanning only a portion of this cluster. In other cases, substrates were extended beyond clusters meeting the formal definition to include neighboring predicted epitopes. In some instances, actual binding activity may have dictated what substrate was made, as with for example the MAGE epitopes reported here, where HLA binding activity was determined for a selection of peptides with predicted affinity, before synthetic substrates were designed.

TABLE 15

GP100: Preferred Epitopes Revealed by Housekeeping Proteasome Digestion

| | | | SEQ ID | HLA Binding Predictions (SYFPEITHI/NIH)† | | | | | |
|---|---|---|---|---|---|---|---|---|---|
| Substrate | Epitope | Sequence | NO | A*0201 | A1 | A3 | B7 | B8 | Comments |

| | | | | | | | | |
|---|---|---|---|---|---|---|---|---|
| 609-644 | 630-638* | LPHSSSHWL | 88 | | | 20/80 | 16/<5 | *The digestion of |
| | | | | | | | | 609-644 and 622-650 |
| | 629-638* | QLPHSSSHWL | 89 | 21/117 | | | | have generated |
| | | | | | | | | the same epitopes. |
| | 614-622 | LIYRRRLMK | 90 | | 32/20 | | | |
| | 613-622 | SLIYRRRLMK | 91 | 14/<5 | 29/60 | | | |
| | 615-622 | IYRRRLMK | 92 | | | | 15/<5 | |
| 622-650 | 630-638* | LPHSSSHWL | 93 | | | 20/80 | 16/<5 | |
| | 629-638* | QLPHSSSHWL | 94 | 21/117 | | | | |

†Scores are given from the two binding prediction programs referenced aboe (see example 3)

TABLE 16A

MAGE-1: Preferred Epitopes Revealed by Housekeeping Proteasome Digesti

| | |
|---|---|
| SEQ ID | HLA Binding Predictions (SYFPEITHI/NIH)† |

| | | | | | | | | | |
|---|---|---|---|---|---|---|---|---|---|
| Substrate | Epitope | Sequence | NO | A*0201 | A1 | A3 | B7 | B8 | Other |

| | | | | | | | | | |
|---|---|---|---|---|---|---|---|---|---|
| 86-109 | 95-102 | ESLFRAVI | 95 | | | | | 16/<5 | |
| | 93-102 | ILESLFRAVI | 96 | 21/<5 | | 20/<5 | | | |
| | 93-101 | ILESLFRAV | 97 | 23/<5 | | | | | |
| | 92-101 | CILESLFRAV | 98 | 23/55 | | | | | |
| | 92-100 | CILESLFRA | 99 | 20/138 | | | | | |
| 263-292 | 263-271 | EFLWGPRAL | 100 | | | | | | A26 (R 21), |
| | | | | | | | | | A24 (NIH 30) |
| | 264-271 | FLWGPRAL | 101 | | | | | 17/<5 | |
| | 264-273 | FLWGPRALAE | 102 | 16/<5 | | 19/<5 | | | |
| | 265-274 | LWGPRALAET | 103 | 16/<5 | | | | | |
| | 268-276 | PRALAETSY | 104 | 15/<5 | | | | | |
| | 267-276 | GPRALAETSY | 105 | 15/<5 | | | <15/<5 | | B4403 (NIH 7); |
| | | | | | | | | | B3501 (NIH 120) |
| | 269-277 | RALAETSYV | 106 | 18/20 | | | | | |
| | 271-279 | LAETSYVKV | 107 | 19/<5 | | | | | |
| | 270-279 | ALAETSYVKV | 108 | 30/427 | | 19/<5<5 | | | |
| | 272-280 | AETSYVKVL | 109 | 15/<5 | | | | | B.b4403 (NIH 36) |
| | 271-280 | LAETSYVKVL | 110 | 18/<5 | | | <15/<5 | | |
| | 274-282 | KVLEYVIKV | 111 | | 26/<5 | | | | B4403 (NIH 14) |
| | 273-282 | ETSYVKVLEY | 112 | | 28/6 | | | | A26 (R 31), B4403 |
| | | | | | | | | | (NIH 14) |
| | 278-286 | KVLEYVIKV | 113 | 26/743 | | 16/<5 | | | |

TABLE 16B

MAGE-1: Preferred Epitopes Revealed by Housekeeping Proteasome Digestion

| | |
|---|---|
| SEQ ID | HLA Binding Predictions (SYFPEITHI/NIH)† |

| | | | | | | | | | |
|---|---|---|---|---|---|---|---|---|---|
| Substrate | Epitope | Sequence | NO | A*0201 | A1 | A3 | B7 | B8 | Other |

| | | | | | | | | | |
|---|---|---|---|---|---|---|---|---|---|
| 168-193 | 168-177 | SYVLVTCLGL | 114 | | | | | | A24 (NIH 300) |
| | 169-177 | YVLVTCLGL | 115 | 20/32 | | 15/<5 | <15/20 | | |
| | 170-177 | VLVTCLGL | 116 | | | | | 17/<5 | |
| 229-258 | 240-248 | TQDLVQEKY | 117 | | 29/<5 | | | | |
| | 239-248 | LTQDLVQEKY | 118 | | 23/<5 | | | | A26 (R 22) |
| | 232-240 | YGEPRKLLT | 119 | | 24/11 | | | | |
| | 243-251 | LVQEKYLEY | 120 | | 21/<5 | 21/<5 | | | A26 (R 28) |
| | 242-251 | DLVQEKYLEY | 121 | | 22/<5 | 19/<5 | | | A26 (R 30) |
| | 230-238 | SAYGEPRKL | 122 | 21/<5 | | | | | B5101 (25/121) |
| 272-297 | 278-286 | KVLEYVTKV | 123 | 26/743 | | 16/<5 | | | |
| | 277-286 | VKVLEYVIIKV | 124 | 17/<5 | | | | | |
| | 276-284 | YVKVLEYVI | 125 | 15/<5 | | 15/<5 | | 17/<5 | |
| | 274-282 | TSYVKVLEY | 126 | | 26/<5 | | | | |
| | 273-282 | ETSYVKVLEY | 127 | | 28/6 | | | | |
| | 283-291 | VIKVSARVR | 128 | | | 20/<5 | | | |
| | 282-291 | YVIKVSARVR | 129 | | | 24/<5 | | | |

†Scores are given from the two binding prediction programs referenced above (see example 3). R indicates a SYFPEITHI score.

TABLE 17A

MAGE-2: Preferred Epitopes Revealed by Housekeeping Proteasome Digestion

| | |
|---|---|
| SEQ ID | HLA Binding Predictions (SYFPEITHI/NIH)† |

| | | | | | | | | | |
|---|---|---|---|---|---|---|---|---|---|
| Substrate | Epitope | Sequence | NO | A*0201 | A1 | A3 | B7 | B8 | Other |

| | | | | | | | | | |
|---|---|---|---|---|---|---|---|---|---|
| 107-126 | 115-122 | ELVHFLLL | 130 | | | | | 18/<5 | |
| | 113-122 | MVELVLIFLLL | 131 | | 21/<5 | | | | A26 (R 22) |
| | 109-116 | ISRKMVEL | 132 | | | | | 17/<5 | |
| | 108-116 | AISRKMVEL | 133 | 25/7 | | 19/<5 | 16/12 | 26/<5 | |
| | 107-116 | AAISRKMVEL | 134 | 221<5 | | | | | |
| | 112-120 | KMVELVHFL | 135 | 27/2800 | | | | | |
| | 109-117 | ISRKMVELV | 136 | 16/<5 | | | | | |
| | 108-117 | AISRKMVELV | 137 | 24/11 | | | | | |
| | 116-124 | LVHFLLLKY | 138 | | 23/<5 | 19/<5 | | | A26 (R 26) |
| | 115-124 | ELVHFLLLKY | 139 | | 24/<5 | 19/5 | | | A26 (R 29) |
| | 111-119 | RKMVELVHF | 140 | | | | | | |
| 145-175 | 158-166 | LQLVFGLEV | 141 | 17/168 | | | | | |
| | 157-166 | YLQLVFGIEV | 142 | 24/1215 | | | | | |
| | 159-167 | QLVFGIEVV | 143 | 25/32 | | 18/<5 | | | |
| | 158-167 | LQLVFGTEVV | 144 | 18/20 | | | | | |
| | 164-172 | IEVVEVVPI | 145 | 161<5 | | | | | |
| | 163-172 | GIEVVEVVPI | 146 | 22/<5 | | | | | |
| | 162-170 | FGIEVVEVV | 147 | 19/<5 | | | | | B5101(24/69.212) |
| | 154-162 | ASEYLQLVF | 148 | | 22/68 | | | | |
| | 153-162 | KASEYLQLVF | 149 | | | 15/<5 | | | |

†Scores are given from the two binding prediction programs referenced above (see example 3). R indicates a SYFPEITHI score.

TABLE 17B

MAGE-2: Preferred Epitopes Revealed by Housekeeping Proteasome Digestion

HLA Binding Predictions (SYFPEITHI/NIH)†

| | | | | | | | | |
|---|---|---|---|---|---|---|---|---|
| Substrate | Epitope | Sequence | A*0201 | A1 | A3 | B7 | B8 | Other |

| | | | | | | | | |
|---|---|---|---|---|---|---|---|---|
| 213-233 | 218-225 | EEIWEEL | 150 | | | | 22/<5 | |
| | 216-225 | APEEKIWEEL | 151 | 15/<5 | | 22/72 | | |
| | 216-223 | APEEKIWE | 152 | | | | 18/<5 | |
| | 220-228 | KIWEELSML | 153 | 26/804 | 16/<5 | | 16/<5 | A26 (R 26) |
| | 219-228 | EKIWEELSML | 154 | | | | | A26 (R 22) |
| 271-291 | 271-278 | FLWGPRAL | 155 | | | | 17/<5 | |
| | 271-279 | FLWGPRALI | 156 | 25/398 | 16/7 | | | |
| | 278-286 | LIETSYVKV | 157 | 23/<5 | | | | |
| | 277-286 | ALIETSYVKV | 158 | 30/427 | 21/<5 | | | |
| | 276-284 | RALIETSYV | 159 | 18/19 | | | B5101 (20/55) | |
| | 279-287 | IETSYVKVL | 160 | 15/<5 | | | | |
| | 278-287 | LLETSYVKVL | 161 | 22/<5 | | | | A26 (R 22) |

†Scores are given from the two binding prediction programs referenced above (see example 3). R indicates a SYFPEITHI score

TABLE 18

MAGE-3: Preferred Epitopes Revealed by Housekeeping Proteasome Digestion

| | |
|---|---|
| SEQ ID | HLA Binding Predictions (SYFPEITHI/NIH)† |

| | | | | | | | | | |
|---|---|---|---|---|---|---|---|---|---|
| Substrate | Epitope | Sequence | NO | A*0201 | A1 | A3 | B7 | B8 | Other |

| | | | | | | | |
|---|---|---|---|---|---|---|---|
| 267-286 | 271-278 | FLWGPRAL | 162 | | | 17/<5 | |
| | 270-278 | EFLWGPRAL | 163 | | | | A26 (R 21); |
| | | | | | | | A24 (NIH 30) |
| | 271-279 | FLWGPRALV | 164 | 27/2655 | 16/21 5 | | |
| | 276-284 | RALVETSYV | 165 | 18/19 | | | B5101 (20/55) |
| | 272-280 | LWGPRALVE | 166 | | 15/<5 | | |
| | 271-280 | FLWGPRALVE | 167 | 15/<5 | 22/<5 | | |
| | 272-281 | LWGPRALVET | 168 | 16/<5 | | | |

†Scores are given from the two binding prediction programs referenced above (see example 3). R indicates a SYFPEITHI score.

TABLE 19A

NY-ESO-1: Preferred Epitopes Revealed by Housekeeping Proteasome Digestion

| | |
|---|---|
| SEQ ID | HLA Binding Predictions (SYFPEITHI/NIH)† |

| | | | | | | | | | |
|---|---|---|---|---|---|---|---|---|---|
| Substrate | Epitope | Sequence | NO | A*0201 | A1 | A3 | B7 | B8 | Other |

| | | | | | | | | | |
|---|---|---|---|---|---|---|---|---|---|
| 81-113 | 82-90 | GPESRLLEF | 169 | | 16/11 | | 18/<5 | 22/<5 | |
| | 83-91 | PESRLLEFY | 170 | | 15/<5 | | | | B4403 (NIH 18) |
| | 82-91 | GPESRLLEFY | 171 | | 25/11 | | | | |
| | 84-92 | ESRLLEFYL | 172 | | | | | 19/8 | |
| | 86-94 | RLLEFYLAM | 173 | 21/430 | | 21/<5 | | | |
| | 88-96 | LEFYLAMPF | 174 | | | | | | B4403 (NIH 60) |
| | 87-96 | LLEFYLAMPF | 175 | | <15/45 | 18/<5 | | | |
| | 93-102 | AMPFATPMEA | 176 | 15/<5 | | | | | |
| | 94-102 | MPFATPMEA | 177 | | | | 17/<5 | | |
| 101-133 | 115-123 | PLPVPGVLL | 178 | 20/<5 | | 17/<5 | 16/<5 | 18/<5 | |
| | 114-123 | PPLPVPGVLL | 179 | | | | 23/12 | | |
| | 116-123* | LPVPGVLL | 180 | | | | | 16/<5 | Comment |
| | 103-112 | ELARRSLAQD | 181 | 15/<5 | 20/<5 | | | *Evidence of the | |
| | 118-126* | VPGVLLKEF | 182 | | | | 17/<5 | 16/<5 | same epitope |
| | 117-126* | PVPGVLLKEF | 183 | | | 16/<5 | | | obtained from two |
| 116-145 | 116-123* | LPVPGVLL | 184 | | | | | 16/<5 | digests. |
| | 127-135 | TVSGNILTI | 185 | 21/<5 | | 19/<5 | | | |
| | 126-135 | FTVSQNILTI | 186 | 20/<5 | | | | | |
| | 120-128 | GVLLKEFTV | 187 | 20/130 | | 18/<5 | | | |
| | 121-130 | VLLKEFTVSG | 188 | 17/<5 | | 18/<5 | | | |
| | 122-130 | LLKEFTVSG | 189 | 20/<5 | | 18/<5 | | | |
| | 118-126* | VPGVLLKEF | 190 | | | | 17/<5 | 16/<5 | |
| | 117-126* | PVPGVLLKEF | 191 | | | 16/<5 | | | |

†Scores are given from the two binding prediction programs referenced above (see example 3).

TABLE 19B

NY-ESO-1: Preferred Epitopes Revealed by Housekeeping Proteasome Digestion

| | |
|---|---|
| SEQ ID | HLA Binding Predictions (SYFPEITHI/NIH)† |

| | | | | | | | | | |
|---|---|---|---|---|---|---|---|---|---|
| Substrate | Epitope | Sequence | NO | A*0201 | A1 | A3 | B7 | B8 | Other |

| | | | | | | | | |
|---|---|---|---|---|---|---|---|---|
| 136-163 | 139-147 | AADHRQLQL | 192 | 17/<5 | 17/<5 | | 22/<5 | |
| (SEQ ID NO | 148-156 | SISSCLQQL | 193 | 24/7 | | | | A26 (R 25) |
| 254) | 147-156 | LSISSCLQQL | 194 | 18/<5 | | | | |
| | 138-147 | TAADHRQLQL | 195 | 18/<5 | | | | |
| 150-177 | 161-169 | WITQCFLPV | 196 | 18/84 | | | | |
| (SEQ ID NO | 157-165 | SLLMWITQC | 197 | 18/42 | | 17/<5 | | |
| 255) | 150-158 | SSCLQQLSL | 198 | 15/<5 | | | | |
| | 154-162 | QQLSLLMWI | 199 | 15/50 | | | | |
| | 151-159 | SCLQQLSLL | 200 | 18/<5 | | | | |
| | 150-159 | SSCLQQLSLL | 201 | 16/<5 | | | | |
| | 163-171 | TQCFLPVFL | 202 | <15/12 | | | | |
| | 162-171 | ITQCFLPVFL | 203 | 18/<5 | | | | A26 (R 19) |

†Scores are given from the two binding prediction programs referenced above (see example 3). R indicates SYFPEITHI score

TABLE 20

PRAME: Preferred Epitopes Revealed by Housekeeping Proteasome Digestion

| | |
|---|---|
| SEQ ID | HLA Binding Predictions (SYFPEITHI/NIH)† |

| | | | | | | | | | |
|---|---|---|---|---|---|---|---|---|---|
| Substrate | Epitope | Sequence | NO | A*0201 | A1 | A3 | B7 | B8 | Other |

| | | | | | | | |
|---|---|---|---|---|---|---|---|
| 211-245 | 219-227 | PMQDIKMIL | 204 | 16/<5 | | 16/n.d. | A26 (R 20) |
| | 218-227 | MPMQDIKMIL | 205 | | <15/240 | | |
| 411-446 | 428-436 | QHLIGLSNL | 206 | 18/<5 | | | |
| | 427-436 | LQHLIGLSNL | 207 | 16/8 | | | |
| | 429-436 | HLIGLSNL | 208 | | | 17/<5 | B15 (R 21) |
| | 431-439 | IGLSNLTHV | 209 | 18/7 | | | B*5101 (R 22) |
| | 430-439 | LIGLSNLTHV | 210 | 24/37 | | | |

†Scores are given from the two binding prediction programs reference above (see example 3). R indicates a SYFPEITHI score.

TABLE 21

PSA: Preferred Epitopes Revealed by Housekeeping

| | |
|---|---|
| SEQ ID | HLA Binding Predictions (SYFPEITHI/NIH)† |

| | | | | | | | | | |
|---|---|---|---|---|---|---|---|---|---|
| Substrate | Epitope | Sequence | NO | A*0201 | A1 | A3 | B7 | B8 | Other |

| | | | | | | | | |
|---|---|---|---|---|---|---|---|---|
| 42-77 | 53-61 | VLVHPQWVL | 211 | 22/112 | | <15/6 | 17<5 | |
| | 52-61 | GVLVHPQWVL | 212 | 17/21 | 16/<5 | <15/30 | | A26 (R 18) |
| | 52-60 | GVLVHPQWV | 213 | 17/124 | | | | |
| | 59-67 | WVLTAAHCI | 214 | 15/16 | | | | |
| | 54-63 | LVHPQWVLTA | 215 | 19/<5 | 20/<5 | | | A26 (R 16) |
| | 53-62 | VLVHPQWVLT | 216 | 17/22 | | | | |
| | 54-62 | LVHPQWVLT | 217 | | 17/n.d. | | | |
| 55-95 | 66-73 | CIRNKSVI | 218 | | | | 26/20 | |
| | 65-73 | HCIRNKSVI | 219 | | | | <15/16 | |
| | 56-64 | HPQWVLTAA | 220 | | | 18/<5 | | |
| | 63-72 | AAHCIRNKSV | 221 | 17/<5 | | | | |

†Scores are given from the two binding prediction programs referenced above (see example 3). R indicates a SYFPEITHI score.

TABLE 22

PSCA: Preferred Epitopes Revealed by Housekeeping Proteasoe Digestion

| | |
|---|---|
| SEQ ID | HLA Binding Predictions (SYFPEITHI/NIH)† |

| | | | | | | | | | |
|---|---|---|---|---|---|---|---|---|---|
| Substrate | Epitope | Sequence | NO | A*0201 | A1 | A3 | B7 | B8 | Other |

| | | | | | | | | |
|---|---|---|---|---|---|---|---|---|
| 93-123* | 116-123 | LLWGPGQL | 222 | | | | 16/<5 | |
| | 115-123 | LLLWGPGQL | 223 | <15/18 | | | | |
| | 114-123 | GLLLWGPGQL | 224 | <15/10 | | | | |
| | 99-107 | ALQPAAAIL | 225 | 26/9 | 22/<5 | <15/12 | 16/<5 | A26 (R 19) |
| | 98-107 | HALQPAAAIL | 226 | 18/<5 | | <15/12 | | |

*L123 is the C-terminus of the natural prote
†Scores are given from the two binding prediction programs referenced above (see example 3).

TABLE 23

Tyrosinase: Preferred Epitopes Revealed by Housekeeping Proteasome Digestion

| | |
|---|---|
| SEQ ID | HLA Binding Predictions (SYFPEITHI/NIH)† |

| | | | | | | | | | |
|---|---|---|---|---|---|---|---|---|---|
| Substrate | Epitope | Sequence | NO | A*0201 | A1 | A3 | B7 | B8 | Other |

| | | | | | | | | | |
|---|---|---|---|---|---|---|---|---|---|
| 128-157 | 128-137 | APEKDKFFAY | 227 | | 29/6 | | 15/<5 | | B4403 (NIH 14) |
| | 129-137 | PEKDKFFAY | 228 | | 18/<5 | | | 21/<5 | |
| | 130-138 | EKDKFFAYL | 229 | | | | 15/<5 | | |
| | 131-138 | KDKIFFAYL | 230 | | | | | 20/<5 | |
| 197-228 | 205-213 | PAFLPWIIRL | 231 | | | | | 15/<5 | |
| | 204-213 | APAFLPWHRL | 232 | | | | 23/360 | | |
| | 207-216 | FLPWIIRLFLL | 1 | 25/1310 | | | | <15/18 | |
| | 208-216 | LPWHRLFLL | 9 | 17/26 | | | 20/80 | 24/16 | |
| | 214-223 | FLLRWEQEIQ | 233 | | | 15/<5 | | | |
| | 212-220 | RLFLLRWEQ | 234 | | | 16/<5 | | | |
| 191-211 | 191-200 | GSETWRDIDF | 235 | | 18/68 | | | | |
| | 192-200 | SEIWRDIDF | 236 | | | | | 16/<5 | B4403 (NIH 400) |
| 207-230 | 207-215 | FLWIIRLFL | 8 | 22/540 | | | <15/6 | 17/<5 | |
| 466-484 | 473-481 | RTWSWLLGA | 237 | 19/13 | | 15/<5 | | | |
| 476-497 | 476-484 | SWLLGAAMV | 238 | 18/<5 | | | | | |
| | 477-486 | WLLGAAMVGA | 239 | 21/194 | | 18/<5 | | | |
| | 478-486 | LLGAAMVGA | 240 | 19/19 | | 16/<5 | | | |

†Scores are given from the two binding prediction programs referenced above (see example 3).

TABLE 24

PSMA: Preferred Epitopes Revealed by Housekeeping Proteasome Digestion

| | |
|---|---|
| SEQ ID | HLA Binding Predictions (SYFPEITHI/NIH)† |

| | | | | | | | | | |
|---|---|---|---|---|---|---|---|---|---|
| Substrate | Epitope | Sequence | NO | A*0201 | A1 | A3 | B7 | B8 | Other |

| | | | | | | | | |
|---|---|---|---|---|---|---|---|---|
| 1-30 | 4-12 | LLHETDSAV | 241 | 25/485 | | 15/<5 | | |
| | 13-21 | ATARRPRWL | 242 | 18/<5 | | | 18/<5 | |
| 53-80 | 53-61 | TPKHNMKAF | 243 | | | | 24/<5 | |
| | 64-73 | ELKAENIKKF | 244 | | | 17/<5 | | A26 (R 30) |
| | 69-77 | NIKKFLH¹NF | 245 | | | | | A26 (R 27) |
| | 68-77 | ENIKKFLH¹NF | 246 | | | | | A26 (R 24) |
| 215-244 | 220-228 | AGAKGVILY | 247 | | 25/<5 | | | |
| 457-489 | 468-477 | PLMYSLVLJNL | 248 | 22/<5 | | | | |
| | 469-477 | LMYSLVHNL | 249 | 27/193 | | <15/9 | | |
| | 463-471 | RVDCTPLMY | 250 | | 32/125 | 25/<5 | | A26 (R 22) |
| | 465-473 | DCTPLMYSL | 251 | | | | | A26 (R 22) |
| 503-533 | 507-515 | SGMPRISKL | 252 | 21/<5 | | | 21<5 | |
| | 506-515 | FSGMPRISKL | 253 | 17/<5 | | | | |

¹This was reported as Y in the SWISSPROT database.
†Scores are given from the two binding prediction programs referenced above (see example 3).

TABLE 25A

MAGE-1: Preferred Epitopes Revealed by Housekeeping Proteasome Di-
gestion

Binding Prediction

| | | | | | | |
|---|---|---|---|---|---|---|
| Substrate | Epitope | Sequence | Seq. Id No. | HLA type | SYFPEITHI | NIH |

| | | | | | | |
|---|---|---|---|---|---|---|
| Mage-1 119-146 | 125-132 | KAEMLESV | 256 | B5101 | 19 | n.a. |
| | 124-132 | TKAEMLESV | 257 | A0201 | 20 | <5 |
| | 123-132 | VTKAEMLESV | 258 | A0201 | 20 | <5 |
| | 128-136 | MLESVIKNY | 259 | A1 | 28 | 45 |
| | | | | A26 | 24 | n.a. |
| | | | | A3 | 17 | 5 |
| | 127-136 | EMLESVIKNY | 260 | A1 | 15 | <1.0 |
| | | | | A26 | 23 | <1.0 |
| | 125-133 | KAEMLESVI | 261 | B5101 | 23 | 100 |
| | | | | A24 | N.A. | 4 |
| Mage-1 143-170 | 146-153 | KASESLQL | 262 | B08 | 16 | <1.0 |
| | | | | B5101 | 17 | N.A. |
| | 145-153 | GKASESLQL | 263 | B2705 | 17 | 1 |
| | | | | B2709 | 16 | N.A. |
| | 147-155 | ASESLQLVF | 264 | A1 | 22 | 68 |
| | 153-161 | LVFGIDVKE | 265 | A26 | 16 | N.A. |
| | | | | A3 | 16 | <1.0 |

TABLE 25B

MAGE-1: Preferred Epitopes Revealed by Housekeeping Proteasome Di-
gestion

| | | | | Binding Prediction +HL,42 | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| Mage-1 99-125 | 114-121 | LLKYRARE | 266 | B8 | 25 | <1.0 |
| | 106-113 | VADLVGFL | 267 | B8 | 16 | <1.0 |
| | | | | B5101 | 21 | N.A. |
| | 105-113 | KVADLVGFL | 268 | A0201 | 23 | 44 |
| | | | | A26 | 25 | N.A. |
| | | | | A3 | 16 | <5 |
| | | | | B0702 | 14 | 20 |
| | | | | B2705 | 14 | 30 |
| | 107-115 | ADLVGFLLL | 269 | A0201 | 17 | <5 |
| | | | | B0702 | 15 | <5 |
| | | | | B2705 | 16 | 1 |
| | 106-115 | VADLVGFLLL | 270 | A0201 | 16 | <5 |
| | | | | A1 | 22 | 3 |
| | 114-123 | LLKYRAREPV | 271 | A0201 | 20 | 2 |

TABLE 26

MAGE-3: Preferred Epitopes Revealed by Housekeeping Proteasome Diges-
tion

Binding Predition

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| Mage-3 267-295 | 271-278 | FLWGPRAL | 162 | B08 | 17 | <5 |
| | 270-278 | EFLWGPRAL | 163 | A26 | 21 | N.A. |
| | | | | A24 | N.A. | 30 |
| | | | | B1510 | 16 | N.A. |
| | 271-279 | FLWGPRALV | 164 | A0201 | 27 | 2655 |
| | | | | A3 | 16 | 2 |
| | 278-286 | LVETSYVKV | 272 | A0201 | 19 | <1.0 |
| | | | | A26 | 17 | N.A. |
| | 277-286 | ALVETSYVKV | 273 | A0201 | 28 | 428 |
| | | | | A26 | 16 | <5 |
| | | | | A3 | 18 | <5 |
| | 285-293 | KVLHHMVKI | 274 | A0201 | 19 | 27 |
| | | | | A3 | 19 | <5 |
| | 276-284 | RALVETSYV | 165 | A0201 | 18 | 20 |
| | 283-291 | YVKVLHHMV | 275 | A0201 | 17 | <1.0 |
| | 275-283 | PRALVETSY | 276 | A1 | 17 | <1.0 |
| | 274-283 | GPRALVETSY | 277 | A1 | 15 | <1.0 |
| | 278-287 | LVETSYVKVL | 278 | A0201 | 18 | <1.0 |
| | 272-281 | LWGPRALVET | 168 | A0201 | 16 | <1.0 |
| | 271-280 | FLWGPRALVE | 167 | A3 | 22 | <5 |

TABLE 27A

Fibronectin ED-B: Preferred Epitopes Revealed by Housekeeping Pro-
teasome Digestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| ED-B 14′-21* | 4′-5** | TIIPEVPQL\ | 279 | A0201 | 27 | 7 |
| | | | | A26 | 28 | N.A. |
| | | | | A3 | 17 | <5 |
| | | | | B8 | 15 | <5 |
| | | | | B1510 | 15 | N.A. |
| | | | | B2705 | 17 | 10 |
| | | | | B2709 | 15 | N.A. |
| | 5′-5** | DTIIPEVPQL† | 280 | A0201 | 20 | <5 |
| | | | | A26 | 32 | N.A. |
| | 1-10 | EVPQLTDLSF | 281 | A26 | 29 | N.A. |

*This substrate contains the 14 amino acids from fibronectin flanking ED-B to the N-terminal side.
**These peptides span the junction between the N-terminus of the ED-B domain and the rest of fibronectin.
†The italicized lettering indicates sequence outside the ED-B domain.

TABLE 27B

Fibronectin ED-B: Preferred Epitopoes Revealed by
Housekeeping Proteasome Digestion

| | |
|---|---|
| Seq. | Binding Prediction |

| | | | | | | |
|---|---|---|---|---|---|---|
| Sub- | Epi- | ID | HLA | | | |
| strate | tope | Sequence | No. | type | SYFPEITHI | NIH |

| | | | | | | |
|---|---|---|---|---|---|---|
| ED-B | 23-30 | TPLNSSTI | 282 | B5101 | 22 | N.A. |
| 8-35 | 18-25 | IGLRWTPL | 283 | B5101 | 18 | N.A. |
| | 17-25 | SIGLRWTPL | 284 | A0201 | 20 | 5 |
| | | | | A26 | 18 | N.A. |
| | | | | B08 | 25 | <5 |
| | 25-33 | LNSSTIIGY | 285 | A1 | 19 | <5 |
| | | | | A26 | 16 | <5 |
| | 24-33 | PLNSSTIIGY | 286 | A1 | 20 | <5 |
| | | | | A26 | 24 | N.A. |
| | | | | A3 | 16 | <5 |
| | 23-31 | TPLNSSTII | 287 | B0702 | 17 | 8 |
| | | | | B5101 | 25 | 440 |

TABLE 27C

Fibronectin ED-B: Preferred Epitopes Revealed by Housekeeping Pro-
teasome Digestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| ED-B 20-49 | 31-38 | IGYRITVV | 288 | B5101 | 25 | N.A. |
| | 30-38 | IIGYRITVV | 289 | A0201 | 23 | 15 |
| | | | | A3 | 17 | <1.0 |
| | | | | B08 | 15 | <1.0 |
| | | | | B5101 | 15 | <1.0 |
| | 29-38 | TIIGYRITVV | 290 | A0201 | 26 | 9 |
| | | | | A26 | 18 | N.A. |
| | | | | A3 | 18 | N.A. |
| | 23-30 | TPLNSSTI | 282 | B5101 | 22 | N.A. |
| | 25-33 | LNSSTIIGY | 285 | A1 | 19 | <5 |
| | | | | A26 | 16 | N.A. |
| | 24-33 | PLNSSTIIGY | 286 | A26 | 24 | N.A. |
| | | | | A3 | 16 | <5 |
| | 31-39 | IGYRITVVA | 291 | A3 | 17 | <5 |
| | 30-39 | IIGYRITVVA | 292 | A0201 | 15 | <5 |
| | | | | A3 | 18 | <5 |
| | 23-31 | TPLNSSTII | 287 | B0702 | 17 | 8 |
| | | | | B5101 | 25 | 440 |

TABLE 28A

CEA: Preferred Epitopes Revealed by Housekeeping Proteasome Di-
gestion

Binding Prediction

| | | | | | | |
|---|---|---|---|---|---|---|
| Substrate | Epitope | Sequence | Seq. ID No. | HLA type | SYFPEITHI | NIH |

| | | | | | | | |
|---|---|---|---|---|---|---|---|
| CEA 176-202 | 184-191 | SLPVSPRL | 293 | B08 | 19 | <5 | |
| | 183-191 | QSLPVSPRL | 294 | A0201 | 15 | <5 | |
| | | | | B1510 | 15 | | |
| | | | | B2705 | 18 | 10 | |
| | | | | B2709 | 15 | | |
| | 186-193 | PVSPRLQL | 295 | B08 | 18 | <5 | |
| | 185-193 | LPVSPRLQL | 296 | B0702 | 26 | 180 | |
| | | | | B08 | 16 | <5 | |
| | | | | B5101 | 19 | 130 | |
| | 184-193 | SLPVSPRLQL | 297 | A0201 | 23 | 21 | |
| | | | | A26 | 18 | N.A. | |
| | | | | A3 | 18 | <5 | |
| | 185-192 | LPVSPRLQ | 298 | B5101 | 17 | N.A. | |
| | 192-200 | QLSNGNRTL | 299 | A0201 | 21 | 4 | |
| | | | | A26 | 16 | N.A. | |
| | | | | A3 | 19 | <5 | |
| | | | | B08 | 17 | <5 | |
| | | | | B1510 | 15 | | |
| | 191-200 | LQLSNGNRTL | 300 | A0201 | 16 | 3 | |
| | 179-187 | WVNNQSLPV | 301 | A0201 | 16 | 28 | |
| | 186-194 | PVSPRLQLS | 302 | A26 | 17 | N.A. | A26 |
| | | | | A3 | 15 | <5 | |

TABLE 28B

CEA: Preferred Epitopes Revealed by Housekeeping Proteasome Diges-
tion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| CEA 354-380 | 362-369 | SLPVSPRL | 303 | B08 | 19 | <1.0 |
| | 361-369 | QSLPVSPRL | 304 | A0201 | 15 | <1.0 |
| | | | | B2705 | 18 | 10 |
| | | | | B2709 | 15 | |
| | 364-371 | PVSPRLQL | 305 | B08 | 18 | <1.0 |
| | 363-371 | LPVSPRLQL | 306 | B0702 | 26 | 180 |
| | | | | B08 | 16 | <1.0 |
| | | | | B5101 | 19 | 130 |
| | 362-371 | SLPVSPRLQL | 307 | A0201 | 23 | 21 |
| | | | | A26 | 16 | N.A. |
| | | | | A24 | N.A. | 6 |
| | | | | A3 | 18 | <5 |
| | 363-370 | LPVSPRLQ | 308 | B5101 | 17 | N.A. |
| | 370-378 | QLSNDNRTL | 309 | A0201 | 22 | 4 |
| | | | | A26 | 16 | N.A. |
| | | | | A3 | 17 | <1.0 |
| | | | | B08 | 17 | <1.0 |
| | 369-378 | LQLSNDNRTL | 310 | A0201 | 16 | 3 |
| | 357-365 | WVNNQSLPV | 311 | A0201 | 16 | 28 |
| | 360-368 | NQSLPVSPR | 312 | B2705 | 14 | 100 |

TABLE 28C

CEA: Preferred Epitopes Revealed by Housekeeping Proteasome Di-
gestion

| | |
|---|---|
| Seq. | Binding Prediction |

| | | | | | | |
|---|---|---|---|---|---|---|
| Substrate | Epitope | Sequence | ID No. | HLA type | SYFPEITHI | NIH |

| | | | | | | |
|---|---|---|---|---|---|---|
| CEA 532-558 | 540-547 | SLPVSPRL | 313 | B08 | 19 | <5 |
| | 539-547 | QSLPVSPRL | 314 | A0201 | 15 | <5 |
| | | | | B1510 | 15 | <5 |
| | | | | B2705 | 18 | 10 |
| | | | | B2709 | 15 | |
| | 542-549 | PVSPRLQL | 315 | B08 | 18 | <5 |
| | 541-549 | LPVSPRLQL | 316 | B0702 | 26 | 180 |
| | | | | B08 | 16 | <1.0 |
| | | | | B5101 | 19 | 130 |
| | 540-549 | SLPVSPRLQL | 317 | A0201 | 23 | 21 |
| | | | | A26 | 18 | N.A. |
| | | | | A3 | 18 | <5 |
| | 541-548 | LPVSPRLQ | 318 | B5101 | 17 | N.A. |
| | 548-556 | QLSNGNRTL | 319 | A0201 | 24 | 4 |
| | | | | A26 | 16 | N.A. |
| | | | | A3 | 19 | <1.0 |
| | | | | B08 | 17 | <1.0 |
| | | | | B1510 | 15 | |
| | 547-556 | LQLSNGNRTL | 320 | A0201 | 16 | 3 |
| | 535-543 | WVNGQSLPV | 321 | A0201 | 18 | 28 |
| | | | | A3 | 15 | <1.0 |
| | 533-541 | LWWVNGQSL | 322 | A0201 | 15 | <5 |

TABLE 28D

CEA: Preferred Epitopes Revealed by Housekeeping Proteasome Di-
gestion

| | |
|---|---|
| Seq. ID | Binding Prediction |

| | | | | | | |
|---|---|---|---|---|---|---|
| Substrate | Epitope | Sequence | No. | HLA type | SYFPEITHI | NIH |

| | | | | | | |
|---|---|---|---|---|---|---|
| CEA 532-558 | 532-541 | YLWWVNGQSL | 323 | A0201 | 25 | 816 |
| (continued) | | | | A26 | 18 | N.A. |
| | 538-546 | GQSLPVSPR | 324 | B2705 | 17 | 100 |

TABLE 29A

HER2/NEU: Preferred Epitopes Revealed by Housekeeping Proteasome
Digestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| Her-2 25-52 | 30-37 | DMKLRLPA | 325 | B08 | 19 | 8 |
| | 28-37 | GTDMKLRLPA | 326 | A1 | 23 | 6 |
| | 42-49 | HLDMLRHL | 327 | B08 | 17 | <5 |
| | 41-49 | THLDMLRHL | 328 | A0201 | 17 | <5 |
| | | | | B1510 | 24 | N.A. |
| | 40-49 | ETHLDMLRHL | 329 | A26 | 29 | N.A. |
| | 36-43 | PASPETHL | 330 | B5101 | 17 | N.A. |
| | 35-43 | LPASPETHL | 331 | A0201 | 15 | <5 |
| | | | | B5101 | 20 | 130 |
| | | | | B5102 | N.A. | 100 |
| | 34-43 | RLPASPETHL | 332 | A0201 | 20 | 21 |
| | 38-46 | SPETHLDML | 333 | A0201 | 15 | <5 |
| | | | | B0702 | 20 | 24 |
| | | | | B08 | 18 | <5 |
| | | | | B5101 | 18 | <5 |
| | 37-46 | ASPETHLDML | 334 | A0201 | 18 | <5 |
| | 42-50 | HLDMLRHLY | 335 | A1 | 29 | 25 |
| | | | | A26 | 20 | N.A. |
| | | | | A3 | 17 | 4 |
| | 41-50 | THLDMLRHLY | 336 | A1 | 18 | <1.0 |

TABLE 29B

HER2/NEU: Preferred Epitopes Revealed by Housekeeing Proteasome Di-
gestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| Her-2 705-732 | 719-726 | ELRKVKVL | 337 | B08 | 24 | 16 |
| | 718-726 | TELRKVKVL | 338 | A0201 | 16 | 1 |
| | | | | B08 | 22 | <5 |
| | | | | B5101 | 16 | <5 |
| | 717-726 | ETELRKVKVL | 339 | A1 | 18 | 2 |
| | | | | A26 | 28 | 6 |
| | 715-723 | LKETELRKV | 340 | A0201 | 17 | <5 |
| | | | | B5101 | 15 | <5 |
| | 714-723 | ILKETELRKV | 341 | A0201 | 29 | 8 |
| | 712-720 | MRILKETEL | 342 | A0201 | 15 | <5 |
| | | | | B08 | 22 | <5 |
| | | | | B2705 | 27 | 2000 |
| | | | | B2709 | 21 | N.A. |
| | 711-720 | QMRILKETEL | 343 | A1 | 18 | 5 |
| | | | | B0702 | 13 | 40 |
| | 717-725 | ETELRKVKV | 344 | A1 | 18 | 5 |
| | | | | A26 | 18 | N.A. |
| | 716-725 | KETELRKVKV | 345 | A0201 | 16 | 19 |
| | 706-714 | MPNQAQMRI | 346 | B0702 | 16 | 8 |
| | | | | B5101 | 22 | 629 |
| | 705-714 | AMPNQAQMRI | 347 | A0201 | 18 | 8 |
| | 706-715 | MPNQAQMRIL | 348 | B0702 | 20 | 80 |

TABLE 29C

HER2/NEU: Preferred Epitopes Revealed by Housekeeping Proteasome
Digestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| Her-2 954-982 | 966-973 | RPRFRELV | 349 | B08 | 20 | 24 |
| | | | | B5101 | 18 | N.A. |
| | 965-973 | CRPRFRELV | 350 | B2709 | 18 | |
| | 968-976 | RFRELVSEF | 351 | A26 | 25 | N.A. |
| | | | | A24 | N.A. | 32 |
| | | | | A3 | 15 | <5 |
| | | | | B08 | 16 | <5 |
| | | | | B2705 | 19 | |
| | 967-976 | PRERELVSEF | 352 | A26 | 18 | N.A. |
| | 964-972 | ECRPRFREL | 353 | A26 | 21 | N.A. |
| | | | | A24 | N.A. | 6 |
| | | | | B0702 | 15 | 40 |
| | | | | B8 | 27 | 640 |
| | | | | B1510 | 16 | <5 |

TABLE 30

NY-ESO-1: Preferred Epitopes Revealed by Housekeeping Proteasome Di-
gestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| NY-ESO-1 51-77 | 67-75 | GAASGLNGC | 354 | A0201 | 15 | <5 |
| | 52-60 | RASGPGGGA | 355 | B0702 | 15 | <5 |
| | 64-72 | PHGGAASGL | 356 | B1510 | 21 | N.A. |
| | 63-72 | GPHGGAASGL | 357 | B0702 | 22 | 80 |
| | 60-69 | APRGPHGGAA | 358 | B0702 | 23 | 60 |

TABLE 31A

PRAME: Preferred Epitopes Revealed by Housekeeping Proteasome Digestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| PRAME 103-135 | 112-119 | VRPRRWKL | 359 | B08 | 19 | |
| | 111-110 | EVRPRRWKL | 360 | A26 | 27 | N.A. |
| | | | | A24 | N.A. | 5 |
| | | | | A3 | 19 | N.A. |
| | | | | B0702 | 15 | (B7)300.00 |
| | | | | B08 | 26 | 160 |
| | 113-121 | RPRRWKLQV | 361 | B0702 | 21 | (B7)40.00 |
| | | | | B5101 | 19 | 110 |
| | 114-112 | PRRWKLQVL | 362 | B08 | N.A. | 160 |
| | | | | B2705 | 23 | 200 |
| | 113-122 | RPRRWKLQVL | 363 | B0702 | 24 | (B7)800.00 |
| | | | | B8 | N.A. | 160 |
| | | | | B5101 | N.A. | 61 |
| | | | | B5102 | N.A. | 61 |
| | | | | A24 | N.A. | 10 |
| | 116-124 | RWKLQVLDL | 364 | B08 | 22 | <5 |
| | | | | B2705 | 17 | 3 |
| | 115-124 | RRWKLQVLDL | 365 | A0201 | 16 | <5 |
| PRAME 161-187 | 174-182 | PVEVLVDLF | 366 | A26 | 25 | N.A. |

TABLE 31B

PRAME: Preferred Epitopes Revealed by Housekeeping Proteasome Di-
gestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| PRAME 185-215 | 199-206 | VKRKKNVL | 367 | B08 | 27 | 8 |
| | 198-206 | KVKRKKNVL | 368 | A0201 | 16 | <1.0 |
| | | | | A26 | 20 | N.A. |
| | | | | A3 | 22 | <1.0 |
| | | | | B08 | 30 | 40 |
| | | | | B2705 | 16 | |
| | 197-206 | EKVKRKKNVL | 369 | A26 | 15 | N.A. |
| | 198-205 | KVKRKKNV | 370 | B08 | 20 | 6 |
| | 201-208 | RKKNVLRL | 371 | B08 | 20 | <5 |
| | 200-208 | KRKKNVLRL | 372 | A0201 | 15 | <1.0 |
| | | | | A26 | 15 | N.A. |
| | | | | B0702 | 15 | <1.0 |
| | | | | B08 | 21 | <1.0 |
| | | | | B2705 | 28 | |
| | | | | B2709 | 25 | |
| | 199-208 | VKRKKNVLRL | 373 | A0201 | 16 | <1.0 |
| | | | | B0702 | 16 | 4 |
| | 189-196 | DELFSYLI | 374 | B5101 | 15 | N.A. |
| | 205-213 | VLRLCCKKL | 375 | A0201 | 22 | 3 |
| | | | | A26 | 17 | N.A. |
| | | | | B08 | 25 | 8 |
| | 204-213 | NVLRLCCKKL | 376 | A0201 | 17 | 7 |
| | | | | A26 | 19 | N.A. |

TABLE 31C

PRAME: Preferred Epitopes Revealed by Housekeeping Proteasome Diges-
tion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| PRAME 185-215 | 194-202 | YLIEKVKRK | 377 | A0201 | 20 | <1.0 |
| (continued) | | | | A26 | 18 | N.A. |
| | | | | A3 | 25 | ta 68 |
| | | | | B08 | 20 | <1.0 |
| | | | | B2705 | 17 | |
| PRAME 71-98 | 74-81 | QAWPFTCL | 378 | B5101 | 17 | n.a. |
| | 73-81 | VQAWPFTCL | 379 | A0201 | 14 | 7 |
| | | | | A24 | n.a. | 5 |
| | | | | B0702 | 13 | 30 |
| | 81-88 | LPLGVLMK | 381 | B5101 | 18 | n.a. |
| | 80-88 | CLPLGVLMK | 382 | A0201 | 17 | <1.0 |
| | | | | A3 | 27 | 120 |
| | 79-88 | TCLPLGVLMK | 383 | A1 | 12 | 10 |
| | | | | A3 | 19 | 3 |
| | 84-92 | GVLMKGQHL | 384 | A0201 | 18 | 7 |
| | | | | A26 | 21 | n.a. |
| | | | | B08 | 21 | 4 |
| | 81-89 | LPLGVLMKG | 385 | B5101 | 20 | 2 |
| | 80-89 | CLPLGVLMKG | 386 | A0201 | 16 | <1.0 |
| | 76-85 | WPFTCLPLGV | 387 | B0702 | 18 | 4 |

TABLE 31D

PRAME: Preferred Epitopes Revealed by Housekeeping Proteasome Di-
gestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| PRAME 39-65 | 51-59 | ELFPPLFMA | 388 | A0201 | 19 | 18 |
| | | | | A26 | 23 | N.A. |
| | 49-57 | PRELFPPLF | 389 | B2705 | 22 | |
| | | | | B2709 | 19 | |
| | 48-57 | LPRELFPPLF | 390 | B0702 | 19 | 4 |
| | 50-58 | RELFPPLFM | 391 | B2705 | 16 | |
| | | | | B2705 | 15 | |
| | 49-58 | PRELFPPLFM | 392 | A1 | 16 | <1.0 |

TABLE 32

PSA: Preferred Epitopes Revealed by Housekeeping Proteasome Diges-
tion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| PSA 232-258 | 239-246 | RPSLYTKV | 393 | B5101 | 21 | N.A. |
| | 238-246 | ERPSLYTKV | 394 | B2705 | 15 | 60 |
| | 236-243 | LPERPSLY | 395 | B5101 | 18 | N.A. |
| | 235-243 | ALPERPSLY | 396 | A1 | 19 | <1.0 |
| | | | | A26 | 22 | N.A. |
| | | | | A3 | 26 | 6 |
| | | | | B08 | 16 | <1.0 |
| | | | | B2705 | 11 | 15 |
| | | | | B2709 | 19 | N.A. |
| | 241-249 | SLYTKVVHY | 397 | A0201 | 20 | <1.0 |
| | | | | A1 | 19 | <1.0 |
| | | | | A26 | 25 | N.A. |
| | | | | A3 | 26 | 60 |
| | | | | B08 | 20 | <1.0 |
| | | | | B2705 | 13 | 75 |
| | 240-249 | PSLYTKVVHY | 398 | A1 | 20 | <1.0 |
| | | | | A26 | 16 | N.A. |
| | 239-247 | RPSLYTKVV | 399 | B0702 | 21 | 4 |
| | | | | B5101 | 23 | 110 |

TABLE 33A

PSMA: Preferred Epitopes Revealed by Housekeeping Proteasome Diges-
tion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| PSMA 202-228 | 211-218 | GNKVKNAQ | 400 | B08 | 22 | <5 |
| | 202-209 | IARYGKVF | 401 | B08 | 18 | <5 |
| | 217-225 | AQLAGAKGV | 402 | A0201 | 16 | 26 |
| | 207-215 | KVFRGNKVK | 403 | A3 | 32 | 15 |
| | 211-219 | GNKVKNAQL | 404 | B8 | 33 | 80 |
| | | | | B2705 | 17 | 20 |
| PSMA 255-282 | 269-277 | TPGYPANEY | 405 | A1 | 16 | <5 |
| | 268-277 | LTPGYPANEY | 406 | A1 | 21 | 1 |
| | | | | A26 | 24 | N.A. |
| | 271-279 | GYPANEYAY | 407 | A1 | 15 | <5 |
| | 270-279 | PGYPANEYAY | 408 | A1 | 19 | <5 |
| | 266-274 | DPLTPGYPA | 409 | B0702 | 21 | 3 |
| | | | | B5101 | 17 | 20 |
| PSMA 483-509 | 492-500 | SLYESWTKK | 410 | A0201 | 17 | <5 |
| | | | | A3 | 27 | 150 |
| | | | | B2705 | 18 | 150 |
| | 491-500 | KSLYESWTKIK | 411 | A3 | 16 | <5 |
| | 486-494 | EGFEGKSLY | 412 | A1 | 19 | <5 |
| | | | | A26 | 21 | N.A. |
| | | | | B2705 | 16 | <5 |
| | 485-494 | DEGFEGKSLY | 413 | A1 | 17 | <5 |
| | | | | A26 | 17 | N.A. |
| | 498-506 | TKKSPSPEF | 414 | B08 | 17 | <5 |

TABLE 33B

PSMA: Preferred Epitopes Revealed by Housekeeping Proteasome Diges-
tion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| PSMA 483-509 | 497-506 | WTKKSPSPEF | 415 | A26 | 24 | N.A. |
| (continued) | 492-501 | SLYESWTKKS | 416 | A0201 | 16 | <5 |
| | | | | A3 | 16 | <5 |
| PSMA 721-749 | 725-732 | WGEVKRQI | 417 | B08 | 17 | <5 |
| | | | | B5101 | 17 | N.A. |
| | 724-732 | AWGEVKRQI | 418 | B5101 | 15 | 6 |
| | 723-732 | KAWGEVKRQI | 419 | A0201 | 16 | <1.0 |
| | 723-730 | KAWGEVKR | 420 | B5101 | 15 | N.A. |
| | 722-730 | SKAWGEVKR | 421 | B2705 | 15 | <5 |
| | 731-739 | QIYVAAFTV | 422 | A0201 | 21 | 177 |
| | | | | A3 | 21 | <1.0 |
| | | | | B5101 | 15 | 5 |
| | 733-741 | YVAAFTVQA | 423 | A0201 | 17 | 6 |
| | | | | A3 | 20 | <1.0 |
| | 725-733 | WGEVKRQIY | 424 | A1 | 26 | 11 |
| | 727-735 | EVKRQLYVA | 425 | A26 | 22 | N.A. |
| | | | | A3 | 18 | <1.0 |
| | 738-746 | TVQAAAETL | 426 | A26 | 18 | N.A. |
| | | | | A3 | 19 | <1.0 |
| | 737-746 | FTVQAAAETL | 427 | A0201 | 17 | <1.0 |
| | | | | A26 | 19 | N.A. |

TABLE 33C

PSMA: Preferred Epitopes Revealed by Housekeeping Proteasome Diges-
tion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| PSMA 721-749 | 729-737 | KRQIYVAAF | 428 | A26 | 16 | N.A. |
| (continued) | | | | B2705 | 24 | 3000 |
| | | | | B2709 | 21 | N.A. |
| | 721-729 | PSKAWGEVK | 429 | A3 | 20 | <1.0 |
| | 723-731 | KAWGEVKRQ | 430 | B5101 | 16 | <1.0 |
| PSMA 95-122 | 100-108 | WKEFGLDSV | 431 | A0201 | 16 | <5 |
| | 99-108 | QWKEFGLDSV | 432 | A0201 | 17 | <5 |
| | 102-111 | EFGLDSVELA | 433 | A26 | 16 | N.A. |

TABLE 34A

SCP-1: Preferred Epitopes Revealed by Housekeeping Proteasome Digestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| SCP-1 117-143 | 126-134 | ELRQKESKL | 434 | A0201 | 20 | <5 |
| | | | | A26 | 26 | N.A. |
| | | | | A3 | 17 | <5 |
| | | | | B07f02 | 13 | (B7)40.00 |
| | | | | B8 | 34 | 320 |
| | 125-134 | AELRQKESKL | 435 | A0201 | 16 | <5 |
| | 133-141 | KLQENRKII | 436 | A0201 | 20 | 61 |
| SCP-1 281-308 | 298-305 QLEEKTKL | 437 | B08 | 28 | 2 | |
| | 297-305 | NQLEEKTKL | 438 | A0201 | 15 | 33 |
| | | | | B2705 | 19 | 200 |
| | 288-296 | LLEESRDKV | 439 | A0201 | 25 | 15 |
| | | | | B2705 | 19 | 200 |
| | 287-296 | FLLEESRDKV | 440 | A0201 | 27 | 2378 |
| | 291-299 | ESRDKVNQL | 441 | A26 | 17 | N.A. |
| | | | | B08 | 29 | 240 |
| | 290-299 | EESRDKVNQL | 442 | A26 | 17 | N.A. |
| SCP-1 471-498 | 475-483 | EKEVHDLEY | 443 | A1 | 31 | 11 |
| | | | A26 | 17 | N.A. | |
| | 474-483 | REKEVHDLEY | 444 | A1 | 21 | <1.0 |
| | 480-488 | DLEYSYCHY | 445 | A1 | 26 | 45 |
| | | | | A26 | 30 | N.A. |
| | | | | A3 | 16 | <5 |
| | 477-485 | EVHDLEYSY | 446 | A1 | 15 | 1 |

TABLE 34B

SCP-1: Preferred Epitopes Revealed by Housekeeping
Proteasome Digestion

Binding Prediction

| | | | | | | |
|---|---|---|---|---|---|---|
| Substrate | Eptiope | Sequence | Seq. ID No. | HLA type | SYFPEITHI | NIH |

| | | | | | | |
|---|---|---|---|---|---|---|
| SCP-1 471-498 | 477-485 | EVHDLEYSY | | A26 | 29 | N.A. |
| (continued) | | | | A3 | 19 | <1.0 |
| | 477-486 | EVHDLEYSYC | 447 | A26 | 22 | N.A. |
| SCP-1 493-520 | 502-509 | KLSSKREL | 448 | B08 | 26 | 4 |
| | 508-515 | ELKNTEYF | 449 | B08 | 24 | <1.0 |
| | 507-515 | RELKNTEYF | 450 | B2705 | 18 | 45 |
| | | | | B4403 | N.A. | 120 |
| | 496-503 | KRGQRPKL | 451 | B08 | 18 | <1.0 |
| | 494-503 | LPKRGQRPKL | 452 | B0702 | 22 | 120 |
| | | | | B8 | N.A. | 16 |
| | | | | B5101 | N.A. | 130 |
| | | | | B3501 | N.A. | 60 |
| | 509-517 | LKNTEYFTL | 453 | A0201 | 15 | <5 |
| | 508-517 | ELKNTEYFTL | 454 | A0201 | 18 | <1.0 |
| | | | | A26 | 27 | N.A. |
| | | | | A3 | 16 | <1.0 |
| | 506-514 | KRELKNTEY | 455 | A1 | 26 | 2 |
| | | | | B2705 | 26 | 3000 |
| | 502-510 | KLSSKRELK | 456 | A3 | 25 | 60 |
| | 498-506 | GQRPKLSSK | 457 | A3 | 22 | 4 |
| | | | | B2705 | 18 | 200 |
| | 497-506 | RGQRPKLSSK | 458 | A3 | 22 | <1.0 |
| | 500-508 | RPKLSSKRE | 459 | B08 | 18 | <1.0 |

TABLE 34C

SCP-1: Preferred Epitope Revealed by Housekeeping
Proteasome Digestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| SCP-1 570-596 | 573-580 | LEYVREEL | 560 | B08 | 19 | <5 |
| | 572-580 | ELEYVREEL | 461 | A0201 | 17 | <1.0 |
| | | | | A26 | 23 | N.A. |
| | | | | A24 | N.A. | 9 |
| | | | | B08 | 20 | N.A. |
| | 571-580 | N ELEYVREEL | 462 | A0201 | 16 | 4 |
| | 579-587 | ELKQKRDEV | 463 | A0201 | 19 | <1.0 |
| | | | | A26 | 18 | N.A. |
| | | | | B08 | 29 | 48 |
| | 575-583 | YVREELKQK | 464 | A26 | 17 | N.A. |
| | | | | A3 | 27 | 2 |
| SCP-1 618-645 | 632-640 | QLNVYEIKV | 465 | A0201 | 24 | 70 |
| | 630-638 | SKQLNVYEI | 466 | A0201 | 17 | <5 |
| | 628-636 | AESKQLNVY | 467 | A1 | 19 | <5 |
| | | | | A26 | 16 | N.A. |
| | 627-636 | TAESKQLNVY | 468 | A1 | 26 | 45 |
| | | | | A26 | 15 | N.A. |

TABLE 34D

SCP-1: Preferred Epitopes Revealed by Housekeeping
Proteasome Digestion

Binding Prediction

| Substrate | Eptiope | Sequence | Seq. ID No. | HLA type | SYFPEITHI | NIH |
|---|---|---|---|---|---|---|
| SCP-1 633-660 | 638-645 | IKVNKLEL | 469 | B08 | 21 | <1.0 |
| | 637-345 | EIKVNKLEL | 470 | A0201 | 17 | <1.0 |
| | | | A26 | 26 | N.A. | |
| | | | B08 | 28 | 8 | |
| | | | B1510 | 15 | N.A. | |
| | 636-645 | YEIKVNKLEL | 471 | A0201 | 17 | 2 |
| | 642-650 | KLELELESA | 472 | A0201 | 20 | 1 |
| | | | A3 | 16 | <1.0 | |
| | 635-643 | VYEIKVNKL | 473 | A0201 | 18 | <1.0 |
| | | | A24 | N.A. | 396 | |
| | | | B08 | 22 | <1.0 | |
| | 634-643 | NVYEIKVNKL | 474 | A0201 | 24 | 56 |
| | | | A26 | 25 | N.A. | |
| | | | A24 | N.A. | 6 | |
| | | | A3 | 15 | <5 | |
| | | | B0702 | 11 | (B7) 20 | |
| | | | B08 | N.A. | 6 | |
| | 646-654 | ELESAKQKF | 475 | A26 | 27 | N.A. |
| SCP-1 640-668 | 642-650 | KLELELESA | 476 | A0201 | 20 | 1 |
| | | | A3 | 16 | <1.0 | |
| | 646-654 | ELESAKQKF | 477 | A26 | 27 | N.A. |

TABLE 34E

SCP-1: Preferred Eptiopes Revealed by Housekeeping

Proteasome Digestion

Binding Prediction

| | | | | | | |
|---|---|---|---|---|---|---|
| Substrate | Eptiope | Sequence | Seq. ID No. | HLA type | SYFPEITHI | NIH |

| | | | | | | |
|---|---|---|---|---|---|---|
| SCP-1 768-796 | 771-778 | KEKLKREA | 478 | B08 | 21 | <5 |
| | 777-785 | EAKENTATL | 479 | A0201 | 18 | <5 |
| | | | | A26 | 18 | N.A. |
| | | | | A24 | N.A. | 5 |
| | | | | B0702 | 13 | 12 |
| | | | | B08 | 28 | 48 |
| | | | | B5101 | 20 | 121 |
| | 776-785 | REAKENTATL | 480 | A0201 | 16 | <5 |
| | 773-782 | KLKREAKENT | 481 | A3 | 17 | <5 |
| SCP-192-125 | 112-119 | EAEKIKKW | 482 | B5101 | 17 | N.A. |
| | 101-109 | GLSRVYSKL | 483 | A0201 | 23 | 32 |
| | | | | A26 | 22 | N.A. |
| | | | | A24 | N.A. | 6 |
| | | | | A3 | 17 | 3 |
| | | | | B08 | 17 | <1.0 |
| | 100-109 | EGLSRVYSKL | 484 | A26 | 21 | N.A. |
| | | | | A24 | N.A. | 9 |
| | 108-116 | KLYKEAEKI | 485 | A0201 | 22 | 57 |
| | | | | A3 | 20 | 9 |
| | | | B5101 | 18 | 5 | |
| | 98-106 | NSEGLSRVY | 486 | A1 | 31 | 68 |
| | 97-106 | ENSEGLSRVY | 487 | A26 | 18 | N.A. |
| | 102-110 | LSRVYSKLY | 488 | A1 | 22 | <1.0 |

TABLE 34F

SCP-1: Preferred Epitopes Revealed by Housekeeping
Proteasome Digestion

| | | | | Dinding Prediction | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| SCP-1 92-125 | 101-110 | GLSRVYSKLY | 489 | A1 | 18 | <1.0 |
| | | | | A26 | 18 | N.A. |
| | | | | A3 | 19 | 18 |
| | 96-105 | LENSEGLSRV | 490 | A0201 | 17 | 5 |
| | 108-117 | KLYKEAEKIK | 491 | A3 | 19 | 18 |
| SCP-1 931-958 | 949-956 | REDRWAVI | 492 | B5101 | 15 | N.A. |
| | 948-956 | MREDRWAVI | 493 | B2705 | 18 | 600 |
| | | | | B2709 | 18 | N.A. |
| | | | | b5101 | 15 | 1 |
| | 947-956 | KMREDRWAVI | 494 | a0201 | 21 | 6 |
| | | | | B08 | N.A. | 15 |
| | 947-955 | KMREDRWAV | 495 | A0201 | 22 | 411 |
| | 934-942 | TTPGSTLKF | 496 | A26 | 25 | N.A. |
| | 933-942 | LTTPGSTLKF | 497 | A26 | 23 | N.A. |
| | 937-945 | GSTLKFGAI | 498 | B08 | 19 | 1 |
| | 945-953 | IRKMREDRW | 499 | B08 | 19 | <5 |
| | 236-243 | RLEMHFKL | 500 | B08 | 16 | <5 |
| SCP-1 232-259 | 235-243 | SRLEMHFKL | 501 | A0201 | 18 | <5 |
| | | | | B2705 | 25 | 2000 |
| | | | | B2709 | 22 | |
| | 242-250 | KLKEDYEKI | 502 | A0201 | 4 | |

TABLE 34G

SCP-1: Preferred Epitopes Revealed by Housekeeping
Proteasome Digestion

Binding Prediction

| | | | | | | |
|---|---|---|---|---|---|---|
| Substrate | Epitope | Sequence | Seq. ID No. | HLA type | SYFPEITHI | NIH |
| SCP-1 232-259 | | | | A26 | 16 | N.A. |
| (continued) | | | | A3 | 158 | 3 |
| | | | | B08 | 24 | <5 |
| | | | | B5101 | 14 | 2 |
| | 249-257 | KIQHLEQEY | 503 | A1 | 15 | <5 |
| | | | | A26 | 23 | N.A. |
| | | | | A3 | 17 | <5 |
| | 248-257 | EKIQHLEQEY | 504 | A1 | 15 | <5 |
| | | | | A26 | 21 | N.A. |
| | 233-242 | ENSRLEMHF | 505 | A26 | 19 | N.A. |
| | 236-245 | RLEMHFKLKE | 506 | A1 | 19 | <5 |
| | | | | A3 | 17 | <5 |
| | 324-331 | LEDIKVSL | 507 | B08 | 20 | <1.0 |
| SCP-1 310-340 | 323-331 | 508 | A0201 | | | |
| | | | | A26 | 25 | N.A. |
| | | | | A24 | N.A. | 10 |
| | | | | A3 | 17 | <1.0 |
| | | | | B08 | 19 | <1.0 |
| | | | | B5101 | 16 | N.A. |
| | 322-331 | KELEDIKVSL | 509 | A0201 | 19 | 22 |
| | 320-327 | LTKELEDI | 500 | B08 | 18 | <5 |
| | 319-327 | HLTKELEDI | 511 | A0201 | 21 | <1.0 |
| | 330-338 | SLQRSVSTQ | 512 | A0201 | 18 | <1.0 |

TABLE 34H

SCP-1: Preferred Epitopes Revealed by Housekeeping
Proteasome Digestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| SCP-1 310-340 | 321-329 | TKELEDIKV | 513 | A1 | 16 | <1.0 |
| (continued) | | | | | | |
| | 320-329 | LTKELEDIKV | 514 | A0201 | 19 | <1.0 |
| | 326-335 | DIKVSLQRSV | 515 | A26 | 18 | N.A. |
| SCP-1 272-305 | 281-288 | KMKDLTFL | 516 | B08 | 20 | 3 |
| | 280-288 | NKMKDLTFL | 517 | A0201 | 15 | 1 |
| | 279-288 | ENKMKDLTFL | 518 | A26 | 19 | N.A. |
| | 288-296 | LLEESRDKV | 519 | A0201 | 25 | 15 |
| | | B5101 | 15 | 3 | | |
| | 287-296 | FLLEESRDKV | 520 | A0201 | 27 | 2378 |
| | 291-299 | ESRDKVNQL | 521 | A26 | 21 | N.A. |
| | | B08 | 29 | 240 | | |
| | 290-299 | EESRDKVNQL | 522 | A26 | 19 | N.A. |
| | 277-285 | EKENT(MKDL | 523 | A26 | 19 | N.A. |
| | | B08 | 23 | <1.0 | | |
| | 276-285 | TEKENKMKDL | 524 | A26 | 15 | N.A. |
| | 279-287 | ENKMKDLTF | 525 | A26 | 18 | N.A. |
| | | B08 | 28 | 4 | | |
| SCP-1 211-239 | 218-225 | IEKIVHTAIF | 526 | B08 | 17 | <5 |
| | 217-225 | NIEKMITAF | 527 | A26 | 26 | N.A. |
| | 216-225 | SNIEKMITAF | 528 | A26 | 19 | N.A. |
| | 223-230 | TAFEELRV | 529 | B5101 | 23 | N.A. |
| | 222-230 | LTAEEELRV | 530 | A0201 | 18 | 2 |
| | 221-230 | MITAFEELRV | 531 | A0201 | 18 | 16 |

TABLE 34I

SCP-1: Preferred Epitopes Revealed by Housekeeping
Proteasome Digestion

Binding Prediction

| | | | | | | |
|---|---|---|---|---|---|---|
| Substrate | Eptiope | Sequence | Seq. ID No. | HLA type | SYFPEITHI | NIH |

| | | | | | | |
|---|---|---|---|---|---|---|
| SCP-1 211-239 | 220-228 | KMITAFEEL | 532 | A0201 | 23 | 50 |
| (continued) | | | | A26 | 15 | N.A. |
| | | | | A24 | N.A. | 16 |
| | 219-228 | EKMITAEEEL | 533 | A26 | 19 | N.A. |
| | 227-235 | ELRVQAENS | 534 | A3 | 16 | <1.0 |
| | | | | B08 | 15 | <1.0 |
| | 213-222 | DLNSNIEKMI | 535 | A0201 | 17 | <1.0 |
| | | | | A26 | 16 | N.A. |
| SCP-1 836-863 | 837-844 | WTSAKNTL | 536 | B08 | 20 | 4 |
| | 846-854 | TPLPKAYTV | 537 | A0201 | 18 | 2 |
| | | | | B0702 | 17 | 4 |
| | | | | B08 | 16 | 2 |
| | | | | B5101 | 25 | 220 |
| | 845-854 | STPLPKAYTV | 538 | A0201 | 19 | <5 |
| | 844-852 | LSTPLPKAY | 539 | A1 | 23 | 8 |
| | 843-852 | TLSTPLPKAY | 540 | A1 | 16 | <1.0 |
| | | | | A26 | 19 | N.A. |
| | | | | A3 | 18 | 2 |
| | 842-850 | NTLSTPLPK | 541 | A3 | 16 | 3 |
| | 841-850 | KNTLSTPLPK | 542 | A3 | 18 | <1.0 |

TABLE 34J

SCP-1: Preferred Epitopes Revealed by Housekeeping
Proteasome Digestion

Binding Prediction

| | | | | | | |
|---|---|---|---|---|---|---|
| Substrate | Epitope | Sequence | Seq. ID No. | HLA type | SYFPEIThI | NIH |

| | | | | | | |
|---|---|---|---|---|---|---|
| SCP-1 819-845 | 828-835 | ISKDKRDY | 543 | B08 | 21 | 3 |
| | | | | A26 | 21 | N.A. |
| | 826-835 | HGISKDKRDY | 544 | A1 | 15 | <5 |
| | 832-840 | KRDYLWTSA | 545 | B2 | 16 | 600 |
| | 829-838 | SKDKRDYLWT | 546 | A1 | 18 | <5 |
| SCP-1 260-288 | 279-286 | ENKMKDLT | 547 | B08 | 22 | 8 |
| | 260-268 | EINDKEKQV | 548 | A0201 | 17 | 3 |
| | | | | A26 | 19 | N.A. |
| | | | | B08 | 17 | <5 |
| | 274-282 | QITEKENL | 549 | A0201 | 17 | 3 |
| | | | | A26 | 22 | N.A. |
| | | | | B08 | 16 | <5 |
| | 269-277 | SLLLIQITE | 550 | A0201 | 16 | <1.0 |
| | | | | A3 | 18 | <1.0 |
| SCP-1 437-464 | 453-460 | FEKIAEEL | 551 | B08 | 21 | <1.0 |
| | 452-460 | QFEKLAEEL | 552 | B2705 | 15 | |
| | 451-460 | KQFEKIAEEL | 553 | A0201 | 16 | 56 |
| | 449-456 | DNKQFEKI | 554 | B08 | 16 | 2 |
| | | | | B5101 | 16 | N.A. |
| | 448-456 | YDNKQFEKI | 555 | B5101 | 16 | 1 |
| | 447-456 | LYDNKQFEKI | 556 | A1 | 15 | <1.0 |

TABLE 34K

SCP-1: Preferred Epitoopes Reealed by Housekeeping
Proteasome Digestion

Binding Prediction

| | | | | | | |
|---|---|---|---|---|---|---|
| Substrate | Epitope | Sequence | Seq. ID No. | HLA type | SYFPEITHI | NIH |

| | | | | | | |
|---|---|---|---|---|---|---|
| SCP-1 437-464 | 440-447 | LGEKETLL | 557 | B5101 | 16 | N.A. |
| (continued) | | | | | | |
| | 439-447 | VLGEKETLL | 558 | A0201 | 24 | 149 |
| | | | | A26 | 19 | N.A. |
| | | | | B08 | 29 | 12 |
| | 438-447 | KVLGEKETLL | 559 | A0201 | 19 | 24 |
| | | | | A26 | 18 | N.A. |
| | | | | A24 | N.A. | 12 |
| | | | | A3 | 18 | <1.0 |
| | | | | B0702 | 14 | 20 |
| SCP-1 383-412 | 390-398 | LLRTEQQRL | 560 | A0201 | 22 | 3 |
| | | | | A26 | 18 | N.A. |
| | | | | B08 | 22 | 1.6 |
| | | | | B2705 | 15 | 30 |
| | 389-398 | ELLRTEQQRL | 561 | A0201 | 19 | 6 |
| | | | | A26 | 24 | N.A. |
| | | | | A3 | 15 | <1.0 |
| | 393-401 | TEQQRLENY | 562 | A1 | 15 | <5 |
| | | | | A26 | 16 | N.A. |
| | 392-401 | RTEQQRLENY | 563 | A1 | 31 | 113 |
| | | | | A26 | 26 | N.A. |
| | 402-410 | EDQLIILTM | 564 | A26 | 26 | N.A. |
| | 397-406 | RLENYEDQLI | 565 | A0201 | 17 | <1.0 |
| | | | | A3 | 15 | <1.0 |

TABLE 34L
SCP-1: Preferred Epitopes Revealed by Housekeeping
Proteasome Digestion

| | | | | Binding Prediction | | |
|---|---|---|---|---|---|---|
| | | | Seq. | | SYFP | |
| Sub- | | | ID | HLA | EI | |
| strate | Epitope | Sequence | No. | type | THI | NIH |
| SCP-1 | 368-375 | KARAAHSF | 566 | B08 | 16 | <1.0 |
| 366-394 | 376-384 | VVTEFETTV | 567 | A0201 | 19 | 161 |
| | | | | A3 | 16 | <1.0 |
| | 375-384 | FVVTEFETTV | 568 | A0201 | 17 | 106 |
| | 377-385 | VTEFETTVC | 569 | A1 | 18 | 2 |
| | 376-385 | VVTEFETTVC | 570 | A3 | 16 | <5 |
| SCP-1 | 344-352 | DLQIATNTI | 571 | A0201 | 22 | <5 |
| 331-357 | | | | A3 | 15 | <1.0 |
| | | | | B5101 | 17 | 11 |
| | 347-355 | IATNTICQL | 572 | A0201 | 19 | 1 |
| | | | | B08 | 16 | <1.0 |
| | | | | B5101 | 20 | 79 |
| | 346-355 | QIATNTICQL | 573 | A0201 | 24 | 7 |
| | | | | A26 | 24 | N.A. |

TABLE 35
SSX-4: Preferred Epitopes Revealed by Housekeeping
Proteasome Digestion

| | | | Seq. | Binding Prediction | | |
|---|---|---|---|---|---|---|
| Sub- | | | ID | | SYFP | |
| strate | Epitope | Sequence | No. | HLA type | EITHI | NIH |
| SSX4 | 57-65 | VMTKLGFKV | 574 | A0201 | 21 | 495 |
| 45-76 | 53-61 | LNYEVMTKL | 575 | A0201 | 17 | 7 |
| | 52-61 | KLNYEVMTKL | 576 | A0201 | 23 | 172 |
| | | | | A26 | 21 | N.A. |
| | | | | A24 | N.A. | 18 |
| | | | | A3 | 14 | 4 |
| | | | | B7 | N.A. | 4 |
| | 66-74 | TLPPFMRSK | 577 | A26 | 16 | N.A. |
| | | | | A3 | 25 | 14 |
| SSX4 | 110-118 | KIMPKKPAE | 578 | A0201 | 15 | <5 |
| 98-124 | | | | A26 | 15 | N.A. |
| | | | | A3 | 16 | <5 |
| | 103-112 | SLQRIFPKIM | 579 | A0201 | 15 | 8 |
| | | | | A26 | 16 | N.A. |
| | | | | A3 | 15 | <5 |

TABLE 36
Tyrosinase: Preferred Epitopes Revealed by House-
keeping Proteasome Digestion

| | | | Seq. | Binding Prediction | | |
|---|---|---|---|---|---|---|
| Sub- | | | ID | HLA | SYFP | |
| strate | Epitope | Sequence | No. | type | EITHI | NIH |
| Tyr | 463-471 | YIKSYLEQA | 580 | A0201 | 18 | <5 |
| 445-474 | | | | A26 | 17 | N.A. |
| | 459-467 | SFQDYIKSY | 581 | A1 | 18 | <5 |
| | | | | A26 | 22 | N.A. |
| | 458-467 | DSFQDYIKSY | 582 | A1 | 19 | <5 |
| | | | | A26 | 24 | N.A. |
| Tyr | 507-514 | LPEEKQPL | 583 | B08 | 28 | 5 |
| 490-518 | | | | B5101 | 18 | N.A. |
| | 506-514 | QLPEEKQPL | 584 | A0201 | 22 | 88 |
| | | | | A26 | 20 | N.A. |
| | | | | A24 | N.A. | 9 |
| | | | | B08 | 18 | <5 |
| | 505-514 | KQLPEEKQPL | 585 | A0201 | 15 | 28 |
| | | | | A24 | N.A. | 17 |
| | 507-515 | LPEEKQPLL | 586 | A0201 | 15 | <5 |
| | | | | B0702 | 21 | 24 |
| | | | | B08 | 28 | 5 |
| | | | | B5101 | 21 | 157 |
| | 506-515 | QLPEEKQPLL | 587 | A0201 | 23 | 88 |
| | | | | A26 | 20 | N.A. |
| | | | | A24 | N.A. | 7 |
| | 497-505 | SLLCHRHKRK | 588 | A3 | 25 | 15 |

### Example 15

Evaluating Likelihood of Epitope Cross-Reactivity on Non-Target Tissues.

As noted above PSA is a member of the kallikrein family of proteases, which is itself a subset of the serine protease family. While the members of this family sharing the greatest degree of sequence identity with PSA also share similar expression profiles, it remains possible that individual epitope sequences might be shared with proteins having distinctly different expression profiles. A first step in evaluating the likelihood of undesirable cross-reactivity is the identification of shared sequences. One way to accomplish this is to conduct a BLAST search of an epitope sequence against the SWISSPROT or Entrez non-redundant peptide sequence databases using the “Search for short nearly exact matches” option; hypertext transfer protocol accessible on the world wide web (http://www) at “ncbi.nlm.nih.gov/blast/index.html”. Thus searching SEQ ID NO. 214, WVLTAAHCI, against SWISSPROT (limited to entries for homo sapiens) one finds four exact matches, including PSA. The other three are from kallikrein 1 (tissue kallikrein), and elastase 2A and 2B. While these nine amino acid segments are identical, the flanking sequences are quite distinct, particularly on the C-terminal side, suggesting that processing may proceed differently and that thus the same epitope may not be liberated from these other proteins. (Please note that kallikrein naming is confused. Thus the kallikrein 1 [accession number P06870] is a different protein than the one [accession number AAD13817] mentioned in the paragraph on PSA above in the section on tumor-associated antigens).

It is possible to test this possibility in several ways. Synthetic peptides containing the epitope sequence embedded in the context of each of these proteins can be subjected to in vitro proteasomal digestion and analysis as described above. Alternatively, cells expressing these other proteins, whether by natural or recombinant expression, can be used as targets in a cytotoxicity (or similar) assay using CD8⁺ T cells that recognize the epitope, in order to determine if the epitope is processed and presented.

### Example 16

Epitope Clusters.

Known and predicted epitopes are generally not evenly distributed across the sequences of protein antigens. As referred to above, we have defined segments of sequence containing a higher than average density of (known or predicted) epitopes as epitope clusters. Among the uses of epitope clusters is the incorporation of their sequence into substrate peptides used in proteasomal digestion analysis as described herein. Epitope clusters can also be useful as vaccine components. A fuller discussion of the definition and uses of epitope clusters is found in U.S. patent application Ser. No. 09/561,571 entitled EPITOPE CLUSTERS, previously incorporated by reference in its entirety.

The following tables (37-60) present 9-mer epitopes predicted for HLA-A2 binding using both the SYFPEITHI and NIH algorithms and the epitope density of regions of overlapping eptiopes, and the epitopes in the whole protein, and the ratio of these two densities. (The ratio must exceed one to be a cluster by the above definition; requiring higher values of this ratio reflect preferred embodiments). Individual 9-mers are ranked by score and identified by the position of their first amino in the complete protein sequence. Each potential cluster from a protein is numbered. The range of amino acid positions within the complete sequence that the cluster cover is indicated as are the rankings of the individual predicted epitopes it is made up of.

TABLE 37
BIMAS-NIH/Parker algorithm Results for gp100

| Rank | Start | Score |
|---|---|---|
| 1 | 619 | 1493 |
| 2 | 602 | 413 |
| 3 | 162 | 226 |
| 4 | 18 | 118 |
| 5 | 178 | 118 |
| 6 | 273 | 117 |
| 7 | 601 | 81 |
| 8 | 243 | 63 |
| 9 | 606 | 60 |
| 10 | 373 | 50 |
| 11 | 544 | 36 |
| 12 | 291 | 29 |
| 13 | 592 | 29 |
| 14 | 268 | 29 |
| 15 | 47 | 27 |
| 16 | 585 | 26 |
| 17 | 576 | 21 |
| 18 | 465 | 21 |
| 19 | 570 | 20 |
| 20 | 9 | 19 |
| 21 | 416 | 19 |
| 22 | 25 | 18 |
| 23 | 566 | 17 |
| 24 | 603 | 15 |
| 25 | 384 | 14 |
| 26 | 13 | 14 |
| 27 | 290 | 12 |
| 28 | 637 | 10 |
| 29 | 639 | 9 |
| 30 | 485 | 9 |
| 31 | 453 | 8 |
| 32 | 102 | 8 |
| 33 | 399 | 8 |
| 34 | 456 | 7 |
| 35 | 113 | 7 |
| 36 | 622 | 7 |
| 37 | 69 | 7 |
| 38 | 604 | 6 |
| 39 | 350 | 6 |
| 40 | 583 | 5 |

TABLE 38
SYFPEITHI (Rammensee algorithm) Results for gp100

| Rank | Start | Score |
|---|---|---|
| 1 | 606 | 30 |
| 2 | 162 | 29 |
| 3 | 456 | 28 |
| 4 | 18 | 28 |
| 5 | 602 | 27 |
| 6 | 598 | 27 |
| 7 | 601 | 26 |
| 8 | 597 | 26 |
| 9 | 13 | 26 |
| 10 | 585 | 25 |
| 11 | 449 | 25 |
| 12 | 4 | 25 |
| 13 | 603 | 24 |
| 14 | 576 | 24 |
| 15 | 453 | 24 |
| 16 | 178 | 24 |
| 17 | 171 | 24 |
| 18 | 11 | 24 |
| 19 | 619 | 23 |
| 20 | 280 | 23 |
| 21 | 268 | 23 |
| 22 | 592 | 22 |
| 23 | 544 | 22 |
| 24 | 465 | 22 |
| 25 | 399 | 22 |
| 26 | 373 | 22 |
| 27 | 273 | 22 |
| 28 | 243 | 22 |
| 29 | 566 | 21 |
| 30 | 563 | 21 |
| 31 | 485 | 21 |
| 32 | 384 | 21 |
| 33 | 350 | 21 |
| 34 | 9 | 21 |
| 35 | 463 | 20 |
| 36 | 397 | 20 |
| 37 | 291 | 20 |
| 38 | 269 | 20 |
| 39 | 2 | 20 |
| 40 | 610 | 19 |
| 41 | 594 | 19 |
| 42 | 591 | 19 |
| 43 | 583 | 19 |
| 44 | 570 | 19 |
| 45 | 488 | 19 |
| 46 | 446 | 19 |
| 47 | 322 | 19 |
| 48 | 267 | 19 |
| 49 | 250 | 19 |
| 50 | 205 | 19 |
| 51 | 180 | 19 |
| 52 | 169 | 19 |
| 53 | 88 | 19 |
| 54 | 47 | 19 |
| 55 | 10 | 19 |
| 56 | 648 | 18 |
| 57 | 605 | 18 |
| 58 | 604 | 18 |
| 59 | 595 | 18 |
| 60 | 571 | 18 |
| 61 | 569 | 18 |
| 62 | 450 | 18 |
| 63 | 409 | 18 |
| 64 | 400 | 18 |
| 65 | 371 | 18 |
| 66 | 343 | 18 |
| 67 | 298 | 18 |
| 68 | 209 | 18 |
| 69 | 102 | 18 |
| 70 | 97 | 18 |
| 71 | 76 | 18 |
| 72 | 69 | 18 |
| 73 | 60 | 18 |
| 74 | 17 | 18 |
| 75 | 613 | 17 |
| 76 | 599 | 17 |
| 77 | 572 | 17 |
| 78 | 557 | 17 |
| 79 | 556 | 17 |
| 80 | 512 | 17 |
| 81 | 406 | 17 |
| 82 | 324 | 17 |
| 83 | 290 | 17 |
| 84 | 101 | 17 |
| 85 | 95 | 17 |
| 86 | 635 | 16 |
| 87 | 588 | 16 |
| 88 | 584 | 16 |
| 89 | 577 | 16 |
| 90 | 559 | 16 |
| 91 | 539 | 16 |
| 92 | 494 | 16 |
| 93 | 482 | 16 |
| 94 | 468 | 16 |
| 95 | 442 | 16 |
| 96 | 413 | 16 |
| 97 | 408 | 16 |
| 98 | 402 | 16 |
| 99 | 286 | 16 |
| 100 | 234 | 16 |
| 101 | 217 | 16 |
| 102 | 211 | 16 |
| 103 | 176 | 16 |
| 104 | 107 | 16 |
| 105 | 96 | 16 |
| 106 | 80 | 16 |
| 107 | 16 | 16 |
| 108 | 14 | 16 |
| 109 | 7 | 16 |

TABLE 39
Prediction of clusters for gp100
Total AAs: 661
Total 9-mers: 653
SYFPEITHI 16: 109 9-mers
NIH 5:40 9-mers

| | | | | Epitopes/AA | | |
|---|---|---|---|---|---|---|
| | Cluster # | AAs | Epitopes (by Rank) | Cluster | Whole Pr | Ratio |
| SYFPEITHI | 1 | 2 to 26 | 39, 12, 109, 34, 55, 11, 9, | 0.440 | 0.165 | 2.668 |
| | | | 108, 107, 74, 4 | | | |
| | 2 | 69—115 | 72, 71, 106, 53, 85, 105, | 0.213 | 0.165 | 1.290 |
| | | | 70, 84, 69, 104 | | | |
| | 3 | 95-115 | 85, 105, 70, 84, 69 | 0.238 | 0.165 | 1.444 |
| | 4 | 162-188 | 2, 52, 17, 103, 16, 51 | 0.222 | 0.165 | 1.348 |
| | 5 | 205-225 | 50, 68, 102, 101 | 0.190 | 0.165 | 1.155 |
| | 6 | 243-258 | 28, 49 | 0.125 | 0.165 | 0.758 |
| | 7 | 267-306 | 48, 21, 38, 27, 20, 99, 83, 37, 67 | 0.225 | 0.165 | 1.364 |
| | 8 | 322-332 | 47, 82 | 0.182 | 0.165 | 1.103 |
| | 9 | 343-358 | 66, 33 | 0.125 | 0.165 | 0.758 |
| | 10 | 371-381 | 65, 26 | 0.182 | 0.165 | 1.103 |
| | 11 | 397-421 | 36, 25, 64, 98, 81, 97, 63, 96 | 0.320 | 0.165 | 1.941 |
| | 12 | 442-476 | 95, 46, 11, 62, 15, 3, 35, 24, 94 | 0.257 | 0.165 | 1.559 |
| | 13 | 482-502 | 93, 31, 45, 93 | 0.190 | 0.165 | 1.155 |
| | 14 | 539-552 | 91, 23 | 0.143 | 0.165 | 0.866 |
| | 15 | 556-627 | 79, 78, 90, 30, 29, 61, 44, 60, 77, 14, | 0.431 | 0.165 | 2.611 |
| | | | 89, 43, 88, 10, 87, 42, 22, 41, 59, 8, | | | |
| | | | 6, 76, 7, 5, 13, 58, 57, 1, 40, 75, 19 | | | |
| NIH | 1 | 9 to 33 | 20, 26, 4, 22 | 0.160 | 0.061 | 2.644 |
| | 2 | 268-281 | 14, 6 | 0.143 | 0.061 | 2.361 |
| | 3 | 290-299 | 27, 12 | 0.200 | 0.061 | 3.305 |
| | 4* | 102-121 | 32, 35 | 0.100 | 0.061 | 1.653 |
| | 5* | 373-392 | 10, 25 | 0.100 | 0.061 | 1.653 |
| | 6 | 453-473 | 31, 34, 18 | 0.143 | 0.061 | 2.361 |
| | 7 | 566-600 | 23, 19, 17, 40, 16, 13 | 0.171 | 0.061 | 2.833 |
| | 8 | 601-614 | 7, 2, 24, 38, 9 | 0.357 | 0.061 | 5.902 |
| | 9 | 619-630 | 1, 36 | 0.17 | 0.061 | 2.754 |
| | 10 | 637-647 | 28, 29 | 0.18 | 0.061 | 3.005 |

*Nearby but not overlapping epitopes

TABLE 40
BIMAS-NIH/Parker algorithm Results for PSMA

| Rank | Start | Score |
|---|---|---|
| 1 | 663 | 1360 |
| 2 | 711 | 1055 |
| 3 | 4 | 485 |
| 4 | 27 | 400 |
| 5 | 26 | 375 |
| 6 | 668 | 261 |
| 7 | 707 | 251 |
| 8 | 469 | 193 |
| 9 | 731 | 177 |
| 10 | 35 | 67 |
| 11 | 33 | 64 |
| 12 | 554 | 59 |
| 13 | 427 | 50 |
| 14 | 115 | 47 |
| 15 | 20 | 40 |
| 16 | 217 | 26 |
| 17 | 583 | 24 |
| 18 | 415 | 19 |
| 19 | 193 | 14 |
| 20 | 240 | 12 |
| 21 | 627 | 11 |
| 22 | 260 | 10 |
| 23 | 130 | 10 |
| 24 | 741 | 9 |
| 25 | 3 | 9 |
| 26 | 733 | 8 |
| 27 | 726 | 7 |
| 28 | 286 | 6 |
| 29 | 174 | 5 |
| 30 | 700 | 5 |

TABLE 41
SYFPEITHI (Rammensee algorithm) Results for PSMA

| Rank | Start | Score |
|---|---|---|
| 1 | 469 | 27 |
| 2 | 27 | 27 |
| 3 | 741 | 26 |
| 4 | 711 | 26 |
| 5 | 354 | 25 |
| 6 | 4 | 25 |
| 7 | 663 | 24 |
| 8 | 130 | 24 |
| 9 | 57 | 24 |
| 10 | 707 | 23 |
| 11 | 260 | 23 |
| 12 | 20 | 23 |
| 13 | 603 | 22 |
| 14 | 218 | 22 |
| 15 | 109 | 22 |
| 16 | 731 | 21 |
| 17 | 668 | 21 |
| 18 | 660 | 21 |
| 19 | 507 | 21 |
| 20 | 454 | 21 |
| 21 | 427 | 21 |
| 22 | 358 | 21 |
| 23 | 284 | 21 |
| 24 | 115 | 21 |
| 25 | 33 | 21 |
| 26 | 606 | 20 |
| 27 | 568 | 20 |
| 28 | 473 | 20 |
| 29 | 461 | 20 |
| 30 | 200 | 20 |
| 31 | 26 | 20 |
| 32 | 3 | 20 |
| 33 | 583 | 19 |
| 34 | 579 | 19 |
| 35 | 554 | 19 |
| 36 | 550 | 19 |
| 37 | 547 | 19 |
| 38 | 390 | 19 |
| 39 | 219 | 19 |
| 40 | 193 | 19 |
| 41 | 700 | 18 |
| 42 | 472 | 18 |
| 43 | 364 | 18 |
| 44 | 317 | 18 |
| 45 | 253 | 18 |
| 46 | 91 | 18 |
| 47 | 61 | 18 |
| 48 | 13 | 18 |
| 49 | 733 | 17 |
| 50 | 673 | 17 |
| 51 | 671 | 17 |
| 52 | 642 | 17 |
| 53 | 571 | 17 |
| 54 | 492 | 17 |
| 55 | 442 | 17 |
| 56 | 441 | 17 |
| 57 | 397 | 17 |
| 58 | 391 | 17 |
| 59 | 357 | 17 |
| 60 | 344 | 17 |
| 61 | 305 | 17 |
| 62 | 304 | 17 |
| 63 | 286 | 17 |
| 64 | 282 | 17 |
| 65 | 169 | 17 |
| 66 | 142 | 17 |
| 67 | 122 | 17 |
| 68 | 738 | 16 |
| 69 | 634 | 16 |
| 70 | 631 | 16 |
| 71 | 515 | 16 |
| 72 | 456 | 16 |
| 73 | 440 | 16 |
| 74 | 385 | 16 |
| 75 | 373 | 16 |
| 76 | 365 | 16 |
| 77 | 361 | 16 |
| 78 | 289 | 16 |
| 79 | 278 | 16 |
| 80 | 258 | 16 |
| 81 | 247 | 16 |
| 82 | 217 | 16 |
| 83 | 107 | 16 |
| 84 | 100 | 16 |
| 85 | 75 | 16 |
| 86 | 37 | 16 |
| 87 | 30 | 16 |
| 88 | 21 | 16 |

TABLE 42
Prediction of clusters for prostate-specific membrane antigen (PSMA)
Total AAs: 750
Total 9-mers: 742
SYFPEITHI 16: 88 9-mers
NIH 5: 30 9-mers

| | | | | Epitopes/AA | | |
|---|---|---|---|---|---|---|
| | Cluster # | Aas | Epitopes (by rank) | Cluster | Whole Pr | Ratio |
| SYFPEITHI | 1 | 3 to 12 | 32, 6 | 0.200 | 0.117 | 1.705 |
| | 2 | 13-45 | 13, 12, 88, 31, 2, 87, 25, 86 | 0.242 | 0.117 | 2.066 |
| | 3 | 57-69 | 9, 47 | 0.154 | 0.117 | 1.311 |
| | 4 | 100-138 | 84, 83, 15, 24, 67, 8 | 0.154 | 0.117 | 1.311 |
| | 5 | 193-208 | 40, 30 | 0.111 | 0.117 | 0.947 |
| | 6 | 217-227 | 82, 14, 39 | 0.273 | 0.117 | 2.324 |
| | 7 | 247-268 | 81, 45, 80, 11 | 0.182 | 0.117 | 1.550 |
| | 8 | 278-297 | 79, 64, 23, 63, 78 | 0.250 | 0.117 | 2.131 |
| | 9 | 354-381 | 5, 59, 22, 77, 43, 76, 75 | 0.250 | 0.117 | 2.131 |
| | 10 | 385-405 | 74, 38, 58, 57 | 0.190 | 0.117 | 1.623 |
| | 11 | 440-450 | 73, 56, 55 | 0.273 | 0.117 | 2.324 |
| | 12 | 454-481 | 20, 72, 29, 1, 42, 28 | 0.214 | 0.117 | 1.826 |
| | 13 | 507-523 | 17, 71 | 0.118 | 0.117 | 1.003 |
| | 14 | 547-562 | 37, 36, 35 | 0.188 | 0.117 | 1.598 |
| | 15 | 568-591 | 27, 53, 34, 33 | 0.167 | 0.117 | 1.420 |
| | 16 | 603-614 | 13, 26 | 0.167 | 0.117 | 1.420 |
| | 17 | 631-650 | 70, 69, 52 | 0.150 | 0.117 | 1.278 |
| | 18 | 660-681 | 18, 7, 17, 51, 50 | 0.227 | 0.117 | 1.937 |
| | 19 | 700-719 | 41, 10, 4 | 0.150 | 0.117 | 1.278 |
| | 20 | 731-749 | 16, 49, 68, 3 | 0.211 | 0.117 | 1.794 |
| NIH | 1 | 3 to 12 | 25, 3 | 0.200 | 0.040 | 5.000 |
| | 2 | 20-43 | 15, 5, 4, 11, 10 | 0.208 | 0.040 | 5.208 |
| | 3* | 415-435 | 18, 13 | 0.095 | 0.040 | 2.381 |
| | 4 | 663-676 | 1, 6 | 0.143 | 0.040 | 3.571 |
| | 5 | 700-715 | 30, 7, 3 | 0.188 | 0.040 | 4.688 |
| | 6 | 726-749 | 27, 9, 26, 24 | 0.167 | 0.040 | 4.167 |

*Nearby but not overlapping epitopes

TABLE 43
BIMAS-NIH/Parker algorithm Results for PSA

| Rank | Start | Score |
|---|---|---|
| 1 | 7 | 607 |
| 2 | 170 | 243 |
| 3 | 52 | 124 |
| 4 | 53 | 112 |
| 5 | 195 | 101 |
| 6 | 165 | 23 |
| 7 | 72 | 18 |
| 8 | 245 | 18 |
| 9 | 2 | 16 |
| 10 | 59 | 16 |
| 11 | 122 | 15 |
| 12 | 125 | 15 |
| 13 | 191 | 13 |
| 14 | 9 | 8 |
| 15 | 14 | 6 |
| 16 | 175 | 5 |
| 17 | 130 | 5 |

TABLE 44
SYFPEITHI (Rammensee algorithm) Results for PSA

| Rank | Start | Score |
|---|---|---|
| 1 | 72 | 26 |
| 2 | 170 | 22 |
| 3 | 53 | 22 |
| 4 | 7 | 22 |
| 5 | 234 | 21 |
| 6 | 166 | 21 |
| 7 | 140 | 21 |
| 8 | 66 | 21 |
| 9 | 241 | 20 |
| 10 | 175 | 20 |
| 11 | 12 | 20 |
| 12 | 41 | 19 |
| 13 | 20 | 19 |
| 14 | 14 | 19 |
| 15 | 130 | 18 |
| 16 | 124 | 18 |
| 17 | 121 | 18 |
| 18 | 47 | 18 |
| 19 | 17 | 18 |
| 20 | 218 | 17 |
| 21 | 133 | 17 |
| 22 | 125 | 17 |
| 23 | 122 | 17 |
| 24 | 118 | 17 |
| 25 | 110 | 17 |
| 26 | 67 | 17 |
| 27 | 52 | 17 |
| 28 | 21 | 17 |
| 29 | 16 | 17 |
| 30 | 2 | 17 |
| 31 | 184 | 16 |
| 32 | 179 | 16 |
| 33 | 158 | 16 |
| 34 | 79 | 16 |
| 35 | 73 | 16 |
| 36 | 4 | 16 |

TABLE 45
Prediction of clusters for prostate specific antigen (PSA)
Total AAs: 261
Total 9-mers: 253
SYFPEITHI 16: 36 9-mers
NIH 5: 17 9-mers

| | | | | Epitopes/AA | | |
|---|---|---|---|---|---|---|
| | Cluster # | AAs | Epitopes (by rank) | Cluster | Whole Pr | Ratio |
| SYFPEITHI | 1 | 2 to 29 | 30, 36, 4, 11, 14, 29, 19, 13, 28 | 0.321 | 0.138 | 2.330 |
| | 2 | 41-61 | 12, 18, 27, 3 | 0.190 | 0.138 | 1.381 |
| | 3 | 66-87 | 8, 26, 1, 35, 34 | 0.227 | 0.138 | 1.648 |
| | 4 | 110-148 | 25, 24, 17, 23, 16, 22, 15, 21, 7 | 0.184 | 0.138 | 1.332 |
| | 5 | 158-192 | 33, 6, 2, 10, 32, 31 | 0.171 | 0.138 | 1.243 |
| | 6 | 234-249 | 5, 9 | 0.125 | 0.138 | 0.906 |
| | 7* | 118-133 | 24, 17, 23, 16, 22 | 0.313 | 0.138 | 2.266 |
| | 8* | 118-138 | 24, 17, 23, 16, 22, 15 | 0.286 | 0.138 | 2.071 |
| NIH | 1 | 2-22 | 9, 1, 14, 15 | 0.190 | 0.065 | 2.924 |
| | 2 | 52-67 | 3, 4, 10 | 0.188 | 0.065 | 2.879 |
| | 3 | 122-138 | 11, 12, 17 | 0.176 | 0.065 | 2.709 |
| | 4 | 165-183 | 6, 2, 16 | 0.158 | 0.065 | 2.424 |
| | 5 | 191-203 | 13, 5 | 0.154 | 0.065 | 2.362 |
| | 6** | 52-80 | 3, 4, 10, 7 | 0.138 | 0.065 | 2.118 |

*These clusters are internal to the less preferred cluster #4.
**Includes a nearby but not overlapping epitope.

TABLE 46
BIMAS-NIH/Parker algorithm Results for PSCA

| Rank | Start | Score |
|---|---|---|
| 1 | 43 | 153 |
| 2 | 5 | 84 |
| 3 | 7 | 79 |
| 4 | 109 | 36 |
| 5 | 105 | 25 |
| 6 | 108 | 24 |
| 7 | 14 | 21 |
| 8 | 20 | 18 |
| 9 | 115 | 17 |
| 10 | 42 | 15 |
| 11 | 36 | 15 |
| 12 | 99 | 9 |
| 13 | 58 | 8 |

TABLE 47
SYFPEITHI (Rammensee algorithm) Results for PSCA

| Rank | Start | Score |
|---|---|---|
| 1 | 108 | 30 |
| 2 | 14 | 30 |
| 3 | 105 | 29 |
| 4 | 5 | 28 |
| 5 | 115 | 26 |
| 6 | 99 | 26 |
| 7 | 7 | 26 |
| 8 | 109 | 24 |
| 9 | 53 | 23 |
| 10 | 107 | 21 |
| 11 | 20 | 21 |
| 12 | 8 | 21 |
| 13 | 13 | 20 |
| 14 | 102 | 19 |
| 15 | 60 | 19 |
| 16 | 57 | 19 |
| 17 | 54 | 19 |
| 18 | 12 | 19 |
| 19 | 4 | 19 |
| 20 | 1 | 19 |
| 21 | 112 | 18 |
| 22 | 101 | 18 |
| 23 | 98 | 18 |
| 24 | 51 | 18 |
| 25 | 43 | 18 |
| 26 | 106 | 17 |
| 27 | 104 | 17 |
| 28 | 83 | 17 |
| 29 | 63 | 17 |
| 30 | 50 | 17 |
| 31 | 3 | 17 |
| 32 | 9 | 16 |
| 33 | 92 | 16 |

TABLE 48
Prediction of clusters for prostate stem cell antigen (PSCA)
Total AAs: 123
Total 9-mers: 115
SYFPEITHI 16: 33;
SYFPEITHI 20: 13
NIH 5: 13

| | | | | Epitopes/AA | | |
|---|---|---|---|---|---|---|
| | Cluster # | AAs | Epitopes (by rank) | Cluster | Whole Pr. | Ratio |
| SYFPEITHI>16 | 1 | 1 to 28 | 20, 31, 19, 4, 7, 12, 33, 18, 13, 2, 11 | 0.393 | 0.268 | 1.464 |
| | 2 | 43-71 | 25, 30, 24, 9, 17, 16, 15, 29 | 0.276 | 0.268 | 1.028 |
| | 3 | 92-123 | 32, 23, 6, 27, 14, 22, 3, 26, 10, | 0.406 | 0.268 | 1.514 |
| | | | 1, 8, 21, 5 | | | |
| SYFPEITHI >20 | 1 | 5 to 28 | 4, 7, 12, 13, 2, 11 | 0.250 | 0.106 | 2.365 |
| | 2 | 99-123 | 6, 3, 10, 1, 8, 5 | 0.240 | 0.106 | 2.271 |
| NIH | 1 | 5 to 28 | 2, 3, 7, 8 | 0.167 | 0.106 | 1.577 |
| | 2 | 36-51 | 11, 10, 1 | 0.188 | 0.106 | 1.774 |
| | 3 | 99-123 | 12, 5, 6, 4, 9 | 0.200 | 0.106 | 1.892 |
| | 4* | 105-116 | 5, 6, 4 | 0.250 | 0.106 | 2.365 |

*This cluster is internal to the less preferred cluster #3.

In tables 49-60 epitope prediction and cluster analysis data for each algorithm are presented together in a single table.

TABLE 49
Prediction of clusters for MAGE-1 (NIH algorithm)
Total AAs: 309
Total 9-mers: 301
NIH 5: 19 9-mers

| | | | Start | | Epitopes/AA | | |
|---|---|---|---|---|---|---|---|
| | | Epitope | Posi- | NIH | | Whole | |
| Cluster # | AAs | Rank | tion | Score | Cluster | Pr. | Ratio |
| 1 | 18-32 | 16 | 18 | 9 | 0.133 | 0.063 | 2.112 |
| | | 19 | 24 | 7 | | | |
| 2 | 101-113 | 14 | 101 | 11 | 0.154 | 0.063 | 2.442 |
| | | 7 | 105 | 44 | | | |
| 3 | 146-159 | 9 | 146 | 32 | 0.143 | 0.063 | 2.263 |
| | | 3 | 151 | 169 | | | |
| 4 | 169-202 | 10 | 169 | 32 | 0.176 | 0.063 | 2.796 |
| | | 13 | 174 | 16 | | | |
| | | 18 | 181 | 8 | | | |
| | | 17 | 187 | 8 | | | |
| | | 6 | 188 | 74 | | | |
| | | 5 | 194 | 110 | | | |
| 5 | 264-277 | 2 | 264 | 190 | 0.143 | 0.063 | 2.263 |
| | | 12 | 269 | 20 | | | |
| 6 | 278-290 | 1 | 278 | 743 | 0.154 | 0.063 | 2.437 |
| | | 11 | 282 | 28 | | | |

TABLE 50
Prediction of clusters for MAGE-1 (SYFPEITHI algorithm)

| | | Epitope | Start | SYFPEITHI | Epitopes/AA | | |
|---|---|---|---|---|---|---|---|
| Cluster # | Aas | Rank | Position | Score | Cluster | Whole | Ratio |
| 1 | 7-49 | 22 | 7 | 19 | 0.233 | 0.153 | 1.522 |
| | | 9 | 15 | 22 | | | |
| | | 27 | 18 | 18 | | | |
| | | 16 | 20 | 20 | | | |
| | | 28 | 22 | 18 | | | |
| | | 29 | 24 | 18 | | | |
| | | 33 | 31 | 17 | | | |
| | | 30 | 35 | 18 | | | |
| | | 2 | 38 | 26 | | | |
| | | 17 | 41 | 20 | | | |
| 2 | 89-132 | 10 | 89 | 22 | 0.273 | 0.153 | 1.783 |
| | | 18 | 92 | 20 | | | |
| | | 7 | 93 | 23 | | | |
| | | 23 | 96 | 19 | | | |
| | | 43 | 98 | 16 | | | |
| | | 4 | 101 | 25 | | | |
| | | 8 | 105 | 23 | | | |
| | | 34 | 107 | 17 | | | |
| | | 35 | 108 | 17 | | | |
| | | 36 | 113 | 17 | | | |
| | | 37 | 118 | 17 | | | |
| | | 19 | 124 | 20 | | | |
| 3 | 167-203 | 44 | 167 | 16 | 0.270 | 0.153 | 1.766 |
| | | 20 | 169 | 20 | | | |
| | | 12 | 174 | 21 | | | |
| | | 24 | 181 | 19 | | | |
| | | 6 | 187 | 24 | | | |
| | | 31 | 188 | 18 | | | |
| | | 25 | 191 | 19 | | | |
| | | 38 | 192 | 17 | | | |
| | | 1 | 194 | 27 | | | |
| | | 13 | 195 | 21 | | | |
| 4 | 230-246 | 14 | 230 | 21 | 0.118 | 0.153 | 0.769 |
| | | 39 | 238 | 17 | | | |
| 5 | 264-297 | 15 | 264 | 21 | 0.235 | 0.153 | 1.538 |
| | | 32 | 269 | 18 | | | |
| | | 40 | 270 | 17 | | | |
| | | 26 | 271 | 19 | | | |
| | | 46 | 275 | 16 | | | |
| | | 3 | 278 | 26 | | | |
| | | 21 | 282 | 20 | | | |
| | | 41 | 289 | 17 | | | |

Total AAs: 309
Total 9-mers: 301
SYFPEITHI 16: 46 9-mers

TABLE 51
Prediction of clusters for MAGE-2 (NIH algorithm)

| | | Epitope | Start | NIH | Epitope/AA | | |
|---|---|---|---|---|---|---|---|
| Cluster # | AAs | Rank | Position | Score | Cluster | Whole Pr. | Ratio |
| 1 | 101-120 | 18 | 101 | 5.373 | 0.150 | 0.065 | 2.310 |
| | | 16 | 108 | 6.756 | | | |
| | | 1 | 112 | 2800.697 | | | |
| 2 | 153-167 | 8 | 153 | 31.883 | 0.200 | 0.065 | 3.080 |
| | | 4 | 158 | 168.552 | | | |
| | | 7 | 159 | 32.138 | | | |
| 3 | 169-211 | 14 | 169 | 8.535 | 0.209 | 0.065 | 3.223 |
| | | 19 | 174 | 5.346 | | | |
| | | 6 | 176 | 49.993 | | | |
| | | 11 | 181 | 15.701 | | | |
| | | 15 | 188 | 7.536 | | | |
| | | 12 | 195 | 12.809 | | | |
| | | 5 | 200 | 88.783 | | | |
| | | 10 | 201 | 16.725 | | | |
| | | 17 | 203 | 5.609 | | | |
| 4 | 271-284 | 3 | 271 | 398.324 | 0.143 | 0.065 | 2.200 |
| | | 9 | 276 | 19.658 | | | |

Total AAs: 314
Total 9-mers: 308
NIH >= 5: 20 9-mers

TABLE 52
Prediction of clusters for MAGE-2 (SYFPEITHI algorithm)

| | | Epitope | Start | SYFPEITHI | Epitopes/AA | | |
|---|---|---|---|---|---|---|---|
| Cluster # | AAs | Rank | Position | Score | Cluster | Whole Pr. | Ratio |
| 1 | 15-32 | 13 | 15 | 21 | 0.278 | 0.169 | 1.645 |
| | | 29 | 18 | 18 | | | |
| | | 43 | 20 | 16 | | | |
| | | 30 | 22 | 18 | | | |
| | | 21 | 24 | 19 | | | |
| 2 | 37-56 | 31 | 37 | 18 | 0.250 | 0.169 | 1.481 |
| | | 16 | 40 | 20 | | | |
| | | 44 | 44 | 16 | | | |
| | | 14 | 45 | 21 | | | |
| | | 22 | 48 | 19 | | | |
| 3 | 96-133 | 36 | 96 | 17 | 0.211 | 0.169 | 1.247 |
| | | 46 | 101 | 16 | | | |
| | | 6 | 108 | 25 | | | |
| | | 47 | 109 | 16 | | | |
| | | 2 | 112 | 27 | | | |
| | | 37 | 120 | 17 | | | |
| | | 38 | 125 | 17 | | | |
| | | 17 | 131 | 20 | | | |
| 4 | 153-216 | 12 | 153 | 22 | 0.344 | 0.169 | 2.036 |
| | | 39 | 158 | 17 | | | |
| | | 7 | 159 | 25 | | | |
| | | 23 | 161 | 19 | | | |
| | | 24 | 162 | 19 | | | |
| | | 48 | 164 | 16 | | | |
| | | 49 | 167 | 16 | | | |
| | | 32 | 170 | 18 | | | |
| | | 50 | 171 | 16 | | | |
| | | 4 | 174 | 26 | | | |
| | | 9 | 176 | 24 | | | |
| | | 51 | 177 | 16 | | | |
| | | 15 | 181 | 21 | | | |
| | | 25 | 188 | 19 | | | |
| | | 18 | 194 | 20 | | | |
| | | 33 | 195 | 18 | | | |
| | | 19 | 198 | 20 | | | |
| | | 3 | 200 | 27 | | | |
| | | 1 | 201 | 28 | | | |
| | | 40 | 202 | 17 | | | |
| | | 10 | 203 | 23 | | | |
| | | 52 | 208 | 16 | | | |
| 5 | 237-254 | 26 | 237 | 19 | 0.167 | 0.169 | 0.987 |
| | | 27 | 245 | 19 | | | |
| | | 34 | 246 | 18 | | | |
| 6 | 271-299 | 8 | 271 | 25 | 0.241 | 0.169 | 1.430 |
| | | 35 | 276 | 18 | | | |
| | | 41 | 277 | 17 | | | |
| | | 11 | 278 | 23 | | | |
| | | 28 | 283 | 19 | | | |
| | | 20 | 285 | 20 | | | |
| | | 42 | 291 | 17 | | | |

Total AAs: 314
Total 9-mers: 308
SYFPEITHI 16: 52 9-mers

TABLE 53
Prediction of clusters for MAGE-3 (NIH algorithm)

| | | Epitope | Start | NIH | Epitopes/AA | | |
|---|---|---|---|---|---|---|---|
| Cluster # | AAs | Rank | Position | Score | Cluster | Whole Pr. | Ratio |
| 1 | 101-120 | 15 | 101 | 11.002 | 0.200 | 0.071 | 2.800 |
| | | 21 | 105 | 6.488 | | | |
| | | 8 | 108 | 49.134 | | | |
| | | 2 | 112 | 339.313 | | | |
| 2 | 153-167 | 18 | 153 | 7.776 | 0.200 | 0.071 | 2.800 |
| | | 6 | 158 | 51.77 | | | |
| | | 22 | 159 | 5.599 | | | |
| 3 | 174-209 | 17 | 174 | 8.832 | 0.194 | 0.071 | 2.722 |
| | | 7 | 176 | 49.993 | | | |
| | | 13 | 181 | 15.701 | | | |
| | | 19 | 188 | 7.536 | | | |
| | | 14 | 195 | 12.809 | | | |
| | | 5 | 200 | 88.783 | | | |
| | | 12 | 201 | 16.725 | | | |
| 4 | 237-251 | 16 | 237 | 10.868 | 0.200 | 0.071 | 2.800 |
| | | 4 | 238 | 148.896 | | | |
| | | 20 | 243 | 6.88 | | | |
| 5 | 271-284 | 1 | 271 | 2655.495 | 0.143 | 0.071 | 2.000 |
| | | 11 | 276 | 19.658 | | | |

Total AAs: 314
Total 9-mers: 308
NIH 5: 22 9-mers

TABLE 54
Prediction of clusters for MAGE-3 (SYFPEITHI algorithm)

| | | Epitope | Start | SYFPEITHI | Epitopes/AA | | |
|---|---|---|---|---|---|---|---|
| Cluster # | AAs | Rank | Position | Score | Cluster | Whole Pr. | Ratio |
| 1 | 15-32 | 12 | 15 | 21 | 0.278 | 0.153 | 1.820 |
| | | 26 | 18 | 18 | | | |
| | | 37 | 20 | 16 | | | |
| | | 27 | 22 | 18 | | | |
| | | 18 | 24 | 19 | | | |
| 2 | 38-56 | 38 | 38 | 16 | 0.263 | 0.153 | 1.725 |
| | | 15 | 40 | 20 | | | |
| | | 39 | 44 | 16 | | | |
| | | 13 | 45 | 21 | | | |
| | | 19 | 48 | 19 | | | |
| 3 | 101-142 | 28 | 101 | 18 | 0.190 | 0.153 | 1.248 |
| | | 40 | 105 | 16 | | | |
| | | 1 | 108 | 31 | | | |
| | | 6 | 112 | 25 | | | |
| | | 31 | 120 | 17 | | | |
| | | 32 | 125 | 17 | | | |
| | | 16 | 131 | 20 | | | |
| | | 41 | 134 | 16 | | | |
| 4 | 153-216 | 20 | 153 | 19 | 0.313 | 0.153 | 2.048 |
| | | 29 | 156 | 18 | | | |
| | | 33 | 158 | 17 | | | |
| | | 21 | 159 | 19 | | | |
| | | 34 | 161 | 17 | | | |
| | | 42 | 164 | 16 | | | |
| | | 43 | 167 | 16 | | | |
| | | 10 | 174 | 22 | | | |
| | | 8 | 176 | 23 | | | |
| | | 14 | 181 | 21 | | | |
| | | 22 | 188 | 19 | | | |
| | | 44 | 193 | 16 | | | |
| | | 11 | 194 | 22 | | | |
| | | 23 | 195 | 19 | | | |
| | | 45 | 197 | 16 | | | |
| | | 17 | 198 | 20 | | | |
| | | 3 | 200 | 27 | | | |
| | | 2 | 201 | 28 | | | |
| | | 35 | 202 | 17 | | | |
| | | 46 | 208 | 16 | | | |
| 5 | 220-230 | 5 | 220 | 26 | 0.182 | 0.153 | 1.191 |
| | | 47 | 222 | 16 | | | |
| 6 | 237-246 | 7 | 237 | 25 | 0.200 | 0.153 | 1.311 |
| | | 9 | 238 | 23 | | | |
| 7 | 271-293 | 4 | 271 | 27 | 0.217 | 0.153 | 1.425 |
| | | 30 | 276 | 18 | | | |
| | | 24 | 278 | 19 | | | |
| | | 36 | 283 | 17 | | | |
| | | 25 | 285 | 19 | | | |

Total AAs: 314
Total 9-mers: 308
SYFPEITHI 16: 47 9-mers

TABLE 55
Prediction of clusters for PRAME (NIH algorithm)

| | | Epitope | Start | NIH | Epitopes/AA | | |
|---|---|---|---|---|---|---|---|
| Cluster # | AAs | Rank | Position | Score | Cluster | Whole Pr. | Ratio |
| 1 | 33-47 | 20 | 33 | 18 | 0.133 | 0.080 | 1.670 |
| | | 17 | 39 | 21 | | | |
| 2 | 71-81 | 9 | 71 | 50 | 0.2 | 0.07984 | 2.505 |
| | | 32 | 73 | 7 | | | |
| 3 | 99-108 | 23 | 100 | 15 | 0.2 | 0.07984 | 2.505 |
| | | 24 | 99 | 13 | | | |
| 4 | 126-135 | 38 | 126 | 5 | 0.2 | 0.07984 | 2.505 |
| | | 35 | 127 | 6 | | | |
| 5 | 224-246 | 5 | 224 | 124 | 0.130 | 0.080 | 1.634 |
| | | 8 | 230 | 63 | | | |
| | | 39 | 238 | 5 | | | |
| 6 | 290-303 | 18 | 290 | 18 | 0.214 | 0.080 | 2.684 |
| | | 14 | 292 | 23 | | | |
| | | 7 | 295 | 66 | | | |
| 7 | 305-324 | 28 | 305 | 10 | 0.200 | 0.080 | 2.505 |
| | | 30 | 308 | 8 | | | |
| | | 25 | 312 | 13 | | | |
| | | 36 | 316 | 6 | | | |
| 8 | 394-409 | 2 | 394 | 182 | 0.188 | 0.080 | 2.348 |
| | | 12 | 397 | 42 | | | |
| | | 31 | 401 | 7 | | | |
| 9 | 422-443 | 10 | 422 | 49 | 0.227 | 0.080 | 2.847 |
| | | 3 | 425 | 182 | | | |
| | | 34 | 431 | 7 | | | |
| | | 29 | 432 | 9 | | | |
| | | 4 | 435 | 160 | | | |
| 10 | 459-487 | 15 | 459 | 21 | 0.172 | 0.080 | 2.159 |
| | | 11 | 462 | 45 | | | |
| | | 22 | 466 | 15 | | | |
| | | 40 | 472 | 5 | | | |
| | | 37 | 479 | 6 | | | |

Total AAs: 509
Total 9-mers: 501
NIH 5: 40 9-mers

TABLE 56
Prediction of clusters for PRAME (SYFPEITHI algorithm)

| | | Epitope | Start | SYFPEITHI | Epitopes/AA | | |
|---|---|---|---|---|---|---|---|
| Cluster # | AAs | Rank | Position | Score | Cluster | Whole Pr. | Ratio |
| 1 | 18-59 | 65 | 18 | 17 | 0.238 | 0.160 | 1.491 |
| | | 50 | 21 | 18 | | | |
| | | 66 | 26 | 17 | | | |
| | | 35 | 33 | 20 | | | |
| | | 22 | 34 | 22 | | | |
| | | 51 | 37 | 18 | | | |
| | | 5 | 39 | 27 | | | |
| | | 23 | 40 | 22 | | | |
| | | 13 | 44 | 24 | | | |
| | | 46 | 51 | 19 | | | |
| 2 | 78-115 | 36 | 78 | 20 | 0.263 | 0.160 | 1.648 |
| | | 67 | 80 | 17 | | | |
| | | 52 | 84 | 18 | | | |
| | | 24 | 86 | 22 | | | |
| | | 53 | 91 | 18 | | | |
| | | 25 | 93 | 22 | | | |
| | | 9 | 99 | 25 | | | |
| | | 8 | 100 | 26 | | | |
| | | 54 | 103 | 18 | | | |
| | | 55 | 107 | 18 | | | |
| 3 | 191-202 | 56 | 191 | 18 | 0.167 | 0.160 | 1.044 |
| | | 38 | 194 | 20 | | | |
| 4 | 205-215 | 26 | 205 | 22 | 0.182 | 0.160 | 1.139 |
| | | 27 | 207 | 22 | | | |
| 5 | 222-238 | 47 | 222 | 19 | 0.235 | 0.160 | 1.474 |
| | | 14 | 224 | 24 | | | |
| | | 69 | 227 | 17 | | | |
| | | 57 | 230 | 18 | | | |
| 6 | 241-273 | 70 | 241 | 17 | 0.212 | 0.160 | 1.328 |
| | | 15 | 248 | 24 | | | |
| | | 71 | 255 | 17 | | | |
| | | 30 | 258 | 21 | | | |
| | | 39 | 259 | 20 | | | |
| | | 58 | 261 | 18 | | | |
| | | 40 | 265 | 20 | | | |
| 7 | 290-342 | 72 | 290 | 17 | 0.208 | 0.160 | 1.300 |
| | | 48 | 293 | 19 | | | |
| | | 31 | 298 | 21 | | | |
| | | 73 | 301 | 17 | | | |
| | | 18 | 305 | 23 | | | |
| | | 6 | 308 | 27 | | | |
| | | 10 | 312 | 25 | | | |
| | | 19 | 316 | 23 | | | |
| | | 28 | 319 | 22 | | | |
| | | 41 | 326 | 20 | | | |
| | | 74 | 334 | 17 | | | |
| 8 | 343-363 | 59 | 343 | 18 | 0.238 | 0.160 | 1.491 |
| | | 60 | 348 | 18 | | | |
| | | 75 | 351 | 17 | | | |
| | | 20 | 353 | 23 | | | |
| | | 76 | 355 | 17 | | | |
| 9 | 364-447 | 49 | 364 | 19 | 0.250 | 0.160 | 1.566 |
| | | 32 | 371 | 21 | | | |
| | | 11 | 372 | 25 | | | |
| | | 61 | 375 | 18 | | | |
| | | 77 | 382 | 17 | | | |
| | | 21 | 390 | 23 | | | |
| | | 78 | 391 | 17 | | | |
| | | 1 | 394 | 30 | | | |
| | | 42 | 397 | 20 | | | |
| | | 62 | 403 | 18 | | | |
| | | 33 | 410 | 21 | | | |
| | | 43 | 418 | 20 | | | |
| | | 34 | 419 | 21 | | | |
| | | 7 | 422 | 27 | | | |
| | | 2 | 425 | 29 | | | |
| | | 79 | 426 | 17 | | | |
| | | 63 | 428 | 18 | | | |
| | | 64 | 431 | 18 | | | |
| | | 12 | 432 | 25 | | | |
| | | 16 | 435 | 24 | | | |
| | | 80 | 439 | 17 | | | |
| 10 | 455-474 | 29 | 455 | 22 | 0.200 | 0.160 | 1.253 |
| | | 17 | 459 | 24 | | | |
| | | 4 | 462 | 28 | | | |
| | | 3 | 466 | 29 | | | |

Total AAs: 509
Total 9-mers: 501
SYFPEITHI 17: 80 9-mers

TABLE 57
Predication of clusters for CEA (NIH algorithm)

| Cluster | | Peptides | Start | | Peptides/AAs | | |
|---|---|---|---|---|---|---|---|
| # | AA | Rank | Position | Score | Cluster | Whole Pr. | Ratio |
| 1 | 17-32 | 5 | 17 | 79.041 | 0.188 | 0.043 | 4.388 |
| | | 7 | 18 | 46.873 | | | |
| | | 20 | 24 | 12.668 | | | |
| 2 | 113-129 | 2 | 113 | 167.991 | 0.118 | 0.043 | 2.753 |
| | | 15 | 121 | 21.362 | | | |
| 3 | 172-187 | 25 | 172 | 9.165 | 0.125 | 0.043 | 2.925 |
| | | 14 | 179 | 27.995 | | | |
| 4 | 278-219 | 30 | 278 | 5.818 | 0.143 | 0.043 | 3.343 |
| | | 17 | 283 | 19.301 | | | |
| 5 | 350-365 | 9 | 350 | 43.075 | 0.125 | 0.043 | 2.925 |
| | | 12 | 357 | 27.995 | | | |
| 6 | 528-543 | 8 | 528 | 43.075 | 0.125 | 0.043 | 2.925 |
| | | 13 | 535 | 27.995 | | | |
| 7 | 631-645 | 23 | 631 | 9.563 | 0.200 | 0.043 | 4.680 |
| | | 19 | 634 | 13.381 | | | |
| | | 24 | 637 | 9.245 | | | |
| 8 | 691-702 | 1 | 691 | 196.407 | 0.167 | 0.043 | 3.900 |
| | | 27 | 694 | 7.769 | | | |

Total AAs: 702
Total 9-mers: 694
NIH 5: 30 9-mers

TABLE 58
Predication of clusters for CEA (SYFPEITHI algorithm)

| | | | | | Peptides/AAs | | |
|---|---|---|---|---|---|---|---|
| Cluster | | Peptides | Start | | | Whole | |
| # | AA | Rank | Position | Score | Cluster | Pr. | Ratio |
| 1 | 5-36 | 67 | 5 | 16 | 0.250 | 0.117 | 2.140 |
| | | 23 | 12 | 19 | | | |
| | | 24 | 16 | 19 | | | |
| | | 9 | 17 | 22 | | | |
| | | 25 | 18 | 19 | | | |
| | | 32 | 19 | 18 | | | |
| | | 68 | 23 | 16 | | | |
| | | 33 | 28 | 18 | | | |
| 2 | 37-62 | 41 | 37 | 17 | 0.269 | 0.117 | 2.305 |
| | | 20 | 44 | 20 | | | |
| | | 26 | 45 | 19 | | | |
| | | 42 | 46 | 17 | | | |
| | | 27 | 50 | 19 | | | |
| | | 43 | 53 | 17 | | | |
| | | 44 | 54 | 17 | | | |
| 3 | 99-115 | 14 | 99 | 21 | 0.235 | 0.117 | 2.014 |
| | | 5 | 100 | 23 | | | |
| | | 45 | 104 | 17 | | | |
| | | 34 | 107 | 18 | | | |
| 4 | 116-129 | 69 | 116 | 16 | 0.143 | 0.117 | 1.223 |
| | | 21 | 121 | 20 | | | |
| 5 | 172-187 | 46 | 172 | 17 | 0.125 | 0.117 | 1.070 |
| | | 70 | 179 | 16 | | | |
| 6 | 192-202 | 3 | 192 | 24 | 0.182 | 0.117 | 1.557 |
| | | 47 | 194 | 17 | | | |
| 7 | 226-241 | 48 | 226 | 17 | 0.188 | 0.117 | 1.605 |
| | | 49 | 229 | 17 | | | |
| | | 15 | 233 | 21 | | | |
| 8 | 307-318 | 11 | 307 | 22 | 0.250 | 0.117 | 2.140 |
| | | 71 | 308 | 16 | | | |
| | | 51 | 310 | 17 | | | |
| 9 | 319-349 | 52 | 319 | 17 | 0.129 | 0.117 | 1.105 |
| | | 53 | 327 | 17 | | | |
| | | 72 | 335 | 16 | | | |
| | | 35 | 341 | 18 | | | |
| 10 | 370-388 | 12 | 370 | 22 | 0.211 | 0.117 | 1.802 |
| | | 54 | 372 | 17 | | | |
| | | 74 | 375 | 16 | | | |
| | | 6 | 380 | 23 | | | |
| 11 | 403-419 | 56 | 403 | 17 | 0.235 | 0.117 | 2.014 |
| | | 57 | 404 | 17 | | | |
| | | 58 | 407 | 17 | | | |
| | | 28 | 411 | 19 | | | |
| 12 | 427-442 | 59 | 427 | 17 | 0.188 | 0.117 | 1.605 |
| | | 75 | 432 | 16 | | | |
| | | 76 | 434 | 16 | | | |
| 13 | 450-462 | 77 | 450 | 16 | 0.154 | 0.117 | 1.317 |
| | | 13 | 454 | 22 | | | |
| 14 | 488-505 | 36 | 488 | 18 | 0.167 | 0.117 | 1.427 |
| | | 18 | 492 | 21 | | | |
| | | 60 | 497 | 17 | | | |
| 15 | 548-558 | 4 | 548 | 24 | 0.182 | 0.117 | 1.557 |
| | | 61 | 550 | 17 | | | |
| 16 | 565-577 | 62 | 565 | 17 | 0.154 | 0.117 | 1.317 |
| | | 19 | 569 | 21 | | | |
| 17 | 579-597 | 78 | 579 | 16 | 0.143 | 0.117 | 1.223 |
| | | 79 | 582 | 16 | | | |
| | | 7 | 589 | 23 | | | |
| 18 | 605-618 | 2 | 605 | 25 | 0.143 | 0.117 | 1.223 |
| | | 38 | 610 | 18 | | | |
| 19 | 631-669 | 29 | 631 | 19 | 0.154 | 0.117 | 1.317 |
| | | 63 | 637 | 17 | | | |
| | | 80 | 644 | 16 | | | |
| | | 64 | 652 | 17 | | | |
| | | 39 | 660 | 18 | | | |
| | | 81 | 661 | 16 | | | |
| 20 | 675-702 | 22 | 675 | 20 | 0.286 | 0.117 | 2.446 |
| | | 30 | 683 | 19 | | | |
| | | 31 | 687 | 19 | | | |
| | | 40 | 688 | 18 | | | |
| | | 65 | 690 | 17 | | | |
| | | 1 | 691 | 31 | | | |
| | | 66 | 692 | 17 | | | |
| | | 8 | 694 | 23 | | | |

Total AAs: 702
Total 9-mers: 694
SYFPEITHI 16: 81 9-mers

TABLE 59
Predication of clusters for SCP-1 (NIH algorithm)

| | | Pep- | Start | | | Peptides/ | |
|---|---|---|---|---|---|---|---|
| Cluster | | tides | Posi- | | | AAs | |
| # | AA | Rank | tion | Score | Cluster | Whole Pr. | Ratio |
| 1 | 101-116 | 15 | 101 | 40.589 | 0.125 | 0.038 | 3.270 |
| | | 13 | 108 | 57.255 | | | |
| 2* | 281-305 | 14 | 281 | 44.944 | 0.12 | 0.038 | 3.139 |
| | | 24 | 288 | 15.203 | | | |
| | | 17 | 297 | 32.857 | | | |
| 3 | 431-447 | 8 | 431 | 80.217 | 0.073 | 0.038 | 1.914 |
| | | 26 | 438 | 11.861 | | | |
| | | 4 | 439 | 148.896 | | | |
| 4 | 557-579 | 11 | 557 | 64.335 | 0.174 | 0.038 | 4.550 |
| | | 19 | 560 | 24.937 | | | |
| | | 6 | 564 | 87.586 | | | |
| | | 18 | 571 | 32.765 | | | |
| 5 | 635-650 | 10 | 635 | 69.552 | 0.125 | 0.038 | 3.270 |
| | | 34 | 642 | 6.542 | | | |
| 6 | 755-767 | 36 | 755 | 5.599 | 0.154 | 0.038 | 4.025 |
| | | 35 | 759 | 5.928 | | | |
| 7 | 838-854 | 2 | 838 | 284.517 | 0.118 | 0.038 | 3.078 |
| | | 28 | 846 | 11.426 | | | |

Total AAs: 976
Total 9-mers: 968
NIH 5: 37 9-mers

TABLE 60
Predication of clusters for SCP-1

| | | Pep- | Start | | | Peptides/ | |
|---|---|---|---|---|---|---|---|
| Cluster | | tides | Posi- | | | AAs | |
| # | AA | Rank | tion | Score | Cluster | Whole Pr. | Ratio |
| 1 | 8-28 | 99 | 8 | 16 | 0.143 | 0.121 | 1.182 |
| | | 77 | 15 | 17 | | | |
| | | 100 | 20 | 16 | | | |
| 2 | 63-80 | 78 | 63 | 17 | 0.222 | 0.121 | 1.838 |
| | | 50 | 66 | 19 | | | |
| | | 102 | 69 | 16 | | | |
| | | 60 | 72 | 18 | | | |
| 3 | 94-123 | 79 | 94 | 17 | 0.133 | 0.121 | 1.103 |
| | | 12 | 101 | 23 | | | |
| | | 17 | 108 | 22 | | | |
| | | 103 | 115 | 16 | | | |
| 4 | 126-158 | 35 | 126 | 20 | 0.182 | 0.121 | 1.504 |
| | | 36 | 133 | 20 | | | |
| | | 51 | 139 | 19 | | | |
| | | 80 | 140 | 17 | | | |
| | | 61 | 143 | 18 | | | |
| | | 37 | 150 | 20 | | | |
| 5 | 161-189 | 38 | 161 | 20 | 0.207 | 0.121 | 1.711 |
| | | 52 | 165 | 19 | | | |
| | | 81 | 171 | 17 | | | |
| | | 82 | 177 | 17 | | | |
| | | 62 | 178 | 18 | | | |
| | | 39 | 181 | 20 | | | |
| 6 | 213-230 | 40 | 213 | 20 | 0.167 | 0.121 | 1.379 |
| | | 13 | 220 | 23 | | | |
| | | 28 | 222 | 21 | | | |
| 7 | 235-250 | 63 | 235 | 18 | 0.125 | 0.121 | 1.034 |
| | | 18 | 242 | 22 | | | |
| 8 | 260-296 | 83 | 260 | 17 | 0.243 | 0.121 | 2.012 |
| | | 105 | 262 | 16 | | | |
| | | 84 | 267 | 17 | | | |
| | | 106 | 269 | 16 | | | |
| | | 41 | 270 | 20 | | | |
| | | 64 | 271 | 18 | | | |
| | | 85 | 274 | 17 | | | |
| | | 19 | 281 | 22 | | | |
| | | 3 | 288 | 25 | | | |
| 9 | 312-338 | 108 | 312 | 16 | 0.148 | 0.121 | 1.225 |
| | | 29 | 319 | 21 | | | |
| | | 30 | 323 | 21 | | | |
| | | 65 | 330 | 18 | | | |
| 10 | 339-355 | 66 | 339 | 18 | 0.235 | 0.121 | 1.946 |
| | | 31 | 340 | 21 | | | |
| | | 42 | 344 | 20 | | | |
| | | 53 | 347 | 19 | | | |
| 11 | 376-447 | 54 | 376 | 19 | 0.194 | 0.121 | 1.608 |
| | | 43 | 382 | 20 | | | |
| | | 44 | 386 | 20 | | | |
| | | 20 | 390 | 22 | | | |
| | | 55 | 397 | 19 | | | |
| | | 6 | 404 | 24 | | | |
| | | 86 | 407 | 17 | | | |
| | | 45 | 411 | 20 | | | |
| | | 67 | 417 | 18 | | | |
| | | 21 | 425 | 22 | | | |
| | | 46 | 431 | 20 | | | |
| | | 68 | 432 | 18 | | | |
| | | 32 | 438 | 21 | | | |
| | | 7 | 439 | 24 | | | |
| 12 | 455-488 | 33 | 455 | 21 | 0.235 | 0.121 | 1.946 |
| | | 47 | 459 | 20 | | | |
| | | 56 | 462 | 19 | | | |
| | | 87 | 463 | 17 | | | |
| | | 88 | 466 | 17 | | | |
| | | 14 | 470 | 23 | | | |
| | | 109 | 473 | 16 | | | |
| | | 34 | 480 | 21 | | | |
| 13 | 515-530 | 57 | 515 | 19 | 0.125 | 0.121 | 1.034 |
| | | 22 | 522 | 22 | | | |
| 14 | 557-590 | 8 | 557 | 24 | 0.147 | 0.121 | 1.216 |
| | | 23 | 564 | 22 | | | |
| | | 9 | 571 | 24 | | | |
| | | 90 | 575 | 17 | | | |
| | | 58 | 582 | 19 | | | |
| 15 | 610-625 | 69 | 610 | 18 | 0.125 | 0.121 | 1.034 |
| | | 91 | 617 | 17 | | | |
| 16 | 633-668 | 92 | 633 | 17 | 0.222 | | |
| | | 10 | 635 | 24 | | | |
| | | 70 | 638 | 18 | | | |
| | | 93 | 640 | 17 | | | |
| | | 48 | 642 | 20 | | | |
| | | 49 | 645 | 20 | | | |
| | | 111 | 652 | 16 | | | |
| | | 112 | 660 | 16 | | | |
| 17 | 674-685 | 71 | 674 | 18 | 0.167 | 0.121 | 1.379 |
| | | 11 | 677 | 24 | | | |
| 18 | 687-702 | 1 | 687 | 26 | 0.125 | 0.121 | 1.034 |
| | | 94 | 694 | 17 | | | |
| 19 | 744-767 | 113 | 744 | 16 | 0.250 | 0.121 | 2.068 |
| | | 95 | 745 | 17 | | | |
| | | 4 | 745 | 25 | | | |
| | | 24 | 752 | 22 | | | |
| | | 2 | 755 | 26 | | | |
| | | 72 | 759 | 18 | | | |
| 20 | 812-827 | 97 | 812 | 17 | 0.125 | 0.121 | 1.034 |
| | | 115 | 819 | 16 | | | |
| 21 | 838-857 | 116 | 838 | 16 | 0.150 | 0.121 | 1.241 |
| | | 25 | 846 | 22 | | | |
| | | 74 | 849 | 18 | | | |
| 22 | 896-913 | 117 | 896 | 16 | 0.222 | 0.121 | 1.838 |
| | | 98 | 899 | 17 | | | |
| | | 26 | 902 | 22 | | | |
| | | 76 | 905 | 18 | | | |

Total AAs: 976
Total 9-mers: 968
Rammensee 16: 118 9-mers

The embodiments of the invention are applicable to and contemplate variations in the sequences of the target antigens provided herein, including those disclosed in the various databases that are accessible by the world wide web. Specifically for the specific sequences disclosed herein, variation in sequences can be found by using the provided accession numbers to access information for each antigen.

```
TYROSINASE PROTEIN; SEQ ID NO 2
                1 MLLAVLYCLL WSFQTSAGHF PRACVSSKNL MEKECCPPWS GDRSPCGQLS
        GRGS CQNILL
              61 SNAPLGPQFP FTGVDDRESW PSVFYNRTCQ CSGNFMGFNC GNCKFGFWGP
NCTERRLLVR
            121 RNIFDLSAPE KDKFFAYLTL AKHTISSDYV IPIGTYGQMK NGSTPMFNDI
NIYDLFVWMH
            181 YYVSMDALLG GSEIWRDIDF AHEAPAFLPW HRLFLLRWEQ EIQKLTGDEN
FTIPYWDWRD
            241 AEKCDICTDE YMGGQHPTNP NLLSPASFFS SWQIVCSRLE EYNSHQSLCN
GTPEGPLRRN
            301 PGNHDKSRTP RLPSSADVEF CLSLTQYESG SMDKAANFSF RNTLEGFASP
LTGIADASQS
            361 SMHNALHIYM NGTMSQVQGS ANDPIFLLHH AFVDSIFEQW LRRHRPLQEV
YPEANAPIGH
            421 NRESYMVPFI PLYRNGDFFI SSKDLGYDYS YLQDSDPDSF QDYIKSYLEQ
ASRIWSWLLG
            481 AAMVGAVLTA LLAGLVSLLC RHKRKQLPEE KQPLLMEKED YHSLYQSHL
SSX-2 PROTEIN; SEQ ID NO 3
                      1 MNGDDAFARR PTVGAQIPEK IQKAFDDIAK YFSKEEWEKM
KASEKIFYVY MKRKYEAMTK
                61 LGFKATLPPF MCNKRAEDFQ GNDLDNDPNR GNQVERPQMT FGRLQGISPK
IMPKKPAEEG
            121 NDSEEVPEAS GPQNDGKELC PPGKPTTSEK IHERSGPKRG EHAWTHRLRE
RKQLVIYEEI
            181 SDPEEDDE
PSMA PROTEIN; SEQ ID NO 4
                    1 MWNLLHETDS AVATARRPRW LCAGALVLAG GFFLLGFLFG
WFIKSSNEAT NITPKHNMKA
                61 FLDELKAENI KKFLYNFTQI PHLAGTEQNF QLAKQIQSQW KEFGLDSVEL
AHYDVLLSYP
            121 NKTHPNYISI INEDGNEIFN TSLFEPPPPG YENVSDIVPP FSAFSPQGMP
EGDLVYVNYA
            181 RTEDFFKLER DMKINCSGKI VIARYGKVFR GNKVKNAQLA GAKGVILYSD
PADYFAPGVK
            241 SYPDGWNLPG GGVQRGNILN LNGAGDPLTP GYPANEYAYR RGIAEAVGLP
SIPVHPIGYY
            301 DAQKLLEKMG GSAPPDSSWR GSLKVPYNVG PGFTGNFSTQ KVKMHIHSTN
EVTRIYNVIG
            361 TLRGAVEPDR YVILGGHRDS WVFGGIDPQS GAAVVHEIVR SFGTLKKEGW
RPRRTILFAS
            421 WDAEEFGLLG STEWAEENSR LLQERGVAYI NADSSIEGNY TLRVDCTPLM
YSLVHNLTKE
            481 LKSPDEGFEG KSLYESWTKK SPSPEFSGMP RISKLGSGND FEVFFQRLGI
ASGRARYTKN
            541 WETNKFSGYP LYHSVYETYE LVEKFYDPMF KYHLTVAQVR GGMVFELANS
IVLPFDCRDY
            601 AVVLRKYADK IYSISMKHPQ EMKTYSVSFD SLFSAVKNFT EIASKFSERL
QDFDKSNPIV
            661 LRMMNDQLMF LERAFIDPLG LPDRPFYRHV IYAPSSHNKY AGESFPGIYD
ALFDIESKVD
            721 PSKAWGEVKR QIYVAAFTVQ AAAETLSEVA
Homo sapiens tyrosinase (oculocutaneous albinism IA) (TYR),
mRNA.;
ACCESSION NM_000372
VERSION NM_000372.1 GI:4507752
SEQ ID NO 2
/translation = “MLLAVLYCLLWSFQTSAGHFPRACVSSKNLMEKECCPPWSGDRS
PCGQLSGRGSCQNILLSNAPLGPQFPFTGVDDRESWPSVFYNRTCQCSGNFMGFNCGN
CKFGFWGPNCTERRLLVRRNIFDLSAPEKDKFFAYLTLAKHTISSDYVIPIGTYGQMK
NGSTPMFNDINIYDLFVWMHYYVSMDALLGGSEIWRDIDFAHEAPAFLPWHRLFLLRW
EQEIQKLTGDENFTIPYWDWRDAEKCDICTDEYMGGQHPTNPNLLSPASFFSSWQIVC
SRLEEYNSHQSLCNGTPEGPLRRNPGNHDKSRTPRLPSSADVEFCLSLTQYESGSMDK
AANFSFRNTLEGFASPLTGIADASQSSMHNALHIYMNGTMSQVQGSANDPIFLLHHAF
VDSIFEQWLRRHRPLQEVYPEANAPIGHNRESYMVPFIPLYRNGDFFISSKDLGYDYS
YLQDSDPDSFQDYIKSYLEQASRIWSWLLGAAMVGAVLTALLAGLVSLLCRHKRKQLP
                                          EEKQPLLMEKEDYHSLYQSHL”
SEQ ID NO 5
ORIGIN
                1 atcactgtag tagtagctgg aaagagaaat ctgtgactcc aattagccag
ttcctgcaga
              61 ccttgtgagg actagaggaa gaatgctcct ggctgttttg tactgcctgc
tgtggagttt
            121 ccagacctcc gctggccatt tccctagagc ctgtgtctcc tctaagaacc
tgatggagaa
            181 ggaatgctgt ccaccgtgga gcggggacag gagtccctgt ggccagcttt
caggcagagg
            241 ttcctgtcag aatatccttc tgtccaatgc accacttggg cctcaatttc
ccttcacagg
            301 ggtggatgac cgggagtcgt ggccttccgt cttttataat aggacctgcc
agtgctctgg
            361 caacttcatg ggattcaact gtggaaactg caagtttggc ttttggggac
caaactgcac
            421 agagagacga ctcttggtga gaagaaacat cttcgatttg agtgccccag
agaaggacaa
            481 attttttgcc tacctcactt tagcaaagca taccatcagc tcagactatg
tcatccccat
            541 agggacctat ggccaaatga aaaatggatc aacacccatg tttaacgaca
tcaatattta
            601 tgacctcttt gtctggatgc attattatgt gtcaatggat gcactgcttg
ggggatctga
            661 aatctggaga gacattgatt ttgcccatga agcaccagct tttctgcctt
ggcatagact
            721 cttcttgttg cggtgggaac aagaaatcca gaagctgaca ggagatgaaa
acttcactat
            781 tccatattgg gactggcggg atgcagaaaa gtgtgacatt tgcacagatg
agtacatggg
            841 aggtcagcac cccacaaatc ctaacttact cagcccagca tcattcttct
cctcttggca
            901 gattgtctgt agccgattgg aggagtacaa cagccatcag tctttatgca
atggaacgcc
            961 cgagggacct ttacggcgta atcctggaaa ccatgacaaa tccagaaccc
caaggctccc
          1021 ctcttcagct gatgtagaat tttgcctgag tttgacccaa tatgaatctg
gttccatgga
          1081 taaagctgcc aatttcagct ttagaaatac actggaagga tttgctagtc
cacttactgg
          1141 gatagcggat gcctctcaaa gcagcatgca caatgccttg cacatctata
tgaatggaac
          1201 aatgtcccag gtacagggat ctgccaacga tcctatcttc cttcttcacc
atgcatttgt
          1261 tgacagtatt tttgagcagt ggctccgaag gcaccgtcct cttcaagaag
tttatccaga
          1321 agccaatgca cccattggac ataaccggga atcctacatg gttcctttta
taccactgta
          1381 cagaaatggt gatttcttta tttcatccaa agatctgggc tatgactata
gctatctaca
          1441 agattcagac ccagactctt ttcaagacta cattaagtcc tatttggaac
aagcgagtcg
          1501 gatctggtca tggctccttg gggcggcgat ggtaggggcc gtcctcactg
ccctgctggc
          1561 agggcttgtg agcttgctgt gtcgtcacaa gagaaagcag cttcctgaag
aaaagcagcc
          1621 actcctcatg gagaaagagg attaccacag cttgtatcag agccatttat
aaaaggctta
          1681 ggcaatagag tagggccaaa aagcctgacc tcactctaac tcaaagtaat
gtccaggttc
          1741 ccagagaata tctgctggta tttttctgta aagaccattt gcaaaattgt
aacctaatac
          1801 aaagtgtagc cttcttccaa ctcaggtaga acacacctgt ctttgtcttg
ctgttttcac
          1861 tcagcccttt taacattttc ccctaagccc atatgtctaa ggaaaggatg
ctatttggta
          1921 atgaggaact gttatttgta tgtgaattaa agtgctctta tttt
Homo sapiens synovial sarcoma, X breakpoint 2 (SSX2), mRNA.
ACCESSION NM_003147
VERSION NM_003147.1 GI:10337582
SEQ ID NO 3
/translation = “MNGDDAFARRPTVGAQIPEKIQKAFDDIAKYFSKEEWEKMKASE
KIFYVYMKRKYEAMTKLGFKATLPPFMCNKRAEDFQGNDLDNDPNRGNQVERPQMTFG
RLQGISPKIMPKKPAEEGNDSEEVPEASGPQNDGKELCPPGKPTTSEKIHERSGPKRG
                                          EHAWTHRLRERKQLVIYEEISDPEEDDE”
SEQ ID NO 6
ORIGIN
                1 ctctctttcg attcttccat actcagagta cgcacggtct gattttctct
ttggattctt
              61 ccaaaatcag agtcagactg ctcccggtgc catgaacgga gacgacgcct
ttgcaaggag
            121 acccacggtt ggtgctcaaa taccagagaa gatccaaaag gccttcgatg
atattgccaa
            181 atacttctct aaggaagagt gggaaaagat gaaagcctcg gagaaaatct
tctatgtgta
            241 tatgaagaga aagtatgagg ctatgactaa actaggtttc aaggccaccc
tcccaccttt
            301 catgtgtaat aaacgggccg aagacttcca ggggaatgat ttggataatg
accctaaccg
            361 tgggaatcag gttgaacgtc ctcagatgac tttcggcagg ctccagggaa
tctccccgaa
            421 gatcatgccc aagaagccag cagaggaagg aaatgattcg gaggaagtgc
cagaagcatc
            481 tggcccacaa aatgatggga aagagctgtg ccccccggga aaaccaacta
cctctgagaa
            541 gattcacgag agatctggac ccaaaagggg ggaacatgcc tggacccaca
gactgcgtga
            601 gagaaaacag ctggtgattt atgaagagat cagcgaccct gaggaagatg
acgagtaact
            661 cccctcaggg atacgacaca tgcccatgat gagaagcaga acgtggtgac
ctttcacgaa
            721 catgggcatg gctgcggacc cctcgtcatc aggtgcatag caagtg
Homo sapiens folate hydrolase (prostate-specific membrane
antigen)
                        1 (FOLH1), mRNA.
ACCESSION NM_004476
VERSION NM_004476.1 GI:4758397
/translation = “MWNLLHETDSAVATARRPRWLCAGALVLAGGFFLLGFLFGWFIK
SSNEATNITPKHNMKAFLDELKAENIKKFLYNFTQIPHLAGTEQNFQLAKQIQSQWKE
FGLDSVELAHYDVLLSYPNKTHPNYISIINEDGNEIFNTSLFEPPPPGYENVSDTVPP
FSAFSPQGMPEGDLVYVNYARTEDFFKLERDMKINCSGKIVIARYGKVFRGNKVKNAQ
LAGAKGVILYSDPADYFAPGVKSYPDGWNLPGGGVQRGNILNLNGAGDPLTPGYPANE
YAYRRGIAEAVGLPSIPVHPIGYYDAQKLLEKMGGSAPPDSSWRGSLKVPYNVGPGFT
GNFSTQKVKMHIHSTNEVTRIYNVIGTLRGAVEPDRYVILGGHRDSWVFGGIDPQSGA
AVVHEIVRSFGTLKKEGWRPRRTILFASWDAEEFGLLGSTEWAEENSRLLQERGVAYI
NADSSIEGNYTLRVDCTPLMYSLVHNLTKELKSPDEGFEGKSLYESWTKKSPSPEFSG
MPRISKLGSGNDFEVFFQRLGIASGRARYTKNWETNKFSGYPLYHSVYETYELVEKFY
DPMFKYHLTVAQVRGGMVFELANSIVLPFDCRDYAVVLRKYADKIYSISMKHPQEMKT
YSVSFDSLFSAVKNFTEIASKFSERLQDFDKSNPIVLRMMNDQLMFLERAFIDPLGLP
DRPFYRHVIYAPSSHNKYAGESFPGIYDALFDIESKVDPSKAWGEVKRQIYVAAFTVQ
                                          AAAETLSEVA”
SEQ ID NO 7
ORIGIN
                1 ctcaaaaggg gccggatttc cttctcctgg aggcagatgt tgcctctctc
tctcgctcgg
              61 attggttcag tgcactctag aaacactgct gtggtggaga aactggaccc
caggtctgga
            121 gcgaattcca gcctgcaggg ctgataagcg aggcattagt gagattgaga
gagactttac
            181 cccgccgtgg tggttggagg gcgcgcagta gagcagcagc acaggcgcgg
gtcccgggag
            241 gccggctctg ctcgcgccga gatgtggaat ctccttcacg aaaccgactc
ggctgtggcc
            301 accgcgcgcc gcccgcgctg gctgtgcgct ggggcgctgg tgctggcggg
tggcttcttt
            361 ctcctcggct tcctcttcgg gtggtttata aaatcctcca atgaagctac
taacattact
            421 ccaaagcata atatgaaagc atttttggat gaattgaaag ctgagaacat
caagaagttc
            481 ttatataatt ttacacagat accacattta gcaggaacag aacaaaactt
tcagcttgca
            541 aagcaaattc aatcccagtg gaaagaattt ggcctggatt ctgttgagct
agcacattat
            601 gatgtcctgt tgtcctaccc aaataagact catcccaact acatctcaat
aattaatgaa
            661 gatggaaatg agattttcaa cacatcatta tttgaaccac ctcctccagg
atatgaaaat
            721 gtttcggata ttgtaccacc tttcagtgct ttctctcctc aaggaatgcc
agagggcgat
            781 ctagtgtatg ttaactatgc acgaactgaa gacttcttta aattggaacg
ggacatgaaa
            841 atcaattgct ctgggaaaat tgtaattgcc agatatggga aagttttcag
aggaaataag
            901 gttaaaaatg cccagctggc aggggccaaa ggagtcattc tctactccga
ccctgctgac
            961 tactttgctc ctggggtgaa gtcctatcca gatggttgga atcttcctgg
aggtggtgtc
          1021 cagcgtggaa atatcctaaa tctgaatggt gcaggagacc ctctcacacc
aggttaccca
          1081 gcaaatgaat atgcttatag gcgtggaatt gcagaggctg ttggtcttcc
aagtattcct
          1141 gttcatccaa ttggatacta tgatgcacag aagctcctag aaaaaatggg
tggctcagca
          1201 ccaccagata gcagctggag aggaagtctc aaagtgccct acaatgttgg
acctggcttt
          1261 actggaaact tttctacaca aaaagtcaag atgcacatcc actctaccaa
tgaagtgaca
          1321 agaatttaca atgtgatagg tactctcaga ggagcagtgg aaccagacag
atatgtcatt
          1381 ctgggaggtc accgggactc atgggtgttt ggtggtattg accctcagag
tggagcagct
          1441 gttgttcatg aaattgtgag gagctttgga acactgaaaa aggaagggtg
gagacctaga
          1501 agaacaattt tgtttgcaag ctgggatgca gaagaatttg gtcttcttgg
ttctactgag
          1561 tgggcagagg agaattcaag actccttcaa gagcgtggcg tggcttatat
taatgctgac
          1621 tcatctatag aaggaaacta cactctgaga gttgattgta caccgctgat
gtacagcttg
          1681 gtacacaacc taacaaaaga gctgaaaagc cctgatgaag gctttgaagg
caaatctctt
          1741 tatgaaagtt ggactaaaaa aagtccttcc ccagagttca gtggcatgcc
caggataagc
          1801 aaattgggat ctggaaatga ttttgaggtg ttcttccaac gacttggaat
tgcttcaggc
          1861 agagcacggt atactaaaaa ttgggaaaca aacaaattca gcggctatcc
actgtatcac
          1921 agtgtctatg aaacatatga gttggtggaa aagttttatg atccaatgtt
taaatatcac
          1981 ctcactgtgg cccaggttcg aggagggatg gtgtttgagc tagccaattc
catagtgctc
          2041 ccttttgatt gtcgagatta tgctgtagtt ttaagaaagt atgctgacaa
aatctacagt
          2101 atttctatga aacatccaca ggaaatgaag acatacagtg tatcatttga
ttcacttttt
          2161 tctgcagtaa agaattttac agaaattgct tccaagttca gtgagagact
ccaggacttt
          2221 gacaaaagca acccaatagt attaagaatg atgaatgatc aactcatgtt
tctggaaaga
          2281 gcatttattg atccattagg gttaccagac aggccttttt ataggcatgt
catctatgct
          2341 ccaagcagcc acaacaagta tgcaggggag tcattcccag gaatttatga
tgctctgttt
          2401 gatattgaaa gcaaagtgga cccttccaag gcctggggag aagtgaagag
acagatttat
          2461 gttgcagcct tcacagtgca ggcagctgca gagactttga gtgaagtagc
ctaagaggat
          2521 tctttagaga atccgtattg aatttgtgtg gtatgtcact cagaaagaat
cgtaatgggt
          2581 atattgataa attttaaaat tggtatattt gaaataaagt tgaatattat
atataaaaaa
          2641 aaaaaaaaaa aaa
Human melanocyte-specific (pmel 17) gene, exons 2-5, and complete
cds.
ACCESSION U20093
VERSION U20093.1 GI:1142634
SEQ ID NO 70
/translation = “MDLVLKRCLLHLAVIGALLAVGATKVPRNQDWLGVSRQLRTKWNRQLYPE
WTEAQRLDCWRGGQVSLKVSNDGPTLIGANASFSIALNFPGSQKVLPDGQVIWVNNTIINGSQVW
GGQPVYPQETDDACIFPDGGPCPSGSWSQKRSFVYVWKTWGQYWQVLGGPVSGLSIGTGRAMLGT
HTMEVTVYHRRGSRSYVPLAHSSSAFTITDQVPFSVSVSQLRALDGGNKHFLRNQPLTFALQLHD
PSGYLAEADLSYTWDFGDSSGTLISRAPVVTHTYLEPGPVTAQVVLQAAIPLTSCGSSPVPGTTD
GHRPTAEAPNTTAGQVPTTEVVGTTPGQAPTAEPSGTTSVQVPTTEVISTAPVQMPTAESTGMTP
EKVPVSEVMGTTLAEMSTPEATGMTPAEVSIVVLSGTTAAQVTTTEWVETTARELPIPEPEGPDA
SSIMSTESITGSLGPLLDGTATLRLVKRQVPLDCVLYRYGSFSVTLDIVQGIESAEILQAVPSGE
GDAFELTVSCQGGLPKEACMEISSPGCQPPAQRLCQPVLPSPACQLVLHQILKGGSGTYCLNVSL
ADTNSLAVVSTQLIMPGQEAGLGQVPLIVGILLVLMAVVLASLIYRRRLMKQDFSVPQLPHSSSH
WLRLPRIFCSCPIGENSPLLSGQQV”
SEQ ID NO 80
ORIGIN
                1 gtgctaaaaa gatgccttct tcatttggct gtgataggtg ctttgtggct
gtgggggcta
              61 caaaagtacc cagaaaccag gactggcttg gtgtctcaag gcaactcaga
accaaagcct
            121 ggaacaggca gctgtatcca gagtggacag aagcccagag acttgactgc
tggagaggtg
            181 gtcaagtgtc cctcaaggtc agtaatgatg ggcctacact gattggtgca
aatgcctcct
            241 tctctattgc cttgaacttc cctggaagcc aaaaggtatt gccagatggg
caggttatct
            301 gggtcaacaa taccatcatc aatgggagcc aggtgtgggg aggacagcca
gtgtatcccc
            361 aggaaactga cgatgcctgc atcttccctg atggtggacc ttgcccatct
ggctcttggt
            421 ctcagaagag aagctttgtt tatgtctgga agacctgggg tgagggactc
ccttctcagc
            481 ctatcatcca cacttgtgtt tacttctttc tacctgatca cctttctttt
ggccgcccct
            541 tccaccttaa cttctgtgat tttctctaat cttcattttc ctcttagatc
ttttctcttt
            601 cttagcacct agcccccttc aagctctatc ataattcttt ctggcaactc
ttggcctcaa
            661 ttgtagtcct accccatgga atgcctcatt aggacccctt ccctgtcccc
ccatatcaca
            721 gccttccaaa caccctcaga agtaatcata cttcctgacc tcccatctcc
agtgccgttt
            781 cgaagcctgt ccctcagtcc cctttgacca gtaatctctt cttccttgct
tttcattcca
            841 aaaatgcttc aggccaatac tggcaagttc tagggggccc agtgtctggg
ctgagcattg
            901 ggacaggcag ggcaatgctg ggcacacaca ccatggaagt gactgtctac
catcgccggg
            961 gatcccggag ctatgtgcct cttgctcatt ccagctcagc cttcaccatt
actggtaagg
          1021 gttcaggaag ggcaaggcca gttgtagggc aaagagaagg cagggaggct
tggatggact
          1081 gcaaaggaga aaggtgaaat gctgtgcaaa cttaaagtag aagggccagg
aagacctagg
          1141 cagagaaatg tgaggcttag tgccagtgaa gggccagcca gtcagcttgg
agttggaggg
          1201 tgtggctgtg aaaggagaag ctgtggctca ggcctggttc tcaccttttc
tggctccaat
          1261 cccagaccag gtgcctttct ccgtgagcgt gtcccagttg cgggccttgg
atggagggaa
          1321 caagcacttc ctgagaaatc agcctctgac ctttgccctc cagctccatg
accccagtgg
          1381 ctatctggct gaagctgacc tctcctacac ctgggacttt ggagacagta
gtggaaccct
          1441 gatctctcgg gcacctgtgg tcactcatac ttacctggag cctggcccag
tcactgccca
          1501 ggtggtcctg caggctgcca ttcctctcac ctcctgtggc tcctccccag
ttccaggcac
          1561 cacagatggg cacaggccaa ctgcagaggc ccctaacacc acagctggcc
aagtgcctac
          1621 tacagaagtt gtgggtacta cacctggtca ggcgccaact gcagagccct
ctggaaccac
          1681 atctgtgcag gtgccaacca ctgaagtcat aagcactgca cctgtgcaga
tgccaactgc
          1741 agagagcaca ggtatgacac ctgagaaggt gccagtttca gaggtcatgg
gtaccacact
          1801 ggcagagatg tcaactccag aggctacagg tatgacacct gcagaggtat
caattgtggt
          1861 gctttctgga accacagctg cacaggtaac aactacagag tgggtggaga
ccacagctag
          1921 agagctacct atccctgagc ctgaaggtcc agatgccagc tcaatcatgt
ctacggaaag
          1981 tattacaggt tccctgggcc ccctgctgga tggtacagcc accttaaggc
tggtgaagag
          2041 acaagtcccc ctggattgtg ttctgtatcg atatggttcc ttttccgtca
ccctggacat
          2101 tgtccagggt attgaaagtg ccgagatcct gcaggctgtg ccgtccggtg
agggggatgc
          2161 atttgagctg actgtgtcct gccaaggcgg gctgcccaag gaagcctgca
tggagatctc
          2221 atcgccaggg tgccagcccc ctgcccagcg gctgtgccag cctgtgctac
ccagcccagc
          2281 ctgccagctg gttctgcacc agatactgaa gggtggctcg gggacatact
gcctcaatgt
          2341 gtctctggct gataccaaca gcctggcagt ggtcagcacc cagcttatca
tgcctggtag
          2401 gtccttggac agagactaag tgaggaggga agtggataga ggggacagct
ggcaagcagc
          2461 agacatgagt gaagcagtgc ctgggattct tctcacaggt caagaagcag
gccttgggca
          2521 ggttccgctg atcgtgggca tcttgctggt gttgatggct gtggtccttg
catctctgat
          2581 atataggcgc agacttatga agcaagactt ctccgtaccc cagttgccac
atagcagcag
          2641 tcactggctg cgtctacccc gcatcttctg ctcttgtccc attggtgaga
atagccccct
          2701 cctcagtggg cagcaggtct gagtactctc atatgatgct gtgattttcc
tggagttgac
          2761 agaaacacct atatttcccc cagtcttccc tgggagacta ctattaactg
aaataaa
//
Homo sapiens kallikrein 3, (prostate specific antigen) (KLK3),
mRNA.
ACCESSION NM_001648
VERSTON NM_001648.1 GI:4502172
SEQ ID NO 78
/translation = “MWVPVVFLTLSVTWIGAAPLILSRIVGGWECEKHSQPWQVLVAS
RGRAVCGGVLVHPQWVLTAAHCIRNKSVILLGRHSLFHPEDTGQVFQVSHSFPHPLYDMSLLKNR
FLRPGDDSSHDLMLLRLSEPAELTDAVKVMDLPTQEPALGTTCYASGWGSIEPEEFLTPKKLQCV
DLHVISNDVCAQVHPQKVTKFMLCAGRWTGGKSTCSGDSGGPLVCNGVLQGITSWGSEPCALPER
PSLYTKVVHYRKWIKDTIVANP”
SEQ ID NO 86
ORIGIN
                1 agccccaagc ttaccacctg cacccggaga gctgtgtgtc accatgtggg
tcccggttgt
              61 cttcctcacc ctgtccgtga cgtggattgg tgctgcaccc ctcatcctgt
ctcggattgt
            121 gggaggctgg gagtgcgaga agcattccca accctggcag gtgcttgtgg
cctctcgtgg
            181 cagggcagtc tgcggcggtg ttctggtgca cccccagtgg gtcctcacag
ctgcccactg
            241 catcaggaac aaaagcgtga tcttgctggg tcggcacagc ctgtttcatc
ctgaagacac
            301 aggccaggta tttcaggtca gccacagctt cccacacccg ctctacgata
tgagcctcct
            361 gaagaatcga ttcctcaggc caggtgatga ctccagccac gacctcatgc
tgctccgcct
            421 gtcagagcct gccgagctca cggatgctgt gaaggtcatg gacctgccca
cccaggagcc
            481 agcactgggg accacctgct acgcctcagg ctggggcagc attgaaccag
aggagttctt
            541 gaccccaaag aaacttcagt gtgtggacct ccatgttatt tccaatgacg
tgtgtgcgca
            601 agttcaccct cagaaggtga ccaagttcat gctgtgtgct ggacgctgga
cagggggcaa
            661 aagcacctgc tcgggtgatt ctgggggccc acttgtctgt aatggtgtgc
ttcaaggtat
            721 cacgtcatgg ggcagtgaac catgtgccct gcccgaaagg ccttccctgt
acaccaaggt
            781 ggtgcattac cggaagtgga tcaaggacac catcgtggcc aacccctgag
cacccctatc
            841 aaccccctat tgtagtaaac ttggaacctt ggaaatgacc aggccaagac
tcaagcctcc
            901 ccagttctac tgacctttgt ccttaggtgt gaggtccagg gttgctagga
aaagaaatca
            961 gcagacacag gtgtagacca gagtgtttct taaatggtgt aattttgtcc
tctctgtgtc
          1021 ctggggaata ctggccatgc ctggagacat atcactcaat ttctctgagg
acacagatag
          1081 gatggggtgt ctgtgttatt tgtggggtac agagatgaaa gaggggtggg
atccacactg
          1141 agagagtgga gagtgacatg tgctggacac tgtccatgaa gcactgagca
gaagctggag
          1201 gcacaacgca ccagacactc acagcaagga tggagctgaa aacataaccc
actctgtcct
          1261 ggaggcactg ggaagcctag agaaggctgt gagccaagga gggagggtct
tcctttggca
            1321 tgggatgggg atgaagtaag gagagggact ggaccccctg gaagctgatt
cactatgggg
            1381 ggaggtgtat tgaagtcctc cagacaaccc tcagatttga tgatttccta
gtagaactca
            1441 cagaaataaa gagctgttat actgtg
//
Human autoimmunogenic cancer/testis antigen NY-ESO-1 mRNA,
complete cds.
ACCESSION U87459
VERSION U87459.1 GI:1890098
SEQ ID NO 74
                      /translation=“MQAEGRGTGGSTGDADGPGGPGIPDGPGGNAGGPGEA
                      GATGGRGPRGAGAARASGPGGGAPRGPHGGAASGLNGCCRCGARGPESRLL
                      EFYLAMPFATPMEAELARRSLAQDAPPLPVPGVLLKEFTVSGNILTIRLTA
                      ADHRQLQLSISSCLQQLSLLMWITQCFLPVFLAQPPSGQRR”
SEQ ID NO 84
ORIGIN
                1 atcctcgtgg gccctgacct tctctctgag agccgggcag aggctccgga
gccatgcagg
              61 ccgaaggccg gggcacaggg ggttcgacgg gcgatgctga tggcccagga
ggccctggca
            121 ttcctgatgg cccagggggc aatgctggcg gcccaggaga ggcgggtgcc
acgggcggca
            181 gaggtccccg gggcgcaggg gcagcaaggg cctcggggcc gggaggaggc
gccccgcggg
            241 gtccgcatgg cggcgcggct tcagggctga atggatgctg cagatgcggg
gccagggggc
            301 cggagagccg cctgcttgag ttctacctcg ccatgccttt cgcgacaccc
atggaagcag
            361 agctggcccg caggagcctg gcccaggatg ccccaccgct tcccgtgcca
ggggtgcttc
            421 tgaaggagtt cactgtgtcc ggcaacatac tgactatccg actgactgct
gcagaccacc
            481 gccaactgca gctctccatc agctcctgtc tccagcagct ttccctgttg
atgtggatca
            541 cgcagtgctt tctgcccgtg tttttggctc agcctccctc agggcagagg
cgctaagccc
            601 agcctggcgc cccttcctag gtcatgcctc ctcccctagg gaatggtccc
agcacgagtg
            661 gccagttcat tgtgggggcc tgattgtttg tcgctggagg aggacggctt
acatgtttgt
            721 ttctgtagaa aataaaactg agctacgaaa aa
//
LAGE-1a protein [Homo sapiens].
ACCESSION CAA11116
PID g3255959
VERSION CAA11116.1 GI:3255959
SEQ ID NO 75
ORIGIN
                1 mqaegrgtgg stgdadgpgg pgipdgpggn aggpgeagat ggrgprgaga
arasgprgga
              61 prgphggaas aqdgrcpcga rrpdsrllel hitmpfsspm eaelvrrils
rdaaplprpg
            121 avlkdftvsg nllfirltaa dhrqlqlsis sclqqlsllm witqcflpvf
laqapsgqrr
            181
//
LACE-1b protein [Homo sapiens].
ACCESSION CAA11117
PID g3255960
VERSION CAA11117.1 GI:3255960
SEQ ID NO 76
ORIGIN
                1 mqaegrgtgg stgdadgpgg pgipdgpggn aggpgeagat ggrgprgaga
arasgprgga
              61 prgphggaas aqdgrcpcga rrpdsrllel hitmpfsspm eaelvrrils
rdaaplprpg
            121 avlkciftvsg nllfmsvwdq dregagrmrv vgwglgsasp egqkardlrt
pkhkvseqrp
            181 gtpgppppeg aqgdgcrgva fnvmfsaphi
//
Human antigen (MACE-1) gene, complete cds.
ACCESSION M77481
VERSION M77481.1 GI:416114
SEQ ID NO 71
                      /translation=“MSLEQRSLHCKPEEALEAQQEALGLVCVQAATSSSSP
                      LVLGTLEEVPTAGSTDPPQSPQGASAFPTTINFTRQRQPSEGSSSREEEGP
                      STSCILESLFRAVITKKVADLVGFLLLKYRAREPVTKAEMLESVIKNYKHC
                      FPEIFGKASESLQLVFGIDVKEADPTGHSYVLVTCLGLSYDGLLGDNQIMP
                      KTGFLIIVLVMIAMEGGHAPEEEIWEELSVMEVYDGREHSAYGEPRKLLTQ
                      DLVQEKYLEYRQVPDSDPARYEFLWGPRALAETSYVKVLEYVIKVSARVRF
                      FFPSLREAALREEEEGV”
SEQ ID NO 81
ORIGIN
                1 ggatccaggc cctgccagga aaaatataag ggccctgcgt gagaacagag
ggggtcatcc
              61 actgcatgag agtggggatg tcacagagtc cagcccaccc tcctggtagc
actgagaagc
            121 cagggctgtg cttgcggtct gcaccctgag ggcccgtgga ttcctcttcc
tggagctcca
            181 ggaaccaggc agtgaggcct tggtctgaga cagtatcctc aggtcacaga
gcagaggatg
            241 cacagggtgt gccagcagtg aatgtttgcc ctgaatgcac accaagggcc
ccacctgcca
            301 caggacacat aggactccac agagtctggc ctcacctccc tactgtcagt
cctgtagaat
            361 cgacctctgc tggccggctg taccctgagt accctctcac ttcctccttc
aggttttcag
            421 gggacaggcc aacccagagg acaggattcc ctggaggcca cagaggagca
ccaaggagaa
            481 gatctgtaag taggcctttg ttagagtctc caaggttcag ttctcagctg
aggcctctca
            541 cacactccct ctctccccag gcctgtgggt cttcattgcc cagctcctgc
ccacactcct
            601 gcctgctgcc ctgacgagag tcatcatgtc tcttgagcag aggagtctgc
actgcaagcc
            661 tgaggaagcc cttgaggccc aacaagaggc cctgggcctg gtgtgtgtgc
aggctgccac
            721 ctcctcctcc tctcctctgg tcctgggcac cctggaggag gtgcccactg
ctgggtcaac
            781 agatcctccc cagagtcctc agggagcctc cgcctttccc actaccatca
acttcactcg
            841 acagaggcaa cccagtgagg gttccagcag ccgtgaagag gaggggccaa
gcacctcttg
            901 tatcctggag tccttgttcc gagcagtaat cactaagaag gtggctgatt
tggttggttt
            961 tctgctcctc aaatatcgag ccagggagcc agtcacaaag gcagaaatgc
tggagagtgt
          1021 catcaaaaat tacaagcact gttttcctga gatcttcggc aaagcctctg
agtccttgca
          1081 gctggtcttt ggcattgacg tgaaggaagc agaccccacc ggccactcct
atgtccttgt
          1141 cacctgccta ggtctctcct atgatggcct gctgggtgat aatcagatca
tgcccaagac
          1201 aggcttcctg ataattgtcc tggtcatgat tgcaatggag ggcggccatg
ctcctgagga
          1261 ggaaatctgg gaggagctga gtgtgatgga ggtgtatgat gggagggagc
acagtgccta
          1321 tggggagccc aggaagctgc tcacccaaga tttggtgcag gaaaagtacc
tggagtaccg
          1381 gcaggtgccg gacagtgatc ccgcacgcta tgagttcctg tggggtccaa
gggccctcgc
          1441 tgaaaccagc tatgtgaaag tccttgagta tgtgatcaag gtcagtgcaa
gagttcgctt
          1501 tttcttccca tccctgcgtg aagcagcttt gagagaggag gaagagggag
tctgagcatg
          1561 agttgcagcc aaggccagtg ggagggggac tgggccagtg caccttccag
ggccgcgtcc
          1621 agcagcttcc cctgcctcgt gtgacatgag gcccattctt cactctgaag
agagcggtca
          1681 gtgttctcag tagtaggttt ctgttctatt gggtgacttg gagatttatc
tttgttctct
          1741 tttggaattg ttcaaatgtt tttttttaag ggatggttga atgaacttca
gcatccaagt
          1801 ttatgaatga cagcagtcac acagttctgt gtatatagtt taagggtaag
agtcttgtgt
          1861 tttattcaga ttgggaaatc cattctattt tgtgaattgg gataataaca
gcagtggaat
          1921 aagtacttag aaatgtgaaa aatgagcagt aaaatagatg agataaagaa
ctaaagaaat
          1981 taagagatag tcaattcttg ccttatacct cagtctattc tgtaaaattt
ttaaagatat
          2041 atgcatacct ggatttcctt ggcttctttg agaatgtaag agaaattaaa
tctgaataaa
          2101 gaattcttcc tgttcactgg ctcttttctt ctccatgcac tgagcatctg
ctttttggaa
          2161 ggccctgggt tagtagtgga gatgctaagg taagccagac tcatacccac
ccatagggtc
          2221 gtagagtcta ggagctgcag tcacgtaatc gaggtggcaa gatgtcctct
aaagatgtag
          2281 ggaaaagtga gagaggggtg agggtgtggg gctccgggtg agagtggtgg
agtgtcaatg
          2341 ccctgagctg gggcattttg ggctttggga aactgcagtt ccttctgggg
gagctgattg
          2401 taatgatctt gggtggatcc
//
Human MAGE-2 gene exons 1-4, complete cds.
ACCESSION L18920
VERSION L18920.1 GI:436180
SEQ ID NO 72
/translation = “MPLEQRSQHCKPEEGLEARGEALGLVGAQAPATEEQQTASSSSTLVEV
TLGEVPAADSPSPPHSPQGASSFSTTINYTLWRQSDEGSSNQEEEGPRMFPDLE
SEFQAAISRKMVELVHFLLLKYRAREPVTKAEMLESVLRNCQDFFPVIFSKASEYLQLVFGI
EVVEVVPISHLYILVTCLGLSYDGLLGDNQVMPKTGLLIIVLAIIAIEGDCAPEEKIWEELS
MLEVFEGREDSVFAHPRKLLMQDLVQENYLEYRQVPGSDPACYEFLWGPRALIETSYVKVLH
HTLKIGGEPHISYPPLHERALREGEE”
SEQ ID NO 82
ORIGIN
                1 attccttcat caaacagcca ggagtgagga agaggaccct cctgagtgag
gactgaggat
              61 ccaccctcac cacatagtgg gaccacagaa tccagctcag cccctcttgt
cagccctggt
            121 acacactggc aatgatctca ccccgagcac acccctcccc ccaatgccac
ttcgggccga
            181 ctcagagtca gagacttggt ctgaggggag cagacacaat cggcagagga
tggcggtcca
            241 ggctcagtct ggcatccaag tcaggacctt gagggatgac caaaggcccc
tcccaccccc
            301 aactcccccg acaccaccag gatctacagc ctcaggatcc ccgtcccaat
ccctacccct
            361 acaccaacac catcttcatg cttaccccca cccccccatc cagatcccca
tccgggcaga
            421 atccggttcc acccttgccg tgaacccagg gaagtcacgg gcccggatgt
gacgccactg
            481 acttgcacat tggaggtcag aggacagcga gattctcgcc ctgagcaacg
gcctgacgtc
            541 ggcggaggga agcaggcgca ggctccgtga ggaggcaagg taagacgccg
agggaggact
            601 gaggcgggcc tcaccccaga cagagggccc ccaataatcc agcgctgcct
ctgctgccgg
            661 gcctggacca ccctgcaggg gaagacttct caggctcagt cgccaccacc
tcaccccgcc
            721 accccccgcc gctttaaccg cagggaactc tggcgtaaga gctttgtgtg
accagggcag
            781 ggctggttag aagtgctcag ggcccagact cagccaggaa tcaaggtcag
gaccccaaga
            841 ggggactgag ggcaacccac cccctaccct cactaccaat cccatccccc
aacaccaacc
            901 ccacccccat ccctcaaaca ccaaccccac ccccaaaccc cattcccatc
tcctccccca
            961 ccaccatcct ggcagaatcc ggctttgccc ctgcaatcaa cccacggaag
ctccgggaat
          1021 ggcggccaag cacgcggatc ctgacgttca catgtacggc taagggaggg
aaggggttgg
          1081 gtctcgtgag tatggccttt gggatgcaga ggaagggccc aggcctcctg
gaagacagtg
          1141 gagtccttag gggacccagc atgccaggac agggggccca ctgtacccct
gtctcaaact
          1201 gagccacctt ttcattcagc cgagggaatc ctagggatgc agacccactt
cagcaggggg
          1261 ttggggccca gcctgcgagg agtcaagggg aggaagaaga gggaggactg
aggggacctt
          1321 ggagtccaga tcagtggcaa ccttgggctg ggggatcctg ggcacagtgg
ccgaatgtgc
          1381 cccgtgctca ttgcaccttc agggtgacag agagttgagg gctgtggtct
gagggctggg
          1441 acttcaggtc agcagaggga ggaatcccag gatctgccgg acccaaggtg
tgcccccttc
          1501 atgaggactg gggatacccc cggcccagaa agaagggatg ccacagagtc
tggaagtccc
          1561 ttgttcttag ctctggggga acctgatcag ggatggccct aagtgacaat
ctcatttgta
          1621 ccacaggcag gaggttgggg aaccctcagg gagataaggt gttggtgtaa
agaggagctg
          1681 tctgctcatt tcagggggtt gggggttgag aaagggcagt ccctggcagg
agtaaagatg
          1741 agtaacccac aggaggccat cataacgttc accctagaac caaaggggtc
agccctggac
          1801 aacgcacgtg ggggtaacag gatgtggccc ctcctcactt gtctttccag
atctcaggga
          1861 gttgatgacc ttgttttcag aaggtgactc aggtcaacac aggggcccca
tctggtcgac
          1921 agatgcagtg gttctaggat ctgccaagca tccaggtgga gagcctgagg
taggattgag
          1981 ggtacccctg ggccagaatg cagcaagggg gccccataga aatctgccct
gcccctgcgg
          2041 ttacttcaga gaccctgggc agggctgtca gctgaagtcc ctccattatc
ctgggatctt
          2101 tgatgtcagg gaaggggagg ccttggtctg aaggggctgg agtcaggtca
gtagagggag
          2161 ggtctcaggc cctgccagga gtggacgtga ggaccaagcg gactcgtcac
ccaggacaca
          2221 tggactccaa tgaatttgga catctctcgt tgtccttcgc gggaggacct
ggtcacgtat
          2281 ggccagatgt gggtcccctc atatccttct gtaccatatc agggatgtga
gttcttgaca
          2341 tgagagattc tcaagccagc aaaagggtgg gattaggccc tacaaggaga
aaggtgaggg
          2401 ccctgagtga gcacagaggg gaccctccac ccaagtagag tggggacctc
acggagtctg
          2461 gccaaccctg ctgagacttc tgggaatccg tggctgtgct tgcagtctgc
acactgaagg
          2521 cccgtgcatt cctctcccag gaatcaggag ctccaggaac caggcagtga
ggccttggtc
          2581 tgagtcagtg tcctcaggtc acagagcaga ggggacgcag acagtgccaa
cactgaaggt
          2641 ttgcctggaa tgcacaccaa gggccccacc cgcccagaac aaatgggact
ccagagggcc
          2701 tggcctcacc ctccctattc tcagtcctgc agcctgagca tgtgctggcc
ggctgtaccc
          2761 tgaggtgccc tcccacttcc tccttcaggt tctgaggggg acaggctgac
aagtaggacc
          2821 cgaggcactg gaggagcatt gaaggagaag atctgtaagt aagcctttgt
cagagcctcc
          2881 aaggttcagt tcagttctca cctaaggcct cacacacgct ccttctctcc
ccaggcctgt
          2941 gggtcttcat tgcccagctc ctgcccgcac tcctgcctgc tgccctgacc
agagtcatca
          3001 tgcctcttga gcagaggagt cagcactgca agcctgaaga aggccttgag
gcccgaggag
          3061 aggccctggg cctggtgggt gcgcaggctc ctgctactga ggagcagcag
accgcttctt
          3121 cctcttctac tctagtggaa gttaccctgg gggaggtgcc tgctgccgac
tcaccgagtc
          3181 ctccccacag tcctcaggga gcctccagct tctcgactac catcaactac
actctttgga
          3241 gacaatccga tgagggctcc agcaaccaag aagaggaggg gccaagaatg
tttcccgacc
          3301 tggagtccga gttccaagca gcaatcagta ggaagatggt tgagttggtt
cattttctgc
          3361 tcctcaagta tcgagccagg gagccggtca caaaggcaga aatgctggag
agtgtcctca
          3421 gaaattgcca ggacttcttt cccgtgatct tcagcaaagc ctccgagtac
ttgcagctgg
          3481 tctttggcat cgaggtggtg gaagtggtcc ccatcagcca cttgtacatc
cttgtcacct
          3541 gcctgggcct ctcctacgat ggcctgctgg gcgacaatca ggtcatgccc
aagacaggcc
          3601 tcctgataat cgtcctggcc ataatcgcaa tagagggcga ctgtgcccct
gaggagaaaa
          3661 tctgggagga gctgagtatg ttggaggtgt ttgaggggag ggaggacagt
gtcttcgcac
          3721 atcccaggaa gctgctcatg caagatctgg tgcaggaaaa ctacctggag
taccggcagg
          3781 tgcccggcag tgatcctgca tgctacgagt tcctgtgggg tccaagggcc
ctcattgaaa
          3841 ccagctatgt gaaagtcctg caccatacac taaagatcgg tggagaacct
cacatttcct
          3901 acccacccct gcatgaacgg gctttgagag agggagaaga gtgagtctca
gcacatgttg
          3961 cagccagggc cagtgggagg gggtctgggc cagtgcacct tccagggccc
catccattag
          4021 cttccactgc ctcgtgtgat atgaggccca ttcctgcctc tttgaagaga
gcagtcagca
          4081 ttcttagcag tgagtttctg ttctgttgga tgactttgag atttatcttt
ctttcctgtt
          4141 ggaattgttc aaatgttcct tttaacaaat ggttggatga acttcagcat
ccaagtttat
          4201 gaatgacagt agtcacacat agtgctgttt atatagttta ggggtaagag
tcctgttttt
          4261 tattcagatt gggaaatcca ttccattttg tgagttgtca cataataaca
gcagtggaat
          4321 atgtatttgc ctatattgtg aacgaattag cagtaaaata catgatacaa
ggaactcaaa
          4381 agatagttaa ttcttgcctt atacctcagt ctattatgta aaattaaaaa
tatgtgtatg
          4441 tttttgcttc tttgagaatg caaaagaaat taaatctgaa taaattcttc
ctgttcactg
          4501 gctcatttct ttaccattca ctcagcatct gctctgtgga aggccctggt
agtagtggg
//
Human MAGE-3 antigen (MAGE-3) gene, complete cds.
ACCESSION U03735
VERSION U03735.1 GI:468825
SEQ ID NO 73
/translation = “MPLEQRSQHCKPEEGLEARGEALGLVGAQAPATEEQEAASSSSTLVEVTLG
EVPAAESPDPPQSPQGASSLPTTMNYPLWSQSYEDSSNQEEEGPSTFPDLESEFQAALSRKVAEL
VHFLLLKYRAREPVTKAEMLGSVVGNWQYFFPVIFSKASSSLQLVFGIELMEVDPIGHLYIFATC
LGLSYDGLLGDNQIMPKAGLLIIVLAIIAREGDCAPEEKIWEELSVLEVFEGREDSILGDPKKLL
TQHFVQENYLEYRQVPGSDPACYEFLWGPRALVETSYVKVLHHMVKISGGPHISYPPLHEWVLRE
GEE”
SEQ ID NO 83
ORIGIN
                1 acgcaggcag tgatgtcacc cagaccacac cccttccccc aatgccactt
cagggggtac
              61 tcagagtcag agacttggtc tgaggggagc agaagcaatc tgcagaggat
ggcggtccag
            121 gctcagccag gcatcaactt caggaccctg agggatgacc gaaggccccg
cccacccacc
            181 cccaactccc ccgaccccac caggatctac agcctcagga cccccgtccc
aatccttacc
            241 ccttgcccca tcaccatctt catgcttacc tccaccccca tccgatcccc
atccaggcag
            301 aatccagttc cacccctgcc cggaacccag ggtagtaccg ttgccaggat
gtgacgccac
            361 tgacttgcgc attggaggtc agaagaccgc gagattctcg ccctgagcaa
cgagcgacgg
            421 cctgacgtcg gcggagggaa gccggcccag gctcggtgag gaggcaaggt
aagacgctga
            481 gggaggactg aggcgggcct cacctcagac agagggcctc aaataatcca
gtgctgcctc
            541 tgctgccggg cctgggccac cccgcagggg aagacttcca ggctgggtcg
ccactacctc
            601 accccgccga cccccgccgc tttagccacg gggaactctg gggacagagc
ttaatgtggc
            661 cagggcaggg ctggttagaa gaggtcaggg cccacgctgt ggcaggaatc
aaggtcagga
            721 ccccgagagg gaactgaggg cagcctaacc accaccctca ccaccattcc
cgtcccccaa
            781 cacccaaccc cacccccatc ccccattccc atccccaccc ccaccectat
cctggcagaa
            841 tccgggcttt gcccctggta tcaagtcacg gaagctccgg gaatggcggc
caggcacgtg
            901 agtcctgagg ttcacatcta cggctaaggg agggaagggg ttcggtatcg
cgagtatggc
            961 cgttgggagg cagcgaaagg gcccaggcct cctggaagac agtggagtcc
tgaggggacc
          1021 cagcatgcca ggacaggggg cccactgtac ccctgtctca aaccgaggca
ccttttcatt
          1081 cggctacggg aatcctaggg atgcagaccc acttcagcag ggggttgggg
cccagccctg
          1141 cgaggagtca tggggaggaa gaagagggag gactgagggg accttggagt
ccagatcagt
          1201 ggcaaccttg ggctggggga tgctgggcac agtggccaaa tgtgctctgt
gctcattgcg
          1261 ccttcagggt gaccagagag ttgagggctg tggtctgaag agtgggactt
caggtcagca
          1321 gagggaggaa tcccaggatc tgcagggccc aaggtgtacc cccaaggggc
ccctatgtgg
          1381 tggacagatg cagtggtcct aggatctgcc aagcatccag gtgaagagac
tgagggagga
          1441 ttgagggtac ccctgggaca gaatgcggac tgggggcccc ataaaaatct
gccctgctcc
          1501 tgctgttacc tcagagagcc tgggcagggc tgtcagctga ggtccctcca
ttatcctagg
          1561 atcactgatg tcagggaagg ggaagccttg gtctgagggg gctgcactca
gggcagtaga
          1621 gggaggctct cagaccctac taggagtgga ggtgaggacc aagcagtctc
ctcacccagg
          1681 gtacatggac ttcaataaat ttggacatct ctcgttgtcc tttccgggag
gacctgggaa
          1741 tgtatggcca gatgtgggtc ccctcatgtt tttctgtacc atatcaggta
tgtgagttct
          1801 tgacatgaga gattctcagg ccagcagaag ggagggatta ggccctataa
ggagaaaggt
          1861 gagggccctg agtgagcaca gaggggatcc tccaccccag tagagtgggg
acctcacaga
          1921 gtctggccaa ccctcctgac agttctggga atccgtggct gcgtttgctg
tctgcacatt
          1981 gggggcccgt ggattcctct cccaggaatc aggagctcca ggaacaaggc
agtgaggact
          2041 tggtctgagg cagtgtcctc aggtcacaga gtagaggggg ctcagatagt
gccaacggtg
          2101 aaggtttgcc ttggattcaa accaagggcc ccacctgccc cagaacacat
ggactccaga
          2161 gcgcctggcc tcaccctcaa tactttcagt cctgcagcct cagcatgcgc
tggccggatg
          2221 taccctgagg tgccctctca cttcctcctt caggttctga ggggacaggc
tgacctggag
          2281 gaccagaggc ccccggagga gcactgaagg agaagatctg taagtaagcc
tttgttagag
          2341 cctccaaggt tccattcagt actcagctga ggtctctcac atgctccctc
tctccccagg
          2401 ccagtgggtc tccattgccc agctcctgcc cacactcccg cctgttgccc
tgaccagagt
          2461 catcatgcct cttgagcaga ggagtcagca ctgcaagcct gaagaaggcc
ttgaggcccg
          2521 aggagaggcc ctgggcctgg tgggtgcgca ggctcctgct actgaggagc
aggaggctgc
          2581 ctcctcctct tctactctag ttgaagtcac cctgggggag gtgcctgctg
ccgagtcacc
          2641 agatcctccc cagagtcctc agggagcctc cagcctcccc actaccatga
actaccctct
          2701 ctggagccaa tcctatgagg actccagcaa ccaagaagag gaggggccaa
gcaccttccc
          2761 tgacctggag tccgagttcc aagcagcact cagtaggaag gtggccgagt
tggttcattt
          2821 tctgctcctc aagtatcgag ccagggagcc ggtcacaaag gcagaaatgc
tggggagtgt
          2881 cgtcggaaat tggcagtatt tctttcctgt gatcttcagc aaagcttcca
gttccttgca
          2941 gctggtcttt ggcatcgagc tgatggaagt ggaccccatc ggccacttgt
acatctttgc
          3001 cacctgcctg ggcctctcct acgatggcct gctgggtgac aatcagatca
tgcccaaggc
          3061 aggcctcctg ataatcgtcc tggccataat cgcaagagag ggcgactgtg
cccctgagga
          3121 gaaaatctgg gaggagctga gtgtgttaga ggtgtttgag gggagggaag
acagtatctt
          3181 gggggatccc aagaagctgc tcacccaaca tttcgtgcag gaaaactacc
tggagtaccg
          3241 gcaggtcccc ggcagtgatc ctgcatgtta tgaattcctg tggggtccaa
gggccctcgt
          3301 tgaaaccagc tatgtgaaag tcctgcacca tatggtaaag atcagtggag
gacctcacat
          3361 ttcctaccca cccctgcatg agtgggtttt gagagagggg gaagagtgag
tctgagcacg
          3421 agttgcagcc agggccagtg ggagggggtc tgggccagtg caccttccgg
ggccgcatcc
          3481 cttagtttcc actgcctcct gtgacgtgag gcccattctt cactctttga
agcgagcagt
          3541 cagcattctt agtagtgggt ttctgttctg ttggatgact ttgagattat
tctttgtttc
          3601 ctgttggagt tgttcaaatg ttccttttaa cggatggttg aatgagcgtc
agcatccagg
          3661 tttatgaatg acagtagtca cacatagtgc tgtttatata gtttaggagt
aagagtcttg
          3721 ttttttactc aaattgggaa atccattcca ttttgtgaat tgtgacataa
taatagcagt
          3781 ggtaaaagta tttgcttaaa attgtgagcg aattagcaat aacatacatg
agataactca
          3841 agaaatcaaa agatagttga ttcttgcctt gtacctcaat ctattctgta
aaattaaaca
          3901 aatatgcaaa ccaggatttc cttgacttct ttgagaatgc aagcgaaatt
aaatctgaat
          3961 aaataattct tcctcttcac tggctcgttt cttttccgtt cactcagcat
ctgctctgtg
          4021 ggaggccctg ggttagtagt ggggatgcta aggtaagcca gactcacgcc
tacccatagg
          4081 gctgtagagc ctaggacctg cagtcatata attaaggtgg tgagaagtcc
tgtaagatgt
          4141 agaggaaatg taagagaggg gtgagggtgt ggcgctccgg gtgagagtag
tggagtgtca
          4201 gtgc
//
Homo sapiens prostate stem cell antigen (PSCA) mRNA, complete
cds.
ACCESSION AF043498
VERSION AF043498.1 GI:2909843
SEQ ID NO 79
/translation = “MKAVLLALLMAGLALQPCTALLCYSCKAQVSNEDCLQVENCTQLGEQC
WTARIRAVGLLTVISKGCSLNCVDDSQDYYVGKKNITCCDTDLCNASGAHALQPAAAILALL
PALGLLLWGPGQL”
SEQ ID NO 87
ORIGIN
                1 agggagaggc agtgaccatg aaggctgtgc tgcttgccct gttgatggca
ggcttggccc
              61 tgcagccagg cactgccctg ctgtgctact cctgcaaagc ccaggtgagc
aacgaggact
            121 gcctgcaggt ggagaactgc acccagctgg gggagcagtg ctggaccgcg
cgcatccgcg
            181 cagttggcct cctgaccgtc atcagcaaag gctgcagctt gaactgcgtg
gatgactcac
            241 aggactacta cgtgggcaag aagaacatca cgtgctgtga caccgacttg
tgcaacgcca
            301 gcggggccca tgccctgcag ccggctgccg ccatccttgc gctgctccct
gcactcggcc
            361 tgctgctctg gggacccggc cagctatagg ctctgggggg ccccgctgca
gcccacactg
            421 ggtgtggtgc cccaggcctt tgtgccactc ctcacagaac ctggcccagt
gggagcctgt
            481 cctggttcct gaggcacatc ctaacgcaag tttgaccatg tatgtttgca
ccccttttcc
            541 ccnaaccctg accttcccat gggccttttc caggattccn accnggcaga
tcagttttag
            601 tganacanat ccgcntgcag atggcccctc caaccntttn tgttgntgtt
tccatggccc
            661 agcattttcc acccttaacc ctgtgttcag gcacttnttc ccccaggaag
ccttccctgc
            721 ccaccccatt tatgaattga gccaggtttg gtccgtggtg tcccccgcac
ccagcagggg
            781 acaggcaatc aggagggccc agtaaaggct gagatgaagt ggactgagta
gaactggagg
            841 acaagagttg acgtgagttc ctgggagttt ccagagatgg ggcctggagg
cctggaggaa
            901 ggggccaggc ctcacatttg tggggntccc gaatggcagc ctgagcacag
cgtaggccct
            961 taataaacac ctgttggata agccaaaaaa
//
GLANDULAR KALLIKREIN 1 PRECURSOR (TISSUE KALLIKREIN)
                        (KIDNEY/PANCREAS/SALIVARY GLAND KALLIKREIN).
ACCESSION P06870
PID g125170
VERSION P06870 GI:125170
SEQ ID NO 600
ORIGIN
                1 mwflvlclal slggtgaapp iqsrivggwe ceqhsqpwqa alyhfstfqc
ggilvhrqwv
              61 ltaahcisdn yqlwlgrhnl fddentaqfv hvsesfphpg fnmsllenht
rqadedyshd
            121 lmllrltepa dtitdavkvv elptqepevg stclasgwgs iepenfsfpd
dlqcvdlkil
            181 pndecekahv qkvtdfmlcv ghleggkdtc vgdsggplmc dgvlqgvtsw
gyvpcgtpnk
            241 psvavrvlsy vkwiedtiae ns
//
ELASTASE 2A PRECURSOR.
ACCESSION P08217
PID g119255
VERSION P08217 GI:119255
SEQ ID NO 601
ORIGIN
                1 mirtlllstl vagalscgdp typpyvtrvv ggeearpnsw pwqvslqyss
ngkwyhtcgg
              61 slianswvlt aahcisssrt yrvglgrhnl yvaesgslav svskivvhkd
wnsnqiskgn
            121 diallklanp vsltdkiqla clppagtilp nnypcyvtgw grlqtngavp
dvlqqgrllv
            181 vdyatcsssa wwgssvktsm icaggdgvis scngdsggpl ncqasdgrwq
vhgivsfgsr
            241 lgcnyyhkps vftrvsnyid winsviann
//
pancreatic elastase IIB [Homo sapiens].
ACCESSION NP_056933
PID g7705648
VERSION NP_056933.1 GI:7705648
SEQ ID NO 602
ORIGIN
                1 mirtlllstl vagalscgvs tyapdmsrml ggeearpnsw pwqvslqyss
ngqwyhtcgg
              61 slianswvlt aahcisssri yrvmlgqhnl yvaesgslav svskivvhkd
wnsnqvskgn
            121 diallklanp vsltdkiqla clppagtilp nnypcyvtgw grlqtngalp
ddlkqgrllv
            181 vdyatcsssg wwgstvktnm icaggdgvic tcngdsggpl ncqasdgrwe
vhgigsltsv
            241 lgcnyyykps iftrvsnynd winsviann
//
PRAME Homo sapiens preferentially expressed antigen in melanoma
(PRAME), mRNA.
ACCESSION NM_006115
VERSION NM_006115.1 GI:5174640
SEQ ID NO 77
/translation = “MERRRLWGSIQSRYISMSVWTSPRRLVELAGQSLLKDEALAIAALELLPRE
LFPPLFMAAFDGRHSQTLKAMVQAWPFTCLPLGVLMKGQHLHLETFKAVLDGLDVLLAQEVRPRR
WKLQVLDLRKNSHQDFWTVWSGNRASLYSFPEPEAAQPMTKKRKVDGLSTEAEQPFIPVEVLVDL
FLKEGACDELFSYLIEKVKRKKNVLRLCCKKLKIFAMPMQDIKMILKMVQLDSIEDLEVTCTWKL
PTLAKFSPYLGQMINLRRLLLSHIHASSYISPEKEEQYIAQFTSQFLSLQCLQALYVDSLFFLRG
RLDQLLRHVMNPLETLSITNCRLSEGDVMHLSQSPSVSQLSVLSLSGVMLTDVSPEPLQALLERA
SATLQDLVFDECGITDDQLLALLPSLSHCSQLTTLSFYGNSISISALQSLLQHLIGLSNLTHVLY
PVPLESYEDIHGTLHLERLAYLHARLRELLCELGRPSMVWLSANPCPHCGDRTFYDPEPILCPCF
MPN”
SEQ ID NO 85
ORIGIN
                1 gcttcagggt acagctcccc cgcagccaga agccgggcct gcagcccctc
agcaccgctc
              61 cgggacaccc cacccgcttc ccaggcgtga cctgtcaaca gcaacttcgc
ggtgtggtga
            121 actctctgag gaaaaaccat tttgattatt actctcagac gtgcgtggca
acaagtgact
            181 gagacctaga aatccaagcg ttggaggtcc tgaggccagc ctaagtcgct
tcaaaatgga
            241 acgaaggcgt ttgtggggtt ccattcagag ccgatacatc agcatgagtg
tgtggacaag
            301 cccacggaga cttgtggagc tggcagggca gagcctgctg aaggatgagg
ccctggccat
            361 tgccgccctg gagttgctgc ccagggagct cttcccgcca ctcttcatgg
cagcctttga
            421 cgggagacac agccagaccc tgaaggcaat ggtgcaggcc tggcccttca
cctgcctccc
            481 tctgggagtg ctgatgaagg gacaacatct tcacctggag accttcaaag
ctgtgcttga
            541 tggacttgat gtgctccttg cccaggaggt tcgccccagg aggtggaaac
ttcaagtgct
            601 ggatttacgg aagaactctc atcaggactt ctggactgta tggtctggaa
acagggccag
            661 tctgtactca tttccagagc cagaagcagc tcagcccatg acaaagaagc
gaaaagtaga
            721 tggtttgagc acagaggcag agcagccctt cattccagta gaggtgctcg
tagacctgtt
            781 cctcaaggaa ggtgcctgtg atgaattgtt ctcctacctc attgagaaag
tgaagcgaaa
            841 gaaaaatgta ctacgcctgt gctgtaagaa gctgaagatt tttgcaatgc
ccatgcagga
            901 tatcaagatg atcctgaaaa tggtgcagct ggactctatt gaagatttgg
aagtgacttg
            961 tacctggaag ctacccacct tggcgaaatt ttctccttac ctgggccaga
tgattaatct
          1021 gcgtagactc ctcctctccc acatocatgo atcttcctac atttccccgg
agaaggaaga
          1081 gcagtatatc gcccagttca cctctcagtt cctcagtctg cagtgcctgc
aggctctcta
          1141 tgtggactct ttatttttcc ttagaggccg cctggatcag ttgctcaggc
acgtgatgaa
          1201 ccccttggaa accctctcaa taactaactg ccggctttcg gaaggggatg
tgatgcatct
          1261 gtcccagagt cccagcgtca gtcagctaag tgtcctgagt ctaagtgggg
tcatgctgac
          1321 cgatgtaagt cccgagcccc tccaagctct gctggagaga gcctctgcca
ccctccagga
          1381 cctggtcttt gatgagtgtg ggatcacgga tgatcagctc cttgccctcc
tgccttccct
          1441 gagccactgc tcccagctta caaccttaag cttctacggg aattccatct
ccatatctgc
          1501 cttgcagagt ctcctgcagc acctcatcgg gctgagcaat ctgacccacg
tgctgtatcc
          1561 tgtccccctg gagagttatg aggacatcca tggtaccctc cacctggaga
ggcttgccta
          1621 tctgcatgcc aggctcaggg agttgctgtg tgagttgggg cggcccagca
tggtctggct
          1681 tagtgccaac ccctgtcctc actgtgggga cagaaccttc tatgacccgg
agcccatcct
          1741 gtgcccctgt ttcatgccta actagctggg tgcacatatc aaatgcttca
ttctgcatac
          1801 ttggacacta aagccaggat gtgcatgcat cttgaagcaa caaagcagcc
acagtttcag
          1861 acaaatgttc agtgtgagtg aggaaaacat gttcagtgag gaaaaaacat
tcagacaaat
          1921 gttcagtgag gaaaaaaagg ggaagttggg gataggcaga tgttgacttg
aggagttaat
          1981 gtgatctttg gggagataca tcttatagag ttagaaatag aatctgaatt
tctaaaggga
          2041 gattctggct tgggaagtac atgtaggagt taatccctgt gtagactgtt
gtaaagaaac
          2101 tgttgaaaat aaagagaagc aatgtgaagc aaaaaaaaaa aaaaaaaa
ED-B domain of Fibronectin
Human fibronectin gene ED-B region.
ACCESSION X07717
VERSION X07717.1 GI:31406
SEQ ID NO 590
/translation = “CTFDNLSPGLEYNVSVYTVKDDKESVPISDTIIPEVPQLTDLSF
VDITDSSIGLRWTPLNSSTIIGYRITVVAAGEGIPIFEDFVDSSVGYYTVTGLEPGID
YDISVITLINGGESAPTTLTQQTAVPPPTDLRFTNIGPDTMRVTW”
SEQ ID NO 591
ORIGIN
                1 ctgcactttt gataacctga gtcccggcct ggagtacaat gtcagtgttt
acactgtcaa
              61 ggatgacaag gaaagtgtcc ctatctctga taccatcatc ccaggtaata
gaaaataagc
            121 tgctatcctg agagtgacat tccaataaga gtggggatta gcatcttaat
ccccagatgc
            181 ttaagggtgt caactatatt tgggatttaa ttccgatctc ccagctgcac
tttccaaaac
            241 caagaagtca aagcagcgat ttggacaaaa tgcttgctgt taacactgct
ttactgtctg
            301 tgcttcactg ggatgctgtg tgttgcagcg agtatgtaat ggagtggcag
ccatggcttt
            361 aactctgtat tgtctgctca catggaagta tgactaaaac actgtcacgt
gtctgtactc
            421 agtactgata ggctcaaagt aatatggtaa atgcatccca tcagtacatt
tctgcccgat
            481 tttacaatcc atatcaattt ccaacagctg cctatttcat cttgcagttt
caaatccttc
            541 tttttgaaaa ttggatttta aaaaaaagtt aagtaaaagt cacaccttca
gggttgttct
            601 ttcttgtggc cttgaaagac aacattgcaa aggcctgtcc taaggatagg
cttgtttgtc
            661 cattgggtta taacataatg aaagcattgg acagatcgtg tccccctttg
gactcttcag
            721 tagaatgctt ttactaacgc taattacatg ttttgattat gaatgaacct
aaaatagtgg
            781 caatggcctt aacctaggcc tgtctttcct cagcctgaat gtgcttttga
atggcacatt
            841 tcacaccata cattcataat gcattagcgt tatggccatg atgttgtcat
gagttttgta
            901 tgggagaaaa aaaatcaatt tatcacccat ttattatttt ttccggttgt
tcatgcaagc
            961 ttattttcta ctaaaacagt tttggaatta ttaaaagcat tgctgatact
tacttcagat
          1021 attatgtcta ggctctaaga atggtttcga catcctaaac agccatatga
tttttaggaa
          1081 tctgaacagt tcaaattgta ccctttaagg atgttttcaa aatgtaaaaa
atatatatat
          1141 atatatatat tccctaaaag aatattcctg tttattcttc tagggaagca
aactgttcat
          1201 gatgcttagg aagtcttttc agagaattta aaacagattg catattacca
tcattgcttt
          1261 aacattccac caattttact actagtaacc tgatatacac tgctttattt
tttcctcttt
          1321 ttttccctct attttccttt tgcctccccc tccctttgct ttgtaactca
atagaggtgc
          1381 cccaactcac tgacctaagc tttgttgata taaccgattc aagcatcggc
ctgaggtgga
          1441 ccccgctaaa ctcttccacc attattgggt accgcatcac agtagttgcg
gcaggagaag
          1501 gtatccctat ttttgaagat tttgtggact cctcagtagg atactacaca
gtcacagggc
          1561 tggagccggg cattgactat gatatcagcg ttatcactct cattaatggc
ggcgagagtg
          1621 cccctactac actgacacaa caaacgggtg aattttgaaa acttctgcgt
ttgagacata
          1681 gatggtgttg catgctgcca ccagttactc cggttaaata tggatgtttc
atgggggaag
          1741 tcagcaattg gccaaagatt cagataggtg gaattggggg gataaggaat
caaatgcatc
          1801 tgctaaactg attggagaaa aacacatgca atatcttcag tacactctca
tttaaaccac
          1861 aagtagatat aaagcctaga gaaatacaga tgtctgctct gttaaatata
aaatagcaaa
          1921 tgttcattca atttgaagac ctagaatttt tcttcttaaa taccaaacac
gaataccaaa
          1981 ttgcgtaagt accaattgat aagaatatat caccaaaatg taccatcatg
ctcttccttc
          2041 taccctttga taaactctac catgctcctt ctttgtagct aaaaacccat
caaaatttag
          2101 ggtagagtgg atgggcattg ttttgaggta ggagaaaagt aaacttggga
ccattctagg
          2161 ttttgttgct gtcactaggt aaagaaacac ctctttaacc acagtctggg
gacaagcatg
          2221 caacatttta aaggttctct gctgtgcatg ggaaaagaaa catgctgaga
accaatttgc
          2281 atgaacatgt tcacttgtaa gtagaattca ctgaatggaa ctgtagctct
agatatctca
          2341 catgggggga agtttaggac cctcttgtct ttttgtctgt gtgcatgtat
ttctttgtaa
          2401 agtactgcta tgtttctctt tgctgtgtgg caacttaagc ctcttcggcc
tgggataaaa
          2461 taatctgcag tggtattaat aatgtacata aagtcaacat atttgaaagt
agattaaaat
          2521 cttttttaaa tatatcaatg atggcaaaaa ggttaaaggg ggcctaacag
tactgtgtgt
          2581 agtgttttat ttttaacagt agtacactat aacttaaaat agacttagat
tagactgttt
          2641 gcatgattat gattctgttt cctttatgca tgaaatattg attttacctt
tccagctact
          2701 tcgttagctt taattttaaa atacattaac tgagtcttcc ttcttgttcg
aaaccagctg
          2761 ttcctcctcc cactgacctg cgattcacca acattggtcc agacaccatg
cgtgtcacct
          2821 ggg
//
CEA Homo sapiens carcinoembryonic antigen-related cell adhesion
molecule 5 (CEACAM5), mRNA.
ACCESSION NM_004363
VERSION NM_004363.1 GI:11386170
SEQ ID NO 592
/translation = “MESPSAPPHRWCIPWQRLLLTASLLTFWNPPTTAKLTIESTPFN
VAEGKEVLLLVHNLPQHLFGYSWYKGERVDGNRQIIGYVIGTQQATPGPAYSGREIIY
PNASLLIQNIIQNDTGFYTLHVIKSDLVNEEATGQFRVYPELPKPSISSNNSKPVEDK
DAVAFTCEPETQDATYLWWVNNQSLPVSPRLQLSNGNRTLTLFNVTRNDTASYKCETQ
NPVSARRSDSVILNVLYGPDAPTISPLNTSYRSGENLNLSCHAASNPPAQYSWFVNGT
FQQSTQELFIPNITVNNSGSYTCQAHNSDTGLNRTTVTTITVYAEPPKPFITSNNSNP
VEDEDAVALTCEPEIQNTTYLWWVNNQSLPVSPRLQLSNDNRTLTLLSVTRNDVGPYE
CGIQNELSVDHSDPVILNVLYGPDDPTISPSYTYYRPGVNLSLSCHAASNPPAQYSWL
IDGNIQQHTQELFISNITEKNSGLYTCQANNSASGHSRTTVKTITVSAELPKPSISSN
NSKPVEDKDAVAFTCEPEAQNTTYLWWVNGQSLPVSPRLQLSNGNRTLTLFNVTRNDA
RAYVCGIQNSVSANRSDPVTLDVLYGPDTPIISPPDSSYLSGANLNLSCHSASNPSPQ
YSWRINGIPQQHTQVLFIAKITPNNNGTYACFVSNLATGRNNSIVKSITVSASGTSPG
                                          LSAGATVGIMIGVLVGVALI”
SEQ ID NO 593
ORIGIN
                1 ctcagggcag agggaggaag gacagcagac cagacagtca cagcagcctt
gacaaaacgt
              61 tcctggaact caagctcttc tccacagagg aggacagagc agacagcaga
gaccatggag
            121 tctccctcgg cccctcccca cagatggtgc atcccctggc agaggctcct
gctcacagcc
            181 tcacttctaa ccttctggaa cccgcccacc actgccaagc tcactattga
atccacgccg
            241 ttcaatgtcg cagaggggaa ggaggtgctt ctacttgtcc acaatctgcc
ccagcatctt
            301 tttggctaca gctggtacaa aggtgaaaga gtggatggca accgtcaaat
tataggatat
            361 gtaataggaa ctcaacaagc taccccaggg cccgcataca gtggtcgaga
gataatatac
            421 cccaatgcat ccctgctgat ccagaacatc atccagaatg acacaggatt
ctacacccta
            481 cacgtcataa agtcagatct tgtgaatgaa gaagcaactg gccagttccg
ggtatacccg
            541 gagatgocca agccctccat ctccagcaac aactccaaac ccgtggagga
caaggatgct
            601 gtggccttca cctgtgaacc tgagactcag gacgcaacct acctgtggtg
ggtaaacaat
            661 cagagcctcc cggtcagtcc caggctgcag ctgtccaatg gcaacaggac
cctcactcta
            721 ttcaatgtca caagaaatga cacagcaagc tacaaatgtg aaacccagaa
cccagtgagt
            781 gccaggcgca gtgattcagt catcctgaat gtcctctatg gcccggatgc
ccccaccatt
            841 tcccctctaa acacatctta cagatcaggg gaaaatctga acctctcctg
ccacgcagcc
            901 tctaacccac ctgcacagta ctcttggttt gtcaatggga ctttccagca
atccacccaa
            961 gagctcttta tccccaacat cactgtgaat aatagtggat cctatacgtg
ccaagcccat
          1021 aactcagaca ctggcctcaa taggaccaca gtcacgacga tcacagtcta
tgcagagcca
          1081 cccaaaccct tcatcaccag caacaactcc aaccccgtgg aggatgagga
tgctgtagcc
          1141 ttaacctgtg aacctgagat tcagaacaca acctacctgt ggtgggtaaa
taatcagagc
          1201 ctcccggtca gtcccaggct gcagctgtcc aatgacaaca ggaccctcac
tctactcagt
          1261 gtcacaagga atgatgtagg accctatgag tgtggaatcc agaacgaatt
aagtgttgac
          1321 cacagcgacc cagtcatcct gaatgtcctc tatggcccag acgaccccac
catttccccc
          1381 tcatacacct attaccgtcc aggggtgaac ctcagcctct cctgccatgc
agcctctaac
          1441 ccacctgcac agtattcttg gctgattgat gggaacatcc agcaacacac
acaagagctc
          1501 tttatctcca acatcactga gaagaacagc ggactctata cctgccaggc
caataactca
          1561 gccagtggcc acagcaggac tacagtcaag acaatcacag tctctgcgga
gctgcccaag
          1621 ccctccatct ccagcaacaa ctccaaaccc gtggaggaca aggatgctgt
ggccttcacc
          1681 tgtgaacctg aggctcagaa cacaacctac ctgtggtggg taaatggtca
gagcctccca
          1741 gtcagtccca ggctgcagct gtccaatggc aacaggaccc tcactctatt
caatgtcaca
          1801 agaaatgacg caagagccta tgtatgtgga atccagaact cagtgagtgc
aaaccgcagt
          1861 gacccagtca ccctggatgt cctctatggg ccggacaccc ccatcatttc
ccccccagac
          1921 tcgtcttacc tttcgggagc gaacctcaac ctctcctgcc actcggcctc
taacccatcc
          1981 ccgcagtatt cttggcgtat caatgggata ccgcagcaac acacacaagt
tctctttatc
          2041 gccaaaatca cgccaaataa taacgggacc tatgcctgtt ttgtctctaa
cttggctact
          2101 ggccgcaata attccatagt caagagcatc acagtctctg catctggaac
ttctcctggt
          2161 ctctcagctg gggccactgt cggcatcatg attggagtgc tggttggggt
tgctctgata
          2221 tagcagccct ggtgtagttt cttcatttca ggaagactga cagttgtttt
gcttcttcct
          2281 taaagcattt gcaacagcta cagtctaaaa ttgcttcttt accaaggata
tttacagaaa
          2341 agactctgac cagagatcga gaccatccta gccaacatcg tgaaacccca
tctctactaa
          2401 aaatacaaaa atgagctggg cttggtggcg cgcacctgta gtcccagtta
ctcgggaggc
          2461 tgaggcagga gaatcgcttg aacccgggag gtggagattg cagtgagccc
agatcgcacc
          2521 actgcactcc agtctggcaa cagagcaaga ctccatctca aaaagaaaag
aaaagaagac
          2581 tctgacctgt actcttgaat acaagtttct gataccactg cactgtctga
gaatttccaa
          2641 aactttaatg aactaactga cagcttcatg aaactgtcca ccaagatcaa
gcagagaaaa
          2701 taattaattt catgggacta aatgaactaa tgaggattgc tgattcttta
aatgtcttgt
          2761 ttcccagatt tcaggaaact ttttttcttt taagctatcc actcttacag
caatttgata
          2821 aaatatactt ttgtgaacaa aaattgagac atttacattt tctccctatg
tggtcgctcc
          2881 agacttggga aactattcat gaatatttat attgtatggt aatatagtta
ttgcacaagt
          2941 tcaataaaaa tctgctcttt gtataacaga aaaa
//
Her2/Neu Human tyrosine kinase-type receptor (HER2) mRNA,
complete cds.
ACCESSION M11730
VERSION M11730.1 GI:183986
SEQ ID NO 594
/translation = “MELAALCRWGLLLALLPPGAASTQVCTGTDMKLRLPASPETHLD
MLRHLYQGCQVVQGNLELTYLPTNASLSFLQDIQEVQGYVLIAHNQVRQVPLQRLRIV
RGTQLFEDNYALAVLDNGDPLNNTTPVTGASPGGLRELQLRSLTEILKGGVLIQRNPQ
LCYQDTILWKDIFHKNNQLALTLIDTNRSRACHPCSPMCKGSRCWGESSEDCQSLTRT
VCAGGCARCKGPLPTDCCHEQCAAGCTGPKHSDCLACLHFNHSGICELHCPALVTYNT
DTFESMPNPEGRYTFGASCVTACPYNYLSTDVGSCTLVCPLHNQEVTAEDGTQRCEKC
SKPCARVCYGLGMEHLREVRAVTSANIQEFAGCKKIFGSLAFLPESFDGDPASNTAPL
QPEQLQVFETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRPEDECVGEG
LACHQLCARGHCWGPGPTQCVNCSQFLRGQECVEECRVLQGLPREYVNARHCLPCHPE
CQPQNGSVTCFGPEADQCVACAHYKDPPFCVARCPSGVKPDLSYMPIWKFPDEEGACQ
PCPINCTHSCVDLDDKGCPAEQRASPLTSIVSAVVGILLVVVLGVVFGILIKRRQQKI
RKYTMRRLLQETELVEPLTPSGAMPNQAQMRILKETELRKVKVLGSGAFGTVYKGIWI
PDGENVKIPVAIKVLRENTSPKANKEILDEAYVMAGVGSPYVSRLLGICLTSTVQLVT
QLMPYGCLLDHVRENRGRLGSQDLLNWCMQIAKGMSYLEDVRLVHRDLAARNVLVKSP
NHVKITDFGLARLLDIDETEYHADGGKVPIKWMALESILRRRFTHQSDVWSYGVTVWE
LMTFGAKPYDGIPAREIPDLLEKGERLPQPPICTIDVYMIMVKCWMIDSECRPRFREL
VSEFSRMARDPQRFVVIQNEDLGPASPLDSTFYRSLLEDDDMGDLVDAEEYLVPQQGF
FCPDPAPGAGGMVHHRHRSSSTRSGGGDLTLGLEPSEEEAPRSPLAPSEGAGSDVFDG
DLGMGAAKGLQSLPTHDPSPLQRYSEDPTVPLPSETDGYVAPLTCSPQPEYVNQPDVR
PQPPSPREGPLPAARPAGATLERAKTLSPGKNGVVKDVFAFGGAVENPEYLTPQGGAA
PQPHPPPAFSPAFDNLYYWDQDPPERGAPPSTFKGTPTAENPEYLGLDVPV”
SEQ ID NO 595
ORIGIN Chromosome 17q21-q22.
                1 aattctcgag ctcgtcgacc ggtcgacgag ctcgagggtc gacgagctcg
agggcgcgcg
              61 cccggccccc acccctcgca gcaccccgcg ccccgcgccc tcccagccgg
gtccagccgg
            121 agccatgggg ccggagccgc agtgagcacc atggagctgg cggccttgtg
ccgctggggg
            181 ctcctcctcg ccctcttgcc ccccggagcc gcgagcaccc aagtgtgcac
cggcacagac
            241 atgaagctgc ggctccctgc cagtcccgag acccacctgg acatgctccg
ccacctctac
            301 cagggctgcc aggtggtgca gggaaacctg gaactcacct acctgcccac
caatgccagc
            361 ctgtccttcc tgcaggatat ccaggaggtg cagggctacg tgctcatcgc
tcacaaccaa
            421 gtgaggcagg tcccactgca gaggctgcgg attgtgcgag gcacccagct
ctttgaggac
            481 aactatgccc tggccgtgct agacaatgga gacccgctga acaataccac
ccctgtcaca
            541 ggggcctccc caggaggcct gcgggagctg cagcttcgaa gcctcacaga
gatcttgaaa
            601 ggaggggtat tgatccagcg gaacccccag ctctgctacc aggacacgat
tttgtggaag
            661 gacatcttcc acaagaacaa ccagctggct ctcacactga tagacaccaa
ccgctctcgg
            721 gcctgccacc cctgttctcc gatgtgtaag ggctcccgct gctggggaga
gagttctgag
            781 gattgtcaga goctgacgcg cactgtctgt gccggtggct gtgcccgctg
caaggggcca
            841 ctgcccactg actgctgcca tgagcagtgt gctgccggct gcacgggccc
caagcactct
            901 gactgcctgg cctgcctcca cttcaaccac agtggcatct gtgagctgca
ctgcccagcc
            961 ctggtcacct acaacacaga cacgtttgag tocatgccca atcccgaggg
ccggtataca
          1021 ttcggcgcca gctgtgtgac tgcctgtccc tacaactacc tttctacgga
cgtgggatcc
          1081 tgcaccctcg tctgccccct gcacaaccaa gaggtgacag cagaggatgg
aacacagcgg
          1141 tgtgagaagt gcagcaagcc ctgtgcccga gtgtgctatg gtctgggcat
ggagcacttg
          1201 cgagaggtga gggcagttac cagtgccaat atccaggagt ttgctggctg
caagaagatc
          1261 tttgggagcc tggcatttct gccggagagc tttgatgggg acccagcctc
caacactgcc
          1321 ccgctccagc cagagcagct ccaagtgttt gagactctgg aagagatcac
aggttaccta
          1381 tacatctcag catggccgga cagcctgcct gacctcagcg tcttccagaa
cctgcaagta
          1441 atccggggac gaattctgca caatggcgcc tactcgctga ccctgcaagg
gctgggcatc
          1501 agctggctgg ggctgcgctc actgagggaa ctgggcagtg gactggccct
catccaccat
          1561 aacacccacc tctgcttcqt gcacacggtg ccctgggacc agctctttcg
gaacccgcac
          1621 caagctctgc tccacactgc caaccggcca gaggacgagt gtgtgggcga
gggcctggcc
          1681 tgccaccagc tgtgcgcccg agggcactgc tggggtccag ggcccaccca
gtgtgtcaac
          1741 tgcagccagt tccttcgggg ccaggagtgc gtggaggaat gccgagtact
gcaggggctc
          1801 cccagggagt atgtgaatgc caggcactgt ttgccgtgcc accctgagtg
tcagccccag
          1861 aatggctcag tgacctgttt tggaccggag gctgaccagt gtgtggcctg
tgcccactat
          1921 aaqgaccctc ccttctgcgt ggcccgctgc cccagcggtg tgaaacctga
cctctcctac
          1981 atgcccatct ggaagtttcc agatgaggag ggcgcatgcc agccttgccc
catcaactgc
          2041 acccactcct gtgtggacct ggatgacaag ggctgccccg cegagoagag
agccagccct
          2101 ctgacgtcca tcgtctctgc ggtggttggc attctgctgg tcgtggtctt
gggggtggtc
          2161 tttgggatcc tcatcaagcg acggcagcag aagatccgga agtacacgat
gcggagactg
          2221 ctgcaggaaa cggagctggt ggagccgctg acacctagcg gagcgatgcc
caaccaggcg
          2281 cagatgcgga tcctgaaaga gacggagctg aggaaggtga aggtgcttgg
atctggcgct
          2341 tttggcacag tctacaaggg catctggatc cctgatgggg agaatgtgaa
aattccagtg
          2401 gccatcaaag tgttgaggga aaacacatcc cccaaagcca acaaagaaat
cttagacgaa
          2461 gcatacgtga tggctggtgt gggctcccca tatgtctccc gccttctggg
catctgcctg
          2521 acatccacgg tgcagctggt gacacagctt atgcoctatg gctgcctctt
agaccatgtc
          2581 cgggaaaacc gcggacgcct gggctcccag gacctgctga actggtgtat
gcagattgcc
          2641 aaggggatga gctacctgga ggatgtgcgg ctcgtacaca gggacttggc
cgctcggaac
          2701 gtgctggtca agagtcccaa ccatgtcaaa attacagact tcgggctggc
tcggctgctg
          2761 gacattgacg agacagagta ccatgcagat gggggcaagg tgcccatcaa
gtggatggcg
          2821 ctggagtcca ttctccgccg gcggttcacc caccagagtg atgtgtggag
ttatggtgtg
          2881 actgtgtggg agctgatgac ttttggggcc aaaccttacg atgggatccc
agcccgggag
          2941 atccctgacc tgctggaaaa gggggagcgg ctgccccagc cccccatctg
caccattgat
          3001 gtctacatga tcatggtcaa atgttggatg attgactctg aatgtcggcc
aagattccgg
          3061 gagttggtgt ctgaattctc ccgcatggcc agggaccccc agcgctttgt
ggtcatccag
          3121 aatgaggact tgggcccagc cagtcccttg gacagcacct tctaccgctc
actgctggag
          3181 gacgatgaca tgggggacct ggtggatgct gaggagtatc tggtacccca
gcagggcttc
          3241 ttctgtccag accctgcccc gggcgctggg ggcatggtcc accacaggca
ccgcagctca
          3301 tctaccagga gtggcggtgg ggacctgaca ctagggctgg agccctctga
agaggaggcc
          3361 cccaggtctc cactggcacc ctccgaaggg gctggctccg atgtatttga
tggtgacctg
          3421 ggaatggggg cagccaaggg gctgcaaagc ctccccacac atgaccccag
ccctctacag
          3481 cggtacagtg aggaccccac agtacccctg ccctctgaga ctgatggcta
cgttgccccc
          3541 ctgacctgca gcccccagcc tgaatatgtg aaccagccag atgttcggcc
ccagccccct
          3601 tcgccccgag agggccctct gcctgctgcc cgacctgctg gtgccactct
ggaaagggcc
          3661 aagactctct ccccagggaa gaatggggtc gtcaaagacg tttttgcctt
tgggggtgcc
          3721 gtggagaacc ccgagtactt gacaccccag ggaggagctg cccctcagcc
ccaccctcct
          3781 cctgccttca gcccagcctt cgacaacctc tattactggg accaggaccc
accagagcgg
          3841 ggggctccac ccagcacctt caaagggaca cctacggcag agaacccaga
gtacctgggt
          3901 ctggacgtgc cagtgtgaac cagaaggcca agtccgcaga agccctgatg
tgtcctcagg
          3961 gagcagggaa ggcctgactt ctgctggcat caagaggtgg gagggccctc
cgaccacttc
          4021 caggggaacc tgccatgcca ggaacctgtc ctaaggaacc ttccttcctg
cttgagttcc
          4081 cagatggctg gaaggggtcc agcctcgttg gaagaggaac agcactgggg
agtctttgtg
          4141 gattctgagg ccctgcccaa tgagactcta gggtccagtg gatgccacag
cccagcttgg
          4201 ccctttcctt ccagatcctg ggtactgaaa gccttaggga agctggcctg
agaggggaag
          4261 cggccctaag ggagtgtcta agaacaaaag cgacccattc agagactgtc
cctgaaacct
          4321 agtactgccc cccatgagga aggaacagca atggtgtcag tatccaggct
ttgtacagag
          4381 tgcttttctg tttagttttt actttttttg ttttgttttt ttaaagacga
aataaagacc
          4441 caggggagaa tgggtgttgt atggggaggc aagtgtgggg ggtccttctc
cacacccact
          4501 ttgtccattt gcaaatatat tttggaaaac
//
H.sapiens mRNA for SCP1 protein.
ACCESSION X95654
VERSION X95654.1 GI:1212982
SEQ ID NO 596
/trans1ation = “MEKQKPFALFVPPRSSSSQVSAVKPQTLGGDSTFFKSFNKCTED
DLEFPFAKTNLSKNGENIDSDPALQKVNFLPVLEQVGNSDCHYQEGLKDSDLENSEGL
SRVFSKLYKEAEKIKKWKVSTEAELRQKESKLQENRKIIEAQRKAIQELQFGNEKVSL
KLEEGIQENKDLIKENNATRHLCNLLKETCARSAEKTKKYEYEREETRQVYMDLNNNI
EKMITAHGELRVQAENSRLEMHFKLKEDYEKIQHLEQEYKKEINDKEKQVSLLLIQIT
EKENKMKDLTFLLEESRDKVNQLEEKTKLQSENLKQSIEKQHHLTKELEDIKVSLQRS
VSTQKALEEDLQIATKTICQLTEEKETQMEESNKARAAHSFVVTEFETTVCSLEELLR
TEQQRLEKNEDQLKILTMELQKKSSELEEMTKLTNNKEVELEELKKVLGEKETLLYEN
KQFEKIAEELKGTEQELIGLLQAREKEVHDLEIQLTAITTSEQYYSKEVKDLKTELEN
EKLKNTELTSHCNKLSLENKELTQETSDMTLELKNQQEDINNNKKQEERMLKQIENLQ
ETETQLRNELEYVREELKQKRDEVKCKLDKSEENCNNLRKQVENKNKYIEELQQENKA
LKKKGTAESKQLNVYEIKVNKLELELESAKQKFGEITDTYQKEIEDKKISEENLLEEV
EKAKVIADEAVKLQKEIDKRCQHKIAEMVALMEKHKHQYDKIIEERDSELGLYKSKEQ
EQSSLRASLEIELSNLKAELLSVKKQLEIEREEKEKLKREAKENTATLKEKKDKKTQT
FLLETPEIYWKLDSKAVPSQTVSRNFTSVDHGISKDKRDYLWTSAKNTLSTPLPKAYT
VKTPTKPKLQQRENLNIPIEESKKKRKMAFEFDINSDSSETTDLLSMVSEEETLKTLY
RNNNPPASHLCVKTPKKAPSSLTTPGPTLKFGAIRKMREDRWAVIAKMDRKKKLKEAE
KLFV”
SEQ ID NO 597
ORIGIN
                1 gccctcatag accgtttgtt gtagttcgcg tgggaacagc aacccacggt
ttcccgatag
              61 ttcttcaaag atatttacaa ccgtaacaga gaaaatggaa aagcaaaagc
cctttgcatt
            121 gttcgtacca ccgagatcaa gcagcagtca ggtgtctgcg gtgaaacctc
agaccctggg
            181 aggcgattcc actttcttca agagtttcaa caaatgtact gaagatgatt
tggagtttcc
            241 atttgcaaag actaatctct ccaaaaatgg ggaaaacatt gattcagatc
ctgctttaca
            301 aaaagttaat ttcttgcccg tgcttgagca ggttggtaat tctgactgtc
actatcagga
            361 aggactaaaa gactctgatt tggagaattc agagggattg agcagagtgt
tttcaaaact
            421 gtataaggag gctgaaaaga taaaaaaatg gaaagtaagt acagaagctg
aactgagaca
            481 gaaagaaagt aagttgcaag aaaacagaaa gataattgaa gcacagcgaa
aagccattca
            541 ggaactgcaa tttggaaatg aaaaagtaag tttgaaatta gaagaaggaa
tacaagaaaa
            601 taaagattta ataaaagaga ataatgccac aaggcattta tgtaatctac
tcaaagaaac
            661 ctgtgctaga tctgcagaaa agacaaagaa atatgaatat gaacgggaag
aaaccaggca
            721 agtttatatg gatctaaata ataacattga gaaaatgata acagctcatg
gggaacttcg
            781 tgtgcaagct gagaattcca gactggaaat gcattttaag ttaaaggaag
attatgaaaa
            841 aatccaacac cttgaacaag aatacaagaa ggaaataaat gacaaggaaa
agcaggtatc
            901 actactattg atccaaatca ctgagaaaga aaataaaatg aaagatttaa
catttctgct
            961 agaggaatcc agagataaag ttaatcaatt agaggaaaag acaaaattac
agagtgaaaa
          1021 cttaaaacaa tcaattgaga aacagcatca tttgactaaa gaactagaag
atattaaagt
          1081 gtcattacaa agaagtgtga gtactcaaaa ggctttagag gaagatttac
agatagcaac
          1141 aaaaacaatt tgtcagctaa ctgaagaaaa agaaactcaa atggaagaat
ctaataaagc
          1201 tagagctgct cattcgtttg tggttactga atttgaaact actgtctgca
gcttggaaga
          1261 attattgaga acagaacagc aaagattgga aaaaaatgaa gatcaattga
aaatacttac
          1321 catggagctt caaaagaaat caagtgagct ggaagagatg actaagctta
caaataacaa
          1381 agaagtagaa cttgaagaat tgaaaaaagt cttgggagaa aaggaaacac
ttttatatga
          1441 aaataaacaa tttgagaaga ttgctgaaga attaaaagga acagaacaag
aactaattgg
          1501 tcttctccaa gacagagaga aagaagtaca tgatttggaa atacagttaa
ctgccattac
          1561 cacaagtgaa cagtattatt caaaagaggt taaagatcta aaaactgagc
ttgaaaacga
          1621 gaagcttaag aatactgaat taacttcaca ctgcaacaag ctttcactag
aaaacaaaga
          1681 gctcacacag gaaacaagtg atatgaccct agaactcaag aatcagcaag
aagatattaa
          1741 taataacaaa aagcaagaag aaaggatgtt gaaacaaata gaaaatcttc
aagaaacaga
          1801 aacccaatta agaaatgaac tagaatatgt gagagaagag ctaaaacaga
aaagagatga
          1861 agttaaatgt aaattggaca agagtgaaga aaattgtaac aatttaagga
aacaagttga
          1921 aaataaaaac aagtatattg aagaacttca gcaggagaat aaggccttga
aaaaaaaagg
          1981 tacagcagaa agcaagcaac tgaatgttta tgagataaag gtcaataaat
tagagttaga
          2041 actagaaagt gccaaacaga aatttggaga aatcacagac acctatcaga
aagaaattga
          2101 ggacaaaaag atatcagaag aaaatctttt ggaagaggtt gagaaagcaa
aagtaatagc
          2161 tgatgaagca gtaaaattac agaaagaaat tgataagcga tgtcaacata
aaatagctga
          2221 aatggtagca cttatggaaa aacataagca ccaatatgat aagatcattg
aagaaagaga
          2281 ctcagaatta ggactttata agagcaaaga acaagaacag tcatcactga
gagcatcttt
          2341 ggagattgaa ctatccaatc tcaaagctga acttttgtct gttaagaagc
aacttgaaat
          2401 agaaagagaa gagaaggaaa aactcaaaag agaggcaaaa gaaaacacag
ctactcttaa
          2461 agaaaaaaaa gacaagaaaa cacaaacatt tttattggaa acacctgaaa
tttattggaa
          2521 attggattct aaagcagttc cttcacaaac tgtatctcga aatttcacat
cagttgatca
          2581 tggcatatcc aaagataaaa gagactatct gtggacatct gccaaaaata
ctttatctac
          2641 accattgcca aaggcatata cagtgaagac accaacaaaa ccaaaactac
agcaaagaga
          2701 aaacttgaat ataccoattg aagaaagtaa aaaaaagaga aaaatggcct
ttgaatttga
          2761 tattaattca gatagttcag aaactactga tcttttgagc atggtttcag
aagaagagac
          2821 attgaaaaca ctgtatagga acaataatcc accagcttct catctttgtg
tcaaaacacc
          2881 aaaaaaggcc ccttcatctc taacaacccc tggacctaca ctgaagtttg
gagctataag
          2941 aaaaatgcgg gaggaccgtt gggctgtaat tgctaaaatg gatagaaaaa
aaaaactaaa
          3001 agaagctgaa aagttatttg tttaatttca gagaatcagt gtagttaagg
agcctaataa
          3061 cgtgaaactt atagttaata ttttgttctt atttgccaga gccacatttt
atctggaagt
          3121 tgagacttaa aaaatacttg catgaatgat ttgtgtttct ttatattttt
agcctaaatg
          3181 ttaactacat attgtctgga aacctgtcat tgtattcaga taattagatg
attatatatt
          3241 gttgttactt tttcttgtat tcatgaaaac tgtttttact aagttttcaa
atttgtaaag
          3301 ttagcctttg aatgctagga atgcattatt gagggtcatt ctttattctt
tactattaaa
          3361 atattttgga tgcaaaaaaa aaaaaaaaaa aaa
//
Homo sapiens synovial sarcoma, X breakpoint 4 (SSX4), mRNA.
ACCESSION NM_005636
VERSION NM_005636.1 GI:5032122
SEQ ID NO 598
/translation = “MNGDDAFARRPRDDAQISEKLRKAFDDIAKYFSKKEWEKMKSSEKIVY
VYMKLNYEVMTKLGFKVTLPPFMRSKRAADFHGNDFGNDRNHRNQVERPQMTFG
SLQRIFPKIMPKKPAEEENGLKEVPEASGPQNDGKQLCPPGNPSTLEKINKTSGPKRG
KHAWTHRLRERKQLVVYEEISDPEEDDE”
SEQ ID NO 599
ORIGIN
                1 atgaacggag acgacgcctt tgcaaggaga cccagggatg atgctcaaat
atcagagaag
              61 ttacgaaagg ccttcgatga tattgccaaa tacttctcta agaaagagtg
ggaaaagatg
            121 aaatcctcgg agaaaatcgt ctatgtgtat atgaagctaa actatgaggt
catgactaaa
            181 ctaggtttca aggtcaccct cccacctttc atgcgtagta aacgggctgc
agacttccac
            241 gggaatgatt ttggtaacga tcgaaaccac aggaatcagg ttgaacgtcc
tcagatgact
            301 ttcggcagcc tccagagaat cttcccgaag atcatgccca agaagccagc
agaggaagaa
            361 aatggtttga aggaagtgcc agaggcatct ggcccacaaa atgatgggaa
acagctgtgc
            421 cccccgggaa atccaagtac cttggagaag attaacaaga catctggacc
caaaaggggg
            481 aaacatgcct ggacccacag actgcgtgag agaaagcagc tggtggttta
tgaagagatc
            541 agcgaccctg aggaagatga cgagtaactc ccctcg
```

All patents and publications mentioned in the specification are indicative of the levels of those skilled in the art to which the invention pertains. The entire contents of all patents and publications discussed herein are incorporated by reference in their entirety to the same extent as if each individual publication was specifically and individually indicated to be incorporated by reference in its entirety.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. An isolated epitope, comprising a component selected from the group consisting of:
(i) a polypeptide having the sequence as disclosed in TABLE 1;
(ii) an epitope cluster comprising the polypeptide of (i);
(iii) a polypeptide having substantial similarity to (i) or (ii);
(iv) a polypeptide having functional similarity to any of (i) through (iii); and
(v) a nucleic acid encoding the polypeptide of any of (i) through (iv).

2. The epitope of claim 1, wherein the epitope is immunologically active.

3. The epitope of claim 1, wherein the polypeptide is less than about 30 amino acids in length.

4. The epitope of claim 1, wherein the polypeptide is 8 to 10 amino acids in length.

5. The epitope of claim 1, wherein the substantial or functional similarity comprises addition of at least one amino acid.

6. The epitope of claim 5, wherein the at least one additional amino acid is at an N-terminus of the polypeptide.

7. The epitope of claim 1, wherein the substantial or functional similarity comprises a substitution of at least one amino acid.

8. The epitope of claim 1, the polypeptide having affinity to an HLA-A2 molecule.

9. The epitope of claim 8, wherein the affinity is determined by an assay of binding.

10. The epitope of claim 8, wherein the affinity is determined by an assay of restriction of epitope recognition.

11. The epitope of claim 8, wherein the affinity is determined by a prediction algorithm.

12. The epitope of claim 1, the polypeptide having affinity to an HLA-B7 or HLA-B51 molecule.

13. The epitope of claim 1, wherein the polypeptide is a housekeeping epitope.

14. The epitope of claim 1, wherein the polypeptide corresponds to an epitope displayed on a tumor cell.

15. The epitope of claim 1, wherein the polypeptide corresponds to an epitope displayed on a neovasculature cell.

16. The epitope of claim 1, wherein the polypeptide is an immune epitope.

17. The epitope of claim 1 wherein the epitope is a nucleic acid.

18. A pharmaceutical composition comprising the polypeptide of claim 1 and a pharmaceutically acceptable adjuvant, carrier, diluent, or excipient.

19. The composition of claim 18, where the adjuvant is a polynucleotide.

20. The composition of claim 19 wherein the polynucleotide comprises a dinucleotide.

21. The composition of claim 20 wherein the dinucleotide is CpG.

22. The composition of claim 18, wherein the adjuvant is encoded by a polynucleotide.

23. The composition of claim 18 wherein the adjuvant is a cytokine.

24. The composition of claim 23 wherein the cytokine is GM-CSF.

25. The composition of claim 18 further comprising a professional antigen-presenting cell (pAPC).

26. The composition of claim 25, wherein the pAPC is a dendritic cell.

27. The composition of claim 18, further comprising a second epitope.

28. The composition of claim 27, wherein the second epitope is a polypeptide.

29. The composition of claim 27, wherein the second epitope is a nucleic acid.

30. The composition of claim 27, wherein the second epitope is a housekeeping epitope.

31. The composition of claim 27, wherein the second epitope is an immune epitope.

32. A pharmaceutical composition comprising the nucleic acid of claim 1 and a pharmaceutically acceptable adjuvant, carrier, diluent, or excipient.

33. A recombinant construct comprising the nucleic acid of claim 1.

34. The construct of claim 33, further comprising a plasmid, a viral vector, or an artificial chromosome.

35. The construct of claim 33, further comprising a sequence encoding at least one feature selected from the group consisting of a second epitope, an IRES, an ISS, an NIS, and ubiquitin.

36. A purified antibody that specifically binds to the epitope of claim 1.

37. A purified antibody that specifically binds to a peptide-MHC protein complex comprising the epitope of claim 1.

38. The antibody of claim 36 or claim 37, wherein the antibody is a monoclonal antibody.

39. A multimeric MHC-peptide complex comprising the epitope of claim 1.

40. An isolated T cell expressing a T cell receptor specific for an MHC-peptide complex, the complex comprising the epitope of claim 1.

41. The T cell of claim 40, produced by an in vitro immunization.

42. The T cell of claim 40, isolated from an immunized animal.

43. A T cell clone comprising the T cell of claim 40.

44. A polyclonal population of T cells comprising the T cell of claim 40.

45. A pharmaceutical composition comprising the T cell of claim 40 and a pharmaceutically acceptable adjuvant, carrier, diluent, or excipient.

46. An isolated protein molecule comprising the binding domain of a T cell receptor specific for an MHC-peptide complex, the complex comprising the epitope of claim 1.

47. The protein of claim 46, wherein the protein is multivalent.

48. An isolated nucleic acid encoding the protein of claim 46.

49. A recombinant construct comprising the nucleic acid of claim 48.

50. A host cell expressing the recombinant construct, the construct comprising the nucleic acid of claim 1, or the construct encoding a protein molecule comprising the binding domain of a T cell receptor specific for an MHC-peptide complex.

51. The host cell of claim 50, wherein the host cell is a dendritic cell, macrophage, tumor cell, or tumor-derived cell.

52. The host cell of claim 50, wherein the host cell is a bacterium, fungus, or protozoan.

53. A pharmaceutical composition comprising the host cell of claim 50 and a pharmaceutically acceptable adjuvant, carrier, diluent, or excipient.

54. A vaccine or immunotherapeutic composition comprising at least one component selected from the group consisting of the epitope of claim 1; the composition of claim 18, 32, or 45, the construct of claim 33; the T cell of claim 40, a host cell expressing a recombinant construct comprising a nucleic acid encoding a T cell receptor binding domain specific for an MHC-peptide complex and a composition comprising the same, and a host cell expressing a recombinant construct comprising the nucleic acid of claim 1 and a composition comprising the same.

55. A method of treating an animal, comprising:
administering to an animal the vaccine or immunotherapeutic composition of claim 54.

56. The method of claim 55, wherein the administering step comprises a mode of delivery selected from the group consisting of transdermal, intranodal, perinodal, oral, intravenous, intradermal, intramuscular, intraperitoneal, mucosal, aerosol inhalation, and instillation.

57. The method of claim 55, further comprising a step of assaying to determine a characteristic indicative of a state of a target cell or target cells.

58. The method of claim 57, comprising a first assaying step and a second assaying step, wherein the first assaying step precedes the administering step, and wherein the second assaying step follows the administering step.

59. The method of claim 58, further comprising a step of comparing the characteristic determined in the first assaying step with the characteristic determined in the second assaying step to obtain a result.

60. The method of claim 59, wherein the result is selected from the group consisting of: evidence of an immune response, a diminution in number of target cells, a loss of mass or size of a tumor comprising target cells, a decrease in number or concentration of an intracellular parasite infecting target cells.

61. A method of evaluating immunogenicity of a vaccine or immunotherapeutic composition, comprising:
administering to an animal the vaccine or immunotherapeutic composition of claim 54; and
evaluating immunogenicity based on a characteristic of the animal.

62. The method of claim 61, wherein the animal is HLA-transgenic.

63. A method of evaluating immunogenicity, comprising:
in vitro stimulation of a T cell with the vaccine or immunotherapeutic composition of claim 54; and
evaluating immunogenicity based on a characteristic of the T cell.

64. The method of claim 63, wherein the stimulation is a primary stimulation.

65. A method of making a passive/adoptive immunotherapeutic, comprising:
combining the T cell of claim 40, or a host cell expressing a recombinant construct comprising a nucleic acid encoding a T cell receptor binding domain specific for an MHC-peptide complex, or a host cell expressing a recombinant construct comprising the nucleic acid of claim 1 with a pharmaceutically acceptable adjuvant, carrier, diluent, or excipient.

66. A method of determining specific T cell frequency comprising the step of contacting T cells with a MHC-peptide complex comprising the epitope of claim 1.

67. The method of claim 66, wherein the contacting step comprises at least one feature selected from the group consisting of immunization, restimulation, detection, and enumeration.

68. The method of claim 66, further comprising ELISPOT analysis, limiting dilution analysis, flow cytometry, in situ hybridization, the polymerase chain reaction or any combination thereof.

69. A method of evaluating immunologic response, comprising the method of claim 66 carried out prior to and subsequent to an immunization step.

70. A method of evaluating immunologic response, comprising:
determining frequency, cytokine production, or cytolytic activity of T cells, prior to and subsequent to a step of stimulation with MHC-peptide complexes comprising the epitope of claim 1.

71. A method of diagnosing a disease comprising:
contacting a subject tissue with at least one component selected from the group consisting of the T cell of claim 40, the host cell of claim 50, the antibody of claim 36, and the protein of claim 46; and
diagnosing the disease based on a characteristic of the tissue or of the component.

72. The method of claim 71, wherein the contacting step takes place in vivo.

73. The method of claim 71, wherein the contacting step takes place in vitro.

74. A method of making a vaccine, comprising:
combining at least one component selected from the group consisting of the epitope of claim 1; the composition of claim 18, 32, 45, or 53; the construct of claim 33; the T cell of claim 40, and the host cell of claim 50, with a pharmaceutically acceptable adjuvant, carrier, diluent, or excipient.

75. A computer readable medium having recorded thereon the sequence of any one of SEQ ID NOS: 1-602, in a machine having a hardware or software that calculates the physical, biochemical, immunologic, or molecular genetic properties of a molecule embodying said sequence.

76. A method of treating an animal comprising combining the method of claim 55 combined with at least one mode of treatment selected from the group of radiation therapy, chemotherapy, biochemotherapy, and surgery.

77. An isolated polypeptide comprising an epitope cluster from a target-associated antigen having the sequence as disclosed in Tables 25-44, wherein the amino acid sequence consists of not more than about 80% of the amino acid sequence of the antigen.

78. A vaccine or immunotherapeutic product comprising the polypeptide of claim 78.

79. An isolated polynucleotide encoding the polypeptide of claim 78.

80. A vaccine or immunotherapeutic product comprising the polynucleotide of claim 80.

81. The polynucleotide of claim 79 or 80, wherein the polynucleotide is DNA.

82. The polynucleotide of claim 79 or 80, wherein the polynucleotide is RNA.
